SEQUENCE LISTING (130 sequences)

SEQ ID NO: 1 - protein, 319 aa, Aspergillus oryzae
MASPTVKLNSGHDMPLVGFGLWKVNNETCADQVYEAIKAGYRLFDGACDYGNEVECGQGVARAIKEGIVKREELFIVSKLWNSFHEGDRVEPICRKQLADWGVDYFDLYIVHFPVALKYVDPAVRYPPGWNSESGKIEFSNATIQETWTAMESLVDKKLARSIGVSNFSAQLLMDLLRYARVRPATLQIEHHPYLTQPRLVEYAQKEGIAVTAYSSFGPLSFLELEVKNAVDTPPLFEHNTIKSLAEKYGKTPAQVLLRWATQRGIAVIPKSNNPTRLSQNLEVTGWDLEKSELEAISSLDKGLRFNDPIGYGMYVPIF

SEQ ID NO: 2 - protein, 317 aa, Candida parapsilosis
MSIKLNSGHEMPIVGFGCWKVTNETAADQIYNAIKVGYRLFDGAQDYGNEKEVGEGINRAIDEGLVSRDELFVVSKLWNNYHDPKNVETALNKTLSDLNLEYLDLFLIHFPIAFKFVPIEEKYPPGFYCGDGDKFHYENVPLLDTWRALESLVQKGKIRSIGISNFNGGLIYDLVRGAKIKPAVLQIEHHPYLQQPRLIEFVQSQGIAITGYSSFGPQSFLELESKKALDTPTLFDHETIKSIASKHKKSSAQVLLRWATQRGIAVIPKSNNPDRLAQNLNVSDFELSKEDLEAINKLDKGLRFNDPWDWDHIPIFV

SEQ ID NO: 3 - protein, 323 aa, Candida shehatae
MSPSPIPAFKLNNGLEMPSIGFGCWKLGKSTAADQVYNAIKAGYRLFDGAEDYGNEQEVGEGVKRAIDEGIVTREEIFLTSKLWNNYHDPKNVETALNKTLKDLKVDYVDLFLIHFPIAFKFVPIEEKYPPGFYCGDGDNFVYEDVPILETWKALEKLVKAGKIRSIGVSNFPGALLLDLFRGATIKPAVLQVEHHPYLQQPKLIEYAQKVGITVTAYSSFGPQSFVEMNQGRALNTPTLFEHDVIKAIAAKHNKVPAEVLLRWSAQRGIAVIPKSNLPERLVQNRSFNDFELTKEDFEEISKLDINLRFNDPWDWDNIPIFV

SEQ ID NO: 4 - protein, 324 aa, Candida tropicalis
MSTTVNTPTIKLNSGYEMPLVGFGCWKVTNATAADQIYNAIKTGYRLFDGAEDYGNEKEVGEGINRAIKDGLVKREELFITSKLWNNFHDPKNVETALNKTLSDLNLDYVDLFLIHFPIAFKFVPIEEKYPPGFYCGDGDNFHYEDVPLLDTWKALEKLVEAGKIKSIGISNFTGALIYDLIRGATIKPAVLQIEHHPYLQQPKLIEYVQKAGIAITGYSSFGPQSFLELESKRALNTPTLFEHETIKSIADKHGKSPAQVLLRWATQRNIAVIPKSNNPERLAQNLSVVDFDLTKDDLDNIAKLDIGLRFNDPWDWDNIPIFV

SEQ ID NO: 5 - protein, 329 aa, Kluyveromyces lactis
MTYLAETVTLNNGEKMPLVGLGCWKMPNDVCADQIYEAIKIGYRLFDGAQDYANEKEVGQGVNRAIKEGLVKREDLVVVSKLWNSFHHPDNVPRALERTLSDLQLDYVDIFYIHFPLAFKPVPFDEKYPPGFYTGKEDEAKGHIEEEQVPLLDTWRALEKLVDQGKIKSLGISNFSGALIQDLLRGARIKPVALQIEHHPYLTQERLIKYVKNAGIQVVAYSSFGPVSFLELENKKALNTPTLFEHDTIKSIASKHKVTPQQVLLRWATQNGIAIIPKSSKKERLLDNLRINDALTLTDDELKQISGLNQNIRFNDPWEWLDNEFPTFI

SEQ ID NO: 6 - protein, 322 aa, Neurospora crassa
MVPAIKLNSGFDMPQVGFGLWKVDGSIASDVVYNAIKAGYRLFDGACDYGNEVECGQGVARAIKEGIVKREELFIVSKLWNTFHDGDRVEPIVRKQLADWGLEYFDLYLIHFPVALEYVDPSVRYPPGWHFDGKSEIRPSKATIQETWTAMESLVEKGLSKSIGVSNFQAQLLYDLLRYAKVRPATLQIEHHPYLVQQNLLNLAKAEGIAVTAYSSFGPASFREFNMEHAQKLQPLLEDPTIKAIGDKYNKDPAQVLLRWATQRGLAIIPKSSREATMKSNLNSLDFDLSEEDIKTISGFDRGIRFNQPTNYFSAENLWIFG

SEQ ID NO: 7 - protein, 317 aa, Pichia guilliermondii
MSIKLNSGYDMPSVGFGCWKVDNATCADTIYNAIKVGYRLFDGAEDYGNEKEVGDGINRALDEGLVARDELFVVSKLWNSFHDPKNVEKALDKTLSDLKVDYLDLFLIHFPIAFKFVPFEEKYPPGFYCGDGDKFHYEDVPLIDTWRALEKLVEKGKIRSIGISNFSGALIQDLLRSAKIKPAVLQIEHHPYLQQPRLVEYVQSQGIAITAYSSFGPQSFVELDHPRVKDVKPLFEHDVIKSVAGKVKKTPAQVLLRWATQRGLAVIPKSNNPDRLLSNLKVNDFDLSQEDFQEISKLDIELRFNNPWDWDKIPTFI

SEQ ID NO: 8 - protein, 318 aa, Pichia stipitis
MPSIKLNSGYDMPAVGFGCWKVDVDTCSEQIYRAIKTGYRLFDGAEDYANEKLVGAGVKKAIDEGIVKREDLFLTSKLWNNYHHPDNVEKALNRTLSDLQVDYVDLFLIHFPVTFKFVPLEEKYPPGFYCGKGDNFDYEDVPILETWKALEKLVKAGKIRSIGVSNFPGALLLDLLRGATIKPSVLQVEHHPYLQQPRLIEFAQSRGIAVTAYSSFGPQSFVELNQGRALNTSPLFENETIKAIAAKHGKSPAQVLLRWSSQRGIAIIPKSNTVPRLLENKDVNSFDLDEQDFADIAKLDINLRFNDPWDWDKIPIFV

SEQ ID NO: 9 - protein, 319 aa, Aspergillus flavus; identical to SEQ ID NO: 1

SEQ ID NO: 10 - protein, 372 aa, Magnaporthe oryzae
MSATNGSAAAAPSKKNIGVFTNPKHDLWINEAEPSLESVQKGSDELKEGQVTIAIRSTGICGSDVHFWHHGCIGPMIVREDHILGHESAGEIIAVHPSVTSLKVGDRVAVEPQVICYECEPCLTGRYNGCEKVDFLSTPPVPGLLRRYVNHPAVWCHKIGDMSWEDGAMLEPLSVALAGIQRAGITLGDPVLVCGAGPIGLITLLCAKAAGACPLVITDIDDGRLKFAKELVPDVITFKVEGRPTAEDAAKSIVEAFGGVEPTLAIECTGVESSIASAIWAVKFGGKVFVIGVGRNEISLPFMRASVREVDLQFQYRYCNTWPRAIRLIQNKVIDLTKLVTHRFPLEDALKAFETAADPKTGAIKVQIQSLE

SEQ ID NO: 11 - protein, 327 aa, Zygosaccharomyces rouxii
MASVVALNNGNKMPLVGLGCWKIPNETCSQQIYDAISVGYRVFDGAQDYGNEKEVGEGVRRAIKDGLVKREELFVVSKLWNSFHHPKNVKLALKRTLSDMGLDYLDLFYIHFPIALKPVSFEEKYPPGLYTGEADAKAGVLSEEPVPILDTYRALEECVEEGLIKSIGVSNFSGSIMLDLLRGARIPPAALQIELHPYLTQERYVKWVQSKGIQVVAYSSFGPQSFVDIGSEVAKATPPLFEHDVVKKIAAKHNVSTSQVLLRWATQQKVAVIPKSSKKERLRQNLLVDQEVTLTGDEIKEISGLNKNLRFNDPFTWSEKTPFPIFD

SEQ ID NO: 12 - protein, 320 aa, Talaromyces stipitatus
MSSPTVKLNSGYDMPLVGFGLWKVNNDTCADQVYAAIKAGYRLFDGACDYGNEKEVGQGIARAIKDGLVKREELFIVSKLWNTFHDGDKVEPIARKQLDDLGLDYFDLYLIHFPVALKWVDPAERYPPGWTAPDGKVEFSKATIQETWQAMESLVDKKLSRSIGISNFSVQLIMDLLRHARIRPATLQIEHHPYLQQKELIKYVQSEGIVITAYSSFGPLSFIELDMSSAHNTPKLFDHDVIKSTSQKHGKTPAQILLRWATQRNIAVIPKSNDPTRLSQNLDVTGWSLEQSDIDAINGLDLGLRFNDPLNYGIYIPIFA

SEQ ID NO: 13 - protein, 321 aa, Podospora anserina
MAPVIKLNSGYDMPQVGFGLWKVDNAIAADVVYNAIKAGYRLFDGACDYGNEVECGKGVARAISEGIVKREDLFIVSKLWNTFHDGERVQPIVKKQLADWGVDYFDLYLIHFPVALEYVDPSVRYPPGWHYEGDEIRPSKATIQETWTAMESLVDAGLARSIGISNFQSQLIYDLLRYAKIRPATLQIEHHPYLTQEELLKLAKREGITVTAYSSFGPASFLEFNMQHAVKLQPLMEDDTIKAIAAKYNRPASQVLLRWATQRGLAVIPKSSRQETMVSNLQNTDFDLSEEDIATISGFNRGIRFNQPSNYFPTELLWIFG

SEQ ID NO: 14 - protein, 319 aa, Pichia pastoris
MATLLKLNNGLKLPQVGLGVWKIPNELTAETVYNAIKQGYRLFDGAEDYGNEKEVGQGVRRAIDEGLVKREDLFIVSKLWNNYHHPDNVGKALDRTLSDLGLDYLDLFYIHFPIAFKFVPLEEKYPPAFYCGDGNNFHYEDVPLLDTYRALERLVDAGRIKSLGVSNFNGALLQDLLRGARIKPVALQIEHHPYLVQQKLIEYAQSEDIVVVAYSSFGPQSFLELKVNKALTAVSLFEHDVIKKIAQAHNRSAGEVLLRWATQRGLAIIPKSSKPERLSSNLHINSFDLTKEDLETISSLDLGLRFNDPWDWDKIPIFA

SEQ ID NO: 15 - protein, 379 aa, Phaeosphaeria nodorum
MVAGRFCRTSINTVRSFTTAVVPRSSFFPPVRTCISRTKAPSFRPTYSNRNFFATMAVNTPYITLNDGNKMPQVGFGLWKVDNATCADTVYNAIKTGYRLFDGACDYGNEVECGQGVARAIKEGLVKREDLFIVSKLWQTFHDYEQVEPITKKQLKDWGIDYFDLYLIHFPVALKYVSPETRYPPGWFSDEANSKVIHSKARLEDTWRAFEDIKSKGLTKSIGVSNYSGALLLDLFTYAKVKPATLQIEHHPYYVQPYLIKLAEEHDIKVTAYSSFGPQSFIECDMKIAADTPLLFDHPVIKKIAEKHSKTPAQILLRWSTQRGLSVIPKSNSQNRLQQNLDVTGFDMSESEIAEISDLDKNLKFNAPTNYGIPCYVFA

SEQ ID NO: 16 - protein, 319 aa, Penicillium chrysogenum
MVAPTVKLSSGYEMPLVGFGLWKVNNDTCADQVYHAIKAGYRLFDGACDYGNEVEAGQGVARAIKEGIVKREELFIVSKLWNSFHEADKVEPIARKQLADWGVDYFDLYIVHFPIALKYLDPSVRYPPSWTTAEGKIEFANAPIHETWGAMETLVDKKLARSIGVSNFSAQLLMDLLRYARVRPATLQIEHHPYLTQTRLVDYAQKEGITVTAYSSFGPLSFLELDLKHAKDTPLLFEHATITSIAEKHGRTPAQVLLRWSTQRNVAVIPKSNNPTRLAQNLTVTDFDLEASELEAISALDKGLRFNDPIAVSLVCVEY

SEQ ID NO: 17 - protein, 399 aa, Meyerozyma guilliermondii
MTKMDHKIVKTSYDGDAVSVEWDGGASAKFDNIWLRDNCHCSECYYDATKQRLLNSCSIPDDIAPIKVDSSPTKLKIVWNHEEHQSEYECRWLVIHSYNPRQIPVTEKVSGEREILAREYWTVKDMEGRLPSVDFKTVMASTDENEEPIKDWCLKIWKHGFCFIDNVPVDPQETEKLCEKLMYIRPTHYGGFWDFTSDLSKNDTAYTNIDISSHTDGTYWSDTPGLQLFHLLMHEGTGGTTSLVDAFHCAEILKKEHPESFELLTRIPVPAHSAGEEKVCIQPDIPQPIFKLDTNGELIQVRWNQSDRSTMDSWENPSEVVKFYRAIKQWHKIISDPANELFYQLRPGQCLIFDNWRCFHSRTEFTGKRRMCGAYINRDDFVSRLKLLNIGRQPVLDAI

SEQ ID NO: 18 - protein, 319 aa, Aspergillus niger
MASPTVKLNSGYDMPLVGFGLWKVNNDTCADQIYHAIKEGYRLFDGACDYGNEVEAGQGIARAIKDGLVKREELFIVSKLWNSFHDGDRVEPICRKQLADWGIDYFDLYIVHFPISLKYVDPAVRYPPGWKSEKDELEFGNATIQETWTAMESLVDKKLARSIGISNFSAQLVMDLLRYARIRPATLQIEHHPYLTQTRLVEYAQKEGLTVTAYSSFGPLSFLELSVQNAVDSPPLFEHQLVKSIAEKHGRTPAQVLLRWATQRGIAVIPKSNNPQRLKQNLDVTGWNLEEEEIKAISGLDRGLRFNDPLGYGLYAPIF

SEQ ID NO: 19 - protein, 319 aa, Aspergillus nidulans
MSPPTVKLNSGYDMPLVGFGLWKVNNDTCADQVYEAIKAGYRLFDGACDYGNEVEAGQGVARAIKEGIVKRSDLFIVSKLWNSFHDGERVEPIARKQLSDWGIDYFDLYIVHFPVSLKYVDPEVRYPPGWENAEGKVELGKATIQETWTAMESLVDKGLARSIGISNFSAQLLLDLLRYARIRPATLQIEHHPYLTQERLVTFAQREGIAVTAYSSFGPLSFLELSVKQAEGAPPLFEHPVIKDIAEKHGKTPAQVLLRWATQRGIAVIPKSNNPARLLQNLDVVGFDLEDGELKAISDLDKGLRFNDPPNYGLPITIF

SEQ ID NO: 20 - protein, 318 aa, Pichia stipitis
MPSIKLNSGYDMPAVGFGCWKVDVDTCSEQIYRAIKTGYRLFDGAEDYANEKLVGAGVKKAIDEGIVKREDLFLTSKLWNNYHHPDNVEKALNRTLSDLQVDYVDLFLIHFPVTFKFVPLEEKYPPGFYCGKGDNFDYEDVPILETWKALEKLVKAGKIRSIGVSNFPGALLLDLLRGATIKPSVLQVEHHPYLQQPRLIEFAQSRGIAVTAYSSFGPQSFVELNQGRALNTSPLFENETIKAIAAKHGKSPAQVLLRWSSQRGIAIIPRSNTVPRLLENKDVNSFDLDEQDFADIAKLDINLRFNDPWDWDKIPIFV

SEQ ID NO: 21 - DNA, 801 bp, Aspergillus niger
atgcctatttccattccatctgcatcctcagttcatgatctgttttctcttaagggcaaggttgttgtgataacaggtgcatctggaccaagagggatgggtattgaagctgctagaggttgtgccgaaatgggtgctaacatcgctctaacctattcatctcgtcctcaaggaggggagaagaacgctgaagaactgagaaatacttacggcgtcaaggctaaagcatatcagtgcaatgtgggcgattggaacagtgtaaagaagttggttgatgatgtcttagctgagtttggacagattgatgctttcatagctaacgccggtaaaacagctagttctggtatcttagacggctcagtggaagattgggaagaggtaatacaaactgacttaactgggacattccactgtgcaaaagccgtcggccctcatttcaagcaaagaggtacaggcagtttcatcatcacttcatcaatgtcaggtcacatagctaacttcccacaagaacaaacctcctacaatgtagcaaaggccggctgtatccacatggccagatcattagccaatgagtggagagattttgctagggttaactctatctctcctggttacattgatactggattgagtgatttcgttgacaaaaagacacaagatttgtggatgtcaatgattccaatgggtagaaacggagatgcaaaagaactaaaaggggcctacgtataccttgcatccgatgcatctacatacacaacaggagctgatttggttattgatggaggctataccgtcagataa

SEQ ID NO: 22 - protein, 358 aa, Aspergillus oryzae
MGAPPKTAQNLSFVLEGIHKVKFEDRPIPQLRDAHDVLVDVRFTGICGSDVHYWEHGSIGQFVVKDPMVLGHESSGVISKVGSAVTTLKVGDHVAMEPGIPCRRCEPCKEGKYNLCEKMAFAATPPYDGTLAKYYVLPEDFCYKLPENINLQEAAVMEPLSVAVHIVKQANVAPGQSVVVFGAGPVGLLCCAVARAFGSPKVIAVDIQKGRLEFAKKYAATAIFEPSKVSALENAERIVNENDLGRGADIVIDASGAEPSVHTGIHVLRPGGTYVQGGMGRNEITFPIMAACTKELNVRGSFRYGSGDYKLAVNLVASGKVSVKELITGVVSFEDAEQAFHEVKAGKGIKTLIAGVDV

SEQ ID NO: 23 - protein, 410 aa, Aspergillus nidulans
MSSQTPTAQNLSFVLEGIHRVKFEDRPIPKLKSPHDVIVNVKYTGICGSDVHYWDHGAIGQFVVKEPMVLGHESSGIVTQIGSAVTSLKVGDHVAMEPGIPCRRCEPCKAGKYNLCEKMAFAATPPYDGTLAKYYTLPEDFCYKLPESISLPEGALMEPLGVAVHIVRQANVTPGQTVVVFGAGPVGLLCCAVAKAFGAIRIIAVDIQKPRLDFAKKFAATATFEPSKAPATENATRMIAENDLGRGADVAIDASGVEPSVHTGIHVLRPGGTYVQGGMGRSEMNFPIMAACTKELNIKGSFRYGSGDYKLAVQLVASGQINVKELITGIVKFEDAEQAFKDVKTGKGIKTLIAGPGAAYKLAVQLVASGQINVKELITGIVKFEDAEQAFKDVKTGKGIKTLIAGPGAA

SEQ ID NO: 24 - protein, 360 aa, Candida albicans
MTNPSLVLNKIDDISFEDYESPEITSPRDVIVEVKKTGICGSDIHYYAHGSIGPFVLRKPMVLGHESAGVVVAVGDDVTNLKVGDKVAIEPGVPSRYSDEYKSGNYHLCPHMAFAATPPVNPDEPNPPGTLCKYYKAPADFLFKLPDHVSLELGAMVEPLTVGVHACKLANLKFGENVVVFGAGPVGLLTAAVAKTIGAKNIMVVDIFDNKLQMAKDMGAATHTFNSKTGDDLVKAFDGIEPSVVLECSGAKQCIYTGVKILKAGGRFVQVGNAGGDVNFPIADFSTRELTLYGSFRYGYGDYQTSIDILDKNYINGKENAPINFELLITHRFKFKDAIKAYDLVRGGNGAVKCLIDGPE

SEQ ID NO: 25 - protein, 364 aa, Candida dubliniensis
MTPNPSLVLNKIDDISFEEYESPEITSPRDVIVEVKKTGICGSDIHYYAHGKIGPFVLRKPMVLGHESAGVVVAVGDDVKNLKVGDNVAIEPGVPSRYSDEYKSGNYHLCPHMAFAATPPVNPDEPNPPGTLCKYYKAPADFLFKLPDHVSLELGAMVEPLTVGVHACKLANLKFGENVVVFGAGPVGLLTAAVAKTIGAKNIMVVDIFDNKLKMAKDMGVATHTFNSKTGGDDRDLVKHFDGIEPSVVLECSGAKQCIYTGVKVLKAGGRFVQVGNAGGDVNFPIADFSTRELALYGSFRYGYGDYQTSIDILDKNYINGKDNAPINFELLITHRFKFKDAIKAYDLVRGGNGAVKCLIDGPE

SEQ ID NO: 26 - protein, 363 aa, Hypocrea jecorina
MATQTINKDAISNLSFVLNKPGDVTFEERPKPTITDPNDVLVAVNYTGICGSDVHYWVHGAIGHFVVKDPMVLGHESAGTVVEVGPAVKSLKPGDRVALEPGYPCRRCSFCRAGKYNLCPDMVFAATPPYHGTLTGLWAAPADFCYKLPDGVSLQEGALIEPLAVAVHIVKQARVQPGQSVVVMGAGPVGLLCAAVAKAYGASTIVSVDIVQSKLDFARGFCSTHTYVSQRISAEDNAKAIKELAGLPGGADVVIDASGAEPSIQTSIHVVRMGGTYVQGGMGKSDITFPIMAMCLKEVTVRGSFRYGAGDYELAVELVRTGRVDVKKLITGTVSFKQAEEAFQKVKSGEAIKILIAGPNEKV

SEQ ID NO: 27 - protein, 383 aa, Neurospora crassa
MATDGKSNLSFVLNKPLDVCFQDKPVPKINSPHDVLVAVNYTGICGSDVHYWLHGAIGHFVVKDPMVLGHESAGTIVAVGDAVKTLSVGDRVALEPGYPCRRCVHCLSGHYNLCPEMRFAATPPYDGTLTGFWTAPADFCYKLPETVSLQEGALIEPLAVAVHITKQAKIQPGQTVVVMGAGPVGLLCAAVAKAYGASKVVSVDIVPSKLEFAKSFAATHTYLSQRVSPEENARNIIAAADLGEGADAVIDASGAEPSIQAALHVVRQGGHYVQGGMGKDNIIFPIMALCIKEVTASGSFRYGSGDYRLAIQLVEQGKVDVKKLVNGVVPFKNAEEAFKKVKEGEVIKILIAGPNEDVEGSLDTTVDEKKLNEAKACGGSGCC

SEQ ID NO: 28 - protein, 353 aa, Nectria haematococca
MASNLSFVLNKPGDVTFEERPKPTLEDPHDVLVAINYTGICGSDVHYWVHGSIGKFVVTDPMVLGHESAGTIVEVGEKVKTLKVGDRVALEPGYPCRRCTNCLAGKYNLCPDMVFAATPPYHGTLTGYWRAPADFCFKLPENVSQQEGALIEPLAVGVHIVKQANVKPGDSVVVMGAGPVGLLCAAVARAYGASKIVSVDIVQSKLDFAKDFAATHTYASQRVSPEENAKNILELAGLPDGADVVIDASGAEPSIQASIHVLKVGGSYVQGGMGKSDITFPIMAMCIKEATVSGSFRYGPGDYPLAIELVATGKVDVKKLVTGIVDFQQAEEAFKKVKEGEAIKVLIKGPNEE

SEQ ID NO: 29 - protein, 380 aa, Pichia angust
MKGLLYYGTNDIRYSETVPEPEIKNPNDVKIKVSYCGICGTDLKEFTYSGGPVFFPKQGTKDKISGYELPLCPGHEFSGTVVEVGSGVTSVKPGDRVAVEATSHCSDRSRYKDTVAQDLGLCMACQSGSPNCCASLSFCGLGGASGGFAEYVVYGEDHMVKLPDSIPDDIGALVEPISVAWHAVERARFQPGQTALVLGGGPIGLATILALQGHHAGKIVCSEPALIRRQFAKELGAEVFDPSTCDDANAVLKAMVPENEGFHAAFDCSGVPQTFTTSIVATGPSGIAVNVAVWGDHPIGFMPMSLTYQEKYATGSMCYTVKDFQEVVKALEDGLISLDKARKMITGKVHLKDGVEKGFKQLIEHKENNVKILVTPNEVS

SEQ ID NO: 30 - protein, 354 aa, Penicillum chrysogenum
MATAQNLSFVLEGIHKVKFEDRPVPELKNPHDVIINVKYTGICGSDVHYWEHGSIGSFVVKDPMVLGHESAGIVSQVGSAVKTLKVGDRVAMEPGISCRRCDPCKAGKYNLCEDMRFAATPPYDGTLAKYYALPEDFCYKLPEHISLQEGALMEPLSVAVHIVRQAGVSPGQTVVVFGAGPVGLLCCAVATAFGASKVIAVDIQQQRLDFAKSYATTSTFMPSNVAAVENAERMKEENGLGAGADVAIDASGAEPSVHTGIHVLRNGGTYVQGGMGRSEILFPIMAACSKELTIKGSFRYGSGDYKLAVGLVSSGKVDVKRLITGTVKFEQAEQAFIEVKAGKGIKTLIGGIDV

SEQ ID NO: 31 - protein, 362 aa, Phaeosphaeria nodorum
MTTKTATQKVELPNPSFVLQAPNKVVYEDRPIPDLPSPYDVIVKPKWTGICGSDVHYWVEGRIGHFVVESPMVLGHESAGIVHKVGDKVKSLKVGDRVAMEPGVPCRRCVRCKEGKYNLCPDMAFAATPPYDGTLARYYALPEDYCYKLPENMSLEEGALIEPTAVAVHITRQASIKPGDSVVVFGAGPVGLLCCAVAKAYGAKKIVTVDINEQRLNFALQYAATDKFSSARVSAEENAKNLIKDCELGPGADVIIDASGAEPCIQTAIHALRMGGTYVQGGMGKPDINFPIMAMCTKELNVKGSFRYGAGDYQTAVDLVAGGRISIKELITGKVKFEDAENAFAQVKKGEGIKLLIEGPEE

SEQ ID NO: 32 - protein, 348 aa, Pichia pastoris
MSDNPSVILKRINEIVIEDRPIPAIEDPHYVKIAIKKTGICGSDVHFYTDGCCGSFKLESPMVLGHESAGIVVEVGSEVKSLRVGDKVACEPGIPSRYSNAYKSGHYNLCPEMAFAATPPIDGTLCRYFLLPEDFCVKLPEHVSLEEGALVEPLSVAVHAARLAKITFGDSVVVFGAGPVGLLVAATARAYGATNVLIVDIFDDKLTLAKDTLQVATHSFNSKNGMDNLLESFEGKHPNVSIDCTGVESCIAAGINALAPRGVHVQVGMGKSEYNNFPLGLICEKECIVKGVFRYCYNDYNLAVELIASGKVEVKGLVTHRFKFTEAVDAYDTVRQGKAIKAIIDGPE

SEQ ID NO: 33 - protein, 351 aa, Zygosaccharomyces rouxii
MTKQDAIVLQKPGVITVDKRDVPEIKDPHYVKLHIKATGICGSDVHYYTQGAIGQFVVKSPMVLGHESSGIVAEVGSAVTNVKVGDRVAIEPGIPSRYSDETMSGNYNLCPHMVFAATPPYDGTLTKYYLAPEDFVYKMPDHLSFEEGALAEPMSVGVHANKLAGTRFGSKVLVSGAGPVGLLAGAVARAFGATEVVFVDIAEEKLERSKQFGATHTVSSSSDEERFVSEVSKVLGGDLPNIVLECSGAQPAIRCGVKACKAGGHYVQVGMGKDDVNFPISAVGSKEITFHGCFRYKKGDFADSVALLSSGRINGKPLISHRFAFDKAPEAYKFNAEHGNEVVKTIITGPE

SEQ ID NO: 34 - protein, 368 aa, Arxula adeninivoran
MAAQVEEQVLNLRAQADHNPSFVLKKPLELGFEERPVPVITDPRDVKIQVKKTGICGSDVHFWQHGRIGDYVVEKPMVLGHESSGVVVEVGSEVTSLKVGDRVAMEPGVPDRRSKEYKMGRYHLCPHVRFAACPPTDGTLCKYYTLPEDFCVKLPENVDFEEGALVEPLSVAVHTARLLGIYPGSKVVVFGAGPIGQLCIGVCKAFGASIIGAVDLFEQKLETAKEFGASHTYVPQKGDSHDETAHKILELLPNKQAPDVVIDASGAEQSINAGIELLERGGTFGQVAMGRTDYIQFAVSRMAMKEIRFQGVFRYTYGDYELATQLIGDGKIPVKKLVTHRRPFEKAEEAYELVKSGVAVKCIIDGPE

SEQ ID NO: 35 - protein, 363 aa, Pichia stipitis
MTANPSLVLNKIDDISFETYDAPEISEPTDVLVQVKKTGICGSDIHFYAHGRIGNFVLTKPMVLGHESAGTVVQVGKGVTSLKVGDNVAIEPGIPSRFSDEYKSGHYNLCPHMAFAATPNSKEGEPNPPGTLCKYFKSPEDFLVKLPDHVSLELGALVEPLSVGVHASKLGSVAFGDYVAVFGAGPVGLLAAAVAKTFGAKGVIVVDIFDNKLKMAKDIGAATHTFNSKTGGSEELIKAFGGNVPNVVLECTGAEPCIKLGVDAIAPGGRFVQVGNAAGPVSFPITVFAMKELTLFGSFRYGFNDYKTAVGIFDTNYQNGRENAPIDFEQLITHRYKFKDAIEAYDLVRAGKGAVKCLIDGPE

SEQ ID NO: 36 - protein, 358 aa, Aspergillus niger
MSTQNTNAQNLSFVLEGIHRVKFEDRPIPEINNPHDVLVNVRFTGICGSDVHYWEHGSIGQFIVKDPMVLGHESSGVVSKVGSAVTSLKVGDCVAMEPGIPCRRCEPCKAGKYNLCVKMAFAATPPYDGTLAKYYVLPEDFCYKLPESITLQEGAIMEPLSVAVHIVKQAGINPGQSVVVFGAGPVGLLCCAVAKAYGASKVIAVDIQKGRLDFAKKYAATATFEPAKAAALENAQRIITENDLGSGADVAIDASGAEPSVHTGIHVLRAGGTYVQGGMGRSEITFPIMAACTKELNVKGSFRYGSGDYKLAVSLVSAGKVNVKELITGVVKFEDAERAFEEVRAGKGIKTLIAGVDS

SEQ ID NO: 37 - protein, 440 aa, Pichia guilliermondii
MSCNFTSSNKFFNFNSLLPFLYTSSRLSSTSSSTGSLGTLIGPGSISIFGRITGFFQCDCGAIAVYKVGVLHPTFFTIMTPNPSLVLNKVNDITFETLEAPTLLEPNEVMVEVKKTGICGSDIHYYSHGKIGDFVLTQPMVLGHESAGVVTAVGLNVKSLKVGDRVAIEPGVPSRFSEEYKSGHYQLCPNIVFAATPDPKHGSPSPPGTLCKYYKSPEDFLVKLPDCVSLELGAMVEPLSVGVHGCKQAKVTFGDVVVVFGGGPVGLLAAAAATKFGAAKVMVVDVIDDKLKMALEVGVATHTFNSKSGGADELVKELGEHPDVVIECTGAEVCINLGIESLKMGGRFAQVGNATRPVSFPIVAFSSRELTLYGSFRYGYNDYKTSVAILEHNYRNGRENAAIDFEKLITHRFKFEDAKKAYDYIRDGNVAVKVIIDGPE

SEQ ID NO: 38 - protein, 364 aa, Candida tropicalis
MTANPSLVLNKVDDISFEEYEAPKLESPRDVIVEVKKTGICGSDIHYYAHGSIGPFILRKPMVLGHESAGVVSAVGSEVTNLKVGDRVAIEPGVPSRFSDETKSGHYHLCPHMSFAATPPVNPDEPNPQGTLCKYYRVPCDFLFKLPDHVSLELGAMVEPLTVGVHGCKLADLKFGEDVVVFGAGPVGLLTAAVARTIGAKRVMVVDIFDNKLKMAKDMGAATHIFNSKTGGDYQDLIKSFDGVQPSVVLECSGAQPCIYMGVKILKAGGRFVQIGNAGGDVNFPIADFSTRELALYGSFRYGYGDYQTSIDILDRNYVNGKDKAPINFELLITHRFKFKDAIKAYDLVRAGNGAVKCLIDGPE

SEQ ID NO: 39 - protein, 354 aa, Kluyveromyces lactis
MSGTQKAVVLQKKGEITFEDIPAPEITDSHYVKIHVKKTGICGSDIHYYTHGSIGEFVVKKPMVLGHESSGVVVEVGKDVTLVQVGDRVAIEPGVPSRYSDETKSGHYNLCPHMAFAATPPYDGTLVKYYLAPEDFLVKLPDHVSFEEGACAEPLAVGVHANRLAETSFGKNVVVFGAGPVGLVTGAVAAAFGASAVVYVDVFENKLERSKDFGATNTINSTKYKSEDELTEVIKSELKGEQPEIAIDCSGAEICIRTAIKVLKAGGSYVQVGMGKDNINFPIAMIGAKELRVLGSFRYYFNDYKIAVKLISEGKVNVKKMITHTFKFEEAIDAYNFNLEHGSEVVKTMIDGPE

SEQ ID NO: 40 - protein, 364 aa, Candida shehatae
MTANPSLVLNKIDDITFESYDAPEITEPTDVLVEVKKTGICGSDIHYYAHGKIGNFVLTKPMVLGHESSGVVTKVGTGVTSLKVGDKVAIEPGIPSRFSDAYKSGHYNLCPHMCFAATPNSTEGEPNPPGTLCKYFKSPEDFLVKLPEHVSLEMGALVEPLSVGVHASKLASVKFGDYVAVFGAGPVGLLAAAVAKTFGAKGVIVIDIFDNKLQMAKDIGAATHIFNSKTGGDAAALVKAFDGHEPTVVLECTGAEPCINQGVAILAQGGRFVQVGNAPGPVKFPITEFATKELTLFGSFRYGFNDYKTSVDIMDTNYKNGKEKAPIDFEQLITHRFKFADAIKAYDLVRAGSGAVKCFIDGPE

SEQ ID NO: 41 - protein, 354 aa, Talaromyces stipitatus
MSLTETKNLSFVLEGIKKVKFEERPIPEIIDPYDVLINVKYTGICGSDVHYWEHGSIGSFVVREPMVLGHESSGVVSKVGSKVTTLKVGDQVAMEPGIPCRRCEPCKSGKYHLCINMAFAATPPYDGTLARYYRLPEDFCYKLPENIPLKEGALIEPLGVAVHVVKQGGVVPGNSVVVFGAGPVGLLCGAVAKAFGASKVIISDIQQSRLDFAKKYIADGTFQPARVSAEENANRLKEEHDILAGADVVLEASGAEPAVHTGIHALRTGGTFVQAGMGRSEINFPIMAVCGKELNFKGSFRYGSGDYKLAVELVATGKVSVKELITGEFKFEDAEQAYIDVKAGKGIKTIIVGL

SEQ ID NO: 42 - protein, 299 aa, Pachysolen tannophilus
AWKGDWPLATKSPLVGGHEGAGVVVGMGSAVKNWKLGDLAGIKWLNGSCMNCEFCMHGDEPNCAHADLSGYTHDGSFQQYATADAVQAGRIPAGTNLSEIAPILCAGVTAYKAIKTAELKPGDWCCISGSGGGLGTLAIQFAKAMGLRVIGIDGGAGKEKLCLDLGAEKYIDFTKTKDIVKDVIAATDGGPHAVINVSVSERAIDASVNYVRPTGTVVLVGLPAGAVCKSEVFSQVVRSVKIKGSYVGNRCDTAEAIDFYVRGLVKSPIKVIGLSELPMVYDLMEKGEILGRYVVDTSR

SEQ ID NO: 43 - protein, 383 aa, Neurospora crassa
MATDGKSNLSFVLNKPLDVCFQDKPVPKINSPHDVLVAVNYTGICGSDVHYWLHGAIGHFVVKDPMVLGHESAGTIVAVGDAVKTLSVGDRVALEPGYPCRRCVHCLSGHYNLCPEMRFAATPPYDGTLTGFWTAPADFCYKLPETVSLQEGALIEPLAVAVHITKQAKIQPGQTVVVMGAGPVGLLCAAVAKAYGASKVVSVARSPSKLEFAKSFAATHTYLSQRVSPEENARNIIAAADLGEGADAVIDASGAEPSIQAALHVVRQGGHYVQGGMGKDNIIFPIMALCIKEVTASGSFRYGSGDYRLAIQLVEQGKVDVKKLVNGVVPFKNAEEAFKKVKEGEVIKILIAGPNEDVEGSLDTTVDEKKLNEAKACGGSGCC

SEQ ID NO: 44 - protein, 570 aa, Aspergillus niger
MQGPLYIGFDLSTQQLKGLVVNSDLKVVYVSKFDFDADSHGFPIKKGVLTNEAEHEVFAPVALWLQALDGVLEGLRKQGMDFSQIKGISGAGQQHGSVYWGENAEKLLKELDASKTLEEQLDGAFSHPFSPNWQDSSTQKECDEFDAALGGQSELAFATGSKAHHRFTGPQIMRFQRKYPDVYKKTSRISLVSSFIASLFLGHIAPMDISDVCGMNLWNIKKGAYDEKLLQLCAGSSGVDDLKRKLGDVPEDGGIHLGPIDRYYVERYGFSPDCTIIPATGDNPATILALPLRASDAMVSLGTSTTFLMSTPSYKPDPATHFFNHPTTAGLYMFMLCYKNGGLARELVRDAVNEKLGEKPSTSWANFDKVTLETPPMGQKADSDPMKLGLFFPRPEIVPNLRSGQWRFDYNPKDGSLQPSNGGWDEPFDEARAIVESQMLSLRLRSRGLTQSPGEGIPAQPRRVYLVGGGSKNKAIAKVAGEILGGSEGVYKLEIGDNACALGAAYKAVWAMERAEGQTFEDLIGKRWHEEEFIEKIADGYQPGVFERYGQAAEGFEKMELEVLRQEGKH

SEQ ID NO: 45 - protein, 619 aa, Candida albicans
MYSFTFTITFIYIYKLFTFFEGYFTFIFYVNNPPPSPAMTDYSNSKSLFLGFDLSTQQLKIIITDENLTPLDTYNVEFDSQFKSKYTKINKGVITGDDGEVISPVAMWLDAINYVFDEMQKSKFPFDKVVGISGSGQQHGSVYWSGEANELLNDLIPCKELSSQLQDAFSWGYSPNWQDHSTVKEAEDFHKAIGKEHLAEISGSRAHLRFTGLQIRKFITRSHSKEYESTSRISLVSSFVTSILLGEIAQLEESDACGMNLYDIQKSQYDEELLALAAGVHTEIDNISKEDPKYKKSIDQLKQKLGEISPITYKSSGKISKYFVDTYGFNSDCKIYSFTGDNLATILSLPLQPNDCLISLGTSTTVLIITSNYEPSSQYHLFKHPTLPDHYMGMLCYCNGSLAREKARDQANKKHNVSDNKSWDKFNEILDHNKDFNGKLGIYFPLGEIIPQAPAQTIRAVLEDNGEITPCELDSHGFTVDDDASAIVDSQTLSCRLRAGPMLSKSSTTKNGKTNSSEELQQLYDNLVDKFGELSTDGKKQSFESLTARPNRCYYVGGASNNTSIITKMGSIFGPTNGNYKVEIPNACALGGAYKASWSYKCELENKMIGYDEYIGKII

SEQ ID NO: 46 - protein, 617 aa, Candida tropicalis
MTTDYSENDKLFLGLDLSTQQLKIIVTNEDLIPLKTYHVEFDAEFKEKYNITKGVVNGEDGEVISPVGMWLDSMNYVFNSMKKDKFPFDKVVGISGSAQQHGSVYWSHEANELLSDLKPEEDLSEQLKDAFSWEYSPNWQDHSTLKEAEAFHEAIGKENLAKITGSRAHLRFTGLQIRKFATRSHVEEYAKTSRISLVSSFLTSVLIGKVTGLEESDACGMNLYDITKSQYNEELLALGAGVHPKIDGVDKNDEKYQKSIDELKQKLGDITPITYESSGDISPYFVDTYGFNKDVKIYSFTGDNLATILSLPLQPNDCLISLGTSTTVLIITENYQPSSQYHLFKHPTMPDSYMGMLCYCNGSLAREKARDEVNKQNKVSDSKSWDKFDEILDNSKHFNHKLGIYFPLGEIIPQAPAQTIRAVLEDGKIIPCELNTHGFSIDDDANAIVESQTLSCRLRAGPMLSNSGDSSSDDESPESTKELENIYKDLTSKFGELYTDGKKQTFESLTARPNRCYYVGGASNNPSIIKKMGSIFGPVNGNYKVEIPNACALGGAYKASWSFACEEKGKMISYADYITKLFDTNDELDQFQVEDKWVEYFEGVGMLAKMEETLLKQ

SEQ ID NO: 47 - protein, 578 aa, Penicillium chrysogenum
MASDSPLYIGFDLSTQQLKGLVVNSDLKVVHAAKFDFDADSKGFPIKKGVLNNEAEHEVFAPVALWLQALDGVLETLRKEGLDFRRVKGISGAGQQHGSVYWGQNAESLLRNLDSSKSLEEQLEGAFSHPYSPNWQDSSTQNECDEFDAALGDRKHLAQATGSKAHHRFTGPQILRFTRKHPDVYKKTSRISLVSSFLASLFLGHIAPFDISDVCGMNLWNIKKGAYDEGLIQLCSGAFGVEDLKQKLGEVPEDGGLHLGSVHAYFVERFGFSPDCTVIPATGDNPATILALPLLPSDAMVSLGTSTTFLMSTPSYKPDPATHFFNHPTTPGLYMFMLCYKNGGLAREHVRDAINESLKDTPAQPWANFDKVALQTAPLGQQSPTDPMKMGLFFPRHEIVPNIPKGQWRFTYDANTGNLKETTDGWNSPQDEARAIIESQLLSCRLRSRDLTENPGGGLPSQPRRVYLVGGGSKNKAIAKIAGEILGGVEGVYSLDVGDNACALGAAYKAVWGIERQPGQTFEDLIGQRWNEAEFIEKIADGYQKGIFEQYGQAVEGFEKMELQVLQQVAEKGDGDDY

SEQ ID NO: 48 - protein, 623 aa, Pichia stipitis
  VARIANT 537: Xaa = Any Amino Acid
MTTTPFDAPDKLFLGFDLSTQQLKIIVTDENLAALKTYNVEFDSINSSVQKGVIAINDEISKGAIISPVYMWLDALDHVFEDMKKDGFPFNKVVGISGSCQQHGSVYWSRTAEKVLSELDAESSLSSQMRSAFTFKHAPNWQDHSTGKELEEFERVIGADALADISGSRAHYRFTGLQIRKLSTRFKPEKYNRTARISLVSSFVASVLLGRITSIEEADACGMNLYDIEKREFNEELLAIAAGVHPELDGVEQDGEIYRAGINELKRKLGPVKPITYESEGDIASYFVTRYGFNPDCKIYSFTGDNLATIISLPLAPNDALISLGTSTTVLIITKNYAPSSQYHLFKHPTMPDHYMGMICYCNGSLAREKVRDEVNEKFNVEDKKSWDKFNEILDKSTDFNNKLGIYFPLGEIVPNAAAQIKRSVLNSKNEIVDVELGDKNWQPEDDVSSIVESQTLSCRLRTGPMLSKSGDSSASSSASPQPEGDGTDLHKVYQDLVKKFGDLFTDGKKQTFESLTARPNRCYYVGGASNNGSIIXKMGSILAPVNGNYKVDIPNACALGGAYKASWSYECEAKKEWIGYDQYINRLFEVSDEMNSFEVKDKWLEYANGVGMLAKMESELKH

SEQ ID NO: 49 - protein, 600 aa, Saccharomyces cerevisiae
MLCSVIQRQTREVSNTMSLDSYYLGFDLSTQQLKCLAINQDLKIVHSETVEFEKDLPHYNTKKGVYIHGDTIECPVAMWLEALDLVLSKYREAKFPLNKVMAVSGSCQQHGSVYWSSQAESLLEQLNKKPEKDLLHYVSSVAFARQTAPNWQDHSTAKQCQEFEECIGGPEKMAQLTGSRAHFRFTGPQILKIAQLEPEAYEKTKTISLVSNFLTSILVGHLVELEEADACGMNLYDIRERKFSDELLHLIDSSSKDKTIRQKLMRAPMKNLIAGTICKYFIEKYGFNTNCKVSPMTGDNLATICSLPLRKNDVLVSLGTSTTVLLVTDKYHPSPNYHLFIHPTLPNHYMGMICYCNGSLARERIRDELNKERENNYEKTNDWTLFNQAVLDDSESSENELGVYFPLGEIVPSVKAINKRVIFNPKTGMIEREVAKFKDKRHDAKNIVESQALSCRVRISPLLSDSNASSQQRLNEDTIVKFDYDESPLRDYLNKRPERTSFVGGASKNDAIVKKFAQVIGATKGNFRLETPNSCALGGCYKAMWSLLYDSNKIAVPFDKFLNDNFPWHVMESISDVDNENWDRYNSKIVPLSELEKTLI

SEQ ID NO: 50 - protein, 617 aa, Pichia pastoris
MVTKEIQNRDSALTESVPNDLYLGFDLSTQQLKITSFEGRSLTHFKTYRVDFDEELSVYGINNGVYVNEETGEINAPVAMWVEALDLIFSKMQKDKFPFGIVKGMSGSCQQHGSVYWSKDAPDLLSSLSPSKDLKSQLCPKAFTFEKSPNWQDHSTGEELEIFERKAGSPENLSKITGSRAHYRFTGSQIRKLAKRVNPELYKETYRISLISSFLSSLLCGRITKIEESDGCGMNIYDIQNSRYDEDLLAVTAAVDPEIDGATEHERQEGVARLKDKLQDLEPVGYRSIGTIAAYFVEKYGFSEDSKVFSFTGDNLATILSLPLHNDDILVSLGTSTTVLLVTETYWPNSNYHVFKHPTVPGSYMVMLCYVNGALARNQIKTSLDKKYNVSDPNDWTKFNEILDKSKPLHGKEELGVYFPKGEIIPNCVAQTKRFSYDAKSKKLVTANWDIEDDVVSIVESQALSCRLRSGPLYHGSDETDQEEESEVIQRLSNFPKISADGKDQRLPDLISHPKKAFYVGGASQNVSIVRKFSEVLGAKEGNYQINLGDACAIGGAFKAVWSDLCETEKAIPYSDFLRKNFHWKENVKPVEADSSLWLQYVDGVGILSEIEQTLEK

SEQ ID NO: 51 - protein, 624 aa, Candida dubliniensis
MTDYSNSKPLFLGFDLSTQQLKIIITNENLTPLNTYNVEFDSQFKSKYKDINKGVITGDDGEVISPVAMWLDAINYVFDEMKKDKFPFNKVSGISGSCQQHGSVYWSEKANELLNDLNPSQELSTQLQDAFSWGYSPNWQDHSTVKEAEEFHKAIGKEHLAEITGSRAHLRFTGLQIRKFVTRSHSKEYKSTSRISLVSSFVTSILLGEIAQLEESDACGMNLYDIQKSQYDEELLALAAGVHPEIDNVSKEDPKYKKSIDQLKQKLGEISPITYKSSGKISKYFVDTYGFNSNCKIYSFTGDNLATILSLPLQHNDCLISLGTSTTVLIITSNYEPSSQYHLFKHPTLPDHYMGMLCYCNGSLAREKARDQVNAKHNISDKKSWDKFNEILDNNKDFNGKLGIYFPLGEIIPQAPAQTIRAVLEDNGEITPCELDSHGFTVDDDASAIVDSQTLSCRLRAGPMLSKSSSSNTTSSKKNGNEKTNTSKELKQLYDNLVNKFGELSTDGKKQSFESLIARPNRCYYVGGASNNTSIIKKMGSIFGPINGNYKVEIPNACALGGAYKASWSYKCELENKMISYDEYIGKLFDTNDELESFKVDDKWEEYFTGVGMLAKMEETLLKQ

SEQ ID NO: 52 - protein, 576 aa, Neurospora crassa
MDVQAIVIQSDLSVVSSAKVDFDGDFGAKYGIKKGVQVNEVDGEVFAPVAMWLEALDLVLQRLQEAKTPLNRIRGISGSCQQHGSVYWSREAEKLLAELQADKQRGDLVDQLKGAFSHPYAPNWQDHSTQAECDKFDEALGTAERLAHATGSAAHHRFTGPQIMRLRRKLPGMYASTSRISLVSSFLASLFIGSVAPMDISDVCGMNLWDIPSNTWSETLLALAAGGSTEGAADLKAKLGEVRLDGGGSMGKISPYFVGKYGFSPDCEIAPFTGDNPATILALPLRPLDAIVSLGTSTTFLMITPVYKPDPSYHFFNHPTTPGQYMFMLCYKNGGLAREKVRDALPAPSNSSKDPWETFNQHALSTPPLDVSSPATDQAKLGLYFYLPEIVPNISAGTWRYECSATDGSNLQPVNQPWPVEKDARIIVESQALSMRLRSQNLVSTPPSTPSGTSSSSSSSALPAQPRRIYLVGGGSLNPAIARIMGDVLGGVDGVYKLDVGGNACALGGAYKAVWAFERRDETETFDELIGKRWKEEGAIRKVDEGYKKGVFEGYGNVLGAFGEMEGKVLEVARNK

SEQ ID NO: 53 - protein, 580 aa, Kluyveromyces lactis
MSESGYYLGFDLSTQQLKCLAIDDQLNIVTTAAIEFDKDFPHYNTRKGVYIKDEGVIDAPVAMWLEAIDLCFERLGKCIDLKKVKSMSGSCQQHGTVFWNCDHLPKDLQPSSNLVKQLASCFSRDVAPNWQDHSTRKQCDELTDKVGGPQELARITGSSSHYRFSGSQIAKVHETEPEVYANTKKISLVSSFLASVLVGDIVPLEEADACGMNLYGIEKHEFNEDLLSVVDEDIASIKRKLFDPPTSSDEPKSLGPVSTYFQEKYGVNPDCQIYPFTGDNLATICSLPLQKNDVLISLGTSTTILLITDQYHSSPNYHLFIHPTVPNHYMGMICYCNGSLAREKIRDDINGESQTHDWTKFNEALLDNSLSNDNEIGLYFPLGEIVPNMDAVTKRCYFKYIDNKVVLTNVNMFPDKRLDAKNIVESQALSCRVRISPLLSEEANAINETQVLKSELKVKFDYDFFPLASYAKRPNRAFFVGGASKNEAIIKTMANVIGAKNGNYRLETANSCALGGCYKALWSLLKEQNPETPSFDRWLNAFFNWERDCEFVCNSDAAKWENYNNKIRTLSEIEREASSH

SEQ ID NO: 54 - protein, 620 aa, Meyerozyma guilliermondii
MTSKSSANYELLKELYLGFDLSTQQLKIIATNGKLDHLGTYNVEFDQEFGEKYEVKKGVRVNEQSGEIVSPVAMWLDAIDFLFGKMKQQNFPFDKVVGISGSGQQHGSVYWSLDAPQLLSNLDASTTLASQLKSAFTFPESPNWQDHSTGEEIKVFEDTVGGPEKLAELTGSRAHYRFTGLQIRKLAVRKNPELYRKTHRISLVSSFVASVLSGEITTIEQAEACGMNIYDIKKHDYDDELLSLAAGVHPKADSASEEEREKGIASLKEKLGEVKKVSYDNCGTISSYFVKKFGLNPSARIYPFTGDNLATIISLPLHPNDILLSLGTSTTVLLVTQNFKPSAQYHLFVHPTMPNHYMGMICYCNGALAREKVRDALNEKYSLEKNSWDKFNEVLDSSKKFDNKLGIYFPLGEIVPNASAQFKRSKLANGKIEDVESWDIDEDVSSIVESQSLSARLRAGPMLNGSDSSNSSTPELDESSSGESSKLKKMYHELHSEFGDLYTDGEKHTYGSLTSRPRNTFFVGGASNNLSIVRKMASILGAMDHNYKVEIPNACALGGAYKASWSHTCEKKNQWINYDDYISQNFHFDDLDPVQVKDEWESYFKGMGMLAKMEENLKHD

SEQ ID NO: 55 - protein, 569 aa, Podospora anserina
MTDNGPLYLGFDLSTQQLKAIVIQSDLSIVSSAKVDFDQDFGAKYKIKKGVLVNEQEGEVFAPVALWLESLDLVLQRLQEQNTPLNCIKGISGSCQQHGSVYWSHEAEQLLGGLTADKSLVDQLTGAFSHPFAPNWQDHSTQHECDKFEETMGTAERLAQATGSAAHHRFTGTQIMRLRHKLPQMYTSTSRISLVSSFLASLFLGSIAPMDISDVCGMNLWDIPSNNWSSPLLDLASGGSPDDLRAKLGEVRQDGGGSMGNVSSYFVNKYNFSPDCGVAPFTGDNPATILALPLRPLDAIVSLGTSTTFLMSTPVYKPDPSYHFFNHPTTPGQYMFMLCYKNGGLAREKVRDVLPSSESGDVWENFNKHALETAPLDVRKEGDRAKLGLYFYLPEIVPNIKAGTWRYTCDANSGEGLEEVREPWAKETDARAIIESQALSMRLRSQKLVTAPREGLPAQPGRVYLVGGGSLNPAITRVLGDALGGADGVYKLDVGGNACALGGAYKAVWAFERGDGEAFDELIGKRWKEEGAIQRVDEGYKKGVFEKYGNVLGAFEKMEEEILKVAKNT

SEQ ID NO: 56 - protein, 572 aa, Aspergillus flavus
MQGPLYIGFDLSTQQLKALVVNSDLKVVYVSKFDFDADSRGFPIKKGVITNEAEHEVYAPVALWLQALDGVLEGLKKQGLDFARVKGISGAGQQHGSVYWGQDAERLLKELDSGKSLEDQLSGAFSHPYSPNWQDSSTQKECDEFDAFLGGADKLANATGSKAHHRFTGPQILRFQRKYPEVYKKTSRISLVSSFLASLFLGHIAPLDISDACGMNLWNIKQGAYDEKLLQLCAGPSGVEDLKRKLGAVPEDGGINLGQIDRYYIERYGFSSDCTIIPATGDNPATILALPLRPSDAMVSLGTSTTFLMSTPNYMPDPATHFFNHPTTAGLYMFMLCYKNGGLAREHIRDAINDKLGMAGDKDPWANFDKITLETAPMGQKKDSDPMKMGLFFPRPEIVPNLRAGQWRFDYNPADGSLHETNGGWNKPADEARAIVESQFLSLRLRSRGLTASPGQGMPAQPRRVYLVGGGSKNKAIAKVAGEILGGSDGVYKLEIGDNACALGAAYKAVWALERKDGQTFEDLIGQRWREEDFIEKIADGYQKGVFEKYGAALEGFEKMELQVLKQEGETR

SEQ ID NO: 57 - protein, 573 aa, Aspergillus fumigatus
MTSQGPLYIGFDLSTQQLKGLVVNSELKVVHISKFDFDADSHGFSIKKGVLTNEAEHEVFAPVALWLQALDGVLNGLRKQGLDFSRVKGISGAGQQHGSVYWGENAESLLKSLDSSKSLEEQLSGAFSHPFSPNWQDASTQKECDEFDAFLGGPEQLAEATGSKAHHRFTGPQILRMQRKYPEVYKKTARISLVSSFLASLLLGHIAPMDISDVCGMNLWDIKKGAYNEKLLGLCAGPFGVEDLKRKLGAVPEDGGLRLGKINRYFVERYGFSSDCEILPSTGDNPATILALPLRPSDAMVSLGTSTTFLMSTPNYKPDPATHFFNHPTTPGLYMFMLCYKNGGLAREHVRDAINEKSGSGASQSWESFDKIMLETPPMGQKTESGPMKMGLFFPRPEIVPNVRSGQWRFTYDPASDALTETEDGWNTPSDEARAIVESQMLSLRLRSRGLTQSPGDGLPPQPRRVYLVGGGSKNKAIAKVAGEILGGSDGVYKLDVGDNACALGAAYKAVWAIERKPGQTFEDLIGQRWREEEFIEKIADGYQKGVFEKYGKAVEGFEKMEQQVLKQEAARK

SEQ ID NO: 58 - protein, 573 aa, Talaromyces stipitatus
MAPGPLYIGFDLSTQQLKGLVVSSDLKVEYEAKFDFDAHSHGFDIKKGVMTNEAEHEVFAPVAMWLQALDSVLKTLKDQGLDFGRIRGISGAGQQHGSVYWSKDAEKLLQSLRSEKSLEEQLADAFSHPYSPNWQDASTQKECDEFDAYLGGPEELAHVTGSKAHHRFTGPQILRFHRKYPEQYKKTSRISLVSSFLASLFLGRIAPFDISDVCGMNLWNITAGSWDDRLLKLCAGQFGVDDLKQKLGDVPEDGGLHLGKIHEYFVERYSFNPDCIIMPSTGDNPSTILALPLNPSDAMVSLGTSTTFLMSTPMYKPDSATHFFNHPTTPGLHMFMLCYKNGGLAREQVRDAINKQVGGNTAGKNPWANFDKAALETPAMGQKSASDTMKMGLFFPRPEIIPNLPSGQWRFNYNPQDKSLEETTSGWDIPLDEARAIVESQFLSLRLRSRGLTTAPAEGLPPQPKRVYLVGGGSKNTAIAKIAGEILGGHDGVYKLDVGENACALGAAYKAVWAIERQPGQTFEDLIGKRWREEEFVEKIADGYQPDVFKKYGVAVGGFERMEQQILQQEGRK

SEQ ID NO: 59 - protein, 581 aa, Aspergillus nidulans
MSSRSSSPLKGPLYIGFDLSTQQLKGLVVNSDLKVVYSSIFDFDADSQGFPIKKGVLTNEAEHEVFAPVALWLQALDSVLDGLKKQGLDFSHVRGISGAGQQHGSVYWGQDAEKLLNGLDAGKRLQEQLEGAFSHPYSPNWQDSSTQKECDEFDEYLGGADKLAEATGSKAHHRFTGPQILRFQKKYPDVYKKTSRISLVSSFLASLFLGHIAPLDISDVCGMNLWNIHKGAYDEDLLKLCAGPHGVEDLKRKLGDVPEDGGIDLGKVHRYYVDRYGFSPECTVIPSTGDNPATILALPLRPSDAMVSLGTSTTFLMSTPSYKADPATHFFNHPTTPGLYMFMLCYKNGGLAREKIRDAINDAKNEKNPSNPWANFDSVALQTPPLGQTSPSDPMKMGLFFPRPEIVPNLRAGQWLFNYDPSTGNLTETLNGEGWNRPADEARAIIESQMLSLRLRSRGLTSSPGGDIPAQPRRVYLVGGGSKNKTIAKIAGEILGGSEGVYKLEIGDNACALGAAYKAVWALERKKDQTFEDLIGARWHEEEFIEKIADGYQKEAFERYGKAVEGFEKMEQRVLEQEGRK

SEQ ID NO: 60 - protein, 572 aa, Aspergillus oryzae
MQGPLYIGFDLSTQQLKALVVNSDLKVVYVSKFDFDADSRGFPIKKGVITNEAEHEVYAPVALWLQALDGVLEGLKKQGLDFARVKGISGAGQQHGSVYWGQDAERLLKELDSGKSLEDQLSGAFSHPYSPNWQDSSTQKECDEFDAFLGGADKLANATGSKAHHRFTGPQILRFQRKYPEVYKKTSRISLVSSFLASLFLGHIAPLDTSDVCGMNLWNIKQGAYDEKLLQLCAGPSGVEDLKRKLGAVPEDGGINLGQIDRYYIERYGFSSDCTIIPATGDNPATILALPLRPSDAMVSLGTSTTFLMSTPNYMPDPATHFFNHPTTAGLYMFMLCYKNGGLAREHIRDAINDKLGMAGDKDPWANFDKITLETAPMGQKKDSDPMKMGLFFPRPEIVPNLRAGQWRFDYNPADGSLHETNGGWNKPADEARAIVESQFLSLRLRSRGLTASPGQGMPAQPRRVYLVGGGSKNKAIAKVAGEILGGSDGVYKLEIGDNACALGAAYKAVWALERKDGQTFEDLIGQRWREEDFIEKIADGYQKGVFEKYGAALEGFEKMELQVLKQEGETR

SEQ ID NO: 61 - protein, 580 aa, Zygosaccharomyces rouxii
MTETNDSFYLGFDLSTQQLKCLAINESLRIVHTETVAFGDELPQYETSKGVYVKGDSIQSPVSMWLEALDLLFSKFTQHGFDLSKVRAVSGSCQQHGSVYWTQKADELLRGLKSTKGSLAEQLSPEAFSRPTAPNWQDHSTGKQCHEFEDAVGGPQELARITGSRAHFRFTGTQILKIAEEEPEAYANTATVSLVSSFLASVLTGQLTSIEEAEACGMNLYDIPKREYHPKLLDLVDKDRKSIESKLKSPPIHCDKPVCLGSICSYFVDKYGFNKDCSVYPFTGDNLATICSLPLEKNDVLVSLGTSTTILLVTDQYHPSADYHLFIHPTLPNHYMGMICYCNGALARERVRDYINGSPTSDWTPFNDALNDTNLNNDDEIGVYFPLGEIVPSVPSVYKRAKFDPSTGHIKEFVDNFADDRHDAKNIVESQALSCRVRISPLLTSGVPVEGLAKDPNVRFDYDDIPLSQYYGRRPRRAFFVGGASKNDAIVNKFIQVLGATEGNYRLETPNSCALGGCYKAIWSHKIHEKQITATFDHFLGEKFPWGEVEHIRDSDDASWHHYNKKILPLSELEASLPKH

SEQ ID NO: 62 - protein, 598 aa, Nectria haematococca
MPFLARSRSNSPELPSDSKPLYLGFDLSTQQLKGIVVDSDLKVVGEAKVDFDKDFGRKYGVQKGVHVIEETGEVYAPVAMWMESLDLVLERLAEAMPVPLSRIRAISGSCQQHGSVFWNGQAYEILHNLDPRLPLAVQLPGALAHPWSPNWQDQSTQNECDAFDAALGGRQKLAEVTGSGAHHRFTGTQIMRLKKDLPQMYARTAHISLVSSWLASVFLGAIAPMDVSDVCGMNLFDMSRQTFSEPLLELAAGSKRDAINLRKKLGEPCLKGEAILGPVSPYFVDRHGFHPDCQITPFTGDNPGTILALPLRPLDAIVSLGTSTTFLMNTPKYKPDGSYHFFNHPTTDGHYMFMLCYKNGGLARERVRDQLPKPENGPTGWETFNKAVEDTPLMGAAKEDDRRKLGLYFYLRETVPNIRAGTWRYSCEPDGSDLQEVKGGWDKETDARMIVESQALSMRLRSQNLVHSPRPGLPAQPRRIYLVGGGSLNPAIARVLGEVLGGSEGVYKLDVGGNACALGGAYKALWAMERQENETFDDLIGKRWTEEGNIQRIDEGFRDGTYQKYGKLLTAFEALENKILAEQAHAPEEDQRRSEEKV

SEQ ID NO: 63 - protein, 427 aa, Aspergillus nidulans
MEILQKKPKNIAIHTSPVHDLRVVDCEIPRLAPDGCLIHVRATGICGSDVHFWKHGRIGPMVVTGDNGLGHESAGVVLQVGDAVTRFKPGKYHACPDVVFFSTPPHHGTLRRYHAHPEAWLHRLPDHVSFEEGALLEPLTVALAGIDRSGLRLADPLVICGAGPIGLVTLLAANAAGAAPIVITDIDSNRLAKAKELVPRVQPVLVQKQESPQELAGRIVQRLGQEARLVLECTGVESSVHAGIYATRFGGTVFVIRVGKDFQNIPFMHMSAKEIDLRFQYRYHDIYPKAISLVNAGLVDLKPLVSHRYKLEDGLEAFATASNTAAKAIKLGTSSREPYSGICPKDEVVPTVLTKPGTRFLRDCTTHIALHGSSPSSNVYGKPGIECLRRSAEHTREQQWTLQFDGCSSLASSGSGERLGQARPEPV

SEQ ID NO: 64 - protein, 386 aa, Aspergillus niger
MATATVLEKANIGVFTNTKHDLWVADAKPTLEEVKNGQGLQPGEVTIEVRSTGICGSDVHFWHAGCIGPMIVTGDHILGHESAGQVVAVAPDVTSLKPGDRVAVEPNIICNACEPCLTGRYNGCENVQFLSTPPVDGLLRRYVNHPAIWCHKIGDMSYEDGALLEPLSVSLAGIERSGLRLGDPCLVTGAGPIGLITLLSARAAGASPIVITDIDEGRLEFAKSLVPDVRTYKVQIGLSAEQNAEGIINVFNDGQGSGPGALRPRIAMECTGVESSVASAIWSVKFGGKVFVIGVGKNEMTVPFMRLSTWEIDLQYQYRYCNTWPRAIRLVRNGVIDLKKLVTHRFLLEDAIKAFETAANPKTGAIKVQIMSSEDDVKAASAGQKI

SEQ ID NO: 65 - protein, 386 aa, Aspergillus niger
MATATVLEKANIGVFTNTKHDLWVADAKPTLEEVKNGQGLQPGEVTIEVRSTGICGSDVHFWHAGCIGPMIVTGDHILGHESAGQVVAVAPDVTSLKPGDRVAVEPNIICNACEPCLTGRYNGCENVQFLSTPPVDGLLRRYVNHPAIWCHKIGDMSYEDGALLEPLSVSLAGIERSGLRLGDPCLVTGAGPIGLITLLSARAAGASPIVITSRDEGRLEFAKSLVPDVRTYKVQIGLSAEQNAEGIINVFNDGQGSGPGALRPRIAMECTGVESSVASAIWSVKFGGKVFVIGVGKNEMTVPFMRLSTWEIDLQYQYRYCNTWPRAIRLVRNGVIDLKKLVTHRFLLEDAIKAFETATNPKTGAIKVQIMSSEDDVKAASAGQKI

SEQ ID NO: 66 - protein, 382 aa, Aspergillus oryzae
MATATVLEKANIGVYTNTNHDLWVAESKPTLEEVKSGESLKPGEVTVQVRSTGICGSDVHFWHAGCIGPMIVTGDHILGHESAGEVIAVASDVTHLKPGDRVAVEPNIPCHACEPCLTGRYNGCEKVLFLSTPPVDGLLRRYVNHPAVWCHKIGDMSYEDGALLEPLSVSLAAIERSGLRLGDPVLVTGAGPIGLITLLSARAAGATPIVITDIDEGRLAFAKSLVPDVITYKVQTNLSAEDNAAGIIDAFNDGQGSAPDALKPKLALECTGVESSVASAIWSVKFGGKVFVIGVGKNEMKIPFMRLSTQEIDLQYQYRYCNTWPRAIRLVRNGVISLKKLVTHRFLLEDALKAFETAADPKTGAIKVQIMSNEEDVKGASA

SEQ ID NO: 67 - protein, 377 aa, Trichoderma longigrachiatum
MSPSAVDDAPKATGAAISVKPNIGVFTNPKHDLWISEAEPSADAVKSGADLKPGEVTIAVRSTGICGSDVHFWHAGCIGPMIVEGDHILGHESAGEVIAVHPTVSSLQIGDRVAIEPNIICNACEPCLTGRYNGCEKVEFLSTPPVPGLLRRYVNHPAVWCHKIGNMSWENGALLEPLSVALAGMQRAKVQLGDPVLVCGAGPIGLVSMLCAAAAGACPLVITDISESRLAFAKEICPRVTTHRIEIGKSAEETAKSIVSSFGGVEPAVTLECTGVESSIAAAIWASKFGGKVFVIGVGKNEISIPFMRASVREVDIQLQYRYSNTWPRAIRLIESGVIDLSKFVTHRFPLEDAVKAFETSADPKSGAIKVMIQSLD

SEQ ID NO: 68 - protein, 377 aa, Trichoderma longigrachiatum
MSPSAVDDAPKATGAAISVKPNIGVFTNPKHDLWISEAEPSADAVKSGADLKPGEVTIAVRSTGICGSDVHFWHAGCIGPMIVEGDHILGHESAGEVIAVHPTVSSLQIGDRVAIEPNIICNACEPCLTGRYNGCEKVEFLSTPPVPGLLRRYVNHPAVWCHKIGNMSWENGALLEPLSVALAGMQRAKVQLGDPVLVCGAGPIGLVSMLCAAAAGACPLVITSRSESRLAFAKEICPRVTTHRIEIGKSAEETAKSIVSSFGGVEPAVTLECTGVESSIAAAIWASKFGGKVFVIGVGKNEISIPFMRASVREVDIQLQYRYSNTWPRAIRLIESGVIDLSKFVTHRFPLEDAVKAFETSTDPKSGAIKVMIQSLD

SEQ ID NO: 69 - protein, 363 aa, Neurospora crassa
MASSASKTNIGVFTNPQHDLWISEASPSLESVQKGEELKEGEVTVAVRSTGICGSDVHFWKHGCIGPMIVECDHVLGHESAGEVIAVHPSVKSIKVGDRVAIEPQVICNACEPCLTGRYNGCERVDFLSTPPVPGLLRRYVNHPAVWCHKIGNMSYENGAMLEPLSVALAGLQRAGVRLGDPVLICGAGPIGLITMLCAKAAGACPLVITDIDEGRLKFAKEICPEVVTHKVERLSAEESAKKIVESFGGIEPAVALECTGVESSIAAAIWAVKFGGKVFVIGVGKNEIQIPFMRASVREVDLQFQYRYCNTWPRAIRLVENGLVDLTRLVTHRFPLEDALKAFETASDPKTGAIKVQIQSLE

SEQ ID NO: 70 - protein, 363 aa, Neurospora crassa
MASSASKTNIGVFTNPQHDLWISEASPSLESVQKGEELKEGEVTVAVRSTGICGSDVHFWKHGCIGPMIVECDHVLGHESAGEVIAVHPSVKSIKVGDRVAIEPQVICNACEPCLTGRYNGCERVDFLSTPPVPGLLRRYVNHPAVWCHKIGNMSYENGAMLEPLSVALAGLQRAGVRLGDPVLICGAGPIGLITMLCAKAAGACPLVITSRDEGRLKFAKEICPEVVTHKVERLSAEESAKKIVESFGGIEPAVALECTGVESSIAAAIWAVKFGGKVFVIGVGKNEIQIPFMRASVREVDLQFQYRYCNTWPRAIRLVENGLVDLTRLVTHRFPLEDALKAFETTSDPKTGAIKVQIQSLE

SEQ ID NO: 71 - protein, 385 aa, Penicillum chrysogenum
MASATVTKTNIGVYTNPKHDLWIADSSPTAEDINAGKGLKAGEVTIEVRSTGICGSDVHFWHAGCIGPMIVTGDHVLGHESAGQVLAVAPDVTHLKVGDRVAVEPNVICNACEPCLTGRYNGCVNVAFLSTPPVDGLLRRYVNHPAVWCHKIGDMSYEDGAMLEPLSVTLAAIERSGLRLGDALLITGAGPIGLISLLSARAAGACPIVITDIDEGRLAFAKSLVPEVRTYKVEIGKSAEECADGIINALNDGQGSGPDALRPKLALECTGVESSVNSAIWSVKFGGKVFVIGVGKNEMTIPFMRLSTQEIDLQYQYRYCNTWPRAIRLIQNGVIDLSKLVTHRYSLENALQAFETASNPKTGAIKVQIMSSEEDVKAATAGQKY

SEQ ID NO: 72 - protein, 385 aa, Penicillum chrysogenum
MASATVTKTNIGVYTNPKHDLWIADSSPTAEDINAGKGLKAGEVTIEVRSTGICGSDVHFWHAGCIGPMIVTGDHVLGHESAGQVLAVAPDVTHLKVGDRVAVEPNVICNACEPCLTGRYNGCVNVAFLSTPPVDGLLRRYVNHPAVWCHKIGDMSYEDGAMLEPLSVTLAAIERSGLRLGDALLITGAGPIGLISLLSARAAGACPIVITSRDEGRLAFAKSLVPEVRTYKVEIGKSAEECADGIINALNDGQGSGPDALRPKLALECTGVESSVNSAIWSVKFGGKVFVIGVGKNEMTIPFMRLSTQEIDLQYQYRYCNTWPRAIRLIQNGVIDLSKLVTHRYSLENALQAFETATNPKTGAIKVQIMSSEEDVKAATAGQKY

SEQ ID NO: 73 - protein, 359 aa, Aspergillus fumigatus
MDVIIRKPQNFAIHTSPSHDLRLVECEIPKLRPDECLVHVRATGICGSDVHFWKHGRIGPMIVTGDNGLGHESAGVVLQIGEAVTRFKPGDRVALECGVPCSKPTCSFCRTGKYHACPDVVFFSTPPHHGTLRRYHAHPEAWLHKIPDNISFEEGSLLEPLSVALAGINRSGLRLADPLVICGAGPIGLITLLAASAAGAEPIVITDIDENRLSKAKELVPRVHPVHVQKQESPQHLGARIVRELGQEAKLVLECTGVESSVHAGIYATRFGGMVFVIGVGKDFQNIPFMHMSAKEIDLRFQYRYHDIYPRAINLVSAGMIDLKPLVSHRYKLEDGLAAFDTASNPAARAIKVQIIDDE

SEQ ID NO: 74 - protein, 373 aa, Botryotinia fuckeliana
MSPSATEITETTMAKPTKSNIGVYTNPAHDLWVAEAEPSLESIEKGDSLKPGEVTVGIRSVGICGSDVHFWHAGCIGPMIVEDTHILGHESAGVVLAVHPSVDSLKVGDRVAVEPNIICGECERCLTGRYNGCEKVLFLSTPPVPGLLRRYVNHPATWCYKIGNMSFEDGAMLEPLSVALAGLERANVKLGDPVLICGAGPIGLITLLCARAAGACPIVITDIDEGRLAFAKELVPSVTTHKVERLSAEEGAKSIVKSFGGIEPAVAMECTGVESSVAAACAVKFGGKVFVVGVGKDEMTLPFMRLSTREVDLQFQYRYCNTWPRAIRLVESGIIDMKKLVTHRFPLEDAIKAFETAANPKTGAIKVQIKNDE

SEQ ID NO: 75 - protein, 372 aa, Magnaporthe oryzae; identical to SEQ ID NO: 10

SEQ ID NO: 76 - protein, 375 aa, Nectria haematococca
MSPSAVDAPATADVKTTLKPNIGVYTNPNHDLWVNAAEPSAESVKSGADLKQGEVSVAIRSTGICGSDVHFWHAGCIGPMIVEGDHILGHESAGEVVAVHPSVTNLKVGDRVAVEPNIPCGTCEPCLTGRYNGCETVQFLSTPPVPGMLRRYINHPAVWCHKIGNMSYENGAMLEPLSVALAGMQRAQVSLGDPVLICGAGPIGLITLLCSAAAGASPIVITDISESRLAFAKELCPRVITHKVERLSAEDSAKAIVNSFGGVEPTIALECTGVESSIAAAIWSVKFGGKVFIIGVGKNEINIPFMRASVREVDIQLQYRYCNTWPRAIRLVESGVIDLSKLVTHRFKLEDALKAFETSADPKSGSIKVMIQSLE

SEQ ID NO: 77 - protein, 373 aa, Podospora anserina
MSTTTTTTKVKASKANIGVFTNPGHDLWIDSAEPSLESVQQGSPELKEGEVTVAIRSTGICGSDVHFWKHGCIGPMIVTCDHVLGHESAGEIIAVHPSVKTLQVGDRVAIEPQVICNECEPCLTGRYNGCEKVDFLSTPPVAGLLRRYVNHKAVWCHKIGDMSYEDGAMLEPLSVALAGMQRAGVRLGDPVLICGAGPIGLITLLCCQAAGACPLVITDIDEGRLKFAKEIAPGVVTVKVEPGLSVEQQAERIVKEGFNGIEPAIALECTGVESSIGAAIWAMKFGGKVFVIGVGRNEIQIPFMRASVREVDLQFQYRYSNTWPRAIRLVQSKVLDMSRLVTHRFPLEEALKAFNTASDPKTGAIKVQIQSLD

SEQ ID NO: 78 - protein, 371 aa, Phaeosphaeria nodorum
MSSTTVTEVKPSKANIGVYTNPAHDLWVAEAEPSLEVVEKGGDLKEGEVLLNVKSTGICGSDIHFWHAGCIGPMIVEDTHILGHESAGTVLAVHPSVSTLKVGDRVAIEPNVICHECEPCLTGRYNGCEKVQFLSTPPVTGLLRRYLKHPAMWCHKLPDNLTFEDGAMLEPLSVALAGMDRANVRLGDPVVICGAGPIGLVTLLCARAAGAAPIVITDIDEGRLKFAKDLVPNVATHKVEFSHSVDDFRNAVIAKMEGVEPAIAMECTGVESSINGAIQAVKFGGKVFVIGVGKNEMKIPFMRLSTREVDLQFQYRYCNTWPKAIRLVKSGVIELSKLVTHRFQLEDAVQAFKTAADPKTGAIKVQIQSLD

SEQ ID NO: 79 - protein, 272 aa, Ambrosiozyma monospora
MTDYIPTFRFDGHLTIVTGACGGLAEALIKGLLAYGSDIALLDIDQEKTAAKQAEYHKYATEELKLKEVPKMGSYACDISDSDTVHKVFAQVAKDFGKLPLHLVNTAGYCENFPCEDYPAKNAEKMVKVNLLGSLYVSQAFAKPLIKEGIKGASVVLIGSMSGAIVNDPQNQVVYNMSKAGVIHLAKTLACEWAKYNIRVNSLNPGYIYGPLTKNVINGNEELYNRWISGIPQQRMSEPKEYIGAVLYLLSESAASYTTGASLLVDGGFTSW

SEQ ID NO: 80 - protein, 266 aa, Aspergillus nidulans
MPQQVPTASHLSDLFSLKGKVVVITGASGPRGMGIEAARGCAEMGANVAITYASRPEGGEKNAAELARDYGVKAKAYKCDVGDFKSVEKLVQDVIAEFGQIDAFIANAGRTASAGVLDGSVKDWEEVVQTDLNGTFHCAKAVGPHFKQRGKGSLVITASMSGHIANYPQEQTSYNVAKAGCIHMARSLANEWRDFARVNSISPGYIDTGLSDFVDKKTQDLWLSMIPMGRHGDAKELKGAYVYLVSDASTYTTGADLVIDGGYTCR

SEQ ID NO: 81 - protein, 266 aa, Aspergillus terreus
MPIPVPSANHLKDLFSLKDKVVVITGASGPRGMGIEAARGCAEMGANVAITYASRPQGGEKNAEELAKAYGVKAKAYKCDVGNFESVEKLVKDVIAEFGQIDAFIANAGRTASSGILDGSVNDWMEVIQTDLTGTFHCAKAVGPHFKQRGTGSLVITASMSGHIANFPQEQTSYNVAKAGCIHLARSLANEWRDFARVNSISPGYIDTGLSDFVPKDVQDLWMSMIPMGRNGDAKELKGAYVYLVSDASTYTTGADLRIDGGYCVR

SEQ ID NO: 82 - protein, 271 aa, Neurospora crassa
MASTTKGNAIPTASKLSDLFSLKGKVVVITGASGPRGMGIEAARGCAEMGASVAITYASRADGAQKNVAELEKEYGIKAKAYKLNVADYAECEKLVKDVIADFGQIDAFIANAGATAKSGVLDGSKEEWDRVIETDLNGTAYCAKAVGPHFKERGRGSFVITSSISGHIANYPQEQTSYNVAKAGCIHMARSLANEWRDFARVNSISPGYIDTGLSDFVDQKTQDLWKSMIPLGRNGDAKELKGAYVYLVSDASSYTTGADILIDGGYTVR

SEQ ID NO: 83 - protein, 280 aa, Candida dubliniensis
MSKETISYTNDALGPLPTKPATIPDNILDAFSLKGKVASVTGSSGGIGWAVAEGYAQAGADVAIWYNSHPADDKAEYLAKTYGVKSKAYKCNVTDFQDVEKVVKQIESDFGTIDIFVANAGVAWTDGPEIDVKGVDKWNKVVNVDLNSVYYCAHVVGPIFRKHGKGSFIFTASMSASIVNVPQLQAAYNAAKAGVKHLSKSLSVEWAPFARVNSVSPGYIATHLSEFADPDVKNKWLQLTPLGREAKPRELVGAYLYLASDAASYTTGADLAVDGGYTVV

SEQ ID NO: 84 - protein, 266 aa, Hypocrea jecorina
MPQPVPTANRLLDLFSLKGKVVVVTGASGPRGMGIEAARGCAEMGADLAITYSSRKEGAEKNAEELTKEYGVKVKVYKVNQSDYNDVERFVNQVVSDFGKIDAFIANAGATANSGVVDGSASDWDHVIQVDLSGTAYCAKAVGAHFKKQGHGSLVITASMSGHVANYPQEQTSYNVAKAGCIHLARSLANEWRDFARVNSISPGYIDTGLSDFIDEKTQELWRSMIPMGRNGDAKELKGAYVYLVSDASSYTTGADIVIDGGYTTR

SEQ ID NO: 85 - protein, 271 aa, Aspergillus terreus
MESVKNSIRWPNPALPDSVFKMFDMHGKVVIITGGSGGIGYQVARALAEAGADIALWYNSSPDAVRLASTLEKDFGVRSEAYKCSVQNFDEVQAATDAVVRDFGGLHVMIANAGIPSKAGGLDDRLEDWQRVVDIDFSGAYYCARAAGQIFRKQGFGNMIFTASMSGHAANVPQQQACYNACKAGVIHLAKSLAVEWAGFARVNCVSPGYIDTPISGDCPFEMKEAWYSLTPMRRDADPRELKGVYLYLASDASTYTTGADVVVDGGYTCR

SEQ ID NO: 86 - protein, 266 aa, Aspergillus niger
MPISIPSASSVHDLFSLKGKVVVITGASGPRGMGIEAARGCAEMGANIALTYSSRPQGGEKNAEELRNTYGVKAKAYQCNVGDWNSVKKLVDDVLAEFGQIDAFIANAGKTASSGILDGSVEDWEEVIQTDLTGTFHCAKAVGPHFKQRGTGSFIITSSMSGHIANFPQEQTSYNVAKAGCIHMARSLANEWRDFARVNSISPGYIDTGLSDFVDKKTQDLWMSMIPMGRNGDAKELKGAYVYLASDASTYTTGADLVIDGGYTVR

SEQ ID NO: 87 - protein, 272 aa, Ambrosiozyma monospora; identical to SEQ ID NO: 79

SEQ ID NO: 88 - protein, 244 aa, Mus musculus
MDLGLAGRRALVTGAGKGIGRSTVLALKAAGAQVVAVSRTREDLDDLVRECPGVEPVCVDLADWEATEQALSNVGPVDLLVNNAAVALLQPFLEVTKEACDTSFNVNLRAVIQVSQIVAKGMIARGVPGAIVNVSSQASQRALTNHTVYCSTKGALDMLTKMMALELGPHKIRVNAVNPTVVMTPMGRTNWSDPHKAKAMLDRIPLGKFAEVENVVDTILFLLSNRSGMTTGSTLPVDGGFLAT

SEQ ID NO: 89 - protein, 244 aa, Cavia porcellus
MDLGLAGRRALVTGAGKGIGRSTVLALKAAGAQVVAVSRTREDLDDLVRECPGVEPVCVDLADWEATEQALSNVGPADLLVNNAAVALLQPFLEVTKEACVTSFNVNLRAVIQVSQIVAKGMIARGVPGAIVNVSSQASQRALTNHTVYCSTKGALYMLTKMMALELGPHKIRVNAVNPTVVMTPMGRTNWSDPHKAKAMLDRIPLGKFAEVENVVDTILFLLSNRSGMTTGSTLPVDGGFLAT

SEQ ID NO: 90 - protein, 562 aa, Neurospora crassa
MAPPKFLGLSGRPLSLAVSTVATTGFLLFGYDQGVMSGIITAPAFNNFFTPTKDNSTMQGLITAIYEIGCLIGAMFVLWTGDLLGRRRNIMVGAFIMALGVIIQVTCQAGSNPFAQLFVGRVVMGIGNGMNTSTIPTYQAECSKTSNRGLLICIEGGVIAFGTLIAYWIDYGASYGPDDLVWRFPIAFQLLFAIFICVPMFYLPESPRWLLSHGRTQEADKVIAALRGYEIDGPETIQERNLIVDSLRASGGFGQKSTPFKALFTGGKTQHFRRLLLGSSSQFMQQVGGCNAVIYYFPILFQDSIGESHNMSMLLGGINMIVYSIFATVSWFAIERVGRRRLFLIGTVGQMLSMVIVFACLIPDDPMKARGAAVGLFTYIAFFGATWLPLPWLYPAEVNPIRTRGKANAVSTCSNWMFNFLIVMVTPIMVDKIGWGTYLFFAVMNGCFLPIIYFFYPETANRSLEEIDIIFAKGFVENMSYVTAAKELPHLTAEEIESYANKYGLVDRDSNGEGGNRHDEEKTRDRPDQSDSDSPAHVEIDVVDEHGVESGFGDGINTKETR

SEQ ID NO: 91 - protein, 544 aa, Pichia stipitis
MSSVEKSAETASYTSQVSASGSAKTNSYLGLRGHKLNFAVSCFAGVGFLLFGYDQGVMGSLLTLPSFENTFPAMKASNNATLQGAVIALYEIGCMSSSLATIYLGDRLGRLKIMFIGCVIVCIGAALQASAFTIAHLTVARIITGLGTGFITSTVPVYQSECSPAKKRGQLIMMEGSLIALGIAISYWIDFGFYFLRNDGLHSSASWRAPIALQCVFAVLLISTVFFFPESPRWLLNKGRTEEAREVFSALYDLPADSEKISIQIEEIQAAIDLERQAGEGFVLKELFTQGPARNLQRVALSCWSQIMQQITGINIITYYAGTIFESYIGMSPFMSRILAALNGTEYFLVSLIAFYTVERLGRRFLLFWGAIAMALVMAGLTVTVKLAGEGNTHAGVGAAVLLFAFNSFFGVSWLGGSWLLPPELLSLKLRAPGAALSTASNWAFNFMVVMITPVGFQSIGSYTYLIFAAINLLMAPVIYFLYPETKGRSLEEMDIIFNQCPVWEPWKVVQIARDLPIMHSEVLDHEKNVIIKKSRIEHVENIS

SEQ ID NO: 92 - protein, 566 aa, Pichia stipitis
MHGGGDGNDITEIIAARRLQIAGKSGVAGLVANSRSFFIAVFASLGGLVYGYNQGMFGQISGMYSFSKAIGVEKIQDNPTLQGLLTSILELGAWVGVLMNGYIADRLGRKKSVVVGVFFFFIGVIVQAVARGGNYDYILGGRFVVGIGVGILSMVVPLYNAEISPPEIRGSLVALQQLAITFGIMISYWITYGTNYIGGTGSGQSKASWLVPICIQLVPALLLGVGIFFMPESPRWLMNEDREDECLSVLSNLRSLSKEDTLVQMEFLEMKAQKLFERELSAKYFPHLQDGSAKSNFLIGFNQYKSMITHYPTFKRVAVACLIMTFQQWTGVNFILYYAPFIFSSLGLSGNTISLLASGVVGIVMFLATIPAVLWVDRLGRKPVLISGAIIMGICHFVVAAILGQFGGNFVNHSGAGWVAVVFVWIFAIGFGYSWGPCAWVLVAEVFPLGLRAKGVSIGASSNWLNNFAVAMSTPDFVAKAKFGAYIFLGLMCIFGAAYVQFFCPETKGRTLEEIDELFGDTSGTSKMEKEIHEQKLKEVGLLQLLGEENASESENSKADVYHVEK

SEQ ID NO: 93 - protein, 335 aa, Saccharomyces cerevisiae
MSEPAQKKQKVANNSLEQLKASGTVVVADTGDFGSIAKFQPQDSTTNPSLILAAAKQPTYAKLIDVAVEYGKKHGKTTEEQVENAVDRLLVEFGKEILKIVPGRVSTEVDARLSFDTQATIEKARHIIKLFEQEGVSKERVLIKIASTWEGIQAAKELEEKDGIHCNLTLLFSFVQAVACAEAQVTLISPFVGRILDWYKSSTGKDYKGEADPGVISVKKIYNYYKKYGYKTIVMGASFRSTDEIKNLAGVDYLTISPALLDKLMNSTEPFPRVLDPVSAKKEAGDKISYIDDESKFRFDLNEDAMATEKLSEGIRKFSADIVTLFDLIEKKVTA

SEQ ID NO: 94 - protein, 681 aa, Saccharomyces cerevisiae
MAQFSDIDKLAVSTLRLLSVDQVESAQSGHPGAPLGLAPVAHVIFKQLRCNPNNEHWINRDRFVLSNGHSCALLYSMLHLLGYDYSIEDLRQFRQVNSRTPGHPEFHSAGVEITSGPLGQGISNAVGMAIAQANFAATYNEDGFPISDSYTFAIVGDGCLQEGVSSETSSLAGHLQLGNLITFYDSNSISIDGKTSYSFDEDVLKRYEAYGWEVMEVDKGDDDMESISSALEKAKLSKDKPTIIKVTTTIGFGSLQQGTAGVHGSALKADDVKQLKKRWGFDPNKSFVVPQEVYDYYKKTVVEPGQKLNEEWDRMFEEYKTKFPEKGKELQRRLNGELPEGWEKHLPKFTPDDDALATRKTSQQVLTNMVQVLPELIGGSADLTPSNLTRWEGAVDFQPPITQLGNYAGRYIRYGVREHGMGAIMNGISAFGANYKPYGGTFLNFVSYAAGAVRLAALSGNPVIWVATHDSIGLGEDGPTHQPIETLAHLRAIPNMHVWRPADGNETSAAYYSAIKSGRTPSVVALSRQNLPQLEHSSFEKALKGGYVIHDVENPDIILVSTGSEVSISIDAAKKLYDTKKIKARVVSLPDFYTFDRQSEEYRFSVLPDGVPIMSFEVLATSSWGKYAHQSFGLDEFGRSGKGPEIYKLFDFTADGVASRAEKTINYYKGKQLLSPMGRAF

SEQ ID NO: 95 - DNA, 819 bp, Ambrosiozyma monospora
atgacagactacatacctacattcagattcgacggtcacttaactatcgtaactggtgcctgtggtggtttagcagaagcattgattaaaggtttgttagcctatggttcagatatagctttgttagatatcgaccaagaaaagactgctgcaaagcaagcagaatatcataagtacgccacagaagaattgaagttgaaggaagttccaaagatgggttcctacgcctgtgatatttctgattcagacaccgttcataaagtatttgcacaagtcgccaaagacttcggtaaattgcctttacacttggttaatactgctggttattgtgaaaactttccatgcgaagattaccctgctaaaaatgcagaaaagatggtaaaggtcaacttgttaggttccttatatgttagtcaagccttcgctaaaccattgatcaaggaaggtattaaaggtgcttccgttgtattaattggttccatgagtggtgcaatagtaaatgaccctcaaaaccaagtcgtttacaacatgagtaaggcaggtgtcatacacttagccaaaacattggcttgcgaatgggcaaagtacaacatcagagttaattctttgaacccaggttacatctacggtcctttgaccaaaaatgtaattaatggtaacgaagaattgtacaacagatggatttctggtataccacaacaaagaatgtcagaacctaaggaatacataggtgctgttttgtacttgttgtctgaatcagcagcctcctatacaacaggtgcttccttattggtagacggtggtttcacttcttggtag

SEQ ID NO: 96 - DNA, 735 bp, Mus musculus
atggatttgggtttggctggtagaagagcattggtaacaggtgctggtaaaggtatcggtagaagtacagtattggcattgaaggcagccggtgctcaagttgtagcagtttctagaaccagagaagatttggatgacttagttagagaatgtccaggtgtagaacctgtttgcgtagatttggctgactgggaagcaacagaacaagccttatcaaatgtaggtccagtcgatttgttagtaaataacgctgcagtcgcattgttgcaaccatttttggaagttacaaaggaagcttgtgacacctccttcaatgttaacttaagagcagttattcaagtaagtcaaatcgtcgccaagggtatgatcgctagaggtgtaccaggtgctattgtcaatgtttcttcacaagcttctcaaagagcattgactaaccatacagtttattgctcaactaaaggtgcattggatatgttaacaaagatgatggccttggaattaggtcctcacaaaattagagtcaatgccgttaacccaaccgtcgttatgactcctatgggtagaactaattggtccgatccacataaagcaaaggccatgttggacagaatacctttgggtaaattcgctgaagttgaaaacgtagtcgatacaattttattcttgttaagtaacagaagtggtatgacaacaggttcaacattgccagtagacggtggtttcttagcaacttag

SEQ ID NO: 97 - DNA, 735 bp, Cavia porcellus
atggacttaggtttggctggtagaagagcattggtcactggtgctggtaaaggtataggtagatccaccgtattggcattgaaggcagccggtgctcaagttgtagcagtttctagaaccagagaagatttggatgacttagttagagaatgtccaggtgtagaacctgtttgcgtagatttggctgactgggaagcaacagaacaagccttatcaaatgttggtccagctgacttgttagtcaataacgctgcagttgcattgttgcaaccatttttggaagttacaaaggaagcctgtgtaacctccttcaatgtcaacttaagagctgtaattcaagtcagtcaaatagtcgccaagggtatgatcgctagaggtgtaccaggtgctattgtcaatgtttcttcacaagcttctcaaagagcattgactaaccatacagtttattgctcaactaaaggtgcattgtacatgttaacaaagatgatggccttggaattaggtcctcacaaaattagagttaatgcagtaaacccaaccgtcgttatgactcctatgggtagaactaattggtccgatccacataaagcaaaggccatgttggacagaatacctttgggtaaattcgctgaagttgaaaacgtagtcgatacaattttattcttgttaagtaacagatctggtatgactactggttcaactttgcctgtcgacggtggtttcttggctacttag

SEQ ID NO: 98 - DNA, 21 bp, Artificial Sequence
  Synthetic Construct
gtttgctgtcttgctatcaag

SEQ ID NO: 99 - DNA, 22 bp, Artificial Sequence
  Synthetic Construct
caacgtatctaccaacgatttg

SEQ ID NO: 100 - DNA, 23 bp, Artificial Sequence
  Synthetic Construct
ctaattcgtagtttttcaagttc

SEQ ID NO: 101 - DNA, 21 bp, Artificial Sequence
  Synthetic Construct
ggacctagacttcaggttgtc

SEQ ID NO: 102 - DNA, 23 bp, Artificial Sequence
  Synthetic Construct
cctttcaaagttattctctactc

SEQ ID NO: 103 - DNA, 22 bp, Artificial Sequence
  Synthetic Construct
caagaaacaatacaatcatctc

SEQ ID NO: 104 - DNA, 24 bp, Artificial Sequence
  Synthetic Construct
gacggtaggtattgattgtaattc

SEQ ID NO: 105 - DNA, 19 bp, Artificial Sequence
  Synthetic Construct
ctttatttgagttgaaaag

SEQ ID NO: 106 - DNA, 23 bp, Artificial Sequence
  Synthetic Construct
cggtcttcaatttctcaagtttc

SEQ ID NO: 107 - DNA, 19 bp, Artificial Sequence
  Synthetic Construct
gagtacatttcaaatgcac

SEQ ID NO: 108 - DNA, 1475 bp, Saccharomyces cerevisiae
tgcctgcaggtcgagatccgggatcgaagaaatgatggtaaatgaaataggaaatcaaggagcatgaaggcaaaagacaaatataagggtcgaacgaaaaataaagtgaaaagtgttgatatgatgtatttggctttgcggcgccgaaaaaacgagtttacgcaattgcacaatcatgctgactctgtggcggacccgcgctcttgccggcccggcgataacgctgggcgtgaggctgtgcccggcggagttttttgcgcctgcattttccaaggtttaccctgcgctaaggggcgagattggagaagcaataagaatgccggttggggttgcgatgatgacgaccacgacaactggtgtcattatttaagttgccgaaagaacctgagtgcatttgcaacatgagtatactagaagaatgagccaagacttgcgagacgcgagtttgccggtggtgcgaacaatagagcgaccatgaccttgaaggtgagacgcgcataaccgctagagtactttgaagaggaaacagcaatagggttgctaccagtataaatagacaggtacatacaacactggaaatggttgtctgtttgagtacgctttcaattcatttgggtgtgcactttattatgttacaatatggaagggaactttacacttctcctatgcacatatattaattaaagtccaatgctagtagagaaggggggtaacacccctccgcgctcttttccgatttttttctaaaccgtggaatatttcggatatccttttgttgtttccgggtgtacaatatggacttcctcttttctggcaaccaaacccatacatcgggattcctataataccttcgttggtctccctaacatgtaggtggcggaggggagatatacaatagaacagataccagacaagacataatgggctaaacaagactacaccaattacactgcctcattgatggtggtacataacgaactaatactgtagccctagacttgatagccatcatcatatcgaagtttcactaccctttttccatttgccatctattgaagtaataataggcgcatgcaacttcttttctttttttttcttttctctctcccccgttgttgtctcaccatatccgcaatgacaaaaaaaatgatggaagacactaaaggaaaaaattaacgacaaagacagcaccaacagatgtcgttgttccagagctgatgaggggtatctcgaagcacacgaaactttttccttccttcattcacgcacactactctctaatgagcaacggtatacggccttccttccagttacttgaatttgaaataaaaaaaagtttgctgtcttgctatcaagtataaatagacctgcaattattaatcttttgtttcctcgtcattgttctcgttccctttcttccttgtttctttttctgcacaatatttcaagctataccaagcatacaatcaactcca

SEQ ID NO: 109 - DNA, 750 bp, Saccharomyces cerevisiae
acgcacagatattataacatctgcacaataggcatttgcaagaattactcgtgagtaaggaaagagtgaggaactatcgcatacctgcatttaaagatgccgatttgggcgcgaatcctttattttggcttcaccctcatactattatcagggccagaaaaaggaagtgtttccctccttcttgaattgatgttaccctcataaagcacgtggcctcttatcgagaaagaaattaccgtcgctcgtgatttgtttgcaaaaagaacaaaactgaaaaaacccagacacgctcgacttcctgtcttcctattgattgcagcttccaatttcgtcacacaacaaggtcctagcgacggctcacaggttttgtaacaagcaatcgaaggttctggaatggcgggaaagggtttagtaccacatgctatgatgcccactgtgatctccagagcaaagttcgttcgatcgtactgttactctctctctttcaaacagaattgtccgaatcgtgtgacaacaacagcctgttctcacacactcttttcttctaaccaagggggtggtttagtttagtagaacctcgtgaaacttacatttacatatatataaacttgcataaattggtcaatgcaagaaatacatatttggtcttttctaattcgtagtttttcaagttcttagatgctttctttttctcttttttacagatcatcaaggaagtaattatctactttttacaacaaatataaaaca

SEQ ID NO: 110 - DNA, 1000 bp, Saccharomyces cerevisiae
aatgctactattttggagattaatctcagtacaaaacaatattaaaaagaggtgaattatttttccccccttattttttttttgttaaaattgatccaaatgtaaataaacaatcacaaggaaaaaaaaaaaaaaaaaaaaaatagccgccatgaccccggatcgtcggttgtgatacggtcagggtagcgccctggtcaaacttcagaactaaaaaaataataaggaagaaaaaaatagctaatttttccggcagaaagattttcgctacccgaaagtttttccggcaagctaaatggaaaaaggaaagattattgaaagagaaagaaagaaaaaaaaaaaatgtacacccagacatcgggcttccacaatttcggctctattgttttccatctctcgcaacggcgggattcctctatggcgtgtgatgtctgtatctgttacttaatccagaaactggcacttgacccaactctgccacgtgggtcgttttgccatcgacagattgggagattttcatagtagaattcagcatgatagctacgtaaatgtgttccgcaccgtcacaaagtgttttctactgttctttcttctttcgttcattcagttgagttgagtgagtgctttgttcaatggatcttagctaaaatgcatattttttctcttggtaaatgaatgcttgtgatgtcttccaagtgatttcctttccttcccatatgatgctaggtacctttagtgtcttcctaaaaaaaaaaaaaggctcgccatcaaaacgatattcgttggcttttttttctgaattataaatactctttggtaacttttcatttccaagaacctcttttttccagttatatcatggtcccctttcaaagttattctctactctttttcatattcattctttttcatcctttggttttttattcttaacttgtttattattctctcttgtttctatttacaagacaccaatcaaaacaaataaaacatcatcaca

SEQ ID NO: 111 - DNA, 655 bp, Saccharomyces cerevisiae
agtttatcattatcaatactcgccatttcaaagaatacgtaaataattaatagtagtgattttcctaactttatttagtcaaaaaattagccttttaattctgctgtaacccgtacatgcccaaaatagggggcgggttacacagaatatataacatcgtaggtgtctgggtgaacagtttattcctggcatccactaaatataatggagcccgctttttaagctggcatccagaaaaaaaaagaatcccagcaccaaaatattgttttcttcaccaaccatcagttcataggtccattctcttagcgcaactacagagaacaggggcacaaacaggcaaaaaacgggcacaacctcaatggagtgatgcaacctgcctggagtaaatgatgacacaaggcaattgacccacgcatgtatctatctcattttcttacaccttctattaccttctgctctctctgatttggaaaaagctgaaaaaaaaggttgaaaccagttccctgaaattattcccctacttgactaataagtatataaagacggtaggtattgattgtaattctgtaaatctatttcttaaacttcttaaattctacttttatagttagtcttttttttagttttaaaacaccagaacttagtttcgacggat

SEQ ID NO: 112 - DNA, 412 bp, Saccharomyces cerevisiae
atagcttcaaaatgtttctactccttttttactcttccagattttctcggactccgcgcatcgccgtaccacttcaaaacacccaagcacagcatactaaatttcccctctttcttcctctagggtgtcgttaattacccgtactaaaggtttggaaaagaaaaaagagaccgcctcgtttctttttcttcgtcgaaaaaggcaataaaaatttttatcacgtttctttttcttgaaaatttttttttttgatttttttctctttcgatgacctcccattgatatttaagttaataaacggtcttcaatttctcaagtttcagtttcatttttcttgttctattacaactttttttacttcttgctcattagaaagaaagcatagcaatctaatctaagttttaattacaaa

SEQ ID NO: 113 - DNA, 324 bp, Saccharomyces cerevisiae
tggacttcttcgccagaggtttggtcaagtctccaatcaaggttgtcggcttgtctaccttgccagaaatttacgaaaagatggaaaagggtcaaatcgttggtagatacgttgttgacacttctaaataagcgaatttcttatgatttatgatttttattattaaataagttataaaaaaaataagtgtatacaaattttaaagtgactcttaggttttaaaacgaaaattcttgttcttgagtaactctttcctgtaggtcaggttgctttctcaggtatagcatgaggtcgctcttattgaccacacctctaccggcatgc

SEQ ID NO: 114 - DNA, 251 bp, Saccharomyces cerevisiae
atcatgtaattagttatgtcacgcttacattcacgccctccccccacatccgctctaaccgaaaaggaaggagttagacaacctgaagtctaggtccctatttatttttttatagttatgttagtattaagaacgttatttatatttcaaatttttcttttttttctgtacagacgcgtgtacgcatgtaacattatactgaaaaccttgcttgagaaggttttgggacgctcgaaggctttaatttgcgg

SEQ ID NO: 115 - DNA, 582 bp, Saccharomyces cerevisiae
ggtttgctgagaagcttgccaaatgattgactttataagaacggctgaccatggtagacggacccggttgatgggcttcatattgagatgattgtattgtttcttgacttctgagagtttttggttttttattatgttctccatgtctcggttcttacgttcgcattgttttatattttatttcatgtttatcaagagctctagaattcatagtcgaccggaccgatgccttcacaatttatagttttcattatcaagtatgcctatattagtatatagcatctttagatgacagtgttcgaagtttcacgaataaaagataatattctactttttgctcccctcgactttgttcccactgtacttttagctcgtacaaaatacaatatacttttcatttctccgtaaacaacatgttttcccatgtaatatccttttctatttttcgttccgttaccaactttacacatactttatatagctattcacttctatacactaaaaaactaagacaattttaattttgctgcctgccatatttcaatttgttataaattcctataatttatcctattagtagct

SEQ ID NO: 116 - DNA, 400 bp, Saccharomyces cerevisiae
aaaaagaatcatgattgaatgaagatattatttttttgaattatattttttaaattttatataaagacatggtttttcttttcaactcaaataaagatttataagttacttaaataacatacattttataaggtattctataaaaagagtattatgttattgttaacctttttgtctccaattgtcgtcataacgatgaggtgttgcatttttggaaacgagattgacatagagtcaaaatttgctaaatttgatccctcccatcgcaagataatcttccctcaaggttatcatgattatcaggatggcgaaaggatacgctaaaaattcaataaaaaattcaatataattttcgtttcccaagaactaacttggaaggttatacatgggtacataaatg

SEQ ID NO: 117 - DNA, 400 bp, Saccharomyces cerevisiae
tttgcgaacacttttattaattcatgatcacgctctaatttgtgcatttgaaatgtactctaattctaattttatatttttaatgatatcttgaaaagtaaatacgtttttaatatatacaaaataatacagtttaattttcaagtttttgatcatttgttctcagaaagttgagtgggacggagacaaagaaactttaaagagaaatgcaaagtgggaagaagtcagttgtttaccgaccgcactgttattcacaaatattccaattttgcctgcagacccacgtctacaaattttggttagtttggtaaatggtaaggatatagtagagcctttttgaaatgggaaatatcttctttttctgtatcccgcttcaaaaagtgtctaatgagtcagttat

SEQ ID NO: 118 - DNA, 600 bp, Saccharomyces cerevisiae
gtgtcgacgctgcgggtatagaaagggttctttactctatagtacctcctcgctcagcatctgcttcttcccaaagatgaacgcggcgttatgtcactaacgacgtgcaccaacttgcggaaagtggaatcccgttccaaaactggcatccactaattgatacatctacacaccgcacgccttttttctgaagcccactttcgtggactttgccatatgcaaaattcatgaagtgtgataccaagtcagcatacacctcactagggtagtttctttggttgtattgatcatttggttcatcgtggttcattaattttttttctccattgctttctggctttgatcttactatcatttggatttttgtcgaaggttgtagaattgtatgtgacaagtggcaccaagcatatataaaaaaaaaaagcattatcttcctaccagagttgattgttaaaaacgtatttatagcaaacgcaattgtaattaattcttattttgtatcttttcttcccttgtctcaatcttttatttttattttatttttcttttcttagtttctttcataacaccaagcaactaatactataacatacaataata

SEQ ID NO: 119 - DNA, 800 bp, Saccharomyces cerevisiae
catgcgactgggtgagcatatgttccgctgatgtgatgtgcaagataaacaagcaaggcagaaactaacttcttcttcatgtaataaacacaccccgcgtttatttacctatctctaaacttcaacaccttatatcataactaatatttcttgagataagcacactgcacccataccttccttaaaaacgtagcttccagtttttggtggttccggcttccttcccgattccgcccgctaaacgcatatttttgttgcctggtggcatttgcaaaatgcataacctatgcatttaaaagattatgtatgctcttctgacttttcgtgtgatgaggctcgtggaaaaaatgaataatttatgaatttgagaacaattttgtgttgttacggtattttactatggaataatcaatcaattgaggattttatgcaaatatcgtttgaatatttttccgaccctttgagtacttttcttcataattgcataatattgtccgctgcccctttttctgttagacggtgtcttgatctacttgctatcgttcaacaccaccttattttctaactattttttttttagctcatttgaatcagcttatggtgatggcacatttttgcataaacctagctgtcctcgttgaacataggaaaaaaaaatatataaacaaggctctttcactctccttgcaatcagatttgggtttgttccctttattttcatatttcttgtcatattcctttctcaattattattttctactcataacctcacgcaaaataacacagtcaaatcaatcaaa

SEQ ID NO: 120 - DNA, 820 bp, Saccharomyces cerevisiae
tccaactggcaccgctggcttgaacaacaataccagccttccaacttctgtaaataacggcggtacgccagtgccaccagtaccgttacctttcggtatacctcctttccccatgtttccaatgcccttcatgcctccaacggctactatcacaaatcctcatcaagctgacgcaagccctaagaaatgaataacaatactgacagtactaaataattgcctacttggcttcacatacgttgcatacgtcgatatagataataatgataatgacagcaggattatcgtaatacgtaatagttgaaaatctcaaaaatgtgtgggtcattacgtaaataatgataggaatgggattcttctatttttcctttttccattctagcagccgtcgggaaaacgtggcatcctctctttcgggctcaattggagtcacgctgccgtgagcatcctctctttccatatctaacaactgagcacgtaaccaatggaaaagcatgagcttagcgttgctccaaaaaagtattggatggttaataccatttgtctgttctcttctgactttgactcctcaaaaaaaaaaaatctacaatcaacagatcgcttcaattacgccctcacaaaaacttttttccttcttcttcgcccacgttaaattttatccctcatgttgtctaacggatttctgcacttgatttattataaaaagacaaagacataatacttctctatcaatttcagttattgttcttccttgcgttattcttctgttcttctttttcttttgtcatatataaccataaccaagtaatacatattcaaa

SEQ ID NO: 121 - DNA, 760 bp, Saccharomyces cerevisiae
tagtcgtgcaatgtatgactttaagatttgtgagcaggaagaaaagggagaatcttctaacgataaacccttgaaaaactgggtagactacgctatgttgagttgctacgcaggctgcacaattacacgagaatgctcccgcctaggatttaaggctaagggacgtgcaatgcagacgacagatctaaatgaccgtgtcggtgaagtgttcgccaaacttttcggttaacacatgcagtgatgcacgcgcgatggtgctaagttacatatatatatatatatatatatatatatatatatagccatagtgatgtctaagtaacctttatggtatatttcttaatgtggaaagatactagcgcgcgcacccacacacaagcttcgtcttttcttgaagaaaagaggaagctcgctaaatgggattccactttccgttccctgccagctgatggaaaaaggttagtggaacgatgaagaataaaaagagagatccactgaggtgaaatttcagctgacagcgagtttcatgatcgtgatgaacaatggtaacgagttgtggctgttgccagggagggtggttctcaacttttaatgtatggccaaatcgctacttgggtttgttatataacaaagaagaaataatgaactgattctcttcctccttcttgtcctttcttaattctgttgtaattaccttcctttgtaattttttttgtaattattcttcttaataatccaaacaaacacacatattacaata

SEQ ID NO: 122 - DNA, 430 bp, Saccharomyces cerevisiae
tatatctaggaacccatcaggttggtggaagattacccgttctaagacttttcagcttcctctattgatgttacacctggacaccccttttctggcatccagtttttaatcttcagtggcatgtgagattctccgaaattaattaaagcaatcacacaattctctcggataccacctcggttgaaactgacaggtggtttgttacgcatgctaatgcaaaggagcctatatacctttggctcggctgctgtaacagggaatataaagggcagcataatttaggagtttagtgaacttgcaacatttactattttcccttcttacgtaaatatttttctttttaattctaaatcaatctttttcaattttttgtttgtattcttttcttgcttaaatctataactacaaaaaacacatacataaactaaaa

SEQ ID NO: 123 - DNA, 400 bp, Saccharomyces cerevisiae
gtctgaagaatgaatgatttgatgatttctttttccctccatttttcttactgaatatatcaatgatatagacttgtatagtttattatttcaaattaagtagctatatatagtcaagataacgtttgtttgacacgattacattattcgtcgacatcttttttcagcctgtcgtggtagcaatttgaggagtattattaattgaataggttcattttgcgctcgcataaacagttttcgtcagggacagtatgttggaatgagtggtaattaatggtgacatgacatgttatagcaataaccttgatgtttacatcgtagtttaatgtacaccccgcgaattcgttcaagtaggagtgcaccaattgcaaagggaaaagctgaatgggcagttcgaata

SEQ ID NO: 124 - DNA, 1206 bp, Saccharomyces cerevisiae
atggcaaaccctttttcgagatggtttctatcagagagacctccaaactgccatgtagccgatttagaaacaagtttagatccccatcaaacgttgttgaaggtgcaaaaatacaaacccgctttaagcgactgggtgcattacatcttcttgggatccatcatgctgtttgtgttcattactaatcccgcaccttggatcttcaagatccttttttattgtttcttgggcactttattcatcattccagctacgtcacagtttttcttcaatgccttgcccatcctaacatgggtggcgctgtatttcacttcatcgtactttccagatgaccgcaggcctcctattactgtcaaagtgttaccagcggtggaaacaattttatacggcgacaatttaagtgatattcttgcaacatcgacgaattcctttttggacattttagcatggttaccgtacggactatttcattatggggccccatttgtcgttgctgccatcttattcgtatttggtccaccaactgttttgcaaggttatgcttttgcatttggttatatgaacctgtttggtgttatcatgcaaaatgtctttccagccgctcccccatggtataaaattctctatggattgcaatcagccaactatgatatgcatggctcgcctggtggattagctagaattgataagctactcggtattaatatgtatactacatgtttttcaaattcctccgtcattttcggtgcttttccttcactgcattccgggtgtgctactatggaagccctgtttttctgttattgttttccaaaattgaagcccttgtttattgcttatgtttgctggttatggtggtcaactatgtatctgacacaccattattttgtagaccttatggcaggttctgtgctgtcatacgttattttccagtacacaaagtacacacatttaccaattgtagatacatctcttttttgcagatggtcatacacttcaattgagaaatacgatatatcaaagagtgatccattggctgcagattcaaacgatatcgaaagtgtccctttgtccaacttggaacttgactttgatcttaatatgactgatgaacccagtgtaagcccttcgttatttgatggatctacttctgtttctcgttcgtccgccacgtctataacgtcactaggtgtaaagagggcttaa

SEQ ID NO: 125 - DNA, 810 bp, Saccharomyces cerevisiae
atgggtaaggaaaagactcacgtttcgaggccgcgattaaattccaacatggatgctgatttatatgggtataaatgggctcgcgataatgtcgggcaatcaggtgcgacaatctatcgattgtatgggaagcccgatgcgccagagttgtttctgaaacatggcaaaggtagcgttgccaatgatgttacagatgagatggtcagactaaactggctgacggaatttatgcctcttccgaccatcaagcattttatccgtactcctgatgatgcatggttactcaccactgcgatccccggcaaaacagcattccaggtattagaagaatatcctgattcaggtgaaaatattgttgatgcgctggcagtgttcctgcgccggttgcattcgattcctgtttgtaattgtccttttaacagcgatcgcgtatttcgtctcgctcaggcgcaatcacgaatgaataacggtttggttgatgcgagtgattttgatgacgagcgtaatggctggcctgttgaacaagtctggaaagaaatgcataagcttttgccattctcaccggattcagtcgtcactcatggtgatttctcacttgataaccttatttttgacgaggggaaattaataggttgtattgatgttggacgagtcggaatcgcagaccgataccaggatcttgccatcctatggaactgcctcggtgagttttctccttcattacagaaacggctttttcaaaaatatggtattgataatcctgatatgaataaattgcagtttcatttgatgctcgatgagtttttctaa

SEQ ID NO: 126 - DNA, 660 bp, Saccharomyces cerevisiae
atggagaaaaaaatcactggatataccaccgttgatatatcccaatggcatcgtaaagaacattttgaggcatttcagtcagttgctcaatgtacctataaccagaccgttcagctggatattacggcctttttaaagaccgtaaagaaaaataagcacaagttttatccggcctttattcacattcttgcccgcctgatgaatgctcatccggaattccgtatggcaatgaaagacggtgagctggtgatatgggatagtgttcacccttgttacaccgttttccatgagcaaactgaaacgttttcatcgctctggagtgaataccacgacgatttccggcagtttctacacatatattcgcaagatgtggcgtgttacggtgaaaacctggcctatttccctaaagggtttattgagaatatgtttttcgtctcagccaatccctgggtgagtttcaccagttttgatttaaacgtggccaatatggacaacttcttcgcccccgttttcaccatgggcaaatattatacgcaaggcgacaaggtgctgatgccgctggcgattcaggttcatcatgccgtctgtgatggcttccatgtcggcagaatgcttaatgaattacaacagtactgcgatgagtggcagggcggggcgtaa

SEQ ID NO: 127 - DNA, 1953 bp, Saccharomyces cerevisiae
atgagtgtgtctaccgccaagaggtcgctggatgtcgtttctccgggttcattagcggagtttgagggttcaaaatctcgtcacgatgaaatagaaaatgaacatagacgtactggtacacgtgatggcgaggatagcgagcaaccgaagaagaagggtagcaaaactagcaaaaagcaagatttggatcctgaaactaagcagaagaggactgcccaaaatcgggccgctcaaagagcttttagggaacgtaaggagaggaagatgaaggaattggagaagaaggtacaaagtttagagagtattcagcagcaaaatgaagtggaagctacttttttgagggaccagttaatcactctggtgaatgagttaaaaaaatatagaccagagacaagaaatgactcaaaagtgctggaatatttagcaaggcgagatcctaatttgcatttttcaaaaaataacgttaaccacagcaatagcgagccaattgacacacccaatgatgacatacaagaaaatgttaaacaaaagatgaatttcacgtttcaatatccgcttgataacgacaacgacaacgacaacagtaaaaatgtggggaaacaattaccttcaccaaatgatccaagtcattcggctcctatgcctataaatcagacacaaaagaaattaagtgacgctacagattcctccagcgctactttggattccctttcaaatagtaacgatgttcttaataacacaccaaactcctccacttcgatggattggttagataatgtaatatatactaacaggtttgtgtcaggtgatgatggcagcaatagtaaaactaagaatttagacagtaatatgttttctaatgactttaattttgaaaaccaatttgatgaacaagtttcggagttttgttcgaaaatgaaccaggtatgtggaacaaggcaatgtcccattcccaagaaacccatctcggctcttgataaagaagttttcgcgtcatcttctatactaagttcaaattctcctgctttaacaaatacttgggaatcacattctaatattacagataatactcctgctaatgtcattgctactgatgctactaaatatgaaaattccttctccggttttggccgacttggtttcgatatgagtgccaatcattacgtcgtgaatgataatagcactggtagcactgatagcactggtagcactggcaataagaacaaaaagaacaataataatagcgatgatgtactcccattcatatccgagtcaccgtttgatatgaaccaagttactaatttttttagtccgggatctaccggcatcggcaataatgctgcctctaacaccaatcccagcctactgcaaagcagcaaagaggatataccttttatcaacgcaaatctggctttcccagacgacaattcaactaatattcaattacaacctttctctgaatctcaatctcaaaataagtttgactacgacatgttttttagagattcatcgaaggaaggtaacaatttatttggagagtttttagaggatgacgatgatgacaaaaaagccgctaatatgtcagacgatgagtcaagtttaatcaagaaccagttaattaacgaagaaccagagcttccgaaacaatatctacaatcggtaccaggaaatgaaagcgaaatctcacaaaaaaatggcagtagtttacagaatgctgacaaaatcaataatggcaatgataacgataatgataatgaagtcgttccatctaaggaaggctctttactaaggtgttcggaaatttgggatagaataacaacacatccgaaatactcagatattgatgtcgatggtttatgttccgagctaatggcaaaggcaaaatgttcagaaagaggggttgtcatcaatgcagaagacgttcaattagctttgaataagcatatgaactaa

SEQ ID NO: 128 - DNA, 943 bp, Saccharomyces cerevisiae
tggaagctgaaacgtctaacggatcttgatttgtgtggacttccttagaagtaaccgaagcacaggcgctaccatgagaattgggtgaatgttgagataattgttgggattccattgttgataaaggctataatattaggtatacagaatatactagaagttctcgtttaaacggtccttttcatcacgtgctataaaaataattataatttaaattttttaatataaatatataaattaaaaatagaaagtaaaaaaagaaattaaagaaaaaatagtttttgttttccgaagatgtaaaagactctagggggatcgccaacaaatactaccttttatcttgctcttcctgctctcaggtattaatgccgaattgtttcatcttgtctgtgtagaagaccacacacgaaaatcctgtgattttacattttacttatcgttaatcgaatgtatatctatttaatctgcttttcttgtctaataaatatatatgtaaagtacgctttttgttgaaattttttaaacctttgtttatttttttttcttcattccgtaactcttctaccttctttatttactttctaaaatccaaatacaaaacataaaaataaataaacacagagtaaattcccaaattattccatcattaaaagatacgaggcgcgtgtaagttacaggcaagcgatccgtccgtttaaacctcgaggatataggaatcctcaaaatggaatctgcaattctacacaattctataaatattattatcatcattttatatgtttatattcattgatcctattacattatcaatccttgcgtttcagcttccactaatttagatgactatttctcatcatttgcgtcatcttctaacaccgtatatgataatatactagtaatgtaaatactagttagtagatgatagttgatttctattccaaca

SEQ ID NO: 129 - protein, 579 aa, Neurospora crassa
MSSHGSHDGASTEKHLATHDIAPTHDAIKIVPKGHGQTATKPGAQEKEVRNAALFAAIKESNIKPWSKESIHLYFAIFVAFCCACANGYDGSLMTGIIAMDKFQNQFHTGDTGPKVSVIFSLYTVGAMVGAPFAAILSDRFGRKKGMFIGGIFIIVGSIIVASSSKLAQFVVGRFVLGLGIAIMTVAAPAYSIEIAPPHWRGRCTGFYNCGWFGGSIPAACITYGCYFIKSNWSWRIPLILQAFTCLIVMSSVFFLPESPRFLFANGRDAEAVAFLVKYHGNGDPNSKLVLLETEEMRDGIRTDGVDKVWWDYRPLFMTHSGRWRMAQVLMISIFGQFSGNGLGYFNTVIFKNIGVTSTSQQLAYNILNSVISAIGALTAVSMTDRMPRRAVLIIGTFMCAAALATNSGLSATLDKQTQRGTQINLNQGMNEQDAKDNAYLHVDSNYAKGALAAYFLFNVIFSFTYTPLQGVIPTEALETTIRGKGLALSGFIVNAMGFINQFAGPIALHNIGYKYIFVFVGWDLIETVAWYFFGVESQGRTLEQLEWVYDQPNPVKASLKVEKVVVQADGHVSEAIVA

SEQ ID NO: 130 - protein, 476 aa, Neurospora crassa
MSLPKDFLWGFATAAYQIEGAIHADGRGPSIWDTFCNIPGKIADGSSGAVACDSYNRTKEDIDLLKSLGATAYRFSISWSRIIPVGGRNDPINQKGIDHYVKFVDDLLEAGITPFITLFHWDLPDGLDKRYGGLLNREEFPLDFEHYARTMFKAIPKCKHWITFNEPWCSSILGYNSGYFAPGHTSDRTKSPVGDSAREPWIVGHNLLIAHGRAVKVYREDFKPTQGGEIGITLNGDATLPWDPEDPLDVEACDRKIEFAISWFADPIYFGKYPDSMRKQLGDRLPEFTPEEVALVKGSNDFYGMNHYTANYIKHKKGVPPEDDFLGNLETLFYNKKGNCIGPETQSFWLRPHAQGFRDLLNWLSKRYGYPKIYVTENGTSLKGENAMPLKQIVEDDFRVKYFNDYVNAMAKAHSEDGVNVKGYLAWSLMDNFEWAEGYETRFGVTYVDYENDQKRYPKKSAKSLKPLFDSLIKKD

(19) United States
(12) Patent Application Publication
(10) Pub. No.: US 2013/0295631 A1
(43) Pub. Date: Nov. 7, 2013

(54) COMBINATORIAL DESIGN OF HIGHLY EFFICIENT HETEROLOGOUS PATHWAYS

(71) Applicants: Huimin Zhao, Champaign, IL (US); Byoungjin Kim, Urbana, IL (US); Jing Du, Champaign, IL (US); Yongbo Yuan, Urbana, IL (US); Dawn Eriksen, Urbana, IL (US); Tong Si, Urbana, IL (US)

(72) Inventors: Huimin Zhao, Champaign, IL (US); Byoungjin Kim, Urbana, IL (US); Jing Du, Champaign, IL (US); Yongbo Yuan, Urbana, IL (US); Dawn Eriksen, Urbana, IL (US); Tong Si, Urbana, IL (US)

(73) Assignee: The Board of Trustees of the University of Illinois, Urbana, IL (US)

(21) Appl. No.: 13/877,144

(22) PCT Filed: Oct. 3, 2011
(86) PCT No.: PCT/US11/54650

§ 371 Date: Jun. 21, 2013

Related U.S. Application Data
(60) Provisional application No. 61/389,109, filed on Oct. 1, 2010.

(51) Int. Cl. C12P 7/06

(52) U.S. Cl. CPC C12P 7/06
USPC 435/161; 435/254.21; 435/254.2; 435/254.23; 435/254.22; 435/254.6; 435/254.11; 435/254.4

(57) ABSTRACT

The present disclosure relates to the production of highly efficient heterologous pathways in host cells by identifying favorable enzyme and/or promoter combinations. In particular the present disclosure provides methods for assembly and selection of multi-step xylose and arabinose/xylose utilization pathways from a library of fungal enzymes. The present disclosure further provides compositions containing favorable enzyme combinations, as well as recombinant yeast expressing such combinations, and methods of use for bioconversion of pentose sugars. Also provided are compositions and methods involving favorable expression patterns identified by utilization of combinations of promoters of varying strengths. Provided herein are methods for assembly and selection of multi-step xylose, arabinose/xylose, and cellobiose utilization pathways from a library of promoters of varying strengths. The present disclosure further provides compositions containing heterologous enzyme-coding polynucleotides under the control of favorable promoters, as well as recombinant yeast expressing such enzymes, and methods of their use for bioconversion of pentose and/or hexose sugars.

### FIELD

The present disclosure relates to the production of highly efficient heterologous pathways in host cells by identifying favorable enzyme and/or promoter combinations. In particular the present disclosure provides methods for assembly and selection of multi-step xylose and arabinose/xylose utilization pathways from a library of fungal enzymes. The present disclosure further provides compositions containing favorable enzyme combinations, as well as recombinant yeast expressing such combinations, and methods of use for bioconversion of pentose sugars. Also provided are compositions and methods involving favorable expression patterns of heterologous enzymes identified by utilization of combinations of promoters of varying strengths. Provided herein are methods for assembly and selection of multi-step xylose, arabinose/xylose, and cellobiose utilization pathways from a library of polynucleotides encoding proteins of multi-step xylose, arabinose/xylose, and/or cellobiose utilization pathways under the control of promoters of varying strengths. The present disclosure further provides compositions containing heterologous enzyme-coding polynucleotides under the control of favorable promoters, as well as recombinant yeast expressing such enzymes, and methods of their use for bioconversion of pentose and/or hexose sugars.

### BACKGROUND

Biofuels are under intensive investigation due to increasing concerns about energy security, sustainability, and global climate change (Lynd et al., Nature Biotechnology, 26:169-172, 2008). Biological conversion of plant-derived lignocellulosic materials into biofuels has been regarded as an attractive alternative to chemical production of fossil fuels (Lynd et al., Science, 251:1318-1323, 1991; and Hahn-Hägerdal et al., Trends in Biotechnology, 24:549-556, 2006). Saccharomyces cerevisiae, also known as baker's yeast, has been used for bioconversion of hexose sugars into ethanol for thousands of years. It is also the most widely used microorganism for large scale industrial fermentation of glucose into ethanol. S. cerevisiae is an excellent organism for bioconversion of lignocellulosic biomass into biofuels (van Maris et al., Antonie van Leeuwenhoek, 90:391-418, 2006). It has a well-studied genetic and physiological background, ample genetic tools, and high tolerance to ethanol and inhibitors present in lignocellulosic hydrolysates (Jeffries et al., Current Opinion in Biotechnology, 17:320-326, 2006). Moreover, the low fermentation pH of S. cerevisiae can also prevent bacterial contamination. Lignocellulosic biomass is composed of cellulose, hemicellulose, and lignin. The hemicellulose component comprises 20-30% of lignocellulosic biomass, and it is primarily composed of five-carbon sugars (pentoses) such as xylose and arabinose (Saha, In Hemicellulose bioconversion, Springer-Verlag Berlin:279-291, 2003). Unfortunately, wild type S. cerevisiae can not utilize pentose sugars (Hector et al., Applied Microbiology and Biotechnology, 80:675-684, 2008).

To overcome this limitation, pentose utilization pathways from pentose-assimilating organisms have been introduced into S. cerevisiae, allowing fermentation of xylose and arabinose (Fonseca et al., FEBS Journal, 274:3589-3600, 2007; Brat et al., Applied and Environmental Microbiology, 75:2304-2311; 2009; Wisselink et al., Applied and Environmental Microbiology, 73:4881-4891, 2007; Wiedemann and Boles, Applied and Environmental Microbiology, 74:2043-2050, 2008; Wisselink et al., Applied and Environmental Microbiology, 75:907-914, 2009; Karhumaa et al., Microbial Cell Factories, 5:18, 2006; and Bettiga et al., Microbial Cell Factories, 8:40, 2009). However, pentose utilization by recombinant S. cerevisiae strains is inefficient due to the low expression level and activity of heterologous genes, redox imbalance resulting from different cofactor preference for oxidation and reduction reactions, and suboptimal metabolic flux through different catalytic steps (Hector et al., supra, 2008). A lot of research has been done to improve the pentose utilization in S. cerevisiae by targeting different aspects of these issues (Jin and Jeffries, Applied Biochemistry and Biotechnology, 105:277-285, 2003; Jin et al., and Applied and Environmental Microbiology, 69:495-503, 2003).

Implementation of concerted strategies to concurrently solve all three problems associated with pentose utilization by yeast has heretofore been unsuccessful. Thus what is needed in the art are improved technologies for production of yeast capable of efficiently catabolizing five-carbon sugars.

Furthermore, host cells such as yeast may be used for various other metabolic processes through the introduction of heterologous genes into the cell. For example, recently a heterologous pathway for cellobiose utilization in S. cerevisiae was developed (Li et al., Mol BioSyst 6, 2129-2132 (2010)). Similar to the problems associated with pentose utilization by recombinant S. cerevisiae strains, many heterologous pathways introduced into a host cell may be inefficient. Thus what is also needed in the art are improved technologies for production of yeast having efficient heterologous pathways for various metabolic processes.

### BRIEF SUMMARY

The present disclosure relates to the production of highly efficient heterologous pathways by identifying favorable enzyme and/or promoter combinations. In particular the present disclosure provides methods for assembly and selection of multi-step xylose and arabinose/xylose utilization pathways from a library of fungal enzymes. The present disclosure further provides compositions containing favorable enzyme combinations, as well as recombinant yeast expressing such combinations, and methods of use for bioconversion of pentose sugars. Also provided are compositions and methods involving favorable expression patterns of heterologous enzymes identified by utilization of combinations of promoters of varying strengths. Provided herein are methods for assembly and selection of multi-step xylose, arabinose/xylose, and cellobiose utilization pathways from a library of polynucleotides encoding proteins of multi-step xylose, arabinose/xylose, and/or cellobiose utilization pathways under the control of promoters of varying strengths. The present disclosure further provides compositions containing heterologous enzyme-coding polynucleotides under the control of favorable promoters, as well as recombinant yeast expressing such enzymes, and methods of their use for bioconversion of pentose and/or hexose sugars.

The present disclosure provides methods of preparing a library of nucleic acids encoding multi-enzyme pathways, comprising: a) providing: i) a first gene expression cassette for each of a plurality of homologues of a first enzyme, wherein the first gene expression cassette comprises an isolated nucleic acid comprising a coding region of the first enzyme in operable combination with a first heterologous promoter and a first heterologous terminator; ii) a second gene expression cassette for each of a plurality of homologues of a second enzyme, wherein the second gene expression cassette comprises an isolated nucleic acid comprising a coding region of the second enzyme in operable combination with a second heterologous promoter and a second heterologous terminator; iii) a third gene expression cassette for each of a plurality of homologues of a third enzyme, wherein the third gene expression cassette comprises an isolated nucleic acid comprising a coding region of the third enzyme, in operable combination with a third heterologous promoter, and a third heterologous terminator; and iv) a linearized yeast expression vector; wherein the first, second and third heterologous promoters comprise three different promoters, and the first, second and third heterologous terminators comprise three different terminators, and wherein an upstream homologous region is adjacent to the 5′ end of the promoters, and a downstream homologous region is a adjacent to the 3′ end of the terminators to facilitate homologous recombination of the gene expression cassettes into a site of interest in the yeast expression vector such that the first gene expression cassette is adjacent to the second gene expression cassette and the second third gene expression cassette is adjacent to the third gene expression cassette; and b) transforming yeast cells with the linearized yeast expression vector and the first, second and third gene expression cassettes to produce a recombinant yeast cell culture comprising a plurality of recombinant yeast cells each comprising a nucleic acid encoding a multi-enzyme pathway comprising one of each of the first, second and third gene expression cassettes adjacent to one another. In some embodiments, the methods further comprise step c) culturing the recombinant yeast cell culture under selective conditions comprising growth under oxygen-limited conditions in media containing a substrate utilized by the multi-enzyme pathway to produce a selected yeast cell culture enriched in a favorable combination of the first, second and third gene expression cassettes for utilization of the substrate. In some embodiments, recombinant yeast cell cultures comprising a favorable combination of gene expression cassettes produce a higher amount of product (e.g., ethanol) per gram substrate (at least 10%, 15%, 20%, 25%, 30%, 35%, 40%, 45%, 50%, 55%, 60%, 65%, 70%, 75%, 80%, 85%, 90%, 95%, 100%, 125%, 150%, 175%, or 200% greater product in grams/per gram substrate) as compared to a reference recombinant yeast cell culture comprising a reference multi-enzyme pathway. In some embodiments, the methods further comprise step d) isolating the nucleic acid encoding the multi-enzyme pathway from the selected yeast cell culture.

Also provided by the present disclosure are methods of preparing a library of nucleic acids encoding xylose utilization pathways, comprising: a) providing: i) a first gene expression cassette for each of a plurality of homologues of a xylose reductase, wherein the first gene expression cassette comprises an isolated nucleic acid comprising a coding region of the xylose reductase in operable combination with a first heterologous promoter and a first heterologous terminator; ii) a second gene expression cassette for each of a plurality of homologues of a xylitol dehydrogenase, wherein the second gene expression cassette comprises an isolated nucleic acid comprising a coding region of the xylitol dehydrogenase in operable combination with a second heterologous promoter and a second heterologous terminator; iii) a third gene expression cassette for each of a plurality of homologues of a xylulokinase, wherein the third gene expression cassette comprises an isolated nucleic acid comprising a coding region of the xylulokinase reductase, in operable combination with a third heterologous promoter, and a third heterologous terminator; and iv) a linearized yeast expression vector; wherein the first, second and third heterologous promoters comprise three different promoters, and the first, second and third heterologous terminators comprise three different terminators, and wherein an upstream homologous region is adjacent to the 5′ end of the promoters, and a downstream homologous region is a adjacent to the 3′ end of the terminators to facilitate homologous recombination of the gene expression cassettes into a site of interest in the yeast expression vector such that the first gene expression cassette is adjacent to the second gene expression cassette and the second gene expression cassette is adjacent to the third gene expression cassette; and b) transforming yeast cells with the linearized yeast expression vector and the first, second and third gene expression cassettes to produce a recombinant yeast cell culture comprising a plurality of recombinant yeast cells each comprising a nucleic acid encoding a xylose utilization pathway comprising one of each of the first, second and third gene expression cassettes adjacent to one another. In some embodiments, the methods further comprise step c) culturing the recombinant yeast cell culture under selective conditions comprising growth under oxygen-limited conditions in media containing xylose to produce a selected yeast cell culture enriched in a favorable combination of the first, second and third gene expression cassettes for anaerobic xylose catabolism. In some embodiments, recombinant yeast cell cultures comprising a favorable combination of gene expression cassettes produce a higher amount of product (e.g., ethanol) per gram xylose (at least 10%, 15%, 20%, 25%, 30%, 35%, 40%, 45%, 50%, 55%, 60%, 65%, 70%, 75%, 80%, 85%, 90%, 95%, 100%, 125%, 150%, 175%, or 200% greater product in grams/per gram xylose) as compared to a reference recombinant yeast cell culture comprising a reference xylose utilization pathway. An exemplary reference recombinant yeast cell culture comprises the scaffold of FIG. 2A. In some embodiments, the methods further comprise step d) isolating the nucleic acid encoding the xylose utilization pathway from the selected yeast cell culture.

Additionally, the present disclosure provides methods of preparing a library of nucleic acids encoding xylose/arabinose utilization pathways, comprising: a) providing: i) a first gene expression cassette for each of a plurality of homologues of a xylose reductase, wherein the first gene expression cassette comprises an isolated nucleic acid comprising a coding region of the xylose reductase in operable combination with a first heterologous promoter and a first heterologous terminator; ii) a second gene expression cassette for each of a plurality of homologues of a xylitol dehydrogenase, wherein the second gene expression cassette comprises an isolated nucleic acid comprising a coding region of the xylitol dehydrogenase in operable combination with a second heterologous promoter and a second heterologous terminator; iii) a third gene expression cassette for each of a plurality of homologues of a xylulokinase, wherein the third gene expression cassette comprises an isolated nucleic acid comprising a coding region of the xylulokinase reductase, in operable combination with a third heterologous promoter, and a third heterologous terminator; iv) a fourth gene expression cassette for each of a plurality of homologues of a L-arabitol 4-dehydrogenase, wherein the fourth gene expression cassette comprises an isolated nucleic acid comprising a coding region of the L-arabitol 4-dehydrogenase in operable combination with a fourth heterologous promoter and a fourth heterologous terminator; v) a fifth gene expression cassette for each of a plurality of homologues of a L-xylulose reductase, wherein the fifth gene expression cassette comprises an isolated nucleic acid comprising a coding region of the L-xylulose reductase in operable combination with a fifth heterologous promoter and a fifth heterologous terminator; and vi) a linearized yeast expression vector; wherein the first, second, third, fourth and fifth heterologous promoters comprise five different promoters, and the first, second, third, fourth and fifth heterologous terminators comprise five different terminators, and wherein an upstream homologous region is adjacent to the 5′ end of the promoters, and a downstream homologous region is a adjacent to the 3′ end of the terminators to facilitate homologous recombination of the gene expression cassette into a site of interest in the yeast expression vector such that the first gene expression cassette is adjacent to the second gene expression cassette, the second gene expression cassette is adjacent to the third gene expression cassette, the third gene expression cassette is adjacent to the fourth gene expression cassette, and the fourth gene expression cassette is adjacent to the fifth gene expression cassette; and b) transforming yeast cells with the linearized yeast expression vector and the first, second, third, fourth and fifth gene expression cassettes to produce a recombinant yeast cell culture comprising a plurality of recombinant yeast cells each comprising a nucleic acid encoding a xylose/arabinose utilization pathway comprising one of each of the first, second, third, fourth and fifth gene expression cassettes adjacent to one another. In some embodiments, the methods further comprise step c) culturing the recombinant yeast cell culture under selective conditions comprising growth under oxygen-limited conditions in media containing xylose and/or arabinose to produce a selected yeast cell culture enriched in a favorable combination of the first, second, third, fourth and fifth gene expression cassettes for anaerobic xylose and/or arabinose catabolism. In some embodiments, recombinant yeast cell cultures comprising a favorable combination of gene expression cassettes produce a higher amount of product (e.g., ethanol) per gram xylose and/or arabinose (at least 10%, 15%, 20%, 25%, 30%, 35%, 40%, 45%, 50%, 55%, 60%, 65%, 70%, 75%, 80%, 85%, 90%, 95%, 100%, 125%, 150%, 175%, or 200% greater product in grams/per gram xylose and/or arabinose) as compared to a reference recombinant yeast cell culture comprising a reference xylose utilization pathway. An exemplary reference recombinant yeast cell culture comprises the scaffold of FIG. 2B. In some embodiments, the methods further comprise step d) isolating the nucleic acid encoding the xylose/arabinose utilization pathway from the selected yeast cell culture.

Moreover the present disclosure provides isolated nucleic acids comprising coding regions of a xylose reductase, a xylitol dehydrogenase, and a xylulokinase, wherein each of the coding regions is in operable combination with a unique heterologous promoter and a unique heterologous terminator, and wherein each of the coding regions is of a different species. The heterologous promoters and terminators are unique in that they are different from other promoters and terminators, respectively of the isolated nucleic acid. In some embodiments, the coding regions of the nucleic acid are codon-optimized for expression in S. cerevisiae. In some embodiments, the xylose reductase coding region is of A. nidulans, the xylitol dehydrogenase coding region is of C. albicans, and the xylulokinase coding region is of S. cerevisiae. In a subset of these embodiments, the A. nidulans xylose reductase coding region encodes a polypeptide containing an amino acid sequence at least 90% identical to SEQ ID NO: 19, the C. albicans xylitol dehydrogenase coding region encodes a polypeptide containing an amino acid sequence at least 90% identical to SEQ ID NO: 24, and the S. cerevisiae xylulokinase coding region encodes a polypeptide containing an amino acid sequence at least 90% identical to SEQ ID NO: 49. In some embodiments, the xylose reductase coding region is of P. guilliermondii, the xylitol dehydrogenase coding region is of P. chrysogenum, and the xylulokinase coding region is of A. oryzae. In a subset of these embodiments, the P. guilliermondii xylose reductase coding region encodes a polypeptide containing an amino acid sequence at least 90% identical to SEQ ID NO: 7, the P. chrysogenum xylitol dehydrogenase coding region encodes a polypeptide containing an amino acid sequence at least 90% identical to SEQ ID NO: 30, and the A. oryzae xylulokinase coding region encodes a polypeptide containing an amino acid sequence at least 90% identical to SEQ ID NO: 60. In some embodiments, the xylose reductase coding region is of A. nidulans, the xylitol dehydrogenase coding region is of A. niger, and the xylulokinase coding region is of P. chrysogenum. In a subset of these embodiments, the A. nidulans xylose reductase coding region encodes a polypeptide containing an amino acid sequence at least 90% identical to SEQ ID NO: 19, the A. niger xylitol dehydrogenase coding region encodes a polypeptide containing an amino acid sequence at least 90% identical to SEQ ID NO: 36, and the P. chrysogenum xylulokinase coding region encodes a polypeptide containing an amino acid sequence at least 90% identical to SEQ ID NO: 47. In some embodiments, the xylose reductase coding region is of C. shehatae, the xylitol dehydrogenase coding region is of C. tropicalis, and the xylulokinase coding region is of P. pastoris. In a subset of these embodiments, the C. shehatae xylose reductase coding region encodes a polypeptide containing an amino acid sequence at least 90% identical to SEQ ID NO: 3, the C. tropicalis xylitol dehydrogenase coding region encodes a polypeptide containing an amino acid sequence at least 90% identical to SEQ ID NO: 38, and the P. pastoris xylulokinase coding region encodes a polypeptide containing an amino acid sequence at least 90% identical to SEQ ID NO: 50. In some embodiments, the xylose reductase coding region is of P. guilliermondii, the xylitol dehydrogenase coding regions of N. crassa, and the xylulokinase coding regions is of P. chrysogenum. In a subset of these embodiments, the P. guilliermondii xylose reductase coding region is at least 95% identical to SEQ ID NO:7, the N. crassa xylitol coding region is at least 95% identical to SEQ ID NO:27, and the P. chrysogenum xylulokinase coding region is at least 95% identical to SEQ ID NO:47. In other embodiments, the xylose reductase coding region is of A. oryzae, the xylitol dehydrogenase coding region is of N. crassa, and the xylulokinase coding region is of P. chrysogenum. In a subset of these embodiments, the A. oryzae xylose reductase coding region is at least 95% identical to SEQ ID NO:1, the N. crassa xylitol coding region is at least 95% identical to SEQ ID NO:27, and the P. chrysogenum xylulokinase coding region is at least 95% identical to SEQ ID NO:47. At least 90% identical indicates that the coding region of interest is at least 90%, 91%, 92%, 93%, 94%, 95%, 96%, 97%, 98%, 99% or 100% identical to the referenced SEQ ID NO.

Also provided by the present disclosure are isolated nucleic acids comprising coding regions of a xylose reductase, a xylitol dehydrogenase, a xylulokinase, a xylose-specific transporter, a transaldolase and a transketolase, wherein each of the coding regions is in operable combination with a unique heterologous promoter and a unique heterologous terminator, and wherein the coding regions are from at least two different species. The heterologous promoters and terminators are unique in that they are different from other promoters and terminators, respectively of the isolated nucleic acid. In some embodiments, the different species comprise at least two or three different fungal species. In some preferred embodiments, the coding regions of the nucleic acid are codon-optimized for expression in S. cerevisiae.

The present disclosure also provides isolated nucleic acids comprising coding regions of a xylose reductase, a xylitol dehydrogenase, a xylulokinase, an L-arabitol 4-dehydrogenase, and a L-xylulose reductase, wherein each of the coding regions is in operable combination with a unique heterologous promoter and a unique heterologous terminator, and wherein the coding regions are from at least two different species. The heterologous promoters and terminators are unique in that they are different from other promoters and terminators, respectively of the isolated nucleic acid. In some embodiments, the different species comprise at least two or three different fungal species. In some preferred embodiments, the coding regions of the nucleic acid are codon-optimized for expression in S. cerevisiae.

Also provided by the present disclosure are isolated nucleic acids comprising coding regions of a xylose reductase, a xylitol dehydrogenase, a xylulokinase, an L-arabitol 4-dehydrogenase, and a L-xylulose reductase, a xylose-specific transporter, an arabinose-specific transporter, a transaldolase and a transketolase wherein each of the coding regions is in operable combination with a unique heterologous promoter and a unique heterologous terminator, and wherein the coding regions are from at least two different species. The heterologous promoters and terminators are unique in that they are different from other promoters and terminators, respectively of the isolated nucleic acid. In some embodiments, the different species comprise at least two or three different fungal species. In some preferred embodiments, the coding regions of the nucleic acid are codon-optimized for expression in S. cerevisiae.

In addition, the present disclosure provides vectors comprising the isolated nucleic acid of any one of the preceding paragraphs. In some embodiments, the vector is selected from the group consisting of an integrative plasmid, a centromeric plasmid, and a episomal plasmid. In further embodiments, the present disclosure provides a host cell comprising the vector. In some preferred embodiments, the host cell is of a microorganism selected from the group consisting of Saccharomyces cerevisiae, Saccharomyces monacensis, Saccharomyces bayanus, Saccharomyces pastorianus, Saccharomyces carlsbergensis, Saccharomyces pombe, Kluyveromyces marxiamus, Kluyveromyces lactis, Kluyveromyces fragilis, Pichia stipitis, Sporotrichum thermophile, Candida shehatae, Candida tropicalis, Neurospora crassa, Trichoderma reesei, and Zymomonas mobilis. In some embodiments, the yeast grows anaerobically on xylose and/or arabinose as a main carbon source at a greater rate than a parental yeast strain from which it was derived and which lacks the vector. Moreover, the present disclosure provides method for productions of ethanol comprising culturing the host cells in a composition comprising xylose and/or arabinose, under conditions suitable for the production of ethanol. In some aspects, the composition comprising xylose and/or arabinose includes plant biomass hydrolysate. In some embodiments, the methods further comprise recovering the ethanol from the culture medium.

The present disclosure also provides methods of preparing a library of gene expression cassettes, comprising: a) amplifying a coding region of an enzyme with a primer pair comprising a forward primer and a reverse primer to produce an amplified coding region, the forward primer comprising a 5′ overhang identical to the 3′ end of a heterologous promoter, and the reverse primer comprising a 5′ overhang identical to the reverse complement of the 3′ end of a heterologous terminator; b) digesting a helper plasmid with a restriction endonuclease to produce a linearized helper plasmid, wherein the helper plasmid comprises the promoter separated from the terminator by a sole recognition site for the restriction endonuclease; c) transforming a yeast cell with the linearized helper plasmid and the amplified coding region to produce a recombinant yeast cell comprising a circular plasmid containing a gene expression cassette comprising the coding region in operable combination with the promoter and the terminator; and d) repeating steps (a) to (c) for each of a plurality of homologues of the enzyme, to produce a library of gene expression cassettes. In some embodiments, the enzyme comprises one or more of the group consisting of a xylose reductase, a xylitol dehydrogenase, a xylulokinase, a L-arabitol 4-dehydrogenase, and a L-xylulose reductase. In some embodiments, an upstream homologous region is adjacent to the 5′ end of the heterologous promoter of the helper plasmid, and a downstream homologous region is a adjacent to the 3′ end of the heterologous terminator of the helper plasmid to facilitate incorporation by homologous recombination of the gene expression cassette into a site of interest in an expression vector.

The present disclosure also provides a method of preparing a library of nucleic acids encoding cellobiose utilization pathways, comprising: a) providing: i) a plurality of first gene expression cassettes for a cellobiose transporter, wherein each of said first gene expression cassettes comprises an isolated nucleic acid comprising a coding region of said cellobiose transporter in operable combination with a first heterologous promoter and a first heterologous terminator; ii) a plurality of second gene expression cassettes for a beta-glucosidase, wherein each of said second gene expression cassettes comprises an isolated nucleic acid comprising a coding region of said beta-glucosidase in operable combination with a second heterologous promoter and a second heterologous terminator; and iii) a linearized yeast expression vector; wherein said first and second heterologous promoters comprise two different promoters, and said first and second heterologous terminators comprise two different terminators, and wherein each of said first and second heterologous promoters comprise a mutation with respect to another of said first and second heterologous promoters of said plurality such that said mutation results in a change in relative expression levels of one of said cellobiose transporter and beta-glucosidase, and wherein an upstream homologous region is adjacent to the 5′ end of said promoters, and a downstream homologous region is a adjacent to the 3′ end of said terminators to facilitate homologous recombination of said gene expression cassettes into a site of interest in said yeast expression vector such that said first gene expression cassette is adjacent to said second gene expression cassette; and b) transforming yeast cells with said linearized yeast expression vector and said first and second gene expression cassettes to produce a recombinant yeast cell culture comprising a plurality of recombinant yeast cells each comprising a nucleic acid encoding a cellobiose utilization pathway comprising one of each of said first and second gene expression cassettes adjacent to one another. In some aspects, the method may further comprise step c) culturing said recombinant yeast cell culture under selective conditions comprising growth under oxygen-limited conditions in media containing cellobiose to produce a selected yeast cell culture enriched in a favorable combination of said first and second gene expression cassettes for anaerobic cellobiose catabolism. In some aspects, the method may further comprise step d) isolating said nucleic acid encoding said cellobiose utilization pathway from said selected yeast cell culture. In some aspects, the heterologous promoters include at two from the group consisting of an ENO2 promoter, a PDC1 promoter, a FBA1 promoter, a GPM1 promoter, a TPI1 promoter, and a TEF1 promoter.

In addition, the present disclosure provides methods of preparing a library of nucleic acids encoding xylose utilization pathways, comprising: a) providing: i) a plurality of first gene expression cassettes for a xylose reductase, wherein each of the first gene expression cassettes comprises an isolated nucleic acid comprising a coding region of the xylose reductase in operable combination with a first heterologous promoter and a first heterologous terminator; ii) a plurality of second gene expression cassettes for a xylitol dehydrogenase, wherein each of the second gene expression cassettes comprises an isolated nucleic acid comprising a coding region of the xylitol dehydrogenase in operable combination with a second heterologous promoter and a second heterologous terminator; iii) a plurality of third gene expression cassettes for a xylulokinase, wherein the third gene expression cassette comprises an isolated nucleic acid comprising a coding region of the xylulokinase reductase, in operable combination with a third heterologous promoter, and a third heterologous terminator; and iv) a linearized yeast expression vector; wherein the first, second and third heterologous promoters comprise three different promoters, and the first, second and third heterologous terminators comprise three different terminators, and wherein each of the first, second and third heterologous promoters comprise a mutation with respect to another of the first, second and third heterologous promoters of the plurality such that the mutation results in a change in relative expression levels of one of the xylose reductase, xylitol dehydrogenase, and xylulokinase reductase, and wherein an upstream homologous region is adjacent to the 5′ end of the promoters, and a downstream homologous region is a adjacent to the 3′ end of the terminators to facilitate homologous recombination of the gene expression cassettes into a site of interest in the yeast expression vector such that the first gene expression cassette is adjacent to the second gene expression cassette and the second third gene expression cassette is adjacent to the third gene expression cassette; and b) transforming yeast cells with the linearized yeast expression vector and the first, second and third gene expression cassettes to produce a recombinant yeast cell culture comprising a plurality of recombinant yeast cells each comprising a nucleic acid encoding a xylose utilization pathway comprising one of each of the first, second and third gene expression cassettes adjacent to one another. In some embodiments, the methods further comprise step c) culturing the recombinant yeast cell culture under selective conditions comprising growth under oxygen-limited conditions in media containing xylose to produce a selected yeast cell culture enriched in a favorable combination of the first, second and third gene expression cassettes for anaerobic xylose catabolism, as compared to a reference recombinant yeast cell culture comprising a reference xylose utilization pathway. An exemplary reference recombinant yeast cell culture comprises the scaffold of FIG. 2A. In some embodiments, the methods further comprise step d) isolating the nucleic acid encoding the xylose utilization pathway from the selected yeast cell culture. In some embodiments, the heterologous promoters comprise three from the group consisting of an ENO2 promoter, a PDC1 promoter, a FBA1 promoter, a GPM1 promoter, a TPI1 promoter, and a TEF1 promoter.

Moreover the present invention provides methods of preparing a library of nucleic acids encoding xylose/arabinose utilization pathways, comprising: a) providing: i) a plurality of first gene expression cassettes for a xylose reductase, wherein each of the first gene expression cassettes comprises an isolated nucleic acid comprising a coding region of the xylose reductase in operable combination with a first heterologous promoter and a first heterologous terminator; ii) a plurality of second gene expression cassettes for a xylitol dehydrogenase, wherein each of the second gene expression cassettes comprises an isolated nucleic acid comprising a coding region of the xylitol dehydrogenase in operable combination with a second heterologous promoter and a second heterologous terminator; iii) a plurality of third gene expression cassettes for a xylulokinase, wherein the third gene expression cassette comprises an isolated nucleic acid comprising a coding region of the xylulokinase reductase, in operable combination with a third heterologous promoter, and a third heterologous terminator; iv) a plurality of fourth gene expression cassettes for an L-arabitol 4-dehydrogenase, wherein the fourth gene expression cassette comprises an isolated nucleic acid comprising a coding region of the L-arabitol 4-dehydrogenase, in operable combination with a fourth heterologous promoter, and a fourth heterologous terminator; v) a plurality of fifth gene expression cassettes for a L-xylulose reductase, wherein the fifth gene expression cassette comprises an isolated nucleic acid comprising a coding region of the L-xylulose reductase, in operable combination with a fifth heterologous promoter, and a fifth heterologous terminator; and vi) a linearized yeast expression vector; wherein the first, second, third, fourth and fifth heterologous promoters comprise five different promoters, and the first, second, third, fourth and fifth heterologous terminators comprise five different terminators, and wherein each of the first, second, third, fourth and fifth heterologous promoters comprise a mutation with respect to another of the first, second, third, fourth and fifth heterologous promoters of the plurality such that the mutation results in a change in relative expression levels of one of the xylose reductase, xylitol dehydrogenase, xylulokinase reductase, L-arabitol 4-dehydrogenase and, and wherein an upstream homologous region is adjacent to the 5′ end of the promoters, and a downstream homologous region is a adjacent to the 3′ end of the terminators to facilitate homologous recombination of the gene expression cassettes into a site of interest in the yeast expression vector such that the first gene expression cassette is adjacent to the second gene expression cassette, the second gene expression cassette is adjacent to the third gene expression cassette, the third gene expression cassette is adjacent to the fourth gene expression cassette, and the fourth gene expression cassette is adjacent to the fifth gene expression cassette; and b) transforming yeast cells with the linearized yeast expression vector and the first, second, third, fourth and fifth gene expression cassettes to produce a recombinant yeast cell culture comprising a plurality of recombinant yeast cells each comprising a nucleic acid encoding a xylose/arabinose utilization pathway comprising one of each of the first, second, third, fourth and fifth gene expression cassettes adjacent to one another. In some embodiments, the methods further comprise step c) culturing the recombinant yeast cell culture under selective conditions comprising growth under oxygen-limited conditions in media containing xylose and/or arabinose to produce a selected yeast cell culture enriched in a favorable combination of the first, second, third, fourth and fifth gene expression cassettes for anaerobic xylose and/or arabinose catabolism as compared to a reference recombinant yeast cell culture comprising a reference xylose utilization pathway. An exemplary reference recombinant yeast cell culture comprises the scaffold of FIG. 2B. In some embodiments, the methods further comprise step d) isolating the nucleic acid encoding the xylose/arabinose utilization pathway from the selected yeast cell culture. In some embodiments, the heterologous promoters comprise five from the group consisting of an ENO2 promoter, a PDC1 promoter, a FBA1 promoter, a GPM1 promoter, a TPI1 promoter, and a TEF1 promoter.

The present disclosure also provides methods of preparing a library of nucleic acids encoding multi-enzyme pathways, comprising: a) providing: i) a plurality of first gene expression cassettes for a first enzyme, wherein each of the first gene expression cassettes comprises an isolated nucleic acid comprising a coding region of the first enzyme in operable combination with a first heterologous promoter and a first heterologous terminator; ii) a plurality of second gene expression cassettes for a second enzyme, wherein each of the second gene expression cassettes comprises an isolated nucleic acid comprising a coding region of the second enzyme in operable combination with a second heterologous promoter and a second heterologous terminator; iii) a plurality of third gene expression cassettes for a third enzyme, wherein the third gene expression cassette comprises an isolated nucleic acid comprising a coding region of the third enzyme, in operable combination with a third heterologous promoter, and a third heterologous terminator; and iv) a linearized yeast expression vector; wherein the first, second and third heterologous promoters comprise three different promoters, and the first, second and third heterologous terminators comprise three different terminators, and wherein each of the first, second and third heterologous promoters comprise a mutation with respect to another of the first, second and third heterologous promoters of the plurality such that the mutation results in a change in relative expression levels of one of the first, second and third enzymes, and wherein an upstream homologous region is adjacent to the 5′ end of the promoters, and a downstream homologous region is a adjacent to the 3′ end of the terminators to facilitate homologous recombination of the gene expression cassettes into a site of interest in the yeast expression vector such that the first gene expression cassette is adjacent to the second gene expression cassette and the second third gene expression cassette is adjacent to the third gene expression cassette; and b) transforming yeast cells with the linearized yeast expression vector and the first, second and third gene expression cassettes to produce a recombinant yeast cell culture comprising a plurality of recombinant yeast cells each comprising a nucleic acid encoding a multi-enzyme pathway comprising one of each of the first, second and third gene expression cassettes adjacent to one another. In some embodiments, the methods further comprise step c) culturing the recombinant yeast cell culture under selective conditions comprising growth under oxygen-limited conditions in media containing a substrate of the pathway to produce a selected yeast cell culture enriched in a favorable combination of the first, second and third gene expression cassettes for anaerobic utilization of the substrate, as compared to a reference recombinant yeast cell culture comprising a reference multi-enzyme pathway. In some embodiments, the methods further comprise step d) isolating the nucleic acid encoding the multi-enzyme pathway from the selected yeast cell culture. In some embodiments, the heterologous promoters include three from the group consisting of an ENO2 promoter, a PDC1 promoter, a FBA1 promoter, a GPM1 promoter, a TPI1 promoter, and a TEF1 promoter.

The disclosure also provides an isolated nucleic acid comprising coding regions of a cellobiose transporter and a beta glucosidase, wherein each of the coding regions is in operable combination with a unique heterologous promoter and a unique heterologous terminator. In some embodiments, the cellobiose transporter and beta glucosidase coding region is of N. crassa. In a subset of these embodiments, the N. crassa cellobiose transporter coding region encodes a polypeptide containing an amino acid sequence at least 90% identical to SEQ ID NO: 129 and the N. crassa beta glucosidase coding region encodes a polypeptide containing an amino acid sequence at least 90% identical to SEQ ID NO: 130. In some aspects, each heterologous promoter of the isolated nucleic acid has a non-naturally occurring nucleotide sequence. In addition, the present disclosure provides vectors comprising an isolated nucleic acid comprising coding regions of a cellobiose transporter and a beta glucosidase, wherein each of the coding regions is in operable combination with a unique heterologous promoter and a unique heterologous terminator. In some embodiments, the vector is selected from the group consisting of an integrative plasmid, a centromeric plasmid, and a episomal plasmid. In further embodiments, the present disclosure provides a host cell comprising the vector. In some preferred embodiments, the host cell is of a microorganism selected from the group consisting of Saccharomyces cerevisiae, Saccharomyces monacensis, Saccharomyces bayanus, Saccharomyces pastorianus, Saccharomyces carlsbergensis, Saccharomyces pombe, Kluyveromyces marxiamus, Kluyveromyces lactis, Kluyveromyces fragilis, Pichia stipitis, Sporotrichum thermophile, Candida shehatae, Candida tropicalis, Neurospora crassa, Trichoderma reesei, and Zymomonas mobilis. In some embodiments, the yeast grows anaerobically on cellobiose as a main carbon source at a greater rate than a parental yeast strain from which it was derived and which lacks the vector. Moreover, the present disclosure provides method for productions of ethanol comprising culturing the host cells in a composition comprising cellobiose, under conditions suitable for the production of ethanol. In some aspects, the composition comprising cellobiose includes plant biomass hydrolysate. In some embodiments, the methods further comprise recovering the ethanol from the culture medium.

### BRIEF DESCRIPTION OF THE DRAWINGS

FIG. 1 shows a scheme for the combinatorial pathway design strategy in a pRS416 backbone. pRS416 is a single-copy shuttle vector for cloning of genes in S. cerevisiae, which is also capable of replication in E. coli (New England Biolabs, Ipswich, Mass.). General scaffolds for the three-gene xylose utilization pathway (1A) and the five-gene arabinose/xylose utilization pathway (1B) were constructed using fungal and other nucleic acid templates. The overlap between adjacent expression cassettes was on the order of 500 to 1000 bp (e.g., about 500 bp to 1.2 kb): about 500 bp between the first promoter and last terminator to the vector backbone; and about 1 kb between enzyme coding regions. The size of the overlap varied due to the use of promoters and terminators of different lengths. All of the DNA fragments except for the vector backbone were generated by single PCR reactions.

FIG. 2A shows the pHZ981 scaffold used for combinatorial design of a three enzyme xylose utilization pathway. FIG. 2B shows the pHZ1002 scaffold used for combinatorial design of a five enzyme xylose/arabinose utilization pathway. The scaffolds were formed by assembling gene expression cassettes into a linearized pRS416 vector by using the DNA assembler method (Shao et al., Nucleic Acids Research, 37:e16, 2009).

FIG. 3 illustrates the assembly of individual gene expression cassettes into a helper plasmid having a pRS414 backbone.

FIG. 4A-B shows the optimal amount of DNA fragments needed for library creation. It was determined to be around 5,000 ng in total, and the resulting library size was around 1.3×10⁴ (transformants/μg DNA).

FIG. 5A-C shows the genetic diversity of various xylose assembly libraries.

FIG. 6A-D shows analyses of cell growth and metabolism of the S. cerevisiae control strain (expressing XR, XDH, and XKS from P. stipitis) and clones isolated through enrichment. (a) Comparison of the cell growth of the control strain and 10 clones from the second and third round of enrichments; (b), (c), and (d) OD (solid diamond), xylose (solid rectangle), xylitol (solid triangle), glycerol (cross), acetate (solid circle), and ethanol (empty circle) concentrations in the culture of (b) control, (c) clone E2.1, and (d) E3.2.

FIG. 7 shows a comparison of the (a) ethanol and (b) xylitol yields in g/g xylose of the recombinant E2.1 and E3.2 clones with that of the control strain expressing P. stipitis XR, XDH, and XKS. E2.# and E3.# represents clones isolated after two and three rounds of enrichment, respectively. In each group of three bars, the left bar is psXP, the middle bar is E2.1, and the right bar is E3.2.

FIG. 8 illustrates the scheme for optimizing the three gene xylose utilization pathway using promoters with varying strengths. A different promoter is used for each pathway enzyme. For the same pathway enzyme, mutants of the same promoter with varying strength are introduced into the cell. Using the DNA assembler method we developed previously, expression cassettes of different pathway enzymes are assembled into a full xylose utilization pathway, resulting in varied expression level of each pathway enzyme.

FIG. 9A provides maps of a single copy vector into which a pentose utilization pathway can be introduced using the DNA assembler method. FIG. 9B provides maps of a multi-copy delta-integrative vector into which a pentose utilization pathway can be introduced using the DNA assembler method. After digesting with rare cutting restriction endonucleases to release the CEN.ARS fragment, the linearized vector is integrated into the delta site of a yeast host strain via homologous recombination. Yeast cells harboring multiple copies of the pentose pathway are obtained by high dose drug selection.

FIG. 10 Enrichment of the control pathway with the pathway library itself in parallel. Diamond: final OD after every 48 hours of culture for the strain with psXR-psXDH-psXKS single copy integration in the genome; triangle: final OD after every 48 hours for E3.2 pathway on single copy plasmid; square: final OD after every 48 hours for E3.2 pathway on single copy chromosomal integration. Ethanol yields after four rounds of enrichment are indicated.

FIG. 11 Enrichment with re-transformation after every two rounds of enrichment. A. Scheme of the library enrichment strategy, B. Final OD of each culture in the YP media supplemented with xylose. Before even round numbers, the yeast plasmids were isolated and retransferred into fresh host cells. C. Plot of the final OD of the cultures right after re-transformation, indicating that the cell growth rate didn't improve after rounds of enrichment.

FIG. 12 Enrichment with re-transformation after every round of enrichment. Cell density (left) and xylose consumption (right) at the end of each round of enrichment. Diamond: INVSc1 with ps-pathway fresh plasmid; square: INVSc1 with ps-pathway enriched with library; triangle: INVSc1 with pathway library

FIG. 13 Relationship between growth rate and colony size distribution. Left: Growth rate of yeast strains harboring different pathway mutants. Inv.lib.1 to inv.lib.8 are the eight random picked strains with different growth rate. Inv.lib.1 to inv.lib.5 are the five strains plated on xylose plate for colony size check. Right: Distribution of colony size of 50 random picked yeast colonies. Plate 1 to plate 5 correspond to the colony size of inv.lib.1 to inv.lib.5. In the graph, the order of the plates, starting at the front, are: 1, 2, 5, 4, and 3. The numbers and black circles shown on the plot left indiate the plate number used to inoculate each liquid culture and the diameter of the black circle represents relative average colony sizes on each plate. Black arrow on the plot right indicates the direction of the increasing size of large colones on each plate and the arrow with the question mark indicates the deviation from hear correlation between colony size and growth rate in liquid media. The plate 5 had the largest (average) colony size, but the clone picked from the plate 5 showed median growth rate (on the left).

FIG. 14 Screening strategy based on colony size. Screening strategy based on colony size. The pathway library is spread on an agar plate containing 2% xylose as the sole carbon source together with a reference pathway consisting of a xylose utilization consisting of psXR, psXDH and psXKS. Colonies on the library plate that have grown to a size bigger than that of the largest colonies on the reference plate are picked and inoculated in media supplemented with 2% and the necessary selection pressure for maintaining the pathway bearing plasmids. The seed cultures are then used to inoculate tubes containing YP media supplemented with 2% xylose to a similar initial OD. Mutant strains bearing fast xylose utilizing pathways are identified by measuring the cell growth rates of the mutant strains. The top ten mutant strains identified using tube cultures are screened again in 50 mL flasks containing 10 ml YP media supplemented with 2% xylose. Flask cultures are analyzed using HPLC and the top mutant strain with the fastest xylose utilization rate and the highest ethanol productivity is identified.

FIG. 15 Specific growth rates and xylose consumption and ethanol yield of the selected recombinants of InvSc1 strain. A) specific growth rates of 80 recombinants selected by the colony size between 20 and 32 hrs culture in YPX (2%) media under aerobic condition. The clones selected for the next screening were shown in dark black. B) Xylose consumption and ethanol yields of the selected 10 clones after 42 hrs in YPX (2%) media under oxygen-limited condition.

FIG. 16 Xylose fermentation profiles of InvSc1 strain expressing control (P. stipitis pathway (psXR-psXDH-psXKS), left) and screened S2 (anXR-caXDH-scXKS) (right) pathways on a single copy plasmid. Square: xylose concentration; diamond: cell density (measured by optical density at 600 nm); triangle: ethanol concentration. The data shown is the mean of the duplicates, and the standard deviation is within 20%.

FIG. 17 Enzyme activities of enzyme homologues. A. Activity of xylose reductase homologues from different sources. In each column pair, the left column shows the activity when NADPH is used as a cofactor, while the right column shows the activity when NADH is used as a cofactor. B. Activity of xylitol dehydrogenases homologues from different sources. The upper portion of each column (lighter gray) shows the activity when NAD is used as a cofactor (primary Y-axis), while the lower portion of each column (darker gray/black) shows the activity when NADP is used as a cofactor (secondary Y-axis). C. Activity of xylulokinase homologues from different sources. All enzyme activity measurements were done, at the very least, in duplication. The error bar indicates the standard deviation of replicated samples. Based on this result, the xylose reductase from Candida shehatae (csXR), the NAD⁺-specific xylitol dehydrogenase from Candida tropicalis (ctXDH), and the xylulokinase from Pichia pastoris (ppXKS) were selected to construct the xylose utilizing pathway in both laboratory and industrial yeast strains.

FIG. 18 Alignment of cloned ppXKS amino acid sequence with its reference sequence from the NCBI database The cloned ppXKS only shares 93% sequence identity with its reference protein. To further verify that the origin of the cloned ppXKS is actually from cDNA isolated from Pichia pastoris and not due to contamination, the amino acid sequence of the cloned ppXKS was subjected to a BLAST search of the non-redundant protein sequence database at NCBI. The result from the BLAST search showed that the top hit with the highest score is indeed the xylulokinase from Pichia pastoris, indicating that the ppXKS cloned herein is from Pichia pastoris cDNA and not contamination.

FIG. 19 Strength of yeast promoters determined under different aeration conditions (Sun et al., Bioengineering Biotechnology “Systematic Characterization of a Panel of Constitutive Promoters for Applications in Pathway Engineering in Saccharomyces cerevisiae” (forthcoming)).

FIG. 20 Promoter mutants created through nucleotide analogue mutagenesis. Strength of promoter mutants was shown using a wild type TEF1 promoter as a reference (relative strength of 100). All promoter strengths were determined by measuring the fluorescent intensity of green florescent protein driven by promoter mutants. All samples were measured in triplicates. Error bars indicate the standard deviation of the replicated samples.

FIG. 21 Scaffold for the promoter-based pathway assembly of xylose utilization pathways. The scaffold for pathway assembly consists of a xylose reductase gene from Candida shehatae flanked with a PDC1 promoter and an ADH1 terminator, followed by a xylitol dehydrogenase gene from Candida tropicalis flanked with a TEF1 promoter and a CYC1 terminator, and a xyulokinase gene from Pichia pastoris flanked with an ENO2 promoter and an ADH2 terminator.

FIG. 22 Assembly of gene expression cassettes on the pRS414 helper plasmids. The helper plasmids were first linearized at the unique KpnI site, and then co-transformed into S. cerevisiae with the PCR fragments of the promoter mutants. The resulting constructs were used for amplification of gene expression cassettes consisting of a promoter, the reading frame of an enzyme homologue, a terminator, and the upstream and downstream homologous regions.

FIG. 23 Xylose fermentation performance of eight colonies randomly picked from the promoter-based pathway library in INVSc1. The cell growth of the mutants (indicated by cell density measured using optical density at 600 nm), xylose consumption, ethanol production, and ethanol yield from xylose were all different for the eight mutants.

FIG. 24 Screening strategy based on colony size. The pathway library is spread on an agar plate containing 2% xylose as the sole carbon source together with a reference pathway consisting of a xylose utilization pathway driven by wild type PDC1, TEF1 and ENO2 promoters. Colonies on the library plate that have grown to a size bigger than that of the largest colonies on the reference plate are picked and inoculated in media supplemented with 2% and the necessary selection pressure for maintaining the pathway bearing plasmids. The seed cultures are then used to inoculate tubes containing YP media supplemented with 2% xylose to a similar initial OD. Mutant strains bearing fast xylose utilizing pathways are identified by measuring the cell growth rates of the mutant strains. The top ten mutant strains identified using tube cultures are screened again in 50 mL flasks containing 10 ml YP media supplemented with 2% xylose. Flask cultures are analyzed using HPLC and the top mutant strain with the fastest xylose utilization rate and the highest ethanol productivity is identified.

FIG. 25 Correlation between xylose consumption and ethanol production with specific growth rate for tube based screening. The 36 hour samples of the fifty largest colonies from the promoter-based pathway library in the Classic strain were analyzed using HPLC. The overall xylose consumption and ethanol concentration was plotted with the specific growth rate of the mutant strains. The top xylose consumer and ethanol producer from flask based screening under oxygen limited conditions are marked in dark black.

FIG. 26 Tube and flask based screening of the promoter-based pathway library in different strain backgrounds. Left: Specific growth rates of the eighty or fifty colonies screened using tubes. The top mutants selected for later flask screening are marked in squares and the strain hosting the control pathway is marked in triangles. Right: Xylose consumption and ethanol yield of the top ten growers in flask based screening before xylose depletion. In both cases, the control strain contains pathways driven by wild type promoters on a single copy plasmid (PDC1p_wt-csXR-ADH1t-TEF1p_wt-ctXDH-CYC1t-ENO2p_wt-ppXKS-ADH2t).

FIG. 27 Xylose consumption rates and ethanol yields of 10 pathways as screened (before retransformation) and after retransformed into fresh host strain, InvSc1. The xylose consumption and ethanol yield after 3 days of fermentation under oxygen limited conditions are shown. In each bar pair, the xylose consumption and ethanol yield before retransformation are shown in the left bar, while those after retransformation are shown in the right bar.

FIG. 28 Optimization of the engineered xylose utilization pathway in S. cerevisiae by promoter optimization. (a) Scheme of the engineered fungal xylose utilization pathway. (b) Xylose fermentation behavior of eight randomly picked colonies from the pathway library. (c) Optimization of the xylose utilization pathway in the Classic strain via promoter optimization. The open symbols are from a strain with wild type promoters and the solid symbols are from a strain with optimized promoters, with an initial OD˜2 (solid line) or OD˜10 (dashed line). Circle: xylose Down triangle: ethanol. (d) Optimization of the xylose utilization pathway in the INVSc1 strain via promoter optimization. The open symbols are from a strain with wild type promoters and the solid symbols are from a strain with optimized promoters. Circle: xylose, down triangle: ethanol. (e) Xylose fermentation of the pathways optimized under different strain background in the INVSc1 strain. Open symbol: the pathway optimized in the INVSc1 strain, Solid symbol: the pathway optimized in the Classic strain. Circle: xylose, Down triangle: ethanol. (f) Xylose fermentation of pathways optimized under different strain background in INVSc1strain. Open symbol: pathway optimized in Classic strain, Solid symbol: pathway optimized in INVSc1 strain. Circle: xylose, down triangle: ethanol.

FIG. 29 Comparison of the fermentation performance of the INVSc1 strains harboring the reference, control pathway (psXR-psXDH-psXKS) either on a single copy plasmid (left) or a single copy chromosomal integration (right).

FIG. 30 Xylose fermentation of the mutant INVSc1 strain S3 on a single copy plasmid (S3 plasmid) or single copy integration (S3 integration) compared to the wild type control strain (WT). The fermentation was done in duplicates. The error bar indicates the standard deviation of the replicates. WT=diamonds, S3 single copy plasmid=squares, S3 single copy integration=triangles.

FIG. 31 Xylose fermentation of the industrial strains harboring optimized mutant xylose utilizing pathways. In the YPD seed culture initial OD˜10 graph, Classic WT YPD OD˜10=diamonds, Classic S7 YPD OD˜10=squares, ATCC WT YPD OD˜10=triangles, ATCC S8 YPD OD˜10=circles. In the YPX seed culture initial OD˜2 graph, Classic WT YPX OD˜2=diamonds, Classic S7 YPX OD˜2=squares, ATCC WT YPX OD˜2=triangles, ATCC S8 YPX OD˜2=circles.

FIG. 32 Xylose fermentation of the industrial strains harboring optimized mutant xylose utilizing pathways. In the YPX seed culture initial OD˜10 graph, Classic S7 YPX OD˜10=square and ATCC S8 YPX OD˜10=circle.

FIG. 33 Scheme for the combinatorial design of the cellobiose pathway.

FIG. 34 Optimization of the engineered cellobiose utilization pathway in S. cerevisiae via promoter optimization. (a) Scheme of the engineered cellobiose utilization pathway. (b) Library screening on an YPAC agar plate. (c) Comparison of cellobiose consumption and ethanol production in 250 mL flask fermentations in industrial Classic strain. The open symbols are from a strain with wild type promoters and the solid symbols are from a strain with optimized promoters. Circle: cellobiose, square: OD (A₆₀₀), down triangle: ethanol. (d) Comparison of cellobiose consumption and ethanol production in 250 mL flask fermentations in laboratory INVSc1 strain. The open symbols are from a strain with wild type promoters and the solid symbols are from a strain with optimized promoters. Circle: cellobiose, square: OD (A₆₀₀), down triangle: ethanol. (e) Cellobiose fermentation of the pathways optimized under different strain background in the Classic strain. (Open symbol: pathway optimized in INVSc1 strain, Solid symbol: pathway optimized in Classic strain. Circle: cellobiose, square: OD (A₆₀₀), down triangle: ethanol. (f) Cellobiose fermentation of the pathways optimized under different strain background in the INVSc1 strain. Open symbol: pathway optimized in Classic strain, Solid symbol: pathway optimized in INVSc1 strain. Circle: cellobiose, square: OD (A₆₀₀), down triangle: ethanol.

FIG. 35 Scheme for construction of helper plasmids and plasmids containing a library of cellobiose pathways.

FIG. 36 Cellobiose cultivation behavior of six recombinants with designed strengths of cellobiose transporter and β-glucosidase. Six different recombinants, each contains a transporter coupled to an ENO promoter and a β-glucosidase coupled to a PDC promoter, were assembled into SalI-NotI digested single copy plasmid expression pRS-kanMX. Culture condition: Recombinants were first seed cultured in YPAD medium to exponential phase, washed cells were then directly transferred into 25 mL YPAC medium (8% cellobiose) in 125 mL flask and shaken with 100 rpm at 30° C. No YPAC pre-culture was performed before main culture to avoid any adaptation. Significant different lag phases were observed.

FIG. 37 Screening of a library of cellobiose utilization mutant pathways in industrial strain Classic using YPAC agar plates. (a) A library of cellobiose utilization pathways containing combinations of 11 ENO2 mutant promoters and 10 PDC1 mutant promoters. (b) The cellobiose pathway consisting of only one combination of ENO2 and PDC1 promoters (ENO 14%-PDC 215%).

FIG. 38 Screening of a library of cellobiose utilization pathways in industrial strain Classic by cultivations in Falcon tubes and shake-flasks. (a) Ethanol concentrations of 80 colonies from YPAC agar plate screening cultured in Falcon tubes. The concentrations ranged from 16.9 to 25.1 g/L. (b) Ethanol concentrations of top 10 strains from tube screening cultured in shake-flasks.

FIG. 39 Comparison of cellobiose consumption and ethanol production in 125 mL shake-flask fermentations between WT and CYT-059 in industrial strain Classic. The open symbols are from a strain with wild type promoters and the solid symbols are from CYT-059 (having optimized promoters). Circle: cellobiose, square: OD (A₆₀₀), down triangle: ethanol.

FIG. 40 Comparison of cellobiose consumption and ethanol production in 125 mL shake-flask fermentations between WT and INV-C3 in laboratory strain INVSc1. The open symbols are from a strain with wild type promoters and the solid symbols are from INV-C3 (having optimized promoters). Circle: cellobiose, square: OD (A₆₀₀), down triangle: ethanol.

FIG. 41 Specific growth rate distribution of the 80 clones picked from the library based on the colony size (A) and xylose fermentation properties of the 10 fast growers selected based on the specific growth rate (B). In each group of 4 bars in (B), the left-most bar is xylose consumption rate, the second from the left is ethanol yield, the third from the left is xylitol yield, and the right-most bar is glycerol yield. The range of the specific growth rates of the fast 10 growers is shown in the far right section in (A).

FIG. 42 Specific growth rate distributions of the 80 clones picked from InvSc1 and ATCC 4124 strain libraries and 50 clones picked from Classic strain library (panels A, C, and E) and xylose fermentation properties of the 10 fast growers in each libary (panels B, D, and F). In panels B, D, and F, in each group of four bars, the left-most bar is xylose consumption rate, the second from the left is ethanol yield, the third from the left is xylitol yield, and the right-most bar is glycerol yield.

FIG. 43 Fermentation profiles on YPX (4%) under oxygen-limited condition and comparison of three selected pathways for each strain: Panel A, InvSc1 strain with pathway #2 (InvSc1-IL2); Panel B, ATCC 4124 strain with pathway #2 (ATCC-AL2); Panel C, Classic strain with pathway #3 (Classic-CL3). * and ** indicate P<0.05 and P<0.005 (n=3), respectively. In Panel D, in each group of three bars, the left-most bar is InvSc1-IL2, the middle bar is ATCC-AL2, and the right bar is Classic-CL3.

FIG. 44 Co-fermentation profiles on YPGX (4% glucose and 4% xylose) under oxygen-limited condition and comparison of three selected pathways for each strain: Panel A, InvSc1 strain with pathway #2 (InvSc1-IL2); Panel B, ATCC 4124 strain with pathway #2 (ATCC-AL2); Panel C, Classic strain with pathway #3 (Classic-CL3). * indicates P<0.05 and P<0.005 (n=3), respectively. In Panel D, in each group of three bars, the left-most bar is InvSc1-IL2, the middle bar is ATCC-AL2, and the right bar is Classic-CL3.

FIG. 45 Panel A: Xylose consumption rates of InvSc1, ATCC 4124, Classic strains transformed with the five pathways found in the screening of Classic strain library, demonstrating the dependency of host strain background. In each group of three bars, the left-most bar is InvSc1-IL2, the middle bar is ATCC-AL2, and the right bar is Classic-CL3. Panel B: Co-fermentation profiles on YPGX (4% glucose and 4% xylose) under oxygen-limited condition of 10 fast growers in ATCC 4124 strain library. In each group of three bars, the left bar is xylitol yield, the middle bar is xylose consumption rate, and the right bar is ethanol yield.

FIG. 46 Xylose and mixed sugar (4% glucose and 4% xylose) fermentation profiles of ATCC-IL2 and ATCC-IL5 which were found by testing the same 10 fast growers in 4% xylose and 4% glucose and 4% xylose mixture (A, B). Cofermentation (7% glucose and 4% xylose) profile of Classic-IL3, (C), and enzyme activities used in the library creation (measured in InvSc1 strain).

FIG. 47 Panel (A) Schematic for use of a pentose utilizing pathway as the selection marker; Panel (B) schematic for use of a separate positive selection marker as the selection marker.

FIG. 48 An overall schematic for heterologous combinatorial pathway assembly, screening, and final pathway identification.

### DETAILED DESCRIPTION

The present disclosure relates to the production of highly efficient heterologous pathways by identifying favorable enzyme and/or promoter combinations. In particular the present disclosure provides methods for assembly and selection of multi-step xylose and arabinose/xylose utilization pathways from a library of fungal enzymes. The present disclosure further provides compositions containing favorable enzyme combinations, as well as recombinant yeast expressing such combinations, and methods of use for bioconversion of pentose sugars. Also provided are compositions and methods involving favorable expression patterns identified by utilization of combinations of promoters of varying strengths. Provided herein are methods for assembly and selection of multi-step xylose, arabinose/xylose, and cellobiose utilization pathways from a library containing polynucleotides encoding proteins of multi-step xylose, arabinose/xylose, and/or cellobiose utilization pathways under the control of promoters of varying strengths. The present disclosure further provides compositions containing heterologous enzyme-coding polynucleotides under the control of favorable promoters, as well as recombinant yeast expressing such enzymes, and methods of their use for bioconversion of pentose and/or hexose sugars.

### EMBODIMENTS

The present disclosure relates to methods of producing libraries of multi-enzyme pathways by providing a plurality of gene expression cassettes for each enzyme of a pathway of interest. In some aspects, each of the plurality of gene expression cassettes contains a nucleic acid containing a varying coding region of a homolog of an enzyme of interest in operable combination with a constant heterologous promoter. In these embodiments, the relative expression level of the enzyme of interest is a function of the sequence of the coding region, which differs from another of the plurality of gene expression cassettes. In other aspects, each of the plurality of gene expression cassettes contains a nucleic acid containing a constant coding region of an enzyme of interest in operable combination with a varying heterologous promoter. In these embodiments, the relative expression level of the enzyme of interest is a function of the sequence of the promoter, which differs from another of the plurality of gene expression cassettes.

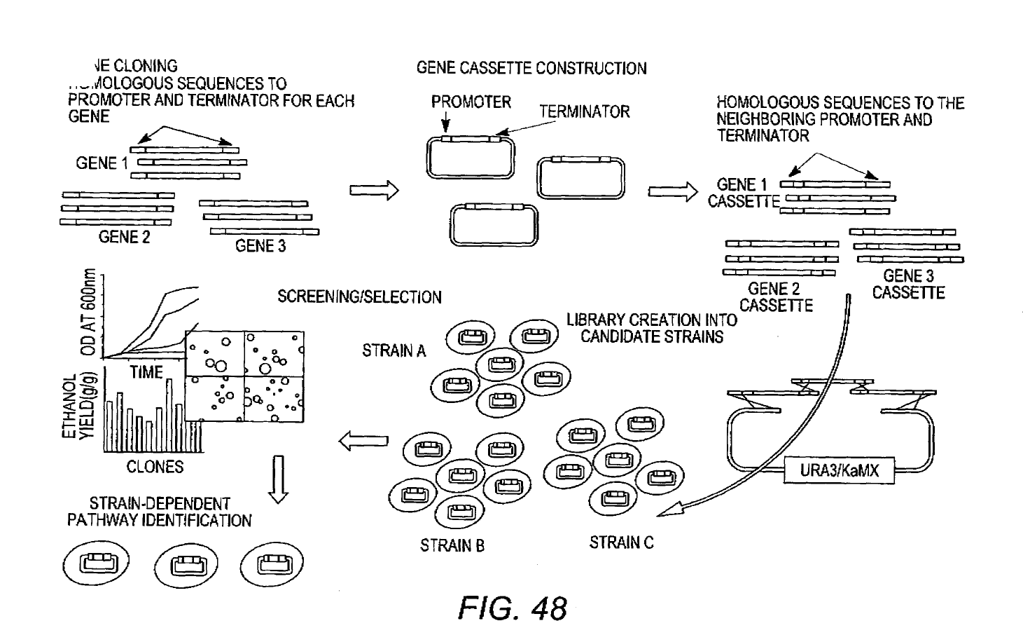

In some embodiments, a heterologous multi-enzyme pathway is prepared according to the schematic outlined in FIG. 48.

In some embodiments, the multi-enzyme pathway is a xylose utilization pathway containing a xylose reductase, a xylitol dehydrogenase, and a xylulokinase. In other embodiments, the multi-enzyme pathway is a xylose/arabinose utilization pathway containing a xylose reductase, a xylitol dehydrogenase, a xylulokinase, an L-arabitol 4-dehydrogenase, and a L-xylulose reductase. In further embodiments, the multi-enzyme pathway further contains additional components such as one or more of a xylose-specific transporter, an arabinose-specific transporter, a transaldolase, and a transketolase. In some embodiments, the multi-enzyme pathway contains a cellodextrin transporter and beta-glucosidase.

Also provided by the present disclosure are isolated polynucleotides containing gene expression cassettes of a xylose or a xylose/arabinose utilization pathway. Also provided by the present disclosure are isolated polynucleotides containing gene expression cassettes of a cellobiose utilization pathway. In still further embodiments, the present disclosure provides vectors and genetically modified host cells (recombinant yeast cells) containing the isolated polynucleotides. In other aspects, the present disclosure provides methods of selecting recombinant yeast cells enriched in favorable combinations of gene expression cassettes for pentose and/or cellobiose utilization. Also provided are methods for culturing the recombinant yeast cells, and methods for producing ethanol through use of the recombinant yeast cells to ferment pentose and/or cellobiose.

Pentose Utilization Pathways

As used herein the term “pentose utilization pathway” refers to three or more proteins that play roles in pentose metabolism. In preferred embodiments the proteins include but are not limited to enzymes. In some embodiments, the proteins further include a pentose-specific transporter. In one embodiment, the pathway is a “xylose-utilization pathway” containing a xylose reductase, a xylitol dehydrogenase, and a xylulokinase. In another embodiment, the pathway is a “arabinose-utilization pathway” containing a xylose reductase, a xylitol dehydrogenase, a xylulokinase, an L-arabitol 4-dehydrogenase, and a L-xylulose reductase. In other embodiments, the pathway further contains one or more of a pentose-specific transporter, a transaldolase, and a transketolase. In still further embodiments, the pathway further contains a xylose isomerase.

The terms “xylose reductase” and “XR” as used herein refer to an enzyme that catalyzes the following reaction: xylose+NAD(P)H+H+=xylitol+NAD(P)+(EC 1.1.1.21). Other names for xylose reductase include “aldehyde reductase” include “aldose reductase,” “polyol dehydrogenase (NADP+),” “ALR2,” “NADPH-aldopentose reductase,” “NADPH-aldose reductase,” and “alditol:NAD(P)+1-oxidoreductase.”

The terms “xylitol dehydrogenase” and “XDH” refer to an enzyme that catalyzes the following reaction: xylitol+NAD+=D-xylulose+NADH+H+ (EC 1.1.1.9). Other names for xylitol dehydrogenase include “D-xylulose reductase,” “NAD-dependent xylitol dehydrogenase,” “erythritol dehydrogenase,” “2,3-cis-polyol(DPN) dehydrogenase (C₃₋₅),” “pentitol-DPN dehydrogenase,” “xylitol-2-dehydrogenase,” and “xylitol: NAD+2-oxidoreductase (D-xylulose-forming).”

The terms “xylulokinase” and “XKS” refer to an enzyme that catalyzes the following reaction: ATP+D-xylulose=ADP+D-xylulose 5-phosphate (EC 2.7.1.17). Other names for xylulokinase include “D-xylulokinase” and “ATP:D-xylulose 5-phosphotransferase.”

The terms “L-arabitol 4-dehydrogenase” and “LAD” refer to an enzyme that catalyzes the following reaction: L-arabinitol+NAD+=L-xylulose+NADH+H+ (EC 1.1.1.12). Other names for L-arabitol 4-dehydrogenase include “pentitol-DPN dehydrogenase,” and “L-arabinitol:NAD+4-oxidoreductase (L-xylulose-forming).”

The terms “L-xylulose reductase” and “LXR” refer to an enzyme that catalyzes the following reaction: L-xylulose+NADPH+H+=xylitol+NADP+(EC 1.1.1.10). Other names for L-xylulose reductase include “xylitol dehydrogenase,” and “xylitol:NADP+4-oxidoreductase (L-xylulo se-forming).”

The term “catalytic activity” or “activity” describes quantitatively the conversion of a given substrate under defined reaction conditions. The term “residual activity” is defined as the ratio of the catalytic activity of the enzyme under a certain set of conditions to the catalytic activity under a different set of conditions. The term “specific activity” describes quantitatively the catalytic activity per amount of enzyme under defined reaction conditions.

The term “hemicellulose” refers to a polymer of short, highly-branched chains of mostly five-carbon pentose sugars (e.g., xylose and arabinose) and to a lesser extent six-carbon hexose sugars (e.g., galactose, glucose and mannose). Hemicelluloses may include, for example, xylan, glucuronoxylan, arabinoxylan, glucomannan, or xyloglucan. Non-limiting examples of sources of hemicellulose include grasses (e.g., switchgrass, Miscanthus), rice hulls, bagasse, cotton, jute, hemp, flax, bamboo, sisal, abaca, straw, leaves, grass clippings, corn stover, corn cobs, distillers grains, legume plants, sorghum, sugar cane, sugar beet pulp, wood chips, sawdust, and biomass crops (e.g., Crambe).

In some embodiments, the pathways of the present disclosure are used in conjunction with one or more additional proteins of interest. Non-limiting examples of proteins of interest include: hemicellulases, alpha-galactosidases, beta-galactosidases, lactases, beta-glucanases, endo-beta-1,4-glucanases, cellulases, xylosidases, xylanases, xyloglucanases, xylan acetyl-esterases, galactanases, endo-mannasases, exo-mannanases, pectinases, pectin lyases, pectinesterases, polygalacturonases, arabinases, rhamnogalacturonases, laccases, reductases, oxidases, phenoloxidases, ligninases, proteases, amylases, phosphatases, lipolytic enzymes, cutinases, and/or other enzymes.

Cellobiose Utilization Pathways

As used herein the term “cellobiose utilization pathway” refers to two or more proteins that play roles in cellobiose metabolism. In one embodiment, the pathway is a “cellobiose utilization pathway” containing a cellodextrin transporter and a beta-glucosidase. In one aspect, the cellodextrin transporter is a cellobiose transporter.

The term “cellodextrin transporter” as used herein refers to a protein that facilitates the transport of one or more types of cellodextrin across a cell membrane. Cellodextrins include, without limitation, cellobiose, cellotriose, cellotetraose, cellopentaose, and cellohexaose.

The term “beta-glucosidase” as used herein refer to a protein that catalyzes the cleavage of beta 1-4 bonds linking two glucose molecules (e.g. as in a cellobiose molecule)

Cellulodextrins may be obtained from the degradation of cellulose. Non-limiting examples of sources of cellulose include grasses (e.g., switchgrass, Miscanthus), rice hulls, bagasse, cotton, jute, hemp, flax, bamboo, sisal, abaca, straw, leaves, grass clippings, corn stover, corn cobs, distillers grains, legume plants, sorghum, sugar cane, sugar beet pulp, wood chips, sawdust, and biomass crops (e.g., Crambe).

In some embodiments, the pathways of the present disclosure are used in conjunction with one or more additional proteins of interest. Non-limiting examples of proteins of interest include: hemicellulases, alpha-galactosidases, beta-galactosidases, lactases, beta-glucanases, endo-beta-1,4-glucanases, cellulases, xylosidases, xylanases, xyloglucanases, xylan acetyl-esterases, galactanases, endo-mannasases, exo-mannanases, pectinases, pectin lyases, pectinesterases, polygalacturonases, arabinases, rhamnogalacturonases, laccases, reductases, oxidases, phenoloxidases, ligninases, proteases, amylases, phosphatases, lipolytic enzymes, cutinases, and/or other enzymes.

Polynucleotides

The terms “polynucleotide” and “nucleic acid” used interchangeably herein, refer to a polymeric form of nucleotides of any length, either ribonucleotides or deoxyribonucleotides. These terms include, but are not limited to, a single-, double- or triple-stranded DNA, genomic DNA, cDNA, RNA, DNA-RNA hybrid, or a polymer containing purine and pyrimidine bases, or other natural, chemically, biochemically modified, non-natural or derivatized nucleotide bases. The following are non-limiting examples of polynucleotides: genes, gene fragments, chromosomal fragments, ESTs, exons, introns, mRNA, tRNA, rRNA, ribozymes, cDNA, recombinant polynucleotides, branched polynucleotides, plasmids, vectors, isolated DNA of any sequence, isolated RNA of any sequence, nucleic acid probes, and primers. Polynucleotides of the present disclosure are prepared by any suitable method known to those of ordinary skill in the art, including, for example, direct chemical synthesis, amplification or cloning. The term “library” as used herein in references to nucleic acids, refers to a collection of isolated nucleic acids.

In one aspect, the disclosure provides an isolated or purified nucleic acid molecule encoding a pentose utilization pathway (three or more proteins that play roles in pentose metabolism). In another aspect, the disclosure provides an isolated or purified nucleic acid molecule encoding one or more proteins of a pentose utilization pathway. In certain embodiments, the recombinant polynucleotides of the disclosure encode polypeptides having at least 50%, or at least about 55%, or at least about 60%, or at least about 65%, or at least about 70%, or at least about 75%, or at least about 80%, or at least about 85%, or at least about 90%, or at least about 91%, or at least about 92%, or at least about 93%, or at least about 94%, or at least about 95%, or at least about 96%, or at least about 97%, or at least about 98%, or at least about 99%, or at least about 100% amino acid residue sequence identity over a specified region, or, when not specified, over the entire sequence of a polypeptide of any of SEQ ID NOS:1-94.

In another aspect, the disclosure provides an isolated or purified nucleic acid molecule encoding a cellobiose utilization pathway (two or more proteins that play roles in cellobiose metabolism). In another aspect, the disclosure provides an isolated or purified nucleic acid molecule encoding one or more proteins of a cellobiose utilization pathway. In certain embodiments, the recombinant polynucleotides of the disclosure encode polypeptides having at least 50%, or at least about 55%, or at least about 60%, or at least about 65%, or at least about 70%, or at least about 75%, or at least about 80%, or at least about 85%, or at least about 90%, or at least about 91%, or at least about 92%, or at least about 93%, or at least about 94%, or at least about 95%, or at least about 96%, or at least about 97%, or at least about 98%, or at least about 99%, or at least about 100% amino acid residue sequence identity over a specified region, or, when not specified, over the entire sequence of a polypeptide of any of SEQ ID NOS:129-130.

In some embodiments, the recombinant polynucleotides of the disclosure have at least at least 50%, or at least about 55%, or at least about 60%, or at least about 65%, or at least about 70%, or at least about 75%, or at least about 80%, or at least about 85%, or at least about 90%, or at least about 95%, or at least about 96%, or at least about 97%, or at least about 98%, or at least about 99%, or at least about 100% nucleic acid sequence identity over a specified region, or, when not specified, over the entire sequence of a promoter or terminator of the Examples.

For sequence comparison, typically one sequence acts as a reference sequence, to which test sequences are compared. When using a sequence comparison algorithm, test and reference sequences are entered into a computer, subsequence coordinates are designated, if necessary, and sequence algorithm program parameters are designated. Default program parameters can be used, or alternative parameters can be designated. The sequence comparison algorithm then calculates the percent sequence identities for the test sequences relative to the reference sequence, based on the program parameters. When comparing two sequences for identity, it is not necessary that the sequences be contiguous, but any gap would carry with it a penalty that would reduce the overall percent identity. For blastn, the default parameters are Gap opening penalty=5 and Gap extension penalty=2. For blastp, the default parameters are Gap opening penalty=11 and Gap extension penalty=1.

A “comparison window”, as used herein, includes reference to a segment of any one of the number of contiguous positions selected from the group consisting of from 20 to 600, usually about 50 to about 200, more usually about 100 to about 150 in which a sequence may be compared to a reference sequence of the same number of contiguous positions after the two sequences are optimally aligned. Methods of alignment of sequences for comparison are well-known in the art. Optimal alignment of sequences for comparison can be conducted using known algorithms (e.g., by the local homology algorithm of Smith and Waterman, Adv Appl Math, 2:482, 1981; by the homology alignment algorithm of Needleman and Wunsch, J Mol Biol, 48:443, 1970; by the search for similarity method of Pearson and Lipman, Proc Natl Acad Sci USA, 85:2444, 1988; by computerized implementations of these algorithms FASTDB (Intelligenetics), BLAST (National Center for Biomedical Information), GAP, BESTFIT, FASTA, and TFASTA in the Wisconsin Genetics Software Package (Genetics Computer Group, Madison, Wis.), or by manual alignment and visual inspection.

A preferred example of an algorithm that is suitable for determining percent sequence identity and sequence similarity is the FASTA algorithm (Pearson and Lipman, Proc Natl Acad Sci USA, 85:2444, 1988; and Pearson, Methods Enzymol, 266:227-258, 1996). Preferred parameters used in a FASTA alignment of DNA sequences to calculate percent identity are optimized, BL50 Matrix 15:−5, k-tuple=2; joining penalty=40, optimization=28; gap penalty-12, gap length penalty=−2; and width=16.

Another preferred example of algorithms suitable for determining percent sequence identity and sequence similarity are the BLAST and BLAST 2.0 algorithms (Altschul et al., Nuc Acids Res, 25:3389-3402, 1977; and Altschul et al., J Mol Biol, 215:403-410, 1990, respectively). BLAST and BLAST 2.0 are used, with the parameters described herein, to determine percent sequence identity for the nucleic acids and proteins of the disclosure. Software for performing BLAST analyses is publicly available through the National Center for Biotechnology Information website. This algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold. These initial neighborhood word hits act as seeds for initiating searches to find longer HSPs containing them. The word hits are extended in both directions along each sequence for as far as the cumulative alignment score can be increased. Cumulative scores are calculated using, for nucleotide sequences, the parameters M (reward score for a pair of matching residues; always >0) and N (penalty score for mismatching residues; always <0). For amino acid sequences, a scoring matrix is used to calculate the cumulative score. Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T, and X determine the sensitivity and speed of the alignment. The BLASTN program (for nucleotide sequences) uses as defaults a word length (W) of 11, an expectation (E) of 10, M=5, N=−4 and a comparison of both strands. For amino acid sequences, the BLASTP program uses as defaults a word length of 3, and expectation (E) of 10, and the BLOSUM62 scoring matrix (Henikoff and Henikoff, Proc Natl Acad Sci USA, 89:10915, 1989) alignments (B) of 50, expectation (E) of 10, M=5, N=−4, and a comparison of both strands.

The BLAST algorithm also performs a statistical analysis of the similarity between two sequences (See, e.g., Karlin and Altschul, Proc Natl Acad Sci USA, 90:5873-5787, 1993). One measure of similarity provided by the BLAST algorithm is the smallest sum probability (P(N)), which provides an indication of the probability by which a match between two nucleotide or amino acid sequences would occur by chance. For example, a nucleic acid is considered similar to a reference sequence if the smallest sum probability in a comparison of the test nucleic acid to the reference nucleic acid is less than about 0.2, more preferably less than about 0.01, and most preferably less than about 0.001.

Another example of a useful algorithm is PILEUP. PILEUP creates a multiple sequence alignment from a group of related sequences using progressive, pairwise alignments to show relationship and percent sequence identity. It also plots a tree or dendogram showing the clustering relationships used to create the alignment. PILEUP uses a simplification of the progressive alignment method (Feng and Doolittle, J Mol Evol, 35:351-360, 1987), employing a method similar to a published method (Higgins and Sharp, CABIOS 5:151-153, 1989). The program can align up to 300 sequences, each of a maximum length of 5,000 nucleotides or amino acids. The multiple alignment procedure begins with the pairwise alignment of the two most similar sequences, producing a cluster of two aligned sequences. This cluster is then aligned to the next most related sequence or cluster of aligned sequences. Two clusters of sequences are aligned by a simple extension of the pairwise alignment of two individual sequences. The final alignment is achieved by a series of progressive, pairwise alignments. The program is run by designating specific sequences and their amino acid or nucleotide coordinates for regions of sequence comparison and by designating the program parameters. Using PILEUP, a reference sequence is compared to other test sequences to determine the percent sequence identity relationship using the following parameters: default gap weight (3.00), default gap length weight (0.10), and weighted end gaps. PILEUP can be obtained from the GCG sequence analysis software package, e.g., version 7.0 (Devereaux et al., Nuc Acids Res, 12:387-395, 1984).

Another preferred example of an algorithm that is suitable for multiple DNA and amino acid sequence alignments is the CLUSTALW program (Thompson et al., Nucl Acids. Res, 22:4673-4680, 1994). ClustalW performs multiple pairwise comparisons between groups of sequences and assembles them into a multiple alignment based on homology. Gap open and Gap extension penalties were 10 and 0.05 respectively. For amino acid alignments, the BLOSUM algorithm can be used as a protein weight matrix (Henikoff and Henikoff, Proc Natl Acad Sci USA, 89:10915-10919, 1992).

Polynucleotides of the disclosure further include polynucleotides that encode conservatively modified variants of the polypeptides of any of SEQ ID NOS:1-94 or 129-130. “Conservatively modified variants” as used herein include individual mutations that result in the substitution of an amino acid with a chemically similar amino acid. Conservative substitution tables providing functionally similar amino acids are well known in the art. Such conservatively modified variants are in addition to and do not exclude polymorphic variants, interspecies homologs, and alleles of the disclosure. The following eight groups contain amino acids that are conservative substitutions for one another: 1. Alanine (A), Glycine (G); 2. Aspartic acid (D), Glutamic acid (E); 3. Asparagine (N), Glutamine (Q); 4. Arginine (R), Lysine (K); 5. Isoleucine (I), Leucine (L), Methionine (M), Valine (V); 6. Phenylalanine (F), Tyrosine (Y), Tryptophan (W); 7. Serine (S), Threonine (T); and 8. Cysteine (C), Methionine (M).

Polynucleotides of the disclosure further include polynucleotides that encode homologs (especially orthologs) of polypeptides of SEQ ID NOS:1-94 or 129-130. As used herein, the terms “homolog” and “homologue” refer to a gene related to a second gene by descent from a common ancestral DNA sequence. The term homolog applies to the relationship between genes separated by a speciation event (e.g., ortholog), and to the relationship between genes separated by a genetic duplication event (e.g., paralog). In preferred embodiments, the term homolog refers to genes having the same or similar function to a parent or reference gene.

The terms “isolated” and “purified” as used herein refers to a material that is removed from at least one component with which it is naturally associated (e.g., removed from its original environment). The term “isolated,” when used in reference to DNA, refers to a DNA molecule that has been removed from its natural genetic milieu and is thus free of extraneous or unwanted coding and/or non-coding sequences. Such isolated molecules are those that are separated from their natural environment and include cDNA and genomic clones. The term “isolated,” when used in reference to a protein, refers to a protein that is found in a condition other than its native environment. In a preferred form, the isolated protein is substantially free of other proteins. In some preferred embodiments, a nucleic acid or protein is said to be purified, for example, if it gives rise to essentially one band in an electrophoretic gel or blot.

The terms “gene expression cassette” and “expression construct” refer to an isolated nucleic acid generated recombinantly or synthetically, with a series of specified nucleic acid elements that permit transcription of a “coding region” in a target cell. The expression cassette can be incorporated into a plasmid, a chromosome, or other nucleic acid fragment. Typically, the expression cassette contains a coding region of a protein in operable combination with a promoter and a terminator.

The terms “coding region,” “open reading frame” and “ORF” refers to a sequence of codons extending from an initiator codon (ATG) to a terminator codon (TAG, TAA or TGA), which can be translated into a polypeptide. As used herein, the term “promoter” refers to a nucleic acid sequence that functions to direct transcription of a downstream polynucleotide. Promoters of the disclosure include any promoter that functions to direct transcription of a downstream polynucleotide in a host cell of the disclosure and include, without limitation, ENO2, PDC1, FBA1, GPM1, TPI1, and TEF1 promoters. As used herein, the term “terminator” refers to a nucleic acid sequence that causes transcription to cease. A nucleic acid is “operably linked” or “in operable combination” when it is placed in an appropriate position relative to another nucleic acid. For instance, a promoter is operably linked to a coding sequence if it affects the transcription of the sequence or a ribosome binding site is operably linked to a coding sequence if it is positioned so as to facilitate translation. Generally, “operably linked” means that the nucleic acids being linked are contiguous, and, in the case of a fusion protein are contiguous and in the same reading frame. Linking is accomplished by ligation at convenient restriction sites or if such sites do not exist, synthetic oligonucleotide adaptors or linkers are used in accordance with conventional practice. As described herein, in preferred embodiments, linking is accomplished by homologous recombination (e.g., DNA assembly in transformed yeast cells).

As used herein, the term “vector” refers to a polynucleotide construct designed to introduce nucleic acids into one or more cell types. Vectors include cloning vectors, expression vectors, shuttle vectors, plasmids, cassettes and the like. As used herein, the term “plasmid” refers to a circular double-stranded DNA construct used as a cloning and/or expression vector. Some plasmids take the form of an extrachromosomal self-replicating genetic element (episomal plasmid) when introduced into a host cell. Other plasmids integrates into a host chromosome (integrative plasmid) when introduced into a host cell, and are thereby replicated along with the host cell genome. Moreover, certain vectors are capable of directing the expression of coding regions genes to which they are operatively linked. Such vectors are referred to herein as “expression vectors” (or simply, “expression vectors”).

The terms “derived from” or “of” when used in reference to a nucleic acid or protein indicates that its sequence is identical or substantially identical to that of an organism of interest. For instance, “a xylose reductase derived from Neurospora crassa” or “a xylose reductase of N. crassa” refers to a xylose reductase enzyme having a sequence identical or substantially identical to a native xylose reductase enzyme of N. crassa. The terms “derived from” and “of” when used in reference to a nucleic acid or protein do not indicate that the nucleic acid or protein in question was necessarily directly purified, isolated or otherwise obtained from an organism of interest. Thus by way of example, an isolated nucleic acid containing a xylose reductase coding region of N. crassa need not be obtained directly from this fungal species, instead the isolated nucleic acid may be prepared synthetically using methods known to one of skill in the art.

As used herein in the context of introducing a nucleic acid sequence into a cell, the term “introduced” refers to any method suitable for transferring the nucleic acid sequence into the cell. Such methods for introduction include but are not limited to protoplast fusion, transfection, transformation, conjugation, and transduction. As used herein, the term “transformed” refers to a cell that has an exogenous polynucleotide sequence integrated into its genome or as an episomal plasmid that is maintained for at least two generations.

Recombinant Host Cells

“Recombinant nucleic acid” or “recombinant polynucleotide” as used herein refers to a polymer of nucleic acids wherein at least one of the following is true: (a) the sequence of nucleic acids is foreign to (i.e., not naturally found in) a given host cell; (b) the sequence may be naturally found in a given host cell, but in an unnatural (e.g., greater than expected) amount; or (c) the sequence of nucleic acids contains two or more subsequences that are not found in the same relationship to each other in nature. For example, regarding instance (c), a recombinant nucleic acid sequence will have two or more sequences from unrelated genes arranged to make a new functional nucleic acid. Specifically, the present disclosure is related to the introduction of an expression vector into a host cell, wherein the expression vector contains a nucleic acid sequence coding for a protein that is not normally found in a host cell or contains a nucleic acid coding for a protein that is normally found in a cell but is under the control of different regulatory sequences. With reference to the host cell's genome, then, the nucleic acid sequence that codes for the protein is recombinant.

The term “recombinant host cell” (or simply “host cell”) refers to a cell into which a recombinant vector has been introduced. It should be understood that such terms are intended to refer not only to the particular subject cell but to the progeny of such a cell. Because certain modifications may occur in succeeding generations due to either mutation or environmental influences, such progeny may not, in fact, be identical to the parent cell, but are still included within the scope of the term “host cell” as used herein.

The disclosure herein relates to host cells containing recombinant polynucleotides encoding polypeptides where the polypeptides are involved in pentose and/or cellobiose utilization. Host cells of the disclosure include any host cell containing one or more nucleic acids of the disclosure. In some aspects, a host cell of the disclosure contains a nucleic acid molecule of the disclosure that contains multiple polynucleotides encoding multiple polypeptides of a pentose and/or cellobiose utilization pathway. In some aspects, a host cell of the disclosure contains two or more nucleic acid molecule of the disclosure, wherein the polynucleotides encoding polypeptides of a pentose and/or cellobiose utilization pathway are on two or more different nucleic acid molecules. In some aspects, a combination of enzymes and/or promoters of interest in a heterologous pathway may be identified according to a method disclosed herein, and polynucleotides encoding the enzymes of interest under the control of promoters of interest are provided in a host cell on a single nucleic acid molecule. In some aspects, a combination of enzymes and/or promoters of interest in a heterologous may be identified according to a method disclosed herein, and polynucleotides encoding the enzymes of interest under the control of promoters of interest are provided in a host cell on more than one nucleic acid molecule. In some aspects, a host cell of the disclosure contains a nucleic acid molecule of the disclosure that contains three polynucleotides encoding a xylose reductase, a xylose dehydrogenase, and a xylulokinase on a single nucleic acid molecule. In some aspects, a host of the disclosure contains two or more nucleic acid molecules of the disclosure that contain three polynucleotides encoding a xylose reductase, a xylose dehydrogenase, and a xylulokinase on two or more nucleic acid molecules. In some aspects, a host cell of the disclosure contains a nucleic acid molecule of the disclosure that contains two polynucleotides encoding a cellodextrin transporter and a beta glucosidase on a single nucleic acid molecule. In some aspects, a host of the disclosure contains two nucleic acid molecules of the disclosure that contain two polynucleotides encoding a cellodextrin transporter and a beta glucosidase on two nucleic acid molecules.

In some aspects, in a host cell containing a recombinant nucleic acid molecule of the disclosure, the nucleic acid(s) is in a plasmid. In some aspects, the plasmid is an integrative plasmid, a centromeric plasmid, or an episomal plasmid. In some aspects, in a host cell containing a recombinant nucleic acid molecule of the disclosure, the nucleic acid(s) is integrated into a host cell chromosome.

Further described herein are methods of increasing growth of a host cell on a medium containing a pentose and/or cellobiose substrate, and methods of co-fermenting cellulose-derived and hemicellulose-derived pentoses.

“Host cell” and “host microorganism” are used interchangeably herein to refer to a living biological cell that can be transformed via insertion of recombinant DNA or RNA. Such recombinant DNA or RNA can be in an expression vector. Thus, a host organism or cell as described herein may be a prokaryotic organism (e.g., an organism of the kingdom Eubacteria) or a eukaryotic cell. As will be appreciated by one of ordinary skill in the art, a prokaryotic cell lacks a membrane-bound nucleus, while a eukaryotic cell has a membrane-bound nucleus.

Any prokaryotic or eukaryotic host cell may be used in the present disclosure so long as it remains viable after being transformed with a sequence of nucleic acids. Preferably, the host cell is not adversely affected by the transduction of the necessary nucleic acid sequences, the subsequent expression of the proteins (e.g., enzymes), or the resulting intermediates. Suitable eukaryotic cells include, but are not limited to yeast cells and filamentous fungal cells. “Fungi” as used herein includes the phyla Ascomycota, Basidiomycota, Chytridiomycota, and Zygomycota, as well as the Oomycota, and mitosporic fungi.

In particular embodiments, the fungal host is a yeast strain. “Yeast” as used herein includes ascosporogenous yeast (Endomycetales), basidiosporogenous yeast, and yeast belonging to the Fungi Imperfecti (Blastomycetes). Since the classification of yeast may change in the future, for the purposes of this disclosure, yeast shall be defined as described in Biology and Activities of Yeast (Skinner et al., eds, Soc. App. Bacteriol. Symposium Series No. 9, 1980).

In preferred embodiments, the yeast host cell is a Candida, Hansenula, Kluyveromyces, Pichia, Saccharomyces, Schizosaccharomyces, or Yarrowia strain. In certain embodiments, the yeast host is a Saccharomyces carlsbergensis, Saccharomyces cerevisiae, Saccharomyces diastaticus, Saccharomyces douglasii, Saccharomyces kluyveri, Saccharomyces norbensis, Saccharomyces monacensis, Saccharomyces bayanus, Saccharomyces pastorianus, Saccharomyces pombe, or Saccharomyces oviformis strain. In other preferred embodiments, the yeast host is Kluyveromyces lactis, Kluyveromyces fragilis, Kluyveromyces marxiamus, Pichia stipitis, Candida shehatae, or Candida tropicalis. In other embodiments, the yeast host is Yarrowia lipolytica, Brettanomyces custersii, or Zygosaccharomyces roux.

In another embodiment, the fungal host is a filamentous fungal strain. “Filamentous fungi” include filamentous forms of the subdivision Eumycota and Oomycota. The filamentous fungi are generally characterized by a mycelial wall composed of chitin, cellulose, glucan, chitosan, mannan, and other complex polysaccharides. Vegetative growth is by hyphal elongation and carbon catabolism is obligately aerobic. In contrast, vegetative growth by yeasts such as Saccharomyces cerevisiae is by budding of a unicellular thallus and carbon catabolism may be fermentative.

In preferred embodiments, the filamentous fungal host is an Acremonium, Aspergillus, Fusarium, Humicola, Mucor, Myceliophthora, Neurospora, Penicillium, Scytalidium, Thielavia, Tolypocladium, or Trichoderma strain. In certain embodiments, the filamentous fungal host is an Aspergillus awamori, Aspergillus foetidus, Aspergillus japonicus, Aspergillus nidulans, Aspergillus niger, or Aspergillus oryzae strain. In other embodiments, the filamentous fungal host is a Fusarium bactridioides, Fusarium cerealis, Fusarium crookwellense, Fusarium culmorum, Fusarium graminearum, Fusarium graminum, Fusarium heterosporum, Fusarium negundi, Fusarium oxysporum, Fusarium reticulatum, Fusarium roseum, Fusarium sambucinum, Fusarium sarcochroum, Fusarium sporotrichioides, Fusarium sulphureum, Fusarium torulosum, Fusarium trichothecioides, or Fusarium venenatum strain. In yet other preferred embodiments, the filamentous fungal host is a Humicola insolens, Humicola lanuginosa, Mucor miehei, Myceliophthora thermophila, Neurospora crassa, Penicillium purpurogenum, Scytalidium thermophilum, Sporotrichum thermophile, or Thielavia terrestris strain. In a further embodiment, the filamentous fungal host is a Trichoderma harzianum, Trichoderma koningii, Trichoderma longibrachiatum, Trichoderma reesei, or Trichoderma viride strain.

In some embodiments of the disclosure, the host cell is a Saccharomyces sp., Kluyveromyces sp., Pichia sp., Sporotrichum sp., Candida sp., Neurospora sp. Trichoderma sp., or Zymomonas sp. In some embodiments, the host cell is of a species selected from but not limited to Saccharomyces cerevisiae, Saccharomyces monacensis, Saccharomyces bayanus, Saccharomyces pastorianus, Saccharomyces carlsbergensis, Saccharomyces pombe, Kluyveromyces marxiamus, Kluyveromyces lactis, Kluyveromyces fragilis, Pichia stipitis, Sporotrichum thermophile, Candida shehatae, Candida tropicalis, Neurospora crassa, Trichoderma reesei, and Zymomonas mobilis. In some embodiments, the Saccharomyces sp. is an industrial Saccharomyces strain commonly used in bioethanol production as well as specific gene polymorphisms that are important for bioethanol production (Argueso et al., Genome Research, 19: 2258-2270, 2009). The host cells of the present disclosure are genetically modified in that recombinant nucleic acids have been introduced into the host cells, and as such the genetically modified host cells do not occur in nature.

In some aspects, the host cells of the present disclosure express proteins of a pentose utilization pathway. In some aspects, the host cells of the present disclosure express proteins of a cellobiose utilization pathway. The coding regions of the desired proteins may be heterologous to the host cell or endogenous to the host cell, but are operatively linked to heterologous promoters and/or terminators resulting in a different expression level of the coding region in the host cell. The term “endogenous” as used herein with reference to a nucleic acid or protein and a particular cell or microorganism refers to a nucleic acid or protein that is present in the cell and was not introduced into the cell using recombinant techniques (e.g., a gene found in the cell when it was originally isolated from nature). In contrast, the term “exogenous” as used herein with reference to a nucleic acid or protein and a particular cell or microorganism refers to a nucleic acid or protein that is not present in the cell (e.g., foreign nucleic acid or protein) and was introduced into the cell using recombinant techniques.

The term “heterologous” as used in reference to a coding region of a protein of interest and flanking sequences such as a 5′ promoter and a 3′ terminator, indicate that the flanking sequences are non-native to the coding region. For instance, a PGK1 promoter and a CYC1 terminator are heterologous to a XDH coding region. In contrast, the term “homologous” as used in reference to a coding region of a protein of interest and flanking sequences such as a 5′ promoter and a 3′ terminator, indicate that the flanking sequences are native to the coding region. For instance, a XDH promoter and a XDH terminator are homologous to a XDH coding region.

“Genetically engineered” or “genetically modified” refer to any recombinant DNA or RNA method used to create a prokaryotic or eukaryotic host cell that expresses a protein at elevated levels, at lowered levels, or in a mutated form. In other words, the host cell has been transfected, transformed, or transduced with a recombinant polynucleotide molecule, and thereby has been altered so as to cause the cell to alter expression of a desired protein. Methods and vectors for genetically engineering host cells are well known in the art; for example various techniques are illustrated in Current Protocols in Molecular Biology, Ausubel et al., eds. (Wiley & Sons, New York, 1988, and quarterly updates).

Genetic modifications that result in an increase in gene expression or function can be referred to as amplification, overproduction, overexpression, activation, enhancement, addition, or up-regulation of a gene. More specifically, reference to increasing the action (or activity) of enzymes or other proteins discussed herein generally refers to any genetic modification of the host cell in question which results in increased expression and/or functionality (biological activity) of the enzymes or proteins and includes higher activity or action of the proteins (e.g., specific activity or in vivo enzymatic activity), reduced inhibition or degradation of the proteins, and overexpression of the proteins. For example, gene copy number can be increased, expression levels can be increased by use of a promoter that gives higher levels of expression than that of the native promoter, or a gene can be altered by genetic engineering or classical mutagenesis to increase the biological activity of an enzyme or action of a protein. Combinations of some of these modifications are also possible.

Genetic modifications which result in a decrease in gene expression, in the function of the gene, or in the function of the gene product (i.e., the protein encoded by the gene) can be referred to as inactivation (complete or partial), deletion, interruption, blockage, silencing, or down-regulation, or attenuation of expression of a gene. For example, a genetic modification in a gene which results in a decrease in the function of the protein encoded by such gene, can be the result of a complete deletion of the gene (i.e., the gene does not exist, and therefore the protein does not exist), a mutation in the gene which results in incomplete or no translation of the protein (e.g., the protein is not expressed), or a mutation in the gene which decreases or abolishes the natural function of the protein (e.g., a protein is expressed which has decreased or no enzymatic activity or action). More specifically, reference to decreasing the action of proteins discussed herein generally refers to any genetic modification in the host cell in question, which results in decreased expression and/or functionality (biological activity) of the proteins and includes decreased activity of the proteins (e.g., decreased transport), increased inhibition or degradation of the proteins as well as a reduction or elimination of expression of the proteins. For example, the action or activity of a protein of the present disclosure can be decreased by blocking or reducing the production of the protein, reducing protein action, or inhibiting the action of the protein. Combinations of some of these modifications are also possible. Blocking or reducing the production of a protein can include placing the gene encoding the protein under the control of a promoter that requires the presence of an inducing compound in the growth medium. By establishing conditions such that the inducer becomes depleted from the medium, the expression of the gene encoding the protein (and therefore, of protein synthesis) could be turned off.

In general, according to the present disclosure, an increase or a decrease in a given characteristic of a multi-enzyme pathway (e.g., enzyme expression) is made with reference to the same characteristic of a reference multi-enzyme pathway (e.g., scaffolds such as those provided for the three gene xylose utilization pathway and the five gene xylose/arabinose utilization pathway), which is measured or established under the same or equivalent conditions. Similarly, an increase or decrease in a characteristic of a genetically modified host cell (e.g., enzyme expression) is made with reference to the same characteristic of a reference host cell (e.g., wild-type host cell of the same species, preferably the same strain or a recombinant host cell of the sam species, preferably the same strain, which has been transformed with an expression vector of a multi-enzyme pathway), under the same or equivalent conditions. Such conditions include the assay or culture conditions (e.g., medium components, temperature, pH, etc.) under which the activity of the protein or other characteristic of the host cell is measured, as well as the type of assay used. As discussed above, equivalent conditions are conditions (e.g., culture conditions) which are similar, but not necessarily identical (e.g., some conservative changes in conditions can be tolerated), and which do not substantially change the effect on cell growth or enzyme expression or biological activity as compared to a comparison made under the same conditions.

Methods of Producing and Culturing Host Cells

The disclosure herein relates to host cells containing recombinant polynucleotides encoding polypeptides of a pentose and/or cellobiose utilization pathway. Further described herein are methods of increasing growth of a host cell on a medium containing a pentose and/or cellobiose, and methods of co-fermenting cellulose-derived and/or hemicellulose-derived pentose and/or cellobiose molecules by providing a host cell containing one or more recombinant polynucleotide(s) encoding polypeptides of a pentose and/or cellobiose utilization pathway.

Methods of producing and culturing host cells of the disclosure may include the introduction or transfer of expression vectors containing the recombinant polynucleotides of the disclosure into the host cell. Such methods for transferring expression vectors into host cells are well known to those of ordinary skill in the art.

The vectors preferably contain one or more selectable markers which permit easy selection of transformed hosts. A selectable marker is a gene encoding a product which provides, for example, biocide or viral resistance, resistance to heavy metals, prototrophy to auxotrophs, and the like. Selection of recombinant cells may be based upon antimicrobial resistance that has been conferred by genes such as the amp, gpt, neo, and hyg genes.

Suitable markers for yeast hosts are, for example, ADE2, HIS3, LEU2, LYS2, MET3, TRP1, and URA3. Selectable markers for use in a filamentous fungal host include, but are not limited to, amdS (acetamidase), argB (ornithine carbamoyltransferase), bar (phosphinothricin acetyltransferase), hph (hygromycin phosphotransferase), niaD (nitrate reductase), pyrG (orotidine-5′-phosphate decarboxylase), sC (sulfate adenyltransferase), and trpC (anthranilate synthase), as well as equivalents thereof. Preferred for use in Aspergillus are the amdS and pyrG genes of Aspergillus nidulans or Aspergillus oryzae and the bar gene of Streptomyces hygroscopicus. Preferred for use in Trichoderma are bar and amdS.

For integration into the host genome, the vector may rely on the sequence of a gene of interest or any other element of the vector for integration of the vector into the genome by homologous or nonhomologous recombination. Alternatively, the vector may contain additional nucleotide sequences for directing integration by homologous recombination into the genome of the host (e.g., delta sequence). The additional nucleotide sequences enable the vector to be integrated into the host genome at a precise location(s) in the chromosome(s). To increase the likelihood of integration at a precise location, the integrational elements should preferably contain a sufficient number of nucleic acids, such as 100 to 10,000 base pairs, preferably more than about 500, 1,000, 1,500 or 2,000 base pairs, which are highly homologous with the corresponding target sequence to enhance the probability of homologous recombination. The integrational elements may be any sequence that is homologous with the target sequence in the genome of the host. Furthermore, the integrational elements may be non-coding or coding nucleotide sequences. On the other hand, the vector may be integrated into the genome of the host by non-homologous recombination.

For autonomous replication, the vector may further contain an origin of replication, enabling the vector to replicate autonomously in the host in question. The origin of replication may be any plasmid replicator mediating autonomous replication in a cell of interest. The term “origin of replication” or “plasmid replicator” is defined herein as a sequence that enables a plasmid or vector to replicate in vivo. Examples of origins of replication for use in a yeast host are the 2 micron origin of replication, ARS1, ARS4, the combination of ARS1 and CEN3, and the combination of ARS4 and CEN6. Examples of origins of replication useful in a filamentous fungal cell are AMA1 and ANS1 (WO 00/24883). Isolation of the AMA1 gene and construction of plasmids or vectors containing the gene can be accomplished according to known methods (WO 00/24883). For other hosts, transformation procedures are described, for example, in Read et al., Appl Environ Microbiol, 73:5088-5096, 2007 for Kluyveromyces, in Osvaldo Delgado et al., FEMS Microbiology Letters 132:23-26, 1995 for Zymomonas, in U.S. Pat. No. 7,501,275 for Pichia, and in WO 2008/040387 for Clostridium.

More than one copy of a gene may be inserted into the host to increase production of the gene product. An increase in the copy number of the gene can be obtained by integrating at least one additional copy of the gene into the host genome or by including an amplifiable selectable marker gene with the nucleotide sequence where cells containing amplified copies of the selectable marker gene, and thereby additional copies of the gene, can be selected for by cultivating the cells in the presence of the appropriate selectable agent.

Once the host cell has been transformed with the expression vector, the host cell is allowed to grow. Methods of the disclosure may include culturing the host cell such that recombinant nucleic acids in the cell are expressed. For microbial hosts, this process entails culturing the cells in a suitable medium. Typically cells are grown at 35° C. in appropriate media. Growth media in the present disclosure include, for example, common commercially prepared media such as Luria Bertani (LB) broth, Sabouraud Dextrose (SD) broth or Yeast medium (YM) broth. Other defined or synthetic growth media may also be used and the appropriate medium for growth of the particular host cell will be known by someone skilled in the art of microbiology or fermentation science.

According to some aspects of the disclosure, the culture media contains a carbon source for the host cell. Such a “carbon source” generally refers to a substrate or compound suitable to be used as a source of carbon for prokaryotic or simple eukaryotic cell growth. Carbon sources can be in various forms, including, but not limited to polymers, carbohydrates, acids, alcohols, aldehydes, ketones, amino acids, peptides, etc. These include, for example, various monosaccharides (e.g., glucose, xylose, arabinose, etc.), disaccharides, oligosaccharides, polysaccharides, a biomass polymer such as cellulose or hemicellulose, saturated or unsaturated fatty acids, succinate, lactate, acetate, ethanol, etc., or mixtures thereof. In some preferred embodiments, the carbon source is a product of photosynthesis, including, but not limited to glucose.

In some embodiments, the carbon source includes a biomass polymer such as cellulose or hemicellulose, and/or carbohydrates derived therefrom. “A biomass polymer” as described herein is any polymer contained in biological material. The biological material may be living or dead. Non-limiting examples of sources of a biomass polymer include grasses (e.g., switchgrass, Miscanthus), rice hulls, bagasse, cotton, jute, hemp, flax, bamboo, sisal, abaca, straw, leaves, grass clippings, corn stover, corn cobs, distillers grains, legume plants, sorghum, sugar cane, sugar beet pulp, wood chips, sawdust, and biomass crops (e.g., Crambe).

In addition to an appropriate carbon source, media must contain suitable minerals, salts, cofactors, buffers and other components, known to those skilled in the art, suitable for the growth of the cultures and promotion of the enzymatic pathways necessary for the fermentation of various sugars and the production of hydrocarbons and hydrocarbon derivatives. Reactions may be performed under aerobic or anaerobic conditions, where aerobic, anoxic, or anaerobic conditions are preferred based on the requirements of the microorganism. As the host cell grows and/or multiplies, it expresses enzymes of the substrate utilization pathway necessary for growth on the substrate.

### EXPERIMENTAL

The present disclosure is described in further detail in the following examples, which are not in any way intended to limit the scope of the disclosure as claimed.

In the experimental disclosure which follows, the following abbreviations apply: LAD (L-arabitol 4-dehydrogenase); LXR (L-xylulose reductase); μL (microliter); NAD (nicotinamide adenine dinucleotide); NADP (nicotinamide adenine dinucleotide phosphate); OE-PCR (overlap extension PCR); ORF (open reading frame); PCR (polymerase chain reaction); SC-Ura (synthetic complete culture lacking uracil); SC-Ura-G (SC-Ura with glucose); TAL (transaldolase); (TKL) transketolase, XDH (xylitol dehydrogenase); XKS (xylulokinase); XR (xylose reductase); YP (yeast extract and peptone); YPA (yeast extract, peptone and adenine hemisulfate); YPX (yeast extract, peptone, and xylose).

### Example 1

### Genome Mining of Enzyme Homologues for Pentose Utilization

To identify enzyme homologues for pathway assembly, an intensive literature search was first performed to identify known xylose reductases, xylitol dehydrogenases, xylulokinases, L-arabitol 4-dehydrogenases, and L-xylulose reductases. Genome mining was also performed at various databases (NCBI, NCBI BLAST, and BRENDA Enzyme databases) to identify genes encoding those enzymes based on annotation and sequence homology. In addition, several codon-optimized genes and mutants with altered cofactor specificity were also cloned and included in the library. Nucleic acids encoding these enzymes were obtained by introduction of mutations into the wildtype gene through site specific mutagenesis, or by synthesis of codon optimized genes by DNA2.0 (Menlo Park, Calif.).

To obtain the open reading frames (ORFs) encoding other enzyme homologues, strains carrying corresponding genes were obtained from various culture collections: Agriculture Research Services ARS (NRRL) Culture Collection; German Resource Centre for Biological Material (DSMZ); and Fungal Genetics Stock Center (FGSC). The strains were cultivated in YP media supplemented with xylose or arabinose and then total RNA and genomic DNA were isolated. The total RNA was reverse transcribed into cDNA and primers were designed based on known gene sequences from the GENBANK to amplify the ORFs.

In total, 20 xylose reductase homologues, 22 xylitol dehydrogenase homologues, 19 xylulokinase homologues, 16 L-arabitol 4-dehydrogenase homologues and 11 L-xylulose reductase homologues were cloned for inclusion in the combinatorial pathway library.

List of Enzyme Homologs:

| Abbreviation | Source | LOCUS | Annotation |
|---|---|---|---|
| aoXR | Aspergillus oryzae | XP_001819987 | NAD(P)H-dependent D-xylose |
| | | | reductase xyl1 |
| pgXR | Pichia guilliermondii¹ | AAD09330 | xylose reductase |
| ctrXR | Candida tropicalis | ABX60132 | xylose reductase |
| klXR | Kluyveromyces lactis | AAA99507 | xylose reductase |
| csXR | Candida shehatae | ABK35120 | xylose reductase |
| psXR | Pichia stipitis | CAA42072 | xylose reductase |
| cpXR | Candida parapsilosis | ABK32844 | xylose reductase |
| afXR | Aspergillus flavus | XYL1_ASPFN | xylose reductase |
| MoXR | Magnaporthe oryzae | XP_363305 | conserved hypothetical protein |
| ZrXR | Zygosaccharomyces | XP_002494646 | hypothetical protein |
| | rouxii | | |
| tsXR | Talaromyces stipitatus | XP_002484051 | D-xylose reductase (Xyl1), putative |
| paXR | Podospora anserina | XP_001912586 | hypothetical protein |
| ppXR | Pichia pastoris | XP_002492973 | Aldose reductase involved in |
| | | | methylglyoxal, D-xylose and |
| | | | arabinose metabolism |
| pnXR | Phaeosphaeria | XP_001803042 | hypothetical protein |
| | nodorum | | |
| pcXR | Penicillium | XP_002561272 | hypothetical protein |
| | chrysogenum | | |
| mgXR | Meyerozyma | ABB87188 | putative gamma-butyrobetaine |
| | guilliermondii | | hydroxylase |
| anXR | Aspergillus niger | XP_001388804 | NAD(P)H-dependent D-xylose |
| | | | reductase xyl1 |
| anidXR | Aspergillus nidulans | XP_658027 | hypothetical protein |
| psXR_m ² | Pichia stipitis | N.A. | K270R mutant of psXR |
| ncXR | Neurospora crassa | XM_958838 | Xylose reductase |
| aoXDH | Aspergillus oryzae | XP_001825523 | D-xylulose reductase |
| anidXDH | Aspergillus nidulans | XP_682333 | hypothetical protein |
| caXDH | Candida albicans | XP_719434 | hypothetical protein |
| cdXDH | Candida dubliniensis | XP_002422539 | xylitol dehydrogenase, putative |
| hjXDH | Hypocrea jecorina | AF428150_1 | xylitol dehydrogenase |
| ncXDH | Neurospora crassa | XP_964807 | hypothetical protein |
| nhXDH | Nectria haematococca | XP_003053965 | predicted protein |
| paXDH | Pichia angust | BAD32688 | glycerol dehydrogenase |
| pcXDH | Penicillum | XP_002568185 | hypothetical protein |
| | chrysogenum | | |
| pnXDH | Phaeosphaeria | XP_001801634 | hypothetical protein |
| | nodorum | | |
| ppXDH | Pichia pastoris | XP_002489933 | hypothetical protein |
| zrXDH | Zygosaccharomyces | XP_002497308 | Sorbitol dehydrogenase |
| | rouxii | | |
| baXDH | Blastobotrys | CAG34729 | xylitol dehydrogenase |
| | adeninivorans | | |
| psXDH | Pichia stipitis³ | XP_001386982 | Xylitol dehydrogenase |
| anXDH | Aspergillus niger | XP_001395093 | D-xylulose reductase A |
| pgXDH | Pichia guilliermondii¹ | XP_001481963 | hypothetical protein |
| ctXDH | Candida tropicalis | XP_002546318 | D-xylulose reductase |
| klXDH | Kluyveromyces lactis | XP_453306 | hypothetical protein |
| csXDH | Candida shehatae | ACI01079 | xylitol dehydrogenase |
| tsXDH | Talaromyces stipitatus | XP_002488234 | xylitol dehydrogenase |
| ptXDH | Pachysolen tannophilus | ACD81475 | alcohol dehydrogenase |
| ncXDH_m | Neurospora crassa | N.A. | ARS mutant of ncXDH |
| anXKS | Aspergillus niger | XP_001391397 | D-xylulose kinase A |
| caXKS | Candida albicans | XP_711437 | potential xylulokinase Xks1p |
| ctXKS | Candida tropicalis | XP_002549576 | hypothetical protein |
| pcXKS | Penicillium | CAP80202 | strong similarity to D-xylulokinase |
| | chrysogenum | | Xks1Saccharomyces cerevisiae |
| psXKS | Pichia stipitis³ | AAF72328 | D-xylulokinase |
| scXKS | Sacchammyces | EDN61781 | xylulokinase |
| | cerevisiae | | |
| ppXKS | Pichia pastoris | XP_002489935 | Xylulokinase, converts D-xylulose |
| | | | and ATP to xylulose 5-phosphate |
| cdXKS | Candida dubliniensis | CAX42363 | xylulokinase, putative |
| ncXKS | Neurospora crassa | XP_001728137 | hypothetical protein |
| klXKS | Kluyveromyces lactis | XP_454390 | hypothetical protein |
| mgXKS | Meyerozyma | XP_001482343 | hypothetical protein |
| | guilliermondii | | |
| paXKS | Podospora anserine | XP_001907775 | hypothetical protein |
| afXKS | Aspergillus flavus | XP_002383697 | D-xylulose kinase |
| afuXKS | Aspergillus fumigatus | XP_753656 | D-xylulose kinase |
| tsXKS | Talaromyces stipitatus | XP_002484260 | D-xylulose kinase |
| anidXKS | Aspergillus nidulans | XP_682059 | hypothetical protein |
| aoXKS | Aspergillus oryzae | XP_001824894 | D-xylulose kinase A |
| zrXKS | Zygosaccharomyces | XP_002498508 | hypothetical protein |
| | rouxii | | |
| nhXKS | Nectria haematococca | XP_003048965 | predicted protein |

¹Meyerozyma guilliermondii
² Watanabe, S., Pack, S. P., Abu Saleh, A., Annaluru, N., Kodaki, T. and Makino, K. (2007) The positive effect of the decreased NADPH-preferring activity of xylose reductase from Pichia stipitis on ethanol production using xylose-fermenting recombinant Saccharomyces cerevisiae. Bioscience Biotechnology and Biochemistry, 71, 1365-1369.
³Scheffersomyces stipitis

The amino acid sequences of the pentose-utilization pathway enzymes are provided below.

Xylose Reductase (XR) Sequences
Xylose reductase homolog of Aspergillus oryzae
(SEQ ID NO: 1):
MASPTVKLNSGHDMPLVGFGLWKVNNETCADQVYEAIKAGYRLFDGACDYGNEVECG
QGVARAIKEGIVKREELFIVSKLWNSFHEGDRVEPICRKQLADWGVDYFDLYIVHFPVA
LKYVDPAVRYPPGWNSESGKIEFSNATIQETWTAMESLVDKKLARSIGVSNFSAQLLMD
LLRYARVRPATLQIEHHPYLTQPRLVEYAQKEGIAVTAYSSFGPLSFLELEVKNAVDTPP
LFEHNTIKSLAEKYGKTPAQVLLRWATQRGIAVIPKSNNPTRLSQNLEVTGWDLEKSELE
AISSLDKGLRFNDPIGYGMYVPIF.
Xylose reductase homolog of Candida parapsilosis
(SEQ ID NO: 2):
MSIKLNSGHEMPIVGFGCWKVTNETAADQIYNAIKVGYRLFDGAQDYGNEKEVGEGIN
RAIDEGLVSRDELFVVSKLWNNYHDPKNVETALNKTLSDLNLEYLDLFLIHFPIAFKFVPI
EEKYPPGFYCGDGDKFHYENVPLLDTWRALESLVQKGKIRSIGISNFNGGLIYDLVRGA
KIKPAVLQIEHHPYLQQPRLIEFVQSQGIAITGYSSFGPQSFLELESKKALDTPTLFDHETI
KSIASKHKKSSAQVLLRWATQRGIAVIPKSNNPDRLAQNLNVSDFELSKEDLEAINKLDK
GLRFNDPWDWDHIPIFV.
Xylose reductase homolog of Candida shehatae
(SEQ ID NO: 3):
MSPSPIPAFKLNNGLEMPSIGFGCWKLGKSTAADQVYNAIKAGYRLFDGAEDYGNEQE
VGEGVKRAIDEGIVTREEIFLTSKLWNNYHDPKNVETALNKTLKDLKVDYVDLFLIHFPI
AFKFVPIEEKYPPGFYCGDGDNFVYEDVPILETWKALEKLVKAGKIRSIGVSNFPGALLL
DLFRGATIKPAVLQVEHHPYLQQPKLIEYAQKVGITVTAYSSFGPQSFVEMNQGRALNT
PTLFEHDVIKAIAAKHNKVPAEVLLRWSAQRGIAVIPKSNLPERLVQNRSFNDFELTKED
FEEISKLDINLRFNDPWDWDNIPIFV.
Xylose reductase homolog of Candida tropicalis
(SEQ ID NO: 4):
MSTTVNTPTIKLNSGYEMPLVGFGCWKVTNATAADQIYNAIKTGYRLFDGAEDYGNEK
EVGEGINRAIKDGLVKREELFITSKLWNNFHDPKNVETALNKTLSDLNLDYVDLFLIHFPI
AFKFVPIEEKYPPGFYCGDGDNFHYEDVPLLDTWKALEKLVEAGKIKSIGISNFTGALIY
DLIRGATIKPAVLQIEHHPYLQQPKLIEYVQKAGIAITGYSSFGPQSFLELESKRALNTPTL
FEHETIKSIADKHGKSPAQVLLRWATQRNIAVIPKSNNPERLAQNLSVVDFDLTKDDLDN
IAKLDIGLRFNDPWDWDNIPIFV.
Xylose reductase homolog of Kluyveromyces lactis
(SEQ ID NO: 5):
MTYLAETVTLNNGEKMPLVGLGCWKMPNDVCADQIYEAIKIGYRLFDGAQDYANEKE
VGQGVNRAIKEGLVKREDLVVVSKLWNSFHHPDNVPRALERTLSDLQLDYVDIFYIHFP
LAFKPVPFDEKYPPGFYTGKEDEAKGHIEEEQVPLLDTWRALEKLVDQGKIKSLGISNFS
GALIQDLLRGARIKPVALQIEHHPYLTQERLIKYVKNAGIQVVAYSSFGPVSFLELENKK
ALNTPTLFEHDTIKSIASKHKVTPQQVLLRWATQNGIAIIPKSSKKERLLDNLRINDALTL
TDDELKQISGLNQNIRFNDPWEWLDNEFPTFI.
Xylose reductase homolog of Neurospora crassa
(SEQ ID NO: 6):
MVPAIKLNSGFDMPQVGFGLWKVDGSIASDVVYNAIKAGYRLFDGACDYGNEVECGQ
GVARAIKEGIVKREELFIVSKLWNTFHDGDRVEPIVRKQLADWGLEYFDLYLIHFPVALE
YVDPSVRYPPGWHFDGKSEIRPSKATIQETWTAMESLVEKGLSKSIGVSNFQAQLLYDL
LRYAKVRPATLQIEHHPYLVQQNLLNLAKAEGIAVTAYSSFGPASFREFNMEHAQKLQP
LLEDPTIKAIGDKYNKDPAQVLLRWATQRGLAIIPKSSREATMKSNLNSLDFDLSEEDIK
TISGFDRGIRFNQPTNYFSAENLWIFG.
Xylose reductase homolog of Pichia guilliermondii
(SEQ ID NO: 7):
MSIKLNSGYDMPSVGFGCWKVDNATCADTIYNAIKVGYRLFDGAEDYGNEKEVGDGIN
RALDEGLVARDELFVVSKLWNSFHDPKNVEKALDKTLSDLKVDYLDLFLIHFPIAFKFV
PFEEKYPPGFYCGDGDKFHYEDVPLIDTWRALEKLVEKGKIRSIGISNFSGALIQDLLRSA
KIKPAVLQIEHHPYLQQPRLVEYVQSQGIAITAYSSFGPQSFVELDHPRVKDVKPLFEHD
VIKSVAGKVKKTPAQVLLRWATQRGLAVIPKSNNPDRLLSNLKVNDFDLSQEDFQEISK
LDIELRFNNPWDWDKIPTFI.
Xylose reductase homolog of Pichia stipitis
(SEQ ID NO: 8):
MPSIKLNSGYDMPAVGFGCWKVDVDTCSEQIYRAIKTGYRLFDGAEDYANEKLVGAG
VKKAIDEGIVKREDLFLTSKLWNNYHHPDNVEKALNRTLSDLQVDYVDLFLIHFPVTFK
FVPLEEKYPPGFYCGKGDNFDYEDVPILETWKALEKLVKAGKIRSIGVSNFPGALLLDLL
RGATIKPSVLQVEHHPYLQQPRLIEFAQSRGIAVTAYSSFGPQSFVELNQGRALNTSPLFE
NETIKAIAAKHGKSPAQVLLRWSSQRGIAIIPKSNTVPRLLENKDVNSFDLDEQDFADIAK
LDINLRFNDPWDWDKIPIFV.
Xylose reductase homolog of Aspergillus flavus, NRRL3357,
Xyl1 (SEQ ID NO: 9):
MASPTVKLNSGHDMPLVGFGLWKVNNETCADQVYEAIKAGYRLFDGACDYGNEVECG
QGVARAIKEGIVKREELFIVSKLWNSFHEGDRVEPICRKQLADWGVDYFDLYIVHFPVA
LKYVDPAVRYPPGWNSESGKIEFSNATIQETWTAMESLVDKKLARSIGVSNFSAQLLMD
LLRYARVRPATLQIEHHPYLTQPRLVEYAQKEGIAVTAYSSFGPLSFLELEVKNAVDTPP
LFEHNTIKSLAEKYGKTPAQVLLRWATQRGIAVIPKSNNPTRLSQNLEVTGWDLEKSELE
AISSLDKGLRFNDPIGYGMYVPIF.
Xylose reductase homolog of Magnaporthe oryzae 70-15
(SEQ ID NO: 10):
MSATNGSAAAAPSKKNIGVFTNPKHDLWINEAEPSLESVQKGSDELKEGQVTIAIRSTGI
CGSDVHFWHHGCIGPMIVREDHILGHESAGEIIAVHPSVTSLKVGDRVAVEPQVICYECE
PCLTGRYNGCEKVDFLSTPPVPGLLRRYVNHPAVWCHKIGDMSWEDGAMLEPLSVALA
GIQRAGITLGDPVLVCGAGPIGLITLLCAKAAGACPLVITDIDDGRLKFAKELVPDVITFK
VEGRPTAEDAAKSIVEAFGGVEPTLAIECTGVESSIASAIWAVKFGGKVFVIGVGRNEISL
PFMRASVREVDLQFQYRYCNTWPRAIRLIQNKVIDLTKLVTHRFPLEDALKAFETAADP
KTGAIKVQIQSLE.
Xylose reductase homolog of Zygosaccharomyces rouxii,
ZYRO0A06336g (SEQ ID NO: 11):
MASVVALNNGNKMPLVGLGCWKIPNETCSQQIYDAISVGYRVFDGAQDYGNEKEVGE
GVRRAIKDGLVKREELFVVSKLWNSFHHPKNVKLALKRTLSDMGLDYLDLFYIHFPIAL
KPVSFEEKYPPGLYTGEADAKAGVLSEEPVPILDTYRALEECVEEGLIKSIGVSNFSGSIM
LDLLRGARIPPAALQIELHPYLTQERYVKWVQSKGIQVVAYSSFGPQSFVDIGSEVAKAT
PPLFEHDVVKKIAAKHNVSTSQVLLRWATQQKVAVIPKSSKKERLRQNLLVDQEVTLTG
DEIKEISGLNKNLRFNDPFTWSEKTPFPIFD.
Xylose reductase homolog of Talaromyces stipitatus, ATCC
10500, Xyl1 (SEQ ID NO: 12):
MSSPTVKLNSGYDMPLVGFGLWKVNNDTCADQVYAAIKAGYRLFDGACDYGNEKEV
GQGIARAIKDGLVKREELFIVSKLWNTFHDGDKVEPIARKQLDDLGLDYFDLYLIHFPVA
LKWVDPAERYPPGWTAPDGKVEFSKATIQETWQAMESLVDKKLSRSIGISNFSVQLIMD
LLRHARIRPATLQIEHHPYLQQKELIKYVQSEGIVITAYSSFGPLSFIELDMSSAHNTPKLF
DHDVIKSTSQKHGKTPAQILLRWATQRNIAVIPKSNDPTRLSQNLDVTGWSLEQSDIDAI
NGLDLGLRFNDPLNYGIYIPIFA.
Xylose reductase homolog of Podospora anserina S mat+
(SEQ ID NO: 13):
MAPVIKLNSGYDMPQVGFGLWKVDNAIAADVVYNAIKAGYRLFDGACDYGNEVECGK
GVARAISEGIVKREDLFIVSKLWNTFHDGERVQPIVKKQLADWGVDYFDLYLIHFPVAL
EYVDPSVRYPPGWHYEGDEIRPSKATIQETWTAMESLVDAGLARSIGISNFQSQLIYDLL
RYAKIRPATLQIEHHPYLTQEELLKLAKREGITVTAYSSFGPASFLEFNMQHAVKLQPLM
EDDTIKAIAAKYNRPASQVLLRWATQRGLAVIPKSSRQETMVSNLQNTDFDLSEEDIATI
SGFNRGIRFNQPSNYFPTELLWIFG.
Xylose reductase homolog of Pichia pastoris GS115
(SEQ ID NO: 14):
MATLLKLNNGLKLPQVGLGVWKIPNELTAETVYNAIKQGYRLFDGAEDYGNEKEVGQ
GVRRAIDEGLVKREDLFIVSKLWNNYHHPDNVGKALDRTLSDLGLDYLDLFYIHFPIAF
KFVPLEEKYPPAFYCGDGNNFHYEDVPLLDTYRALERLVDAGRIKSLGVSNFNGALLQD
LLRGARIKPVALQIEHHPYLVQQKLIEYAQSEDIVVVAYSSFGPQSFLELKVNKALTAVS
LFEHDVIKKIAQAHNRSAGEVLLRWATQRGLAIIPKSSKPERLSSNLHINSFDLTKEDLETI
SSLDLGLRFNDPWDWDKIPIFA.
Xylose reductase homolog of Phaeosphaeria nodorum SN15
(SEQ ID NO: 15):
MVAGRFCRTSINTVRSFTTAVVPRSSFFPPVRTCISRTKAPSFRPTYSNRNFFATMAVNTP
YITLNDGNKMPQVGFGLWKVDNATCADTVYNAIKTGYRLFDGACDYGNEVECGQGV
ARAIKEGLVKREDLFIVSKLWQTFHDYEQVEPITKKQLKDWGIDYFDLYLIHFPVALKY
VSPETRYPPGWFSDEANSKVIHSKARLEDTWRAFEDIKSKGLTKSIGVSNYSGALLLDLF
TYAKVKPATLQIEHHPYYVQPYLIKLAEEHDIKVTAYSSFGPQSFIECDMKIAADTPLLFD
HPVIKKIAEKHSKTPAQILLRWSTQRGLSVIPKSNSQNRLQQNLDVTGFDMSESEIAEISD
LDKNLKFNAPTNYGIPCYVFA.
Xylose reductase homolog of Penicillium chrysogenum
Wisconsin 54-1255 (SEQ ID NO: 16):
MVAPTVKLSSGYEMPLVGFGLWKVNNDTCADQVYHAIKAGYRLFDGACDYGNEVEA
GQGVARAIKEGIVKREELFIVSKLWNSFHEADKVEPIARKQLADWGVDYFDLYIVHFPIA
LKYLDPSVRYPPSWTTAEGKIEFANAPIHETWGAMETLVDKKLARSIGVSNFSAQLLMD
LLRYARVRPATLQIEHHPYLTQTRLVDYAQKEGITVTAYSSFGPLSFLELDLKHAKDTPL
LFEHATITSIAEKHGRTPAQVLLRWSTQRNVAVIPKSNNPTRLAQNLTVTDFDLEASELE
AISALDKGLRFNDPIAVSLVCVEY.
Xylose reductase homolog of Meyerozyma guilliermondii,
anamorph of Candida guilliermondii (SEQ ID NO: 17):
MTKMDHKIVKTSYDGDAVSVEWDGGASAKFDNIWLRDNCHCSECYYDATKQRLLNSC
SIPDDIAPIKVDSSPTKLKIVWNHEEHQSEYECRWLVIHSYNPRQIPVTEKVSGEREILARE
YWTVKDMEGRLPSVDFKTVMASTDENEEPIKDWCLKIWKHGFCFIDNVPVDPQETEKL
CEKLMYIRPTHYGGFWDFTSDLSKNDTAYTNIDISSHTDGTYWSDTPGLQLFHLLMHEG
TGGTTSLVDAFHCAEILKKEHPESFELLTRIPVPAHSAGEEKVCIQPDIPQPIFKLDTNGELI
QVRWNQSDRSTMDSWENPSEVVKFYRAIKQWHKIISDPANELFYQLRPGQCLIFDNWR
CFHSRTEFTGKRRMCGAYINRDDFVSRLKLLNIGRQPVLDAI.
Xylose reductase homolog of Aspergillus niger
(SEQ ID NO: 18):
MASPTVKLNSGYDMPLVGFGLWKVNNDTCADQIYHAIKEGYRLFDGACDYGNEVEAG
QGIARAIKDGLVKREELFIVSKLWNSFHDGDRVEPICRKQLADWGIDYFDLYIVHFPISLK
YVDPAVRYPPGWKSEKDELEFGNATIQETWTAMESLVDKKLARSIGISNFSAQLVMDLL
RYARIRPATLQIEHHPYLTQTRLVEYAQKEGLTVTAYSSFGPLSFLELSVQNAVDSPPLFE
HQLVKSIAEKHGRTPAQVLLRWATQRGIAVIPKSNNPQRLKQNLDVTGWNLEEEEIKAI
SGLDRGLRFNDPLGYGLYAPIF.
Xylose reductase homolog of Aspergillus nidulans FGSC A4
(SEQ ID NO: 19):
MSPPTVKLNSGYDMPLVGFGLWKVNNDTCADQVYEAIKAGYRLFDGACDYGNEVEA
GQGVARAIKEGIVKRSDLFIVSKLWNSFHDGERVEPIARKQLSDWGIDYFDLYIVHFPVS
LKYVDPEVRYPPGWENAEGKVELGKATIQETWTAMESLVDKGLARSIGISNFSAQLLLD
LLRYARIRPATLQIEHHPYLTQERLVTFAQREGIAVTAYSSFGPLSFLELSVKQAEGAPPL
FEHPVIKDIAEKHGKTPAQVLLRWATQRGIAVIPKSNNPARLLQNLDVVGFDLEDGELK
AISDLDKGLRFNDPPNYGLPITIF.
Xylose reductase homolog of Pichia stipitis, K270R mutant
(SEQ ID NO: 20):
MPSIKLNSGYDMPAVGFGCWKVDVDTCSEQIYRAIKTGYRLFDGAEDYANEKLVGAG
VKKAIDEGIVKREDLFLTSKLWNNYHHPDNVEKALNRTLSDLQVDYVDLFLIHFPVTFK
FVPLEEKYPPGFYCGKGDNFDYEDVPILETWKALEKLVKAGKIRSIGVSNFPGALLLDLL
RGATIKPSVLQVEHHPYLQQPRLIEFAQSRGIAVTAYSSFGPQSFVELNQGRALNTSPLFE
NETIKAIAAKHGKSPAQVLLRWSSQRGIAIIPRSNTVPRLLENKDVNSFDLDEQDFADIAK
LDINLRFNDPWDWDKIPIFV.
Xylitol Dehydrogenase (XDH) Sequences
Xylitol dehydrogenase homolog of Aspergillus oryzae
(SEQ ID NO: 22):
MGAPPKTAQNLSFVLEGIHKVKFEDRPIPQLRDAHDVLVDVRFTGICGSDVHYWEHGSI
GQFVVKDPMVLGHESSGVISKVGSAVTTLKVGDHVAMEPGIPCRRCEPCKEGKYNLCE
KMAFAATPPYDGTLAKYYVLPEDFCYKLPENINLQEAAVMEPLSVAVHIVKQANVAPG
QSVVVFGAGPVGLLCCAVARAFGSPKVIAVDIQKGRLEFAKKYAATAIFEPSKVSALEN
AERIVNENDLGRGADIVIDASGAEPSVHTGIHVLRPGGTYVQGGMGRNEITFPIMAACTK
ELNVRGSFRYGSGDYKLAVNLVASGKVSVKELITGVVSFEDAEQAFHEVKAGKGIKTLI
AGVDV.
Xylitol dehydrogenase homolog of Aspergillus nidulans
(SEQ ID NO: 23):
MSSQTPTAQNLSFVLEGIHRVKFEDRPIPKLKSPHDVIVNVKYTGICGSDVHYWDHGAIG
QFVVKEPMVLGHESSGIVTQIGSAVTSLKVGDHVAMEPGIPCRRCEPCKAGKYNLCEK
MAFAATPPYDGTLAKYYTLPEDFCYKLPESISLPEGALMEPLGVAVHIVRQANVTPGQT
VVVFGAGPVGLLCCAVAKAFGAIRIIAVDIQKPRLDFAKKFAATATFEPSKAPATENATR
MIAENDLGRGADVAIDASGVEPSVHTGIHVLRPGGTYVQGGMGRSEMNFPIMAACTKE
LNIKGSFRYGSGDYKLAVQLVASGQINVKELITGIVKFEDAEQAFKDVKTGKGIKTLIAG
PGAAYKLAVQLVASGQINVKELITGIVKFEDAEQAFKDVKTGKGIKTLIAGPGAA.
Xylitol dehydrogenase homolog of Candida albicans
(SEQ ID NO: 24):
MTNPSLVLNKIDDISFEDYESPEITSPRDVIVEVKKTGICGSDIHYYAHGSIGPFVLRKPMV
LGHESAGVVVAVGDDVTNLKVGDKVAIEPGVPSRYSDEYKSGNYHLCPHMAFAATPP
VNPDEPNPPGTLCKYYKAPADFLFKLPDHVSLELGAMVEPLTVGVHACKLANLKFGEN
VVVFGAGPVGLLTAAVAKTIGAKNIMVVDIFDNKLQMAKDMGAATHTFNSKTGDDLV
KAFDGIEPSVVLECSGAKQCIYTGVKILKAGGRFVQVGNAGGDVNFPIADFSTRELTLYG
SFRYGYGDYQTSIDILDKNYINGKENAPINFELLITHRFKFKDAIKAYDLVRGGNGAVKC
LIDGPE.
Xylitol dehydrogenase homolog of Candida dubliniensis
(SEQ ID NO: 25):
MTPNPSLVLNKIDDISFEEYESPEITSPRDVIVEVKKTGICGSDIHYYAHGKIGPFVLRKPM
VLGHESAGVVVAVGDDVKNLKVGDNVAIEPGVPSRYSDEYKSGNYHLCPHMAFAATP
PVNPDEPNPPGTLCKYYKAPADFLFKLPDHVSLELGAMVEPLTVGVHACKLANLKFGE
NVVVFGAGPVGLLTAAVAKTIGAKNIMVVDIFDNKLKMAKDMGVATHTFNSKTGGDD
RDLVKHFDGIEPSVVLECSGAKQCIYTGVKVLKAGGRFVQVGNAGGDVNFPIADFSTRE
LALYGSFRYGYGDYQTSIDILDKNYINGKDNAPINFELLITHRFKFKDAIKAYDLVRGGN
GAVKCLIDGPE.
Xylitol dehydrogenase homolog of Hypocrea jecorina
(SEQ ID NO: 26):
MATQTINKDAISNLSFVLNKPGDVTFEERPKPTITDPNDVLVAVNYTGICGSDVHYWVH
GAIGHFVVKDPMVLGHESAGTVVEVGPAVKSLKPGDRVALEPGYPCRRCSFCRAGKYN
LCPDMVFAATPPYHGTLTGLWAAPADFCYKLPDGVSLQEGALIEPLAVAVHIVKQARV
QPGQSVVVMGAGPVGLLCAAVAKAYGASTIVSVDIVQSKLDFARGFCSTHTYVSQRISA
EDNAKAIKELAGLPGGADVVIDASGAEPSIQTSIHVVRMGGTYVQGGMGKSDITFPIMA
MCLKEVTVRGSFRYGAGDYELAVELVRTGRVDVKKLITGTVSFKQAEEAFQKVKSGEA
IKILIAGPNEKV.
Xylitol dehydrogenase homolog of Neurospora crassa
(SEQ ID NO: 27):
MATDGKSNLSFVLNKPLDVCFQDKPVPKINSPHDVLVAVNYTGICGSDVHYWLHGAIG
HFVVKDPMVLGHESAGTIVAVGDAVKTLSVGDRVALEPGYPCRRCVHCLSGHYNLCPE
MRFAATPPYDGTLTGFWTAPADFCYKLPETVSLQEGALIEPLAVAVHITKQAKIQPGQT
VVVMGAGPVGLLCAAVAKAYGASKVVSVDIVPSKLEFAKSFAATHTYLSQRVSPEENA
RNIIAAADLGEGADAVIDASGAEPSIQAALHVVRQGGHYVQGGMGKDNIIFPIMALCIKE
VTASGSFRYGSGDYRLAIQLVEQGKVDVKKLVNGVVPFKNAEEAFKKVKEGEVIKILIA
GPNEDVEGSLDTTVDEKKLNEAKACGGSGCC.
Xylitol dehydrogenase homolog of Nectria haematococca
(SEQ ID NO: 28):
MASNLSFVLNKPGDVTFEERPKPTLEDPHDVLVAINYTGICGSDVHYWVHGSIGKFVVT
DPMVLGHESAGTIVEVGEKVKTLKVGDRVALEPGYPCRRCTNCLAGKYNLCPDMVFA
ATPPYHGTLTGYWRAPADFCFKLPENVSQQEGALIEPLAVGVHIVKQANVKPGDSVVV
MGAGPVGLLCAAVARAYGASKIVSVDIVQSKLDFAKDFAATHTYASQRVSPEENAKNI
LELAGLPDGADVVIDASGAEPSIQASIHVLKVGGSYVQGGMGKSDITFPIMAMCIKEATV
SGSFRYGPGDYPLAIELVATGKVDVKKLVTGIVDFQQAEEAFKKVKEGEAIKVLIKGPN
EE.
Xylitol dehydrogenase homolog of Pichia angust
(SEQ ID NO: 29):
MKGLLYYGTNDIRYSETVPEPEIKNPNDVKIKVSYCGICGTDLKEFTYSGGPVFFPKQGT
KDKISGYELPLCPGHEFSGTVVEVGSGVTSVKPGDRVAVEATSHCSDRSRYKDTVAQDL
GLCMACQSGSPNCCASLSFCGLGGASGGFAEYVVYGEDHMVKLPDSIPDDIGALVEPIS
VAWHAVERARFQPGQTALVLGGGPIGLATILALQGHHAGKIVCSEPALIRRQFAKELGA
EVFDPSTCDDANAVLKAMVPENEGFHAAFDCSGVPQTFTTSIVATGPSGIAVNVAVWG
DHPIGFMPMSLTYQEKYATGSMCYTVKDFQEVVKALEDGLISLDKARKMITGKVHLKD
GVEKGFKQLIEHKENNVKILVTPNEVS.
Xylitol dehydrogenase homolog of Penicillum chrysogenum
(SEQ ID NO: 30):
MATAQNLSFVLEGIHKVKFEDRPVPELKNPHDVIINVKYTGICGSDVHYWEHGSIGSFV
VKDPMVLGHESAGIVSQVGSAVKTLKVGDRVAMEPGISCRRCDPCKAGKYNLCEDMR
FAATPPYDGTLAKYYALPEDFCYKLPEHISLQEGALMEPLSVAVHIVRQAGVSPGQTVV
VFGAGPVGLLCCAVATAFGASKVIAVDIQQQRLDFAKSYATTSTFMPSNVAAVENAER
MKEENGLGAGADVAIDASGAEPSVHTGIHVLRNGGTYVQGGMGRSEILFPIMAACSKEL
TIKGSFRYGSGDYKLAVGLVSSGKVDVKRLITGTVKFEQAEQAFIEVKAGKGIKTLIGGI
DV.
Xylitol dehydrogenase homolog of Phaeosphaeria nodorum
(SEQ ID NO: 31):
MTTKTATQKVELPNPSFVLQAPNKVVYEDRPIPDLPSPYDVIVKPKWTGICGSDVHYWV
EGRIGHFVVESPMVLGHESAGIVHKVGDKVKSLKVGDRVAMEPGVPCRRCVRCKEGK
YNLCPDMAFAATPPYDGTLARYYALPEDYCYKLPENMSLEEGALIEPTAVAVHITRQAS
IKPGDSVVVFGAGPVGLLCCAVAKAYGAKKIVTVDINEQRLNFALQYAATDKFSSARVS
AEENAKNLIKDCELGPGADVIIDASGAEPCIQTAIHALRMGGTYVQGGMGKPDINFPIMA
MCTKELNVKGSFRYGAGDYQTAVDLVAGGRISIKELITGKVKFEDAENAFAQVKKGEGI
KLLIEGPEE.
Xylitol dehydrogenase homolog of Pichia pastoris
(SEQ ID NO: 32):
MSDNPSVILKRINEIVIEDRPIPAIEDPHYVKIAIKKTGICGSDVHFYTDGCCGSFKLESPM
VLGHESAGIVVEVGSEVKSLRVGDKVACEPGIPSRYSNAYKSGHYNLCPEMAFAATPPI
DGTLCRYFLLPEDFCVKLPEHVSLEEGALVEPLSVAVHAARLAKITFGDSVVVFGAGPV
GLLVAATARAYGATNVLIVDIFDDKLTLAKDTLQVATHSFNSKNGMDNLLESFEGKHP
NVSIDCTGVESCIAAGINALAPRGVHVQVGMGKSEYNNFPLGLICEKECIVKGVFRYCY
NDYNLAVELIASGKVEVKGLVTHRFKFTEAVDAYDTVRQGKAIKAIIDGPE.
Xylitol dehydrogenase homolog of Zygosaccharomyces rouxii
(SEQ ID NO: 33):
MTKQDAIVLQKPGVITVDKRDVPEIKDPHYVKLHIKATGICGSDVHYYTQGAIGQFVVK
SPMVLGHESSGIVAEVGSAVTNVKVGDRVAIEPGIPSRYSDETMSGNYNLCPHMVFAAT
PPYDGTLTKYYLAPEDFVYKMPDHLSFEEGALAEPMSVGVHANKLAGTRFGSKVLVSG
AGPVGLLAGAVARAFGATEVVFVDIAEEKLERSKQFGATHTVSSSSDEERFVSEVSKVL
GGDLPNIVLECSGAQPAIRCGVKACKAGGHYVQVGMGKDDVNFPISAVGSKEITFHGCF
RYKKGDFADSVALLSSGRINGKPLISHRFAFDKAPEAYKFNAEHGNEVVKTIITGPE.
Xylitol dehydrogenase homolog of Arxula adeninivoran
(SEQ ID NO: 34):
MAAQVEEQVLNLRAQADHNPSFVLKKPLELGFEERPVPVITDPRDVKIQVKKTGICGSD
VHFWQHGRIGDYVVEKPMVLGHESSGVVVEVGSEVTSLKVGDRVAMEPGVPDRRSKE
YKMGRYHLCPHVRFAACPPTDGTLCKYYTLPEDFCVKLPENVDFEEGALVEPLSVAVH
TARLLGIYPGSKVVVFGAGPIGQLCIGVCKAFGASIIGAVDLFEQKLETAKEFGASHTYV
PQKGDSHDETAHKILELLPNKQAPDVVIDASGAEQSINAGIELLERGGTFGQVAMGRTD
YIQFAVSRMAMKEIRFQGVFRYTYGDYELATQLIGDGKIPVKKLVTHRRPFEKAEEAYE
LVKSGVAVKCIIDGPE.
Xylitol dehydrogenase homolog of Pichia stipitis
(SEQ ID NO: 35):
MTANPSLVLNKIDDISFETYDAPEISEPTDVLVQVKKTGICGSDIHFYAHGRIGNFVLTKP
MVLGHESAGTVVQVGKGVTSLKVGDNVAIEPGIPSRFSDEYKSGHYNLCPHMAFAATP
NSKEGEPNPPGTLCKYFKSPEDFLVKLPDHVSLELGALVEPLSVGVHASKLGSVAFGDY
VAVFGAGPVGLLAAAVAKTFGAKGVIVVDIFDNKLKMAKDIGAATHTFNSKTGGSEELI
KAFGGNVPNVVLECTGAEPCIKLGVDAIAPGGRFVQVGNAAGPVSFPITVFAMKELTLF
GSFRYGFNDYKTAVGIFDTNYQNGRENAPIDFEQLITHRYKFKDAIEAYDLVRAGKGAV
KCLIDGPE.
Xylitol dehydrogenase homolog of Aspergillus niger
(SEQ ID NO: 36):
MSTQNTNAQNLSFVLEGIHRVKFEDRPIPEINNPHDVLVNVRFTGICGSDVHYWEHGSIG
QFIVKDPMVLGHESSGVVSKVGSAVTSLKVGDCVAMEPGIPCRRCEPCKAGKYNLCVK
MAFAATPPYDGTLAKYYVLPEDFCYKLPESITLQEGAIMEPLSVAVHIVKQAGINPGQSV
VVFGAGPVGLLCCAVAKAYGASKVIAVDIQKGRLDFAKKYAATATFEPAKAAALENA
QRIITENDLGSGADVAIDASGAEPSVHTGIHVLRAGGTYVQGGMGRSEITFPIMAACTKE
LNVKGSFRYGSGDYKLAVSLVSAGKVNVKELITGVVKFEDAERAFEEVRAGKGIKTLIA
GVDS.
Xylitol dehydrogenase homolog of Pichia guilliermondii
(SEQ ID NO: 37):
MSCNFTSSNKFFNFNSLLPFLYTSSRLSSTSSSTGSLGTLIGPGSISIFGRITGFFQCDCGAIA
VYKVGVLHPTFFTIMTPNPSLVLNKVNDITFETLEAPTLLEPNEVMVEVKKTGICGSDIH
YYSHGKIGDFVLTQPMVLGHESAGVVTAVGLNVKSLKVGDRVAIEPGVPSRFSEEYKSG
HYQLCPNIVFAATPDPKHGSPSPPGTLCKYYKSPEDFLVKLPDCVSLELGAMVEPLSVGV
HGCKQAKVTFGDVVVVFGGGPVGLLAAAAATKFGAAKVMVVDVIDDKLKMALEVGV
ATHTFNSKSGGADELVKELGEHPDVVIECTGAEVCINLGIESLKMGGRFAQVGNATRPV
SFPIVAFSSRELTLYGSFRYGYNDYKTSVAILEHNYRNGRENAAIDFEKLITHRFKFEDAK
KAYDYIRDGNVAVKVIIDGPE.
Xylitol dehydrogenase homolog of Candida tropicalis
(SEQ ID NO: 38):
MTANPSLVLNKVDDISFEEYEAPKLESPRDVIVEVKKTGICGSDIHYYAHGSIGPFILRKP
MVLGHESAGVVSAVGSEVTNLKVGDRVAIEPGVPSRFSDETKSGHYHLCPHMSFAATPP
VNPDEPNPQGTLCKYYRVPCDFLFKLPDHVSLELGAMVEPLTVGVHGCKLADLKFGED
VVVFGAGPVGLLTAAVARTIGAKRVMVVDIFDNKLKMAKDMGAATHIFNSKTGGDYQ
DLIKSFDGVQPSVVLECSGAQPCIYMGVKILKAGGRFVQIGNAGGDVNFPIADFSTRELA
LYGSFRYGYGDYQTSIDILDRNYVNGKDKAPINFELLITHRFKFKDAIKAYDLVRAGNG
AVKCLIDGPE.
Xylitol dehydrogenase homolog of Kluyveromyces lactis
(SEQ ID NO: 39):
MSGTQKAVVLQKKGEITFEDIPAPEITDSHYVKIHVKKTGICGSDIHYYTHGSIGEFVVKK
PMVLGHESSGVVVEVGKDVTLVQVGDRVAIEPGVPSRYSDETKSGHYNLCPHMAFAAT
PPYDGTLVKYYLAPEDFLVKLPDHVSFEEGACAEPLAVGVHANRLAETSFGKNVVVFG
AGPVGLVTGAVAAAFGASAVVYVDVFENKLERSKDFGATNTINSTKYKSEDELTEVIKS
ELKGEQPEIAIDCSGAEICIRTAIKVLKAGGSYVQVGMGKDNINFPIAMIGAKELRVLGSF
RYYFNDYKIAVKLISEGKVNVKKMITHTFKFEEAIDAYNFNLEHGSEVVKTMIDGPE.
Xylitol dehydrogenase homolog of Candida shehatae
(SEQ ID NO: 40):
MTANPSLVLNKIDDITFESYDAPEITEPTDVLVEVKKTGICGSDIHYYAHGKIGNFVLTKP
MVLGHESSGVVTKVGTGVTSLKVGDKVAIEPGIPSRFSDAYKSGHYNLCPHMCFAATP
NSTEGEPNPPGTLCKYFKSPEDFLVKLPEHVSLEMGALVEPLSVGVHASKLASVKFGDY
VAVFGAGPVGLLAAAVAKTFGAKGVIVIDIFDNKLQMAKDIGAATHIFNSKTGGDAAA
LVKAFDGHEPTVVLECTGAEPCINQGVAILAQGGRFVQVGNAPGPVKFPITEFATKELTL
FGSFRYGFNDYKTSVDIMDTNYKNGKEKAPIDFEQLITHRFKFADAIKAYDLVRAGSGA
VKCFIDGPE.
Xylitol dehydrogenase homolog of Talaromyces stipitatus
(SEQ ID NO: 41):
MSLTETKNLSFVLEGIKKVKFEERPIPEIIDPYDVLINVKYTGICGSDVHYWEHGSIGSFV
VREPMVLGHESSGVVSKVGSKVTTLKVGDQVAMEPGIPCRRCEPCKSGKYHLCINMAF
AATPPYDGTLARYYRLPEDFCYKLPENIPLKEGALIEPLGVAVHVVKQGGVVPGNSVVV
FGAGPVGLLCGAVAKAFGASKVIISDIQQSRLDFAKKYIADGTFQPARVSAEENANRLK
EEHDILAGADVVLEASGAEPAVHTGIHALRTGGTFVQAGMGRSEINFPIMAVCGKELNF
KGSFRYGSGDYKLAVELVATGKVSVKELITGEFKFEDAEQAYIDVKAGKGIKTIIVGL.
Xylitol dehydrogenase homolog of Pachysolen tannophilus
(SEQ ID NO: 42):
AWKGDWPLATKSPLVGGHEGAGVVVGMGSAVKNWKLGDLAGIKWLNGSCMNCEFC
MHGDEPNCAHADLSGYTHDGSFQQYATADAVQAGRIPAGTNLSEIAPILCAGVTAYKAI
KTAELKPGDWCCISGSGGGLGTLAIQFAKAMGLRVIGIDGGAGKEKLCLDLGAEKYIDF
TKTKDIVKDVIAATDGGPHAVINVSVSERAIDASVNYVRPTGTVVLVGLPAGAVCKSEV
FSQVVRSVKIKGSYVGNRCDTAEAIDFYVRGLVKSPIKVIGLSELPMVYDLMEKGEILGR
YVVDTSR.
Xylitol dehydrogenase homolog of Neurospora crassa, ARS
mutant (SEQ ID NO: 43):
MATDGKSNLSFVLNKPLDVCFQDKPVPKINSPHDVLVAVNYTGICGSDVHYWLHGAIG
HFVVKDPMVLGHESAGTIVAVGDAVKTLSVGDRVALEPGYPCRRCVHCLSGHYNLCPE
MRFAATPPYDGTLTGFWTAPADFCYKLPETVSLQEGALIEPLAVAVHITKQAKIQPGQT
VVVMGAGPVGLLCAAVAKAYGASKVVSVARSPSKLEFAKSFAATHTYLSQRVSPEENA
RNIIAAADLGEGADAVIDASGAEPSIQAALHVVRQGGHYVQGGMGKDNIIFPIMALCIKE
VTASGSFRYGSGDYRLAIQLVEQGKVDVKKLVNGVVPFKNAEEAFKKVKEGEVIKILIA
GPNEDVEGSLDTTVDEKKLNEAKACGGSGCC.
Xylulokinase (XKS) Sequences
Xylulokinase homolog of Aspergillus niger (SEQ ID NO:44):
MQGPLYIGFDLSTQQLKGLVVNSDLKVVYVSKFDFDADSHGFPIKKGVLTNEAEHEVFA
PVALWLQALDGVLEGLRKQGMDFSQIKGISGAGQQHGSVYWGENAEKLLKELDASKT
LEEQLDGAFSHPFSPNWQDSSTQKECDEFDAALGGQSELAFATGSKAHHRFTGPQIMRF
QRKYPDVYKKTSRISLVSSFIASLFLGHIAPMDISDVCGMNLWNIKKGAYDEKLLQLCA
GSSGVDDLKRKLGDVPEDGGIHLGPIDRYYVERYGFSPDCTIIPATGDNPATILALPLRAS
DAMVSLGTSTTFLMSTPSYKPDPATHFFNHPTTAGLYMFMLCYKNGGLARELVRDAVN
EKLGEKPSTSWANFDKVTLETPPMGQKADSDPMKLGLFFPRPEIVPNLRSGQWRFDYNP
KDGSLQPSNGGWDEPFDEARAIVESQMLSLRLRSRGLTQSPGEGIPAQPRRVYLVGGGS
KNKAIAKVAGEILGGSEGVYKLEIGDNACALGAAYKAVWAMERAEGQTFEDLIGKRW
HEEEFIEKIADGYQPGVFERYGQAAEGFEKMELEVLRQEGKH.
Xylulokinase homolog of Candida albicans (SEQ ID NO: 45):
MYSFTFTITFIYIYKLFTFFEGYFTFIFYVNNPPPSPAMTDYSNSKSLFLGFDLSTQQLKIIIT
DENLTPLDTYNVEFDSQFKSKYTKINKGVITGDDGEVISPVAMWLDAINYVFDEMQKSK
FPFDKVVGISGSGQQHGSVYWSGEANELLNDLIPCKELSSQLQDAFSWGYSPNWQDHST
VKEAEDFHKAIGKEHLAEISGSRAHLRFTGLQIRKFITRSHSKEYESTSRISLVSSFVTSILL
GEIAQLEESDACGMNLYDIQKSQYDEELLALAAGVHTEIDNISKEDPKYKKSIDQLKQKL
GEISPITYKSSGKISKYFVDTYGFNSDCKIYSFTGDNLATILSLPLQPNDCLISLGTSTTVLII
TSNYEPSSQYHLFKHPTLPDHYMGMLCYCNGSLAREKARDQANKKHNVSDNKSWDKF
NEILDHNKDFNGKLGIYFPLGEIIPQAPAQTIRAVLEDNGEITPCELDSHGFTVDDDASAIV
DSQTLSCRLRAGPMLSKSSTTKNGKTNSSEELQQLYDNLVDKFGELSTDGKKQSFESLT
ARPNRCYYVGGASNNTSIITKMGSIFGPTNGNYKVEIPNACALGGAYKASWSYKCELEN
KMIGYDEYIGKII.
Xylulokinase homolog of Candida tropicalis (SEQ ID NO: 46):
MTTDYSENDKLFLGLDLSTQQLKIIVTNEDLIPLKTYHVEFDAEFKEKYNITKGVVNGED
GEVISPVGMWLDSMNYVFNSMKKDKFPFDKVVGISGSAQQHGSVYWSHEANELLSDL
KPEEDLSEQLKDAFSWEYSPNWQDHSTLKEAEAFHEAIGKENLAKITGSRAHLRFTGLQI
RKFATRSHVEEYAKTSRISLVSSFLTSVLIGKVTGLEESDACGMNLYDITKSQYNEELLA
LGAGVHPKIDGVDKNDEKYQKSIDELKQKLGDITPITYESSGDISPYFVDTYGFNKDVKI
YSFTGDNLATILSLPLQPNDCLISLGTSTTVLIITENYQPSSQYHLFKHPTMPDSYMGMLC
YCNGSLAREKARDEVNKQNKVSDSKSWDKFDEILDNSKHFNHKLGIYFPLGEIIPQAPA
QTIRAVLEDGKIIPCELNTHGFSIDDDANAIVESQTLSCRLRAGPMLSNSGDSSSDDESPES
TKELENIYKDLTSKFGELYTDGKKQTFESLTARPNRCYYVGGASNNPSIIKKMGSIFGPV
NGNYKVEIPNACALGGAYKASWSFACEEKGKMISYADYITKLFDTNDELDQFQVEDKW
VEYFEGVGMLAKMEETLLKQ.
Xylulokinase homolog of Penicillium chrysogenum (SEQ ID NO: 47):
MASDSPLYIGFDLSTQQLKGLVVNSDLKVVHAAKFDFDADSKGFPIKKGVLNNEAEHE
VFAPVALWLQALDGVLETLRKEGLDFRRVKGISGAGQQHGSVYWGQNAESLLRNLDSS
KSLEEQLEGAFSHPYSPNWQDSSTQNECDEFDAALGDRKHLAQATGSKAHHRFTGPQIL
RFTRKHPDVYKKTSRISLVSSFLASLFLGHIAPFDISDVCGMNLWNIKKGAYDEGLIQLCS
GAFGVEDLKQKLGEVPEDGGLHLGSVHAYFVERFGFSPDCTVIPATGDNPATILALPLLP
SDAMVSLGTSTTFLMSTPSYKPDPATHFFNHPTTPGLYMFMLCYKNGGLAREHVRDAIN
ESLKDTPAQPWANFDKVALQTAPLGQQSPTDPMKMGLFFPRHEIVPNIPKGQWRFTYD
ANTGNLKETTDGWNSPQDEARAIIESQLLSCRLRSRDLTENPGGGLPSQPRRVYLVGGG
SKNKAIAKIAGEILGGVEGVYSLDVGDNACALGAAYKAVWGIERQPGQTFEDLIGQRW
NEAEFIEKIADGYQKGIFEQYGQAVEGFEKMELQVLQQVAEKGDGDDY.
Xylulokinase homolog of Pichia stipitis (SEQ ID NO: 48):
MTTTPFDAPDKLFLGFDLSTQQLKIIVTDENLAALKTYNVEFDSINSSVQKGVIAINDEIS
KGAIISPVYMWLDALDHVFEDMKKDGFPFNKVVGISGSCQQHGSVYWSRTAEKVLSEL
DAESSLSSQMRSAFTFKHAPNWQDHSTGKELEEFERVIGADALADISGSRAHYRFTGLQI
RKLSTRFKPEKYNRTARISLVSSFVASVLLGRITSIEEADACGMNLYDIEKREFNEELLAI
AAGVHPELDGVEQDGEIYRAGINELKRKLGPVKPITYESEGDIASYFVTRYGFNPDCKIY
SFTGDNLATIISLPLAPNDALISLGTSTTVLIITKNYAPSSQYHLFKHPTMPDHYMGMICY
CNGSLAREKVRDEVNEKFNVEDKKSWDKFNEILDKSTDFNNKLGIYFPLGEIVPNAAAQ
IKRSVLNSKNEIVDVELGDKNWQPEDDVSSIVESQTLSCRLRTGPMLSKSGDSSASSSAS
PQPEGDGTDLHKVYQDLVKKFGDLFTDGKKQTFESLTARPNRCYYVGGASNNGSIIXK
MGSILAPVNGNYKVDIPNACALGGAYKASWSYECEAKKEWIGYDQYINRLFEVSDEMN
SFEVKDKWLEYANGVGMLAKMESELKH.
Xylulokinase homolog of Saccharomyces cerevisiae
(SEQ ID NO: 49):
MLCSVIQRQTREVSNTMSLDSYYLGFDLSTQQLKCLAINQDLKIVHSETVEFEKDLPHY
NTKKGVYIHGDTIECPVAMWLEALDLVLSKYREAKFPLNKVMAVSGSCQQHGSVYWS
SQAESLLEQLNKKPEKDLLHYVSSVAFARQTAPNWQDHSTAKQCQEFEECIGGPEKMA
QLTGSRAHFRFTGPQILKIAQLEPEAYEKTKTISLVSNFLTSILVGHLVELEEADACGMNL
YDIRERKFSDELLHLIDSSSKDKTIRQKLMRAPMKNLIAGTICKYFIEKYGFNTNCKVSP
MTGDNLATICSLPLRKNDVLVSLGTSTTVLLVTDKYHPSPNYHLFIHPTLPNHYMGMIC
YCNGSLARERIRDELNKERENNYEKTNDWTLFNQAVLDDSESSENELGVYFPLGEIVPS
VKAINKRVIFNPKTGMIEREVAKFKDKRHDAKNIVESQALSCRVRISPLLSDSNASSQQR
LNEDTIVKFDYDESPLRDYLNKRPERTSFVGGASKNDAIVKKFAQVIGATKGNFRLETPN
SCALGGCYKAMWSLLYDSNKIAVPFDKFLNDNFPWHVMESISDVDNENWDRYNSKIVP
LSELEKTLI.
Xylulokinase homolog of Pichia pastoris (SEQ ID NO: 50):
MVTKEIQNRDSALTESVPNDLYLGFDLSTQQLKITSFEGRSLTHFKTYRVDFDEELSVYG
INNGVYVNEETGEINAPVAMWVEALDLIFSKMQKDKFPFGIVKGMSGSCQQHGSVYWS
KDAPDLLSSLSPSKDLKSQLCPKAFTFEKSPNWQDHSTGEELEIFERKAGSPENLSKITGS
RAHYRFTGSQIRKLAKRVNPELYKETYRISLISSFLSSLLCGRITKIEESDGCGMNIYDIQN
SRYDEDLLAVTAAVDPEIDGATEHERQEGVARLKDKLQDLEPVGYRSIGTIAAYFVEKY
GFSEDSKVFSFTGDNLATILSLPLHNDDILVSLGTSTTVLLVTETYWPNSNYHVFKHPTV
PGSYMVMLCYVNGALARNQIKTSLDKKYNVSDPNDWTKFNEILDKSKPLHGKEELGVY
FPKGEIIPNCVAQTKRFSYDAKSKKLVTANWDIEDDVVSIVESQALSCRLRSGPLYHGSD
ETDQEEESEVIQRLSNFPKISADGKDQRLPDLISHPKKAFYVGGASQNVSIVRKFSEVLGA
KEGNYQINLGDACAIGGAFKAVWSDLCETEKAIPYSDFLRKNFHWKENVKPVEADSSL
WLQYVDGVGILSEIEQTLEK.
Xylulokinase homolog of Candida dubliniensis (SEQ ID NO: 51):
MTDYSNSKPLFLGFDLSTQQLKIIITNENLTPLNTYNVEFDSQFKSKYKDINKGVITGDDG
EVISPVAMWLDAINYVFDEMKKDKFPFNKVSGISGSCQQHGSVYWSEKANELLNDLNP
SQELSTQLQDAFSWGYSPNWQDHSTVKEAEEFHKAIGKEHLAEITGSRAHLRFTGLQIR
KFVTRSHSKEYKSTSRISLVSSFVTSILLGEIAQLEESDACGMNLYDIQKSQYDEELLALA
AGVHPEIDNVSKEDPKYKKSIDQLKQKLGEISPITYKSSGKISKYFVDTYGFNSNCKIYSF
TGDNLATILSLPLQHNDCLISLGTSTTVLIITSNYEPSSQYHLFKHPTLPDHYMGMLCYCN
GSLAREKARDQVNAKHNISDKKSWDKFNEILDNNKDFNGKLGIYFPLGEIIPQAPAQTIR
AVLEDNGEITPCELDSHGFTVDDDASAIVDSQTLSCRLRAGPMLSKSSSSNTTSSKKNGN
EKTNTSKELKQLYDNLVNKFGELSTDGKKQSFESLIARPNRCYYVGGASNNTSIIKKMG
SIFGPINGNYKVEIPNACALGGAYKASWSYKCELENKMISYDEYIGKLFDTNDELESFKV
DDKWEEYFTGVGMLAKMEETLLKQ.
Xylulokinase homolog of Neurospora crassa OR74A
(SEQ ID NO: 52):
MDVQAIVIQSDLSVVSSAKVDFDGDFGAKYGIKKGVQVNEVDGEVFAPVAMWLEALD
LVLQRLQEAKTPLNRIRGISGSCQQHGSVYWSREAEKLLAELQADKQRGDLVDQLKGA
FSHPYAPNWQDHSTQAECDKFDEALGTAERLAHATGSAAHHRFTGPQIMRLRRKLPGM
YASTSRISLVSSFLASLFIGSVAPMDISDVCGMNLWDIPSNTWSETLLALAAGGSTEGAA
DLKAKLGEVRLDGGGSMGKISPYFVGKYGFSPDCEIAPFTGDNPATILALPLRPLDAIVS
LGTSTTFLMITPVYKPDPSYHFFNHPTTPGQYMFMLCYKNGGLAREKVRDALPAPSNSS
KDPWETFNQHALSTPPLDVSSPATDQAKLGLYFYLPEIVPNISAGTWRYECSATDGSNL
QPVNQPWPVEKDARIIVESQALSMRLRSQNLVSTPPSTPSGTSSSSSSSALPAQPRRIYLV
GGGSLNPAIARIMGDVLGGVDGVYKLDVGGNACALGGAYKAVWAFERRDETETFDELI
GKRWKEEGAIRKVDEGYKKGVFEGYGNVLGAFGEMEGKVLEVARNK.
Xylulokinase homolog of Kluyveromyces lactis NRRL Y-1140
(SEQ ID NO: 53):
MSESGYYLGFDLSTQQLKCLAIDDQLNIVTTAAIEFDKDFPHYNTRKGVYIKDEGVIDAP
VAMWLEAIDLCFERLGKCIDLKKVKSMSGSCQQHGTVFWNCDHLPKDLQPSSNLVKQL
ASCFSRDVAPNWQDHSTRKQCDELTDKVGGPQELARITGSSSHYRFSGSQIAKVHETEP
EVYANTKKISLVSSFLASVLVGDIVPLEEADACGMNLYGIEKHEFNEDLLSVVDEDIASI
KRKLFDPPTSSDEPKSLGPVSTYFQEKYGVNPDCQIYPFTGDNLATICSLPLQKNDVLISL
GTSTTILLITDQYHSSPNYHLFIHPTVPNHYMGMICYCNGSLAREKIRDDINGESQTHDW
TKFNEALLDNSLSNDNEIGLYFPLGEIVPNMDAVTKRCYFKYIDNKVVLTNVNMFPDKR
LDAKNIVESQALSCRVRISPLLSEEANAINETQVLKSELKVKFDYDFFPLASYAKRPNRA
FFVGGASKNEAIIKTMANVIGAKNGNYRLETANSCALGGCYKALWSLLKEQNPETPSFD
RWLNAFFNWERDCEFVCNSDAAKWENYNNKIRTLSEIEREASSH.
Xylulokinase homolog of Meyerozyma guilliermondii ATCC 6260
(SEQ ID NO: 54):
MTSKSSANYELLKELYLGFDLSTQQLKIIATNGKLDHLGTYNVEFDQEFGEKYEVKKGV
RVNEQSGEIVSPVAMWLDAIDFLFGKMKQQNFPFDKVVGISGSGQQHGSVYWSLDAPQ
LLSNLDASTTLASQLKSAFTFPESPNWQDHSTGEEIKVFEDTVGGPEKLAELTGSRAHYR
FTGLQIRKLAVRKNPELYRKTHRISLVSSFVASVLSGEITTIEQAEACGMNIYDIKKHDYD
DELLSLAAGVHPKADSASEEEREKGIASLKEKLGEVKKVSYDNCGTISSYFVKKFGLNPS
ARIYPFTGDNLATIISLPLHPNDILLSLGTSTTVLLVTQNFKPSAQYHLFVHPTMPNHYMG
MICYCNGALAREKVRDALNEKYSLEKNSWDKFNEVLDSSKKFDNKLGIYFPLGEIVPNA
SAQFKRSKLANGKIEDVESWDIDEDVSSIVESQSLSARLRAGPMLNGSDSSNSSTPELDES
SSGESSKLKKMYHELHSEFGDLYTDGEKHTYGSLTSRPRNTFFVGGASNNLSIVRKMASI
LGAMDHNYKVEIPNACALGGAYKASWSHTCEKKNQWINYDDYISQNFHFDDLDPVQV
KDEWESYFKGMGMLAKMEENLKHD.
Xylulokinase homolog of Podospora anserina S mat+
(SEQ ID NO: 55):
MTDNGPLYLGFDLSTQQLKAIVIQSDLSIVSSAKVDFDQDFGAKYKIKKGVLVNEQEGE
VFAPVALWLESLDLVLQRLQEQNTPLNCIKGISGSCQQHGSVYWSHEAEQLLGGLTADK
SLVDQLTGAFSHPFAPNWQDHSTQHECDKFEETMGTAERLAQATGSAAHHRFTGTQIM
RLRHKLPQMYTSTSRISLVSSFLASLFLGSIAPMDISDVCGMNLWDIPSNNWSSPLLDLAS
GGSPDDLRAKLGEVRQDGGGSMGNVSSYFVNKYNFSPDCGVAPFTGDNPATILALPLRP
LDAIVSLGTSTTFLMSTPVYKPDPSYHFFNHPTTPGQYMFMLCYKNGGLAREKVRDVLP
SSESGDVWENFNKHALETAPLDVRKEGDRAKLGLYFYLPEIVPNIKAGTWRYTCDANS
GEGLEEVREPWAKETDARAIIESQALSMRLRSQKLVTAPREGLPAQPGRVYLVGGGSLN
PAITRVLGDALGGADGVYKLDVGGNACALGGAYKAVWAFERGDGEAFDELIGKRWKE
EGAIQRVDEGYKKGVFEKYGNVLGAFEKMEEEILKVAKNT.
Xylulokinase homolog of Aspergillus flavus NRRL3357
(SEQ ID NO: 56):
MQGPLYIGFDLSTQQLKALVVNSDLKVVYVSKFDFDADSRGFPIKKGVITNEAEHEVYA
PVALWLQALDGVLEGLKKQGLDFARVKGISGAGQQHGSVYWGQDAERLLKELDSGKS
LEDQLSGAFSHPYSPNWQDSSTQKECDEFDAFLGGADKLANATGSKAHHRFTGPQILRF
QRKYPEVYKKTSRISLVSSFLASLFLGHIAPLDISDACGMNLWNIKQGAYDEKLLQLCAG
PSGVEDLKRKLGAVPEDGGINLGQIDRYYIERYGFSSDCTIIPATGDNPATILALPLRPSD
AMVSLGTSTTFLMSTPNYMPDPATHFFNHPTTAGLYMFMLCYKNGGLAREHIRDAIND
KLGMAGDKDPWANFDKITLETAPMGQKKDSDPMKMGLFFPRPEIVPNLRAGQWRFDY
NPADGSLHETNGGWNKPADEARAIVESQFLSLRLRSRGLTASPGQGMPAQPRRVYLVG
GGSKNKAIAKVAGEILGGSDGVYKLEIGDNACALGAAYKAVWALERKDGQTFEDLIGQ
RWREEDFIEKIADGYQKGVFEKYGAALEGFEKMELQVLKQEGETR.
Xylulokinase homolog of Aspergillus fumigatus Af293
(SEQ ID NO: 57):
MTSQGPLYIGFDLSTQQLKGLVVNSELKVVHISKFDFDADSHGFSIKKGVLTNEAEHEVF
APVALWLQALDGVLNGLRKQGLDFSRVKGISGAGQQHGSVYWGENAESLLKSLDSSKS
LEEQLSGAFSHPFSPNWQDASTQKECDEFDAFLGGPEQLAEATGSKAHHRFTGPQILRM
QRKYPEVYKKTARISLVSSFLASLLLGHIAPMDISDVCGMNLWDIKKGAYNEKLLGLCA
GPFGVEDLKRKLGAVPEDGGLRLGKINRYFVERYGFSSDCEILPSTGDNPATILALPLRPS
DAMVSLGTSTTFLMSTPNYKPDPATHFFNHPTTPGLYMFMLCYKNGGLAREHVRDAIN
EKSGSGASQSWESFDKIMLETPPMGQKTESGPMKMGLFFPRPEIVPNVRSGQWRFTYDP
ASDALTETEDGWNTPSDEARAIVESQMLSLRLRSRGLTQSPGDGLPPQPRRVYLVGGGS
KNKAIAKVAGEILGGSDGVYKLDVGDNACALGAAYKAVWAIERKPGQTFEDLIGQRW
REEEFIEKIADGYQKGVFEKYGKAVEGFEKMEQQVLKQEAARK.
Xylulokinase homolog of Talaromyces stipitatus ATCC 10500
(SEQ ID NO: 58):
MAPGPLYIGFDLSTQQLKGLVVSSDLKVEYEAKFDFDAHSHGFDIKKGVMTNEAEHEV
FAPVAMWLQALDSVLKTLKDQGLDFGRIRGISGAGQQHGSVYWSKDAEKLLQSLRSEK
SLEEQLADAFSHPYSPNWQDASTQKECDEFDAYLGGPEELAHVTGSKAHHRFTGPQILR
FHRKYPEQYKKTSRISLVSSFLASLFLGRIAPFDISDVCGMNLWNITAGSWDDRLLKLCA
GQFGVDDLKQKLGDVPEDGGLHLGKIHEYFVERYSFNPDCIIMPSTGDNPSTILALPLNP
SDAMVSLGTSTTFLMSTPMYKPDSATHFFNHPTTPGLHMFMLCYKNGGLAREQVRDAI
NKQVGGNTAGKNPWANFDKAALETPAMGQKSASDTMKMGLFFPRPEIIPNLPSGQWRF
NYNPQDKSLEETTSGWDIPLDEARAIVESQFLSLRLRSRGLTTAPAEGLPPQPKRVYLVG
GGSKNTAIAKIAGEILGGHDGVYKLDVGENACALGAAYKAVWAIERQPGQTFEDLIGK
RWREEEFVEKIADGYQPDVFKKYGVAVGGFERMEQQILQQEGRK.
Xylulokinase homolog of Aspergillus nidulans FGSC A4
(SEQ ID NO: 59):
MSSRSSSPLKGPLYIGFDLSTQQLKGLVVNSDLKVVYSSIFDFDADSQGFPIKKGVLTNE
AEHEVFAPVALWLQALDSVLDGLKKQGLDFSHVRGISGAGQQHGSVYWGQDAEKLLN
GLDAGKRLQEQLEGAFSHPYSPNWQDSSTQKECDEFDEYLGGADKLAEATGSKAHHRF
TGPQILRFQKKYPDVYKKTSRISLVSSFLASLFLGHIAPLDISDVCGMNLWNIHKGAYDE
DLLKLCAGPHGVEDLKRKLGDVPEDGGIDLGKVHRYYVDRYGFSPECTVIPSTGDNPAT
ILALPLRPSDAMVSLGTSTTFLMSTPSYKADPATHFFNHPTTPGLYMFMLCYKNGGLAR
EKIRDAINDAKNEKNPSNPWANFDSVALQTPPLGQTSPSDPMKMGLFFPRPEIVPNLRAG
QWLFNYDPSTGNLTETLNGEGWNRPADEARAIIESQMLSLRLRSRGLTSSPGGDIPAQPR
RVYLVGGGSKNKTIAKIAGEILGGSEGVYKLEIGDNACALGAAYKAVWALERKKDQTF
EDLIGARWHEEEFIEKIADGYQKEAFERYGKAVEGFEKMEQRVLEQEGRK.
Xylulokinase homolog of Aspergillus oryzae RIB40
(SEQ ID NO: 60):
MQGPLYIGFDLSTQQLKALVVNSDLKVVYVSKFDFDADSRGFPIKKGVITNEAEHEVYA
PVALWLQALDGVLEGLKKQGLDFARVKGISGAGQQHGSVYWGQDAERLLKELDSGKS
LEDQLSGAFSHPYSPNWQDSSTQKECDEFDAFLGGADKLANATGSKAHHRFTGPQILRF
QRKYPEVYKKTSRISLVSSFLASLFLGHIAPLDTSDVCGMNLWNIKQGAYDEKLLQLCA
GPSGVEDLKRKLGAVPEDGGINLGQIDRYYIERYGFSSDCTIIPATGDNPATILALPLRPS
DAMVSLGTSTTFLMSTPNYMPDPATHFFNHPTTAGLYMFMLCYKNGGLAREHIRDAIN
DKLGMAGDKDPWANFDKITLETAPMGQKKDSDPMKMGLFFPRPEIVPNLRAGQWRFD
YNPADGSLHETNGGWNKPADEARAIVESQFLSLRLRSRGLTASPGQGMPAQPRRVYLV
GGGSKNKAIAKVAGEILGGSDGVYKLEIGDNACALGAAYKAVWALERKDGQTFEDLIG
QRWREEDFIEKIADGYQKGVFEKYGAALEGFEKMELQVLKQEGETR.
Xylulokinase homolog of Zygosaccharomyces rouxii
(SEQ ID NO: 61):
MTETNDSFYLGFDLSTQQLKCLAINESLRIVHTETVAFGDELPQYETSKGVYVKGDSIQS
PVSMWLEALDLLFSKFTQHGFDLSKVRAVSGSCQQHGSVYWTQKADELLRGLKSTKGS
LAEQLSPEAFSRPTAPNWQDHSTGKQCHEFEDAVGGPQELARITGSRAHFRFTGTQILKI
AEEEPEAYANTATVSLVSSFLASVLTGQLTSIEEAEACGMNLYDIPKREYHPKLLDLVDK
DRKSIESKLKSPPIHCDKPVCLGSICSYFVDKYGFNKDCSVYPFTGDNLATICSLPLEKND
VLVSLGTSTTILLVTDQYHPSADYHLFIHPTLPNHYMGMICYCNGALARERVRDYINGSP
TSDWTPFNDALNDTNLNNDDEIGVYFPLGEIVPSVPSVYKRAKFDPSTGHIKEFVDNFAD
DRHDAKNIVESQALSCRVRISPLLTSGVPVEGLAKDPNVRFDYDDIPLSQYYGRRPRRAF
FVGGASKNDAIVNKFIQVLGATEGNYRLETPNSCALGGCYKAIwSHKIHEKQITATFDHF
LGEKFPWGEVEHIRDSDDASWHHYNKKILPLSELEASLPKH.
Xylulokinase homolog of Nectria haematococca mpVI 77-13-4
(SEQ ID NO: 62):
MPFLARSRSNSPELPSDSKPLYLGFDLSTQQLKGIVVDSDLKVVGEAKVDFDKDFGRKY
GVQKGVHVIEETGEVYAPVAMWMESLDLVLERLAEAMPVPLSRIRAISGSCQQHGSVF
WNGQAYEILHNLDPRLPLAVQLPGALAHPWSPNWQDQSTQNECDAFDAALGGRQKLA
EVTGSGAHHRFTGTQIMRLKKDLPQMYARTAHISLVSSWLASVFLGAIAPMDVSDVCG
MNLFDMSRQTFSEPLLELAAGSKRDAINLRKKLGEPCLKGEAILGPVSPYFVDRHGFHP
DCQITPFTGDNPGTILALPLRPLDAIVSLGTSTTFLMNTPKYKPDGSYHFFNHPTTDGHY
MFMLCYKNGGLARERVRDQLPKPENGPTGWETFNKAVEDTPLMGAAKEDDRRKLGL
YFYLRETVPNIRAGTWRYSCEPDGSDLQEVKGGWDKETDARMIVESQALSMRLRSQNL
VHSPRPGLPAQPRRIYLVGGGSLNPAIARVLGEVLGGSEGVYKLDVGGNACALGGAYK
ALWAMERQENETFDDLIGKRWTEEGNIQRIDEGFRDGTYQKYGKLLTAFEALENKILAE
QAHAPEEDQRRSEEKV.
L-Arabitol Dehydrogenase (LAD) Sequences
L-Arabitol dehydrogenase homolog of Aspergillus nidulans
FGSC A4 (SEQ ID NO: 63):
MEILQKKPKNIAIHTSPVHDLRVVDCEIPRLAPDGCLIHVRATGICGSDVHFWKHGRIGP
MVVTGDNGLGHESAGVVLQVGDAVTRFKPGKYHACPDVVFFSTPPHHGTLRRYHAHP
EAWLHRLPDHVSFEEGALLEPLTVALAGIDRSGLRLADPLVICGAGPIGLVTLLAANAAG
AAPIVITDIDSNRLAKAKELVPRVQPVLVQKQESPQELAGRIVQRLGQEARLVLECTGVE
SSVHAGIYATRFGGTVFVIRVGKDFQNIPFMHMSAKEIDLRFQYRYHDIYPKAISLVNAG
LVDLKPLVSHRYKLEDGLEAFATASNTAAKAIKLGTSSREPYSGICPKDEVVPTVLTKPG
TRFLRDCTTHIALHGSSPSSNVYGKPGIECLRRSAEHTREQQWTLQFDGCSSLASSGSGE
RLGQARPEPV.
L-Arabitol dehydrogenase homolog of Aspergillus niger
(SEQ ID NO: 64):
MATATVLEKANIGVFTNTKHDLWVADAKPTLEEVKNGQGLQPGEVTIEVRSTGICGSD
VHFWHAGCIGPMIVTGDHILGHESAGQVVAVAPDVTSLKPGDRVAVEPNIICNACEPCL
TGRYNGCENVQFLSTPPVDGLLRRYVNHPAIWCHKIGDMSYEDGALLEPLSVSLAGIER
SGLRLGDPCLVTGAGPIGLITLLSARAAGASPIVITDIDEGRLEFAKSLVPDVRTYKVQIGL
SAEQNAEGIINVFNDGQGSGPGALRPRIAMECTGVESSVASAIWSVKFGGKVFVIGVGK
NEMTVPFMRLSTWEIDLQYQYRYCNTWPRAIRLVRNGVIDLKKLVTHRFLLEDAIKAFE
TAANPKTGAIKVQIMSSEDDVKAASAGQKI.
L-Arabitol dehydrogenase homolog of Aspergillus niger, SRT
mutant (SEQ ID NO: 65):
MATATVLEKANIGVFTNTKHDLWVADAKPTLEEVKNGQGLQPGEVTIEVRSTGICGSD
VHFWHAGCIGPMIVTGDHILGHESAGQVVAVAPDVTSLKPGDRVAVEPNIICNACEPCL
TGRYNGCENVQFLSTPPVDGLLRRYVNHPAIWCHKIGDMSYEDGALLEPLSVSLAGIER
SGLRLGDPCLVTGAGPIGLITLLSARAAGASPIVITSRDEGRLEFAKSLVPDVRTYKVQIG
LSAEQNAEGIINVFNDGQGSGPGALRPRIAMECTGVESSVASAIWSVKFGGKVFVIGVG
KNEMTVPFMRLSTWEIDLQYQYRYCNTWPRAIRLVRNGVIDLKKLVTHRFLLEDAIKAF
ETATNPKTGAIKVQIMSSEDDVKAASAGQKI.
L-Arabitol dehydrogenase homolog of Aspergillus oryzae
(SEQ ID NO: 66):
MATATVLEKANIGVYTNTNHDLWVAESKPTLEEVKSGESLKPGEVTVQVRSTGICGSD
VHFWHAGCIGPMIVTGDHILGHESAGEVIAVASDVTHLKPGDRVAVEPNIPCHACEPCL
TGRYNGCEKVLFLSTPPVDGLLRRYVNHPAVWCHKIGDMSYEDGALLEPLSVSLAAIER
SGLRLGDPVLVTGAGPIGLITLLSARAAGATPIVITDIDEGRLAFAKSLVPDVITYKVQTN
LSAEDNAAGIIDAFNDGQGSAPDALKPKLALECTGVESSVASAIWSVKFGGKVFVIGVG
KNEMKIPFMRLSTQEIDLQYQYRYCNTWPRAIRLVRNGVISLKKLVTHRFLLEDALKAF
ETAADPKTGAIKVQIMSNEEDVKGASA.
L-Arabitol dehydrogenase homolog of Trichoderma
longigrachiatum (SEQ ID NO: 67):
MSPSAVDDAPKATGAAISVKPNIGVFTNPKHDLWISEAEPSADAVKSGADLKPGEVTIA
VRSTGICGSDVHFWHAGCIGPMIVEGDHILGHESAGEVIAVHPTVSSLQIGDRVAIEPNIIC
NACEPCLTGRYNGCEKVEFLSTPPVPGLLRRYVNHPAVWCHKIGNMSWENGALLEPLS
VALAGMQRAKVQLGDPVLVCGAGPIGLVSMLCAAAAGACPLVITDISESRLAFAKEICP
RVTTHRIEIGKSAEETAKSIVSSFGGVEPAVTLECTGVESSIAAAIWASKFGGKVFVIGVG
KNEISIPFMRASVREVDIQLQYRYSNTWPRAIRLIESGVIDLSKFVTHRFPLEDAVKAFET
SADPKSGAIKVMIQSLD.
L-Arabitol dehydrogenase homolog of Trichoderma
longigrachiatum SRT mutant (SEQ ID NO: 68):
MSPSAVDDAPKATGAAISVKPNIGVFTNPKHDLWISEAEPSADAVKSGADLKPGEVTIA
VRSTGICGSDVHFWHAGCIGPMIVEGDHILGHESAGEVIAVHPTVSSLQIGDRVAIEPNIIC
NACEPCLTGRYNGCEKVEFLSTPPVPGLLRRYVNHPAVWCHKIGNMSWENGALLEPLS
VALAGMQRAKVQLGDPVLVCGAGPIGLVSMLCAAAAGACPLVITSRSESRLAFAKEICP
RVTTHRIEIGKSAEETAKSIVSSFGGVEPAVTLECTGVESSIAAAIWASKFGGKVFVIGVG
KNEISIPFMRASVREVDIQLQYRYSNTWPRAIRLIESGVIDLSKFVTHRFPLEDAVKAFET
STDPKSGAIKVMIQSLD.
L-Arabitol dehydrogenase homolog of Neurospora crassa OR74A
(SEQ ID NO: 69):
MASSASKTNIGVFTNPQHDLWISEASPSLESVQKGEELKEGEVTVAVRSTGICGSDVHF
WKHGCIGPMIVECDHVLGHESAGEVIAVHPSVKSIKVGDRVAIEPQVICNACEPCLTGRY
NGCERVDFLSTPPVPGLLRRYVNHPAVWCHKIGNMSYENGAMLEPLSVALAGLQRAG
VRLGDPVLICGAGPIGLITMLCAKAAGACPLVITDIDEGRLKFAKEICPEVVTHKVERLSA
EESAKKIVESFGGIEPAVALECTGVESSIAAAIWAVKFGGKVFVIGVGKNEIQIPFMRASV
REVDLQFQYRYCNTWPRAIRLVENGLVDLTRLVTHRFPLEDALKAFETASDPKTGAIKV
QIQSLE.
L-Arabitol dehydrogenase homolog of Neurospora crassa OR74A
SRT mutant (SEQ ID NO: 70):
MASSASKTNIGVFTNPQHDLWISEASPSLESVQKGEELKEGEVTVAVRSTGICGSDVHF
WKHGCIGPMIVECDHVLGHESAGEVIAVHPSVKSIKVGDRVAIEPQVICNACEPCLTGRY
NGCERVDFLSTPPVPGLLRRYVNHPAVWCHKIGNMSYENGAMLEPLSVALAGLQRAG
VRLGDPVLICGAGPIGLITMLCAKAAGACPLVITSRDEGRLKFAKEICPEVVTHKVERLS
AEESAKKIVESFGGIEPAVALECTGVESSIAAAIWAVKFGGKVFVIGVGKNEIQIPFMRAS
VREVDLQFQYRYCNTWPRAIRLVENGLVDLTRLVTHRFPLEDALKAFETTSDPKTGAIK
VQIQSLE.
L-Arabitol dehydrogenase homolog of Penicillum chrysogenum
(SEQ ID NO: 71):
MASATVTKTNIGVYTNPKHDLWIADSSPTAEDINAGKGLKAGEVTIEVRSTGICGSDVHF
WHAGCIGPMIVTGDHVLGHESAGQVLAVAPDVTHLKVGDRVAVEPNVICNACEPCLTG
RYNGCVNVAFLSTPPVDGLLRRYVNHPAVWCHKIGDMSYEDGAMLEPLSVTLAAIERS
GLRLGDALLITGAGPIGLISLLSARAAGACPIVITDIDEGRLAFAKSLVPEVRTYKVEIGKS
AEECADGIINALNDGQGSGPDALRPKLALECTGVESSVNSAIWSVKFGGKVFVIGVGKN
EMTIPFMRLSTQEIDLQYQYRYCNTWPRAIRLIQNGVIDLSKLVTHRYSLENALQAFETA
SNPKTGAIKVQIMSSEEDVKAATAGQKY.
L-Arabitol dehydrogenase homolog of Penicillum chrysogenum
SRT mutant (SEQ ID NO: 72):
MASATVTKTNIGVYTNPKHDLWIADSSPTAEDINAGKGLKAGEVTIEVRSTGICGSDVHF
WHAGCIGPMIVTGDHVLGHESAGQVLAVAPDVTHLKVGDRVAVEPNVICNACEPCLTG
RYNGCVNVAFLSTPPVDGLLRRYVNHPAVWCHKIGDMSYEDGAMLEPLSVTLAAIERS
GLRLGDALLITGAGPIGLISLLSARAAGACPIVITSRDEGRLAFAKSLVPEVRTYKVEIGKS
AEECADGIINALNDGQGSGPDALRPKLALECTGVESSVNSAIWSVKFGGKVFVIGVGKN
EMTIPFMRLSTQEIDLQYQYRYCNTWPRAIRLIQNGVIDLSKLVTHRYSLENALQAFETA
TNPKTGAIKVQIMSSEEDVKAATAGQKY.
L-Arabitol dehydrogenase homolog of Aspergillus fumigatus
A1163 (SEQ ID NO: 73):
MDVIIRKPQNFAIHTSPSHDLRLVECEIPKLRPDECLVHVRATGICGSDVHFWKHGRIGP
MIVTGDNGLGHESAGVVLQIGEAVTRFKPGDRVALECGVPCSKPTCSFCRTGKYHACPD
VVFFSTPPHHGTLRRYHAHPEAWLHKIPDNISFEEGSLLEPLSVALAGINRSGLRLADPLV
ICGAGPIGLITLLAASAAGAEPIVITDIDENRLSKAKELVPRVHPVHVQKQESPQHLGARI
VRELGQEAKLVLECTGVESSVHAGIYATRFGGMVFVIGVGKDFQNIPFMHMSAKEIDLR
FQYRYHDIYPRAINLVSAGMIDLKPLVSHRYKLEDGLAAFDTASNPAARAIKVQIIDDE.
L-Arabitol dehydrogenase homolog of Botryotinia fuckeliana
B05.10 (SEQ ID NO: 74):
MSPSATEITETTMAKPTKSNIGVYTNPAHDLWVAEAEPSLESIEKGDSLKPGEVTVGIRS
VGICGSDVHFWHAGCIGPMIVEDTHILGHESAGVVLAVHPSVDSLKVGDRVAVEPNIIC
GECERCLTGRYNGCEKVLFLSTPPVPGLLRRYVNHPATWCYKIGNMSFEDGAMLEPLS
VALAGLERANVKLGDPVLICGAGPIGLITLLCARAAGACPIVITDIDEGRLAFAKELVPSV
TTHKVERLSAEEGAKSIVKSFGGIEPAVAMECTGVESSVAAACAVKFGGKVFVVGVGK
DEMTLPFMRLSTREVDLQFQYRYCNTWPRAIRLVESGIIDMKKLVTHRFPLEDAIKAFET
AANPKTGAIKVQIKNDE.
L-Arabitol dehydrogenase homolog of Magnaporthe oryzae
70-15 (SEQ ID NO: 75):
MSATNGSAAAAPSKKNIGVFTNPKHDLWINEAEPSLESVQKGSDELKEGQVTIAIRSTGI
CGSDVHFWHHGCIGPMIVREDHILGHESAGEIIAVHPSVTSLKVGDRVAVEPQVICYECE
PCLTGRYNGCEKVDFLSTPPVPGLLRRYVNHPAVWCHKIGDMSWEDGAMLEPLSVALA
GIQRAGITLGDPVLVCGAGPIGLITLLCAKAAGACPLVITDIDDGRLKFAKELVPDVITFK
VEGRPTAEDAAKSIVEAFGGVEPTLAIECTGVESSIASAIWAVKFGGKVFVIGVGRNEISL
PFMRASVREVDLQFQYRYCNTWPRAIRLIQNKVIDLTKLVTHRFPLEDALKAFETAADP
KTGAIKVQIQSLE.
L-Arabitol dehydrogenase homolog of Nectria haematococca
mpVI 77-13-4 (SEQ ID NO: 76):
MSPSAVDAPATADVKTTLKPNIGVYTNPNHDLWVNAAEPSAESVKSGADLKQGEVSVA
IRSTGICGSDVHFWHAGCIGPMIVEGDHILGHESAGEVVAVHPSVTNLKVGDRVAVEPNI
PCGTCEPCLTGRYNGCETVQFLSTPPVPGMLRRYINHPAVWCHKIGNMSYENGAMLEP
LSVALAGMQRAQVSLGDPVLICGAGPIGLITLLCSAAAGASPIVITDISESRLAFAKELCP
RVITHKVERLSAEDSAKAIVNSFGGVEPTIALECTGVESSIAAAIWSVKFGGKVFIIGVGK
NEINIPFMRASVREVDIQLQYRYCNTWPRAIRLVESGVIDLSKLVTHRFKLEDALKAFET
SADPKSGSIKVMIQSLE.
L-Arabitol dehydrogenase homolog of Podospora anserina,
DSM980 (SEQ ID NO: 77):
MSTTTTTTKVKASKANIGVFTNPGHDLWIDSAEPSLESVQQGSPELKEGEVTVAIRSTGI
CGSDVHFWKHGCIGPMIVTCDHVLGHESAGEIIAVHPSVKTLQVGDRVAIEPQVICNECE
PCLTGRYNGCEKVDFLSTPPVAGLLRRYVNHKAVWCHKIGDMSYEDGAMLEPLSVAL
AGMQRAGVRLGDPVLICGAGPIGLITLLCCQAAGACPLVITDIDEGRLKFAKEIAPGVVT
VKVEPGLSVEQQAERIVKEGFNGIEPAIALECTGVESSIGAAIWAMKFGGKVFVIGVGRN
EIQIPFMRASVREVDLQFQYRYSNTWPRAIRLVQSKVLDMSRLVTHRFPLEEALKAFNT
ASDPKTGAIKVQIQSLD.
L-Arabitol dehydrogenase homolog of Haeosphaeria nodorum
SN15 (SEQ ID NO: 78):
MSSTTVTEVKPSKANIGVYTNPAHDLWVAEAEPSLEVVEKGGDLKEGEVLLNVKSTGIC
GSDIHFWHAGCIGPMIVEDTHILGHESAGTVLAVHPSVSTLKVGDRVAIEPNVICHECEP
CLTGRYNGCEKVQFLSTPPVTGLLRRYLKHPAMWCHKLPDNLTFEDGAMLEPLSVALA
GMDRANVRLGDPVVICGAGPIGLVTLLCARAAGAAPIVITDIDEGRLKFAKDLVPNVAT
HKVEFSHSVDDFRNAVIAKMEGVEPAIAMECTGVESSINGAIQAVKFGGKVFVIGVGKN
EMKIPFMRLSTREVDLQFQYRYCNTWPKAIRLVKSGVIELSKLVTHRFQLEDAVQAFKT
AADPKTGAIKVQIQSLD.
L-Xylulose Reductase (LXR) Sequences
L-Xylulose reductase homolog of Ambrosiozyma monospora
(SEQ ID NO: 79):
MTDYIPTFRFDGHLTIVTGACGGLAEALIKGLLAYGSDIALLDIDQEKTAAKQAEYHKY
ATEELKLKEVPKMGSYACDISDSDTVHKVFAQVAKDFGKLPLHLVNTAGYCENFPCED
YPAKNAEKMVKVNLLGSLYVSQAFAKPLIKEGIKGASVVLIGSMSGAIVNDPQNQVVY
NMSKAGVIHLAKTLACEWAKYNIRVNSLNPGYIYGPLTKNVINGNEELYNRWISGIPQQ
RMSEPKEYIGAVLYLLSESAASYTTGASLLVDGGFTSW.
L-Xylulose reductase homolog of Aspergillus nidulans
(SEQ ID NO: 80):
MPQQVPTASHLSDLFSLKGKVVVITGASGPRGMGIEAARGCAEMGANVAITYASRPEG
GEKNAAELARDYGVKAKAYKCDVGDFKSVEKLVQDVIAEFGQIDAFIANAGRTASAGV
LDGSVKDWEEVVQTDLNGTFHCAKAVGPHFKQRGKGSLVITASMSGHIANYPQEQTSY
NVAKAGCIHMARSLANEWRDFARVNSISPGYIDTGLSDFVDKKTQDLWLSMIPMGRHG
DAKELKGAYVYLVSDASTYTTGADLVIDGGYTCR.
L-Xylulose reductase homolog of Aspergillus terreus NIH2624
(SEQ ID NO: 81):
MPIPVPSANHLKDLFSLKDKVVVITGASGPRGMGIEAARGCAEMGANVAITYASRPQGG
EKNAEELAKAYGVKAKAYKCDVGNFESVEKLVKDVIAEFGQIDAFIANAGRTASSGILD
GSVNDWMEVIQTDLTGTFHCAKAVGPHFKQRGTGSLVITASMSGHIANFPQEQTSYNV
AKAGCIHLARSLANEWRDFARVNSISPGYIDTGLSDFVPKDVQDLWMSMIPMGRNGDA
KELKGAYVYLVSDASTYTTGADLRIDGGYCVR.
L-Xylulose reductase homolog of Neurospora crassa OR74A
(SEQ ID NO: 82):
MASTTKGNAIPTASKLSDLFSLKGKVVVITGASGPRGMGIEAARGCAEMGASVAITYAS
RADGAQKNVAELEKEYGIKAKAYKLNVADYAECEKLVKDVIADFGQIDAFIANAGATA
KSGVLDGSKEEWDRVIETDLNGTAYCAKAVGPHFKERGRGSFVITSSISGHIANYPQEQT
SYNVAKAGCIHMARSLANEWRDFARVNSISPGYIDTGLSDFVDQKTQDLWKSMIPLGR
NGDAKELKGAYVYLVSDASSYTTGADILIDGGYTVR.
L-Xylulose reductase homolog of Candida dubliniensis
(SEQ ID NO: 83):
MSKETISYTNDALGPLPTKPATIPDNILDAFSLKGKVASVTGSSGGIGWAVAEGYAQAG
ADVAIWYNSHPADDKAEYLAKTYGVKSKAYKCNVTDFQDVEKVVKQIESDFGTIDIFV
ANAGVAWTDGPEIDVKGVDKWNKVVNVDLNSVYYCAHVVGPIFRKHGKGSFIFTASM
SASIVNVPQLQAAYNAAKAGVKHLSKSLSVEWAPFARVNSVSPGYIATHLSEFADPDVK
NKWLQLTPLGREAKPRELVGAYLYLASDAASYTTGADLAVDGGYTVV.
L-Xylulose reductase homolog of Hypocrea jecorina
(SEQ ID NO: 84):
MPQPVPTANRLLDLFSLKGKVVVVTGASGPRGMGIEAARGCAEMGADLAITYSSRKEG
AEKNAEELTKEYGVKVKVYKVNQSDYNDVERFVNQVVSDFGKIDAFIANAGATANSG
VVDGSASDWDHVIQVDLSGTAYCAKAVGAHFKKQGHGSLVITASMSGHVANYPQEQT
SYNVAKAGCIHLARSLANEWRDFARVNSISPGYIDTGLSDFIDEKTQELWRSMIPMGRN
GDAKELKGAYVYLVSDASSYTTGADIVIDGGYTTR.
L-Xylulose reductase homolog of Aspergillus terreus
NIH2624 (SEQ ID NO: 85):
MESVKNSIRWPNPALPDSVFKMFDMHGKVVIITGGSGGIGYQVARALAEAGADIALWY
NSSPDAVRLASTLEKDFGVRSEAYKCSVQNFDEVQAATDAVVRDFGGLHVMIANAGIP
SKAGGLDDRLEDWQRVVDIDFSGAYYCARAAGQIFRKQGFGNMIFTASMSGHAANVP
QQQACYNACKAGVIHLAKSLAVEWAGFARVNCVSPGYIDTPISGDCPFEMKEAWYSLT
PMRRDADPRELKGVYLYLASDASTYTTGADVVVDGGYTCR.
L-Xylulose reductase homolog of Aspergillus niger
(SEQ ID NO: 86):
MPISIPSASSVHDLFSLKGKVVVITGASGPRGMGIEAARGCAEMGANIALTYSSRPQGGE
KNAEELRNTYGVKAKAYQCNVGDWNSVKKLVDDVLAEFGQIDAFIANAGKTASSGILD
GSVEDWEEVIQTDLTGTFHCAKAVGPHFKQRGTGSFIITSSMSGHIANFPQEQTSYNVAK
AGCIHMARSLANEWRDFARVNSISPGYIDTGLSDFVDKKTQDLWMSMIPMGRNGDAKE
LKGAYVYLASDASTYTTGADLVIDGGYTVR.
Nucleic acid sequence encoding L-xylulose reductase homolog of Aspergillus niger,
which has been codon optimized for expression in S. cerevisiae (SEQ ID NO: 21):
atgcctatttccattccatctgcatcctcagttcatgatctgttttctcttaagggcaaggttgttgtgataacaggtgcatctggaccaagaggga
tgggtattgaagctgctagaggttgtgccgaaatgggtgctaacatcgctctaacctattcatctcgtcctcaaggaggggagaagaacgct
gaagaactgagaaatacttacggcgtcaaggctaaagcatatcagtgcaatgtgggcgattggaacagtgtaaagaagttggttgatgatgt
cttagctgagtttggacagattgatgctttcatagctaacgccggtaaaacagctagttctggtatcttagacggctcagtggaagattgggaa
gaggtaatacaaactgacttaactgggacattccactgtgcaaaagccgtcggccctcatttcaagcaaagaggtacaggcagtttcatcatc
acttcatcaatgtcaggtcacatagctaacttcccacaagaacaaacctcctacaatgtagcaaaggccggctgtatccacatggccagatca
ttagccaatgagtggagagattttgctagggttaactctatctctcctggttacattgatactggattgagtgatttcgttgacaaaaagacacaa
gatttgtggatgtcaatgattccaatgggtagaaacggagatgcaaaagaactaaaaggggcctacgtataccttgcatccgatgcatctaca
tacacaacaggagctgatttggttattgatggaggctataccgtcagataa.
L-xylulose reductase homolog of Ambrosiozyma monospora
(SEQ ID NO: 87):
MTDYIPTFRFDGHLTIVTGACGGLAEALIKGLLAYGSDIALLDIDQEKTAAKQAEYHKY
ATEELKLKEVPKMGSYACDISDSDTVHKVFAQVAKDFGKLPLHLVNTAGYCENFPCED
YPAKNAEKMVKVNLLGSLYVSQAFAKPLIKEGIKGASVVLIGSMSGAIVNDPQNQVVY
NMSKAGVIHLAKTLACEWAKYNIRVNSLNPGYIYGPLTKNVINGNEELYNRWISGIPQQ
RMSEPKEYIGAVLYLLSESAASYTTGASLLVDGGFTSW.
Nucleic acid sequence encoding L-xylulose reductase homolog of Ambrosiozyma
monospora, which has been codon optimized for expression in S. cerevisiae
(SEQ ID NO: 95):
atgacagactacatacctacattcagattcgacggtcacttaactatcgtaactggtgcctgtggtggtttagcagaagcattgattaaaggttt
gttagcctatggttcagatatagctttgttagatatcgaccaagaaaagactgctgcaaagcaagcagaatatcataagtacgccacagaaga
attgaagttgaaggaagttccaaagatgggttcctacgcctgtgatatttctgattcagacaccgttcataaagtatttgcacaagtcgccaaag
acttcggtaaattgcctttacacttggttaatactgctggttattgtgaaaactttccatgcgaagattaccctgctaaaaatgcagaaaagatggt
aaaggtcaacttgttaggttccttatatgttagtcaagccttcgctaaaccattgatcaaggaaggtattaaaggtgcttccgttgtattaattggtt
ccatgagtggtgcaatagtaaatgaccctcaaaaccaagtcgtttacaacatgagtaaggcaggtgtcatacacttagccaaaacattggctt
gcgaatgggcaaagtacaacatcagagttaattctttgaacccaggttacatctacggtcctttgaccaaaaatgtaattaatggtaacgaaga
attgtacaacagatggatttctggtataccacaacaaagaatgtcagaacctaaggaatacataggtgctgttttgtacttgttgtctgaatcagc
agcctcctatacaacaggtgcttccttattggtagacggtggtttcacttcttggtag.
L-xylulose reductase homolog (dicarbonyl/L-xylulose
reductase) of Mus musculus (SEQ ID NO: 88):
MDLGLAGRRALVTGAGKGIGRSTVLALKAAGAQVVAVSRTREDLDDLVRECPGVEPV
CVDLADWEATEQALSNVGPVDLLVNNAAVALLQPFLEVTKEACDTSFNVNLRAVIQVS
QIVAKGMIARGVPGAIVNVSSQASQRALTNHTVYCSTKGALDMLTKMMALELGPHKIR
VNAVNPTVVMTPMGRTNWSDPHKAKAMLDRIPLGKFAEVENVVDTILFLLSNRSGMTT
GSTLPVDGGFLAT.
Nucleic acid sequence encoding L-xylulose reductase homolog of Mus musculus,
which has been codon optimized for expression in S. cerevisiae (SEQ ID NO: 96):
atggatttgggtttggctggtagaagagcattggtaacaggtgctggtaaaggtatcggtagaagtacagtattggcattgaaggcagccgg
tgctcaagttgtagcagtttctagaaccagagaagatttggatgacttagttagagaatgtccaggtgtagaacctgtttgcgtagatttggctg
actgggaagcaacagaacaagccttatcaaatgtaggtccagtcgatttgttagtaaataacgctgcagtcgcattgttgcaaccatttttgga
agttacaaaggaagcttgtgacacctccttcaatgttaacttaagagcagttattcaagtaagtcaaatcgtcgccaagggtatgatcgctaga
ggtgtaccaggtgctattgtcaatgtttcttcacaagcttctcaaagagcattgactaaccatacagtttattgctcaactaaaggtgcattggata
tgttaacaaagatgatggccttggaattaggtcctcacaaaattagagtcaatgccgttaacccaaccgtcgttatgactcctatgggtagaact
aattggtccgatccacataaagcaaaggccatgttggacagaatacctttgggtaaattcgctgaagttgaaaacgtagtcgatacaattttatt
cttgttaagtaacagaagtggtatgacaacaggttcaacattgccagtagacggtggtttcttagcaacttag.
L-xylulose reductase homolog (dicarbonyl/L-xylulose reductase)
of Cavia porcellus (SEQ ID NO: 89):
MDLGLAGRRALVTGAGKGIGRSTVLALKAAGAQVVAVSRTREDLDDLVRECPGVEPV
CVDLADWEATEQALSNVGPADLLVNNAAVALLQPFLEVTKEACVTSFNVNLRAVIQVS
QIVAKGMIARGVPGAIVNVSSQASQRALTNHTVYCSTKGALYMLTKMMALELGPHKIR
VNAVNPTVVMTPMGRTNWSDPHKAKAMLDRIPLGKFAEVENVVDTILFLLSNRSGMTT
GSTLPVDGGFLAT.
Nucleic acid sequence encoding L-xylulose reductase homolog of Cavia porcellus,
which has been codon optimized for expression in S. cerevisiae (SEQ ID NO: 97):
atggacttaggtttggctggtagaagagcattggtcactggtgctggtaaaggtataggtagatccaccgtattggcattgaaggcagccggt
gctcaagttgtagcagtttctagaaccagagaagatttggatgacttagttagagaatgtccaggtgtagaacctgtttgcgtagatttggctga
ctgggaagcaacagaacaagccttatcaaatgttggtccagctgacttgttagtcaataacgctgcagttgcattgttgcaaccatttttggaag
ttacaaaggaagcctgtgtaacctccttcaatgtcaacttaagagctgtaattcaagtcagtcaaatagtcgccaagggtatgatcgctagagg
tgtaccaggtgctattgtcaatgtttcttcacaagcttctcaaagagcattgactaaccatacagtttattgctcaactaaaggtgcattgtacatgt
taacaaagatgatggccttggaattaggtcctcacaaaattagagttaatgcagtaaacccaaccgtcgttatgactcctatgggtagaactaa
ttggtccgatccacataaagcaaaggccatgttggacagaatacctttgggtaaattcgctgaagttgaaaacgtagtcgatacaattttattctt
gttaagtaacagatctggtatgactactggttcaactttgcctgtcgacggtggtttcttggctacttag.

### Example 2

### Cloning of Homologous Genes Involved in Pentose Utilization

Strains, Media, and Cultivation Conditions.

S. cerevisiae L2612 (MATα leu2-3 leu2-112 ura3-52 trp1-298 can1 cyn1 gal⁺) was kindly provided by Y. S. Jin (Jin et al., Applied and Environmental Microbiology, 69:495-503, 2003; and Ni et al., Applied and Environmental Microbiology, 73:2061-2066, 2007). Escherichia coli DH5α (Cell Media Facility, University of Illinois at Urbana-Champaign, Urbana, Ill.) was used for recombinant DNA manipulation. Yeast strains were cultivated in either synthetic dropout media (0.17% Difco yeast nitrogen base without amino acids and ammonium sulfate, 0.5% ammonium sulfate, 0.083% amino acid drop out mix) or YPA media supplemented with sugar as carbon source (1% yeast extract, 2% peptone, 0.01% adenine hemisulfate). E. coli strains were cultured in Luria broth (LB) (Fisher Scientific, Pittsburgh, Pa.). S. cerevisiae strains were cultured at 30° C. and 250 rpm for aerobic growth, and 30° C. and 100 rpm for oxygen-limited condition. E. coli strains were cultured at 37° C. and 250 rpm unless specified otherwise. All restriction enzymes were purchased from New England Biolabs (Ipswich, Mass.). All chemicals were purchased from Sigma Aldrich (St. Louis, Mo.) or Fisher Scientific (Pittsburgh, Pa.).

Plasmid and Strain Construction.

Most of the cloning work was done using the yeast homologous recombination mediated DNA assembler method (Shao et al., Nucleic Acids Research, 37:e16, 2009). DNA fragments flanked with regions homologous to adjacent DNA fragments were generated with polymerase chain reaction (PCR). The PCR-amplified DNA fragments were subsequently purified and co-transferred into S. cerevisiae along with the pRS414 backbone. Different auxotrophic markers were used for the individual gene cloning vector, and the final pathway assembly vector, to reduce problems associated with template contamination. To confirm the correct clones from transformants, yeast plasmids were isolated using a Zymoprep II yeast plasmid isolation kit (Zymo Research, Orange, Calif.) and transferred into E. coli.

Plasmids from E. coli were then isolated and insert sequence was confirmed using diagnostic PCR. XR expression cassette sequences were confirmed using the primer pair: ADH1p-Seq-for: 5′-GTTTGCTGTC TTGCTATCAA G-3′ (SEQ ID NO:98); and ADH1t-Seq-rev: 5′-CAACGTATCT ACCAACGATT TG-3′ (SEQ ID NO:99). XDH expression cassette sequences were confirmed using the primer pair: PGK1p-Seq-for: 5′-CTAATTCGTA GTTTTTCAAG TTC-3′ (SEQ ID NO:100); and CYC1t-Seq-rev: 5′-GGACCTAGAC TTCAGGTTGT C-3′ (SEQ ID NO:101). XKS expression cassette sequences were confirmed using the primer pair: PYK1p-Seq-for: 5′-CCTTTCAAAG TTATTCTCTA CTC-3′ (SEQ ID NO:102); and ADH2t-Seq-rev: 5′-CAAGAAACAA TACAATCATC TC-3′ (SEQ ID NO:103). LAD expression cassette sequences were confirmed using the primer pair: GPDp-Seq-for: 5′-GACGGTAGGT ATTGATTGTA ATTC-3′ (SEQ ID NO:104); and PYK1t-Seq-rev: 5′-CTTTATTTGA GTTGAAAAG-3′ (SEQ ID NO:105). LXR expression cassette sequences were confirmed using the primer pair: TEF1p-Seq-for: 5′-CGGTCTTCAA TTTCTCAAGT TTC-3′ (SEQ ID NO:106); and HXT7t-seq-rev: 5′-GAGTACATTT CAAATGCAC-3′ (SEQ ID NO:107). Constructs yielding PCR products of the predicted size were confirmed to be correct.

Cloning of Enzyme Homologues into Vectors.

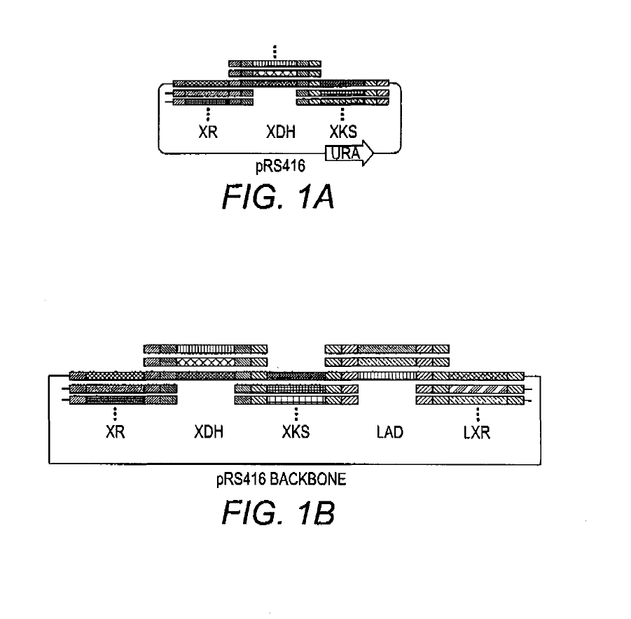

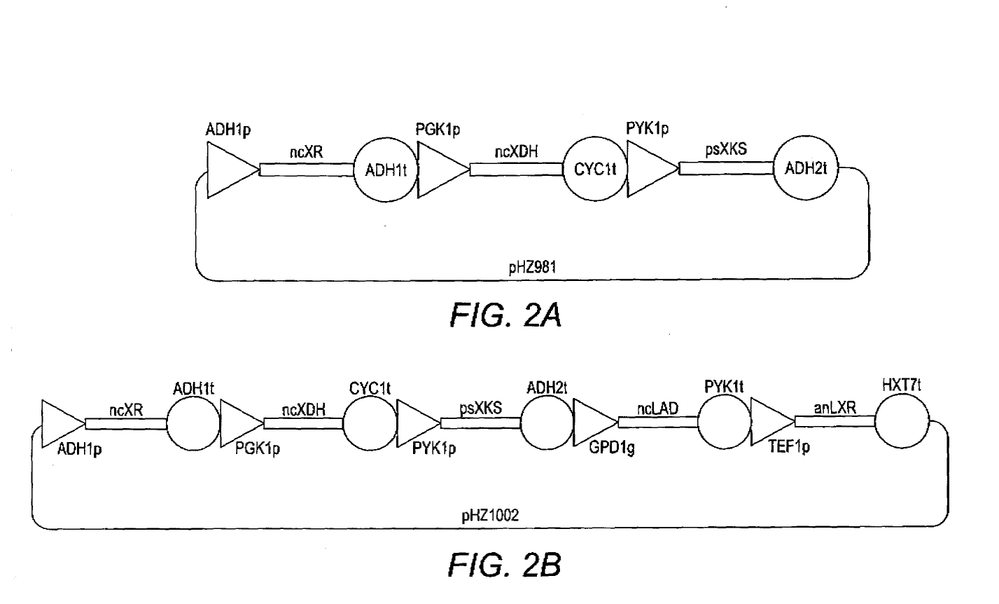

To construct the scaffolds, expression cassettes of pentose-utilization pathway genes were assembled (FIG. 1) into the pRS416 single copy shuttle vector using the yeast homologous recombination mediated DNA assembler method (Shao et al., Nucleic Acids Research, 37:e16, 2009). Two general scaffolds (FIGS. 2A and 2B) for the three-gene xylose utilization pathway and the five-gene arabinose/xylose utilization pathway were constructed using fungal and other nucleic acid templates. In the DNA assembler method, for each individual gene in a pathway, an expression cassette including a promoter, a structural gene, and a terminator was PCR-amplified. The 5′-end of the first gene expression cassette was designed to overlap with the vector while the 3′-end was designed to overlap with the second cassette. Each successive cassette was designed to overlap with the two flanking ones, and the 3′-end of the last cassette overlapped with the vector. Unlike the conventional cloning approach that relies on site-specific digestion and ligation, homologous recombination aligns complimentary sequences and enables the exchange between homologous elements.

For the three gene xylose utilization pathway, expression cassettes were prepared for the xylose reductase (XR) and the xylitol dehydrogenase (XDH) from Neurospora crassa, and the xylulokinase (XKS) from Pichia stipitis. Specifically, the N. crassa xylose reductase ORF was assembled with an ADH1 promoter (1,500 bp) and an ADH1 terminator (327 bp) using overlapping extension PCR (OE-PCR) to generate a XR gene expression cassette. Similarly, the N. crassa xylitol dehydrogenase ORF was assembled with a PGK1 promoter (750 bp) and a CYC1 terminator (250 bp) by OE-PCR to generate a XDH gene cassette, while the P. stipitis xylulokinase ORF was assembled with a PYK1 promoter (1,000 bp) and an ADH2 terminator (400 bp) by OE-PCR to generate a XKS gene cassette. The resultant gene expression cassettes were then assembled using the DNA assembler method into a linearized pRS416 plasmid to generate the pHZ981 xylose scaffold shown in FIG. 2A. Similarly, as shown in FIG. 2B, the pHZ1002 xylose/arabinose scaffold was assembled by addition of the N. crassa L-arabitol 4-dehydrogenase (LAD) ORF flanked by the GPD1 promoter (655 bp) and the PYK1 terminator (400 bp), as well as the Aspergillus niger L-xylulose reductase (LXR) ORF flanked by the TEF1 promoter (412 bp) and the HXT7 terminator (400 bp).

TABLE 2-1
Enzyme Sequences for Scaffold Construction

| | GenBank | SEQ |
|---|---|---|
| Fungal Enzymes | Accession No. | ID NO: |
| ncXR | CAA42072 | 6 |
| (N. crassa xylose reductase) | | |
| ncXDH | AAD28251 | 27 |
| (N. crassa xylitol dehydrogenase) | | |
| psXKS | XP_001387325 | 48 |
| (P. stipitis xylulokinase) | | |
| ncLAD | XP_965783 | 69 |
| (N. crassa L-arabitol 4-dehydrogenase) | | |
| anLXR_opt | XP_001397074 | 86 |
| (A. niger L-xylulose reductase) | | |

The promoters and terminators were PCR-amplified individually from the genomic DNA isolated from S. cerevisiae (Saccharomyces cerevisiae YSG50 (MATα, ade2-1, ade3A22, ura3-1, his3-11, 15, trp1-1, leu2-3,112 and can1-100)) using the Wizard Genomic DNA isolation kit from Promega (Madison, Wis.). The nucleic acid sequences of the yeast promoters and terminators are provided below.

The ADH1 promoter is set forth as SEQ ID NO: 108:
tgcctgcaggtcgagatccgggatcgaagaaatgatggtaaatgaaataggaaatcaaggagcatgaaggcaaaagacaaatataagggt
cgaacgaaaaataaagtgaaaagtgttgatatgatgtatttggctttgcggcgccgaaaaaacgagtttacgcaattgcacaatcatgctgact
ctgtggcggacccgcgctcttgccggcccggcgataacgctgggcgtgaggctgtgcccggcggagttttttgcgcctgcattttccaagg
tttaccctgcgctaaggggcgagattggagaagcaataagaatgccggttggggttgcgatgatgacgaccacgacaactggtgtcattatt
taagttgccgaaagaacctgagtgcatttgcaacatgagtatactagaagaatgagccaagacttgcgagacgcgagtttgccggtggtgc
gaacaatagagcgaccatgaccttgaaggtgagacgcgcataaccgctagagtactttgaagaggaaacagcaatagggttgctaccagt
ataaatagacaggtacatacaacactggaaatggttgtctgtttgagtacgctttcaattcatttgggtgtgcactttattatgttacaatatggaa
gggaactttacacttctcctatgcacatatattaattaaagtccaatgctagtagagaaggggggtaacacccctccgcgctcttttccgatttttt
tctaaaccgtggaatatttcggatatccttttgttgtttccgggtgtacaatatggacttcctcttttctggcaaccaaacccatacatcgggattcc
tataataccttcgttggtctccctaacatgtaggtggcggaggggagatatacaatagaacagataccagacaagacataatgggctaaaca
agactacaccaattacactgcctcattgatggtggtacataacgaactaatactgtagccctagacttgatagccatcatcatatcgaagtttca
ctaccctttttccatttgccatctattgaagtaataataggcgcatgcaacttcttttctttttttttcttttctctctcccccgttgttgtctcacc
atatccgcaatgacaaaaaaaatgatggaagacactaaaggaaaaaattaacgacaaagacagcaccaacagatgtcgttgttccagagctgatga
ggggtatctcgaagcacacgaaactttttccttccttcattcacgcacactactctctaatgagcaacggtatacggccttccttccagttacttg
aatttgaaataaaaaaaagtttgctgtcttgctatcaagtataaatagacctgcaattattaatcttttgtttcctcgtcattgttctcgttcccttt
cttccttgtttctttttctgcacaatatttcaagctataccaagcatacaatcaactcca.
The PGK1 promoter is set forth as SEQ ID NO: 109:
acgcacagatattataacatctgcacaataggcatttgcaagaattactcgtgagtaaggaaagagtgaggaactatcgcatacctgcatttaa
agatgccgatttgggcgcgaatcctttattttggcttcaccctcatactattatcagggccagaaaaaggaagtgtttccctccttcttgaattgat
gttaccctcataaagcacgtggcctcttatcgagaaagaaattaccgtcgctcgtgatttgtttgcaaaaagaacaaaactgaaaaaacccag
acacgctcgacttcctgtcttcctattgattgcagcttccaatttcgtcacacaacaaggtcctagcgacggctcacaggttttgtaacaagcaa
tcgaaggttctggaatggcgggaaagggtttagtaccacatgctatgatgcccactgtgatctccagagcaaagttcgttcgatcgtactgtta
ctctctctctttcaaacagaattgtccgaatcgtgtgacaacaacagcctgttctcacacactcttttcttctaaccaagggggtggtttagtttagt
agaacctcgtgaaacttacatttacatatatataaacttgcataaattggtcaatgcaagaaatacatatttggtcttttctaattcgtagtttttca
agttcttagatgctttctttttctcttttttacagatcatcaaggaagtaattatctactttttacaacaaatataaaaca.
The PYK1 promoter is set forth as SEQ ID NO: 110:
aatgctactattttggagattaatctcagtacaaaacaatattaaaaagaggtgaattatttttccccccttattttttttttgttaaaattgatcca
aatgtaaataaacaatcacaaggaaaaaaaaaaaaaaaaaaaaaatagccgccatgaccccggatcgtcggttgtgatacggtcagggtagcg
ccctggtcaaacttcagaactaaaaaaataataaggaagaaaaaaatagctaatttttccggcagaaagattttcgctacccgaaagtttttcc
ggcaagctaaatggaaaaaggaaagattattgaaagagaaagaaagaaaaaaaaaaaatgtacacccagacatcgggcttccacaatttc
ggctctattgttttccatctctcgcaacggcgggattcctctatggcgtgtgatgtctgtatctgttacttaatccagaaactggcacttgacccaa
ctctgccacgtgggtcgttttgccatcgacagattgggagattttcatagtagaattcagcatgatagctacgtaaatgtgttccgcaccgtcac
aaagtgttttctactgttctttcttctttcgttcattcagttgagttgagtgagtgctttgttcaatggatcttagctaaaatgcatattttttctc
ttggtaaatgaatgcttgtgatgtcttccaagtgatttcctttccttcccatatgatgctaggtacctttagtgtcttcctaaaaaaaaaaaaaggc
tcgccatcaaaacgatattcgttggcttttttttctgaattataaatactctttggtaacttttcatttccaagaacctcttttttccagttatatc
atggtcccctttcaaagttattctctactctttttcatattcattctttttcatcctttggttttttattcttaacttgtttattattctctcttg
tttctatttacaagacaccaatcaaaacaaataaaacatcatcaca.
The GPD1 promoter is set forth as SEQ ID NO: 111:
agtttatcattatcaatactcgccatttcaaagaatacgtaaataattaatagtagtgattttcctaactttatttagtcaaaaaattagcctttta
attctgctgtaacccgtacatgcccaaaatagggggcgggttacacagaatatataacatcgtaggtgtctgggtgaacagtttattcctggcatcca
ctaaatataatggagcccgctttttaagctggcatccagaaaaaaaaagaatcccagcaccaaaatattgttttcttcaccaaccatcagttcat
aggtccattctcttagcgcaactacagagaacaggggcacaaacaggcaaaaaacgggcacaacctcaatggagtgatgcaacctgcct
ggagtaaatgatgacacaaggcaattgacccacgcatgtatctatctcattttcttacaccttctattaccttctgctctctctgatttggaaaaag
ctgaaaaaaaaggttgaaaccagttccctgaaattattcccctacttgactaataagtatataaagacggtaggtattgattgtaattctgtaaatc
tatttcttaaacttcttaaattctacttttatagttagtcttttttttagttttaaaacaccagaacttagtttcgacggat.
The TEF1 promoter is set forth as SEQ ID NO: 112:
atagcttcaaaatgtttctactccttttttactcttccagattttctcggactccgcgcatcgccgtaccacttcaaaacacccaagcacagcatac
taaatttcccctctttcttcctctagggtgtcgttaattacccgtactaaaggtttggaaaagaaaaaagagaccgcctcgtttctttttcttcgtcg
aaaaaggcaataaaaatttttatcacgtttctttttcttgaaaatttttttttttgatttttttctctttcgatgacctcccattgatatttaagttaa
taaacggtcttcaatttctcaagtttcagtttcatttttcttgttctattacaactttttttacttcttgctcattagaaagaaagcatagcaatctaa
tctaagttttaattacaaa.
The ADH1 terminator is set forth as SEQ ID NO: 113:
tggacttcttcgccagaggtttggtcaagtctccaatcaaggttgtcggcttgtctaccttgccagaaatttacgaaaagatggaaaagggtca
aatcgttggtagatacgttgttgacacttctaaataagcgaatttcttatgatttatgatttttattattaaataagttataaaaaaaataagtgtata
caaattttaaagtgactcttaggttttaaaacgaaaattcttgttcttgagtaactctttcctgtaggtcaggttgctttctcaggtatagcatgaggt
cgctcttattgaccacacctctaccggcatgc.
The CYC1 terminator is set forth as SEQ ID NO: 114:
atcatgtaattagttatgtcacgcttacattcacgccctccccccacatccgctctaaccgaaaaggaaggagttagacaacctgaagtctag
gtccctatttatttttttatagttatgttagtattaagaacgttatttatatttcaaatttttcttttttttctgtacagacgcgtgtacgcatgtaaca
ttatactgaaaaccttgcttgagaaggttttgggacgctcgaaggctttaatttgcgg.
The ADH2 terminator is set forth as SEQ ID NO: 115:
ggtttgctgagaagcttgccaaatgattgactttataagaacggctgaccatggtagacggacccggttgatgggcttcatattgagatgattg
tattgtttcttgacttctgagagtttttggttttttattatgttctccatgtctcggttcttacgttcgcattgttttatattttatttcatgtttatc
aagagctctagaattcatagtcgaccggaccgatgccttcacaatttatagttttcattatcaagtatgcctatattagtatatagatctttacgatga
cagtgttcgaagtttcacgaataaaagataatattctactttttgctcccctcgactttgttcccactgtacttttagctcgtacaaaatacaatatac
ttttcatttctccgtaaacaacatgttttcccatgtaatatccttttctatttttcgttccgttaccaactttacacatactttatatagctattcact
tctatacactaaaaaactaagacaattttaattttgctgcctgccatatttcaatttgttataaattcctataatttatcctattagtagct.
The PYK1 terminator is set forth as SEQ ID NO: 116:
aaaaagaatcatgattgaatgaagatattatttttttgaattatattttttaaattttatataaagacatggtttttcttttcaactcaaataaagatt
tataagttacttaaataacatacattttataaggtattctataaaaagagtattatgttattgttaacctttttgtctccaattgtcgtcataacgatg
aggtgttgcatttttggaaacgagattgacatagagtcaaaatttgctaaatttgatccctcccatcgcaagataatcttccctcaaggttatcatgat
tatcaggatggcgaaaggatacgctaaaaattcaataaaaaattcaatataattttcgtttcccaagaactaacttggaaggttatacatgggtacata
aatg.
The HXT7 terminator is set forth as SEQ ID NO: 117:
tttgcgaacacttttattaattcatgatcacgctctaatttgtgcatttgaaatgtactctaattctaattttatatttttaatgatatcttgaaaagt
aaatacgtttttaatatatacaaaataatacagtttaattttcaagtttttgatcatttgttctcagaaagttgagtgggacggagacaaagaaacttt
aaagagaaatgcaaagtgggaagaagtcagttgtttaccgaccgcactgttattcacaaatattccaattttgcctgcagacccacgtctacaaattt
tggttagtttggtaaatggtaaggatatagtagagcctttttgaaatgggaaatatcttctttttctgtatcccgcttcaaaaagtgtctaatgagtc
agttat.

Hereafter, the scaffolds for the pentose utilization pathways, namely the combination of promoters and terminators for each catalytic step, remained consistent. Fixed scaffolds provided many advantages for subsequent investigation. First of all, all five promoters used in this study have been tested in various nutrition and aeration conditions and the expression levels proved to be similar and constitutive. As such, the difference in the expression level and enzyme activity should be mainly dependent on the properties of the enzyme homologues. Second, the fixed scaffold ensures that during the random assembly of the pathway, shuffling of different enzyme homologues only occurs within the enzyme cassette of the same catalytic step. In other words, all of the resultant variant pathways in the library have complete three-gene or complete five-gene pathways. Third, because the length of yeast promoters and terminators are around 400 to 1,000 bp, the promoter and terminator of the adjacent enzyme provided a fixed DNA sequence of around 1,000 bp in length. In the later steps, these fixed DNA sequences were included in both of the neighboring gene expression cassettes to generate longer homologous ends, which resulted in higher assembly efficiency for library creation.

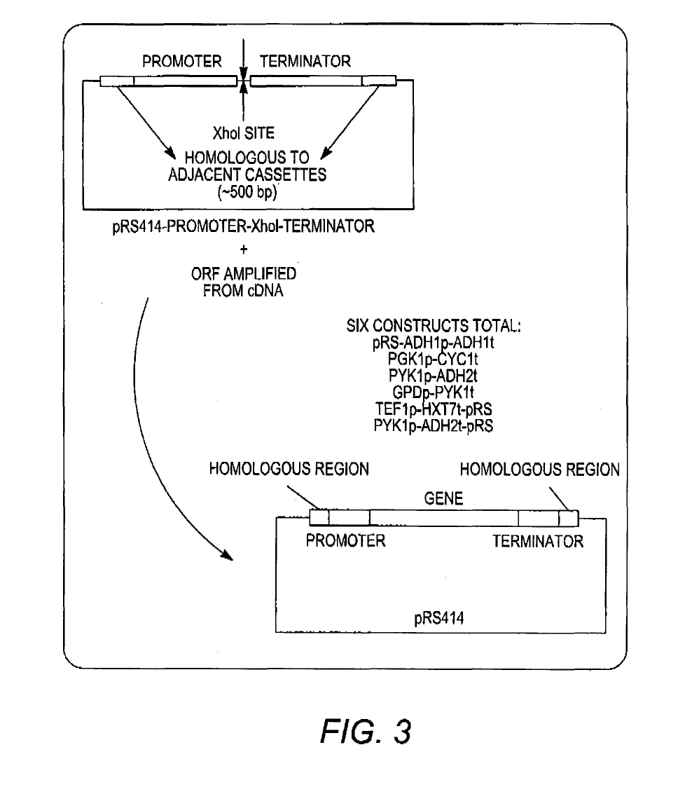

To facilitate the cloning of enzyme homologues for pathway assembly, the ORFs of the enzyme homologues were cloned into helper plasmids. Primers were designed according to the gene sequences found in GENBANK, and were subsequently used to amplify the ORFs from cDNA. The cDNAs were obtained from reverse transcription of total RNA isolated from fungal strains cultivated in YP media supplemented with xylose or arabinose. The PCR products were purified by size fractionation followed by gel extraction and then cloned into linearized pRS414 helper plasmids by yeast homologous recombination based cloning. Helper plasmids were constructed for each catalytic step of the pentose utilization pathway. A promoter with a DNA fragment (˜500 bp) homologous to the upstream adjacent sequence and a terminator with a DNA fragment (˜500 bp) homologous to the downstream adjacent sequence were assembled into a pRS414 single copy plasmid using the DNA assembler method. A unique XhoI site was engineered between the promoter and terminator to facilitate the linearization of the helper plasmids for the cloning of enzyme homologue ORFs (FIG. 3). The correctly assembled pathways were confirmed by diagnostic PCR using primers annealing to the end of the promoter and the beginning of the terminator.

For the cloning of XR homologues, an ADH1 promoter, a unique XhoI cutting site, an ADH1 terminator, and the first 480 bp of a PGK1 promoter were assembled into a pRS414 single copy shuttle vector. Similarly, an ADH1 terminator, a PGK1 promoter, a unique XhoI site, a CYC1 terminator, and the first 404 bp of a PYK1 promoter were assembled into a pRS414 vector for the cloning of XDH homologues. For the cloning XKS homologues, a CYC1 terminator, a PYK1 promoter, a unique XhoI site, and an ADH2 terminator were assembled into a pRS414 vector. Primers were designed according to the gene sequences in GenBank for the amplification of the ORFs of enzyme homologues. A DNA sequence of approximately 45 bp in length was introduced at the 5′ end of the ORF to be homologous to the 3′ end of the promoter sequence as well as at the 3′ end of the ORF in order to be homologous to the 5′ end of the terminator sequence for the homologous recombination-based cloning.

Obtaining Gene Expression Cassettes for Assembly of Enzyme Pathways.

To obtain the gene expression cassettes for random pathway assembly, PCR was used to amplify the whole gene expression cassette including the homologous region upstream of the promoter, the promoter, the target ORF, the terminator, and the homologous region downstream of the terminator. The sizes of the resultant fragments were confirmed using agarose gel electrophoresis and the DNA fragments of the correct size were purified using a PCR purification kit. The concentrations of purified DNA fragments were determined using Nanodrop (NanoDrop Technologies, Wilmington, Del.).

### Example 3

### Combinatorial Pathway Assembly of a Three Gene Xylose Utilization Library

To create a library of pentose utilization pathways, DNA fragments encoding different enzyme homologues were mixed together and co-transferred into S. cerevisiae L2612 with a linearized pRS416 plasmid. Because for each catalytic step, up to about 20 enzyme homologues were involved in the assembly of the library, the number of different DNA fragments was large. For example, for a three-gene xylose utilization pathway, 20 homologues of xylose reductase, 22 homologues of xylitol dehydrogenase, and 19 homologues of xylulokinase were used for assembly of an exemplary library. Together with the linearized backbone (e.g., pRS416 linearized with BamHI and EcoRI), there were a total of 62 DNA fragments employed in the library creation. To ensure the high efficiency of the DNA assembler method, a large quantity of DNA for each fragment is desirable. On the other hand, because the amount of DNA that can be introduced into yeast cells is limited, the introduction of an excessive amount of DNA into yeast cells results in inefficient DNA assembly and waste of DNA fragments.

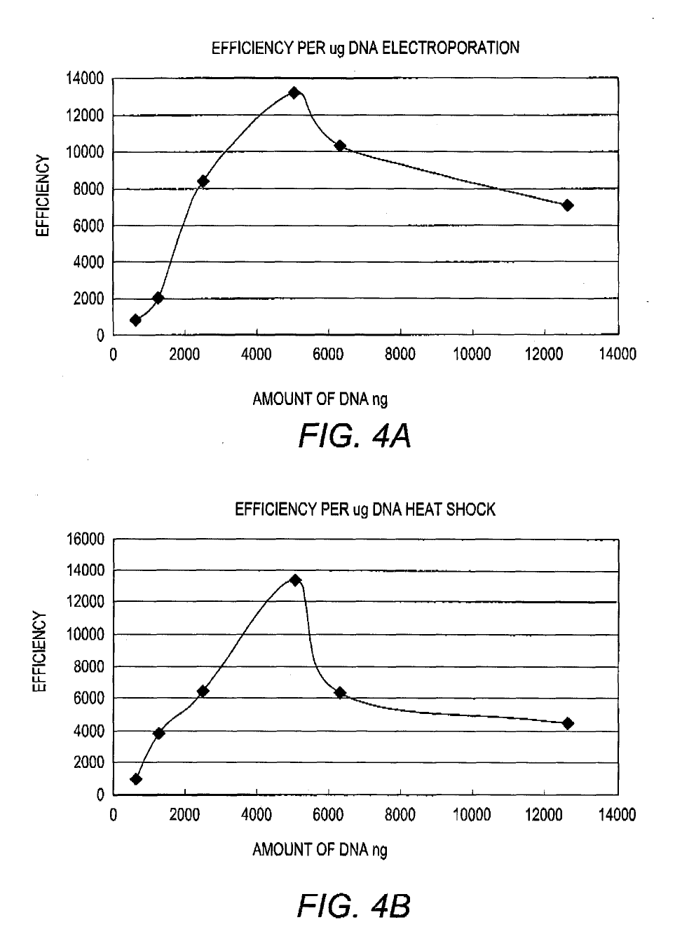

Different amounts of DNA fragments were used for library creation, and the resulting library sizes were calculated, in order to determine the optimal amount of DNA fragments for pathway assembly. Equal amounts of DNA (ng) of all the fragments were mixed and transferred into yeast using electroporation or heat shock transformation. The resulting library sizes were plotted in FIG. 4. The library sizes were determined by plating an aliquot (10 μl) of the transformant on SC-Ura+glucose plate and counting number of colonies. The overall library size was calculated based on the colony number, volume plated (10 μl), and total volume (1 ml). The transformation efficiency (transformants per microgram of DNA) was calculated from the library size and quantity of DNA used for the transformation. The optimal amount of total DNA was determined to be around 5,000 ng, resulting in a library size of around 1.3×10⁴. The transformation efficiency showed a similar trend independent of the transformation method. When a larger library size was needed, multiple transformations were performed and the resultant transformants were combined.

### Example 4

### Characterization of a Small Three Gene Xylose Utilization Library

To characterize the efficiency and diversity of the combinatorial pathway assembly method, a small library of recombinant yeast containing the three-gene xylose utilization pathway was created and evaluated. Specifically, 8 homologues of xylose reductase (XR), 10 homologues of xylitol dehydrogenase (XDH) and 6 homologues of xylulokinase (XKS) were subjected to the DNA assembler. The homologues used for construction of this small three gene pathway included the following: XRs of Aspergillus oryzae, Candida tropicalis, Pichia stipitis, Pichia guilliermondii, Kluyveromyces lactis, Candida shehatae, Candida parapsilosis, and Neurospora crass; XDHs of Pachysolen tannophilus, Aspergillus niger, Aspergillus oryzae, Candida guilliermondii, Candida shehatae, Candida tropicalis, Kluyveromyces lactis, Neurospora crassa, Pichia stipitis, and Talaromyces stipitatus; and XKSs of Candida albicans, Penicillium chrysogenum, Candida tropicalis, Saccharomyces cerevisiae, Pichia stipitis, and Aspergillus niger.

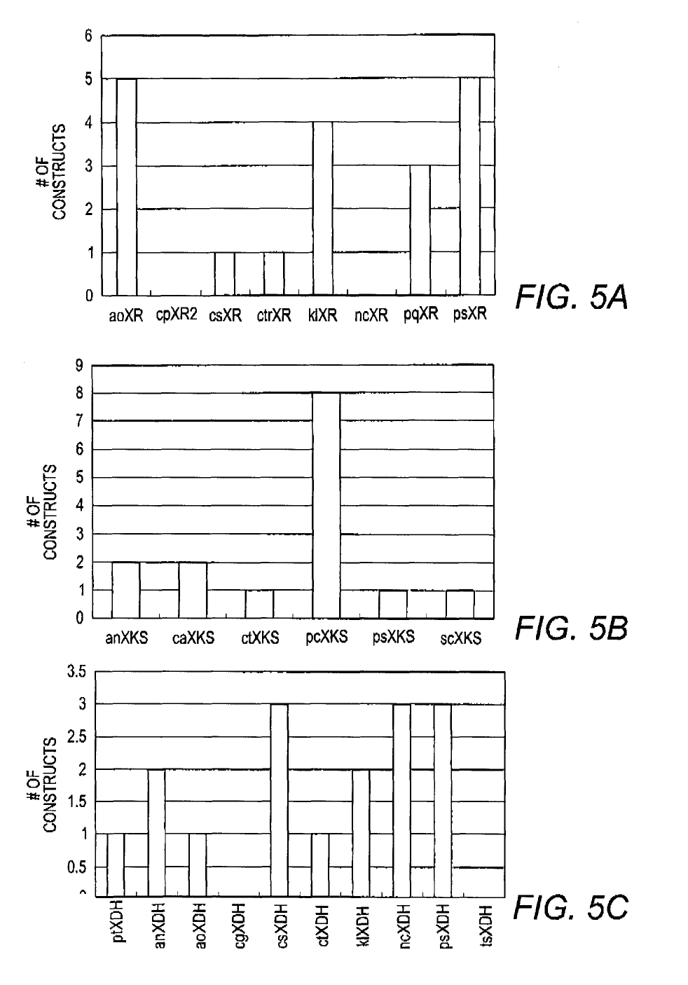

After DNA transformation, transformants were spread on a SC-Ura plate supplemented with 2% glucose, and 20 single colonies were randomly picked for subsequent analysis. These 20 transformants were first grown up in liquid SC-Ura medium supplemented with 2% glucose, and then the yeast plasmids were isolated using Zymoprep II yeast plasmid isolation kit (Zymo Research). The resulting yeast plasmids were transferred into E. coli and the corresponding plasmids were isolated from E. coli using a Qiagen Miniprep kit (Qiagen, Valencia, Calif.). The correct assembly of the three-gene xylose utilization pathway was checked using diagnostic PCR with primers annealing to the promoter and the terminator regions. All 20 constructs were found to have a correctly assembled three-gene pathway. The 20 constructs were sequenced to identify the enzyme homologues assembled into the recombinant pathway, to measure the diversity of the small library. All 20 constructs were found to have different combinations of enzyme homologues, and multiple different enzyme homologues were represented for each of the three catalytic steps of the pathway (FIG. 5).

### Example 5

### Combinatorial Pathway Assembly of a Five Gene Arabinose/Xylose Utilization Library

To create a library of yeast strains containing the five-gene arabinose/xylose utilization pathway, DNA fragments homologous to the adjacent sequences are mixed and transferred into S. cerevisiae with the linearized pRS416 shuttle plasmid. After DNA transformation, a small amount of the transformation mixture is spread on a SC-Ura plate supplemented with glucose to determine the library size. The rest of the transformation mixture is first cultivated in the liquid SC-Ura medium supplemented with glucose overnight, and then washed and inoculated into the YP and SC-Ura liquid media supplemented with xylose or arabinose for enrichment.

### Example 6

### Library Enrichment

A three gene library was enriched to obtain clones containing the optimized xylose utilization pathway (XR-XDH-XKS). First, the library was inoculated in YP media containing 2% xylose (YPX) and grown under oxygen-limited conditions. When the culture reached the late exponential growth phase (OD≈10), a portion of the culture (1%) was used to inoculate fresh medium. This sequential culture transfer was repeated three times to enrich the clones which can grow on xylose under oxygen-limited conditions with a high ethanol yield. At each round of enrichment, 10 μL of culture was plated on an agar plate containing synthetic media supplemented with glucose (2%) and lacking urea (SC-Ura-G).

### Example 7

### Characterization of the Enriched Populations

Ten randomly selected clones from the second (E#2) and third rounds (E#3) of enrichment of Example 6, were grown in culture tubes containing the SC-Ura-G media. Based on the growth rates, five clones each from E#2 and E#3 were selected and sequenced to identify the pathway genes. The growth rates and metabolism of those ten clones were determined and compared with the control strain containing three well-studied genes, XR, XDH, and XKS from Pichia stipitis (pRS426-psXP). Cells were grown in YPX under the oxygen-limited condition.

TABLE 7-1
Sequence of Heterologous Enzymes
of Randomly Selected Yeast Clones

| Clones | XR | XDH | XKS |
|---|---|---|---|
| E2.1^{a} | P. guilliermondii | N. crassa | P. chrysogenum |
| E2.6 | A. oryzae | N. crassa | P. chrysogenum |
| E2.7 | A. oryzae | N. crassa | P. chrysogenum |
| E2.8 | A. oryzae | N. crassa | P. chrysogenum |
| E2.9 | A. oryzae | N. crassa | P. chrysogenum |
| E3.2^{b} | A. oryzae | N. crassa | P. chrysogenum |
| E3.3 | A. oryzae | N. crassa | P. chrysogenum |
| E3.5 | A. oryzae | n.d.^{c} | P. chrysogenum |
| E3.6 | A. oryzae | N. crassa | P. chrysogenum |
| E3.8 | A. oryzae | N. crassa | P. chrysogenum |

^{a}E2.# indicates clones from second round of enrichment
^{b}E3.# indicates clones from the third round of enrichment
^{c}n.d. represents not determined.

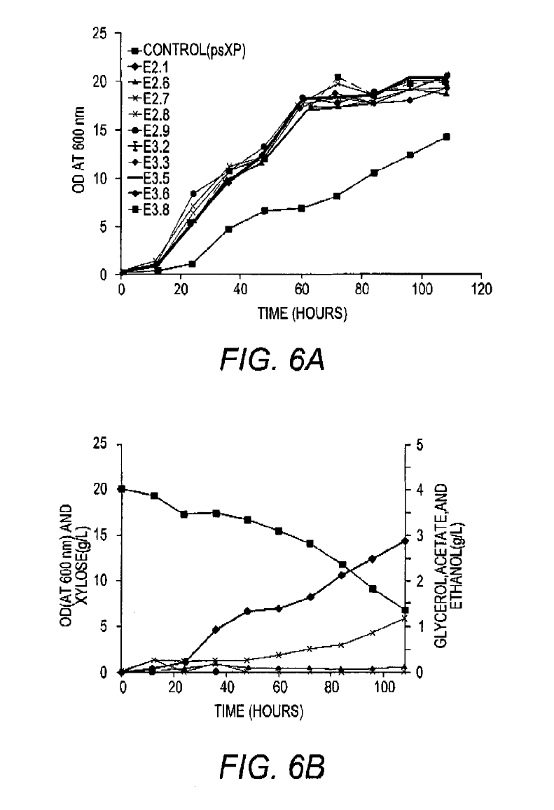

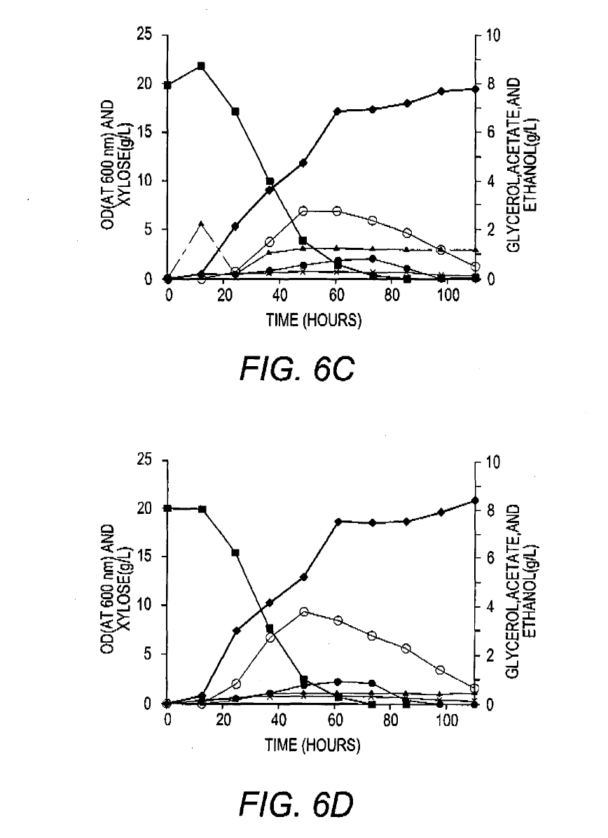

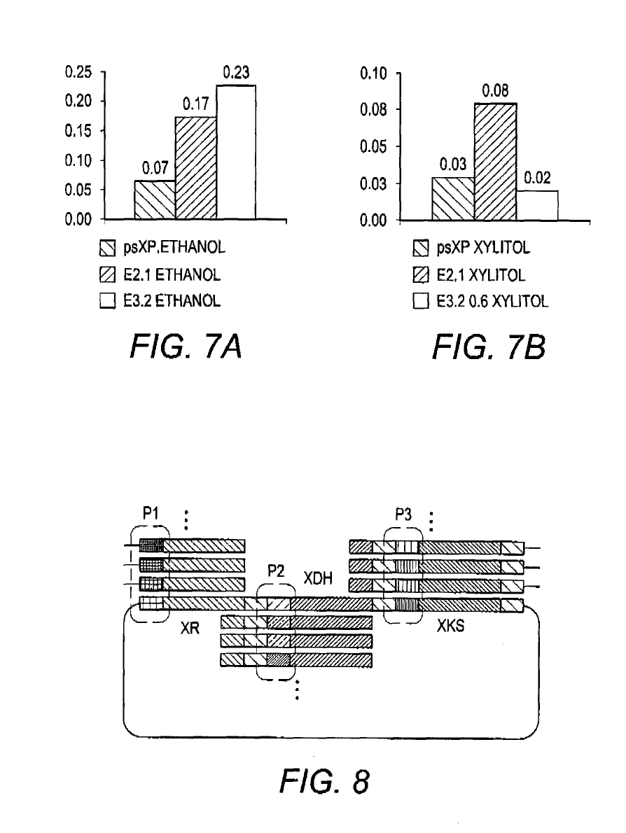

Enrichment under oxygen-limited conditions resulted in the identification and isolation of clones containing an optimized three gene xylose pathway consisting of XR of Aspergillus oryzae (aoXR), XDH of N. crassa (ncXDH), and XKS of Penicillium chrysogenum (pcXKS). Only the clone E2.1 had XR originated from Pichia guilliermondii (pgXR), and this homolog was not represented after the 3^{rd} round of enrichment. Based on the sequence analysis, there were only two distinct pathways found among the 10 clones: one containing pgXR-ncXDH-pcXKS (E2.1); and the other containing the aoXR-ncXDH-pcXKS (E3.2). These two pathways permitted the recombinant strains to grow faster on xylose than the control strain (FIG. 6A). While the growth of the control strain continued during the 108 hrs of fermentation, the growth of isolated clones reached a plateau after 60 hrs (FIG. 6A). The control strain consumed less than 15 g of xylose after 108 hrs and the ethanol yield was negligible as determined by the formation of glycerol as a by-product (FIG. 6B). Clones E2.1 and E3.2 completely consumed the xylose (remaining xylose<1.0 g/L) after 108 hrs and showed higher ethanol production after 48 hrs (FIGS. 6C and 6D). The clone E3.2 showed an ethanol yield of 0.22 g/g sugar after 48 hrs (FIG. 7A). The E2.1 clone showed a lower ethanol yield, 0.17 g/g sugar, but a higher xylitol yield (FIG. 7B) than the E3.2 clone.

TABLE 7-1
Homologues of Two Optimized Three-Gene Xylose Pathways
E2.1 enriched pathway

| | |
|---|---|
| pgXR | ABB87187 (SEQ ID NO: 7) |
| (P. guilliermondii xylose reductase) | |
| ncXDH | XP_964807 (SEQ ID NO: 27) |
| (N. crassa xylitol dehydrogenase) | |
| pcXKS | CAP80202 (SEQ ID NO: 47) |
| (P. chrysogenum xylulokinase) | |

E3.2 enriched pathway

| | |
|---|---|
| aoXR | ACX46082 (SEQ ID NO: 1) |
| (A. oryzae xylose reductase) | |
| ncXDH | XP_964807 (SEQ ID NO: 27) |
| (N. crassa xylitol dehydrogenase) | |
| pcXKS | CAP80202 (SEQ ID NO: 47) |
| (P. chrysogenum xylulokinase) | |

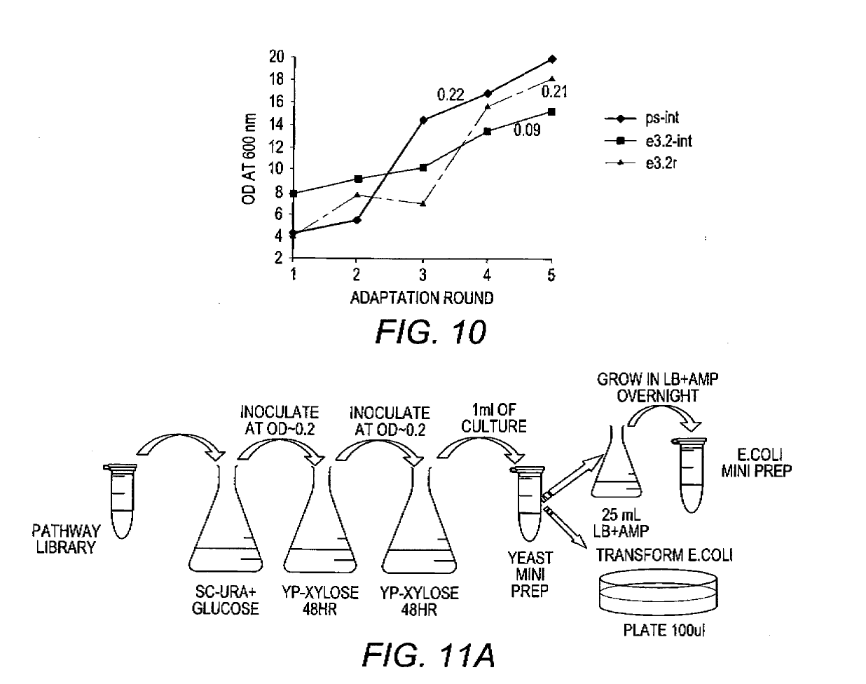

However, after isolation of the plasmid from E2.1 and E3.2 strains, and transformation of these plasmids into fresh host cells, the advantage of the enriched pathway significantly decreased (data not shown). As a control experiment, serial transfer experiments were carried out for the control pathway and the pathway library in parallel. Surprisingly, the growth and fermentation ability of the control pathway was also significantly improved after the serial transfer experiment. It appeared that the improvement of the strain performance was more likely to be from host strain adaptation rather than pathway mutant selection. (FIG. 10)

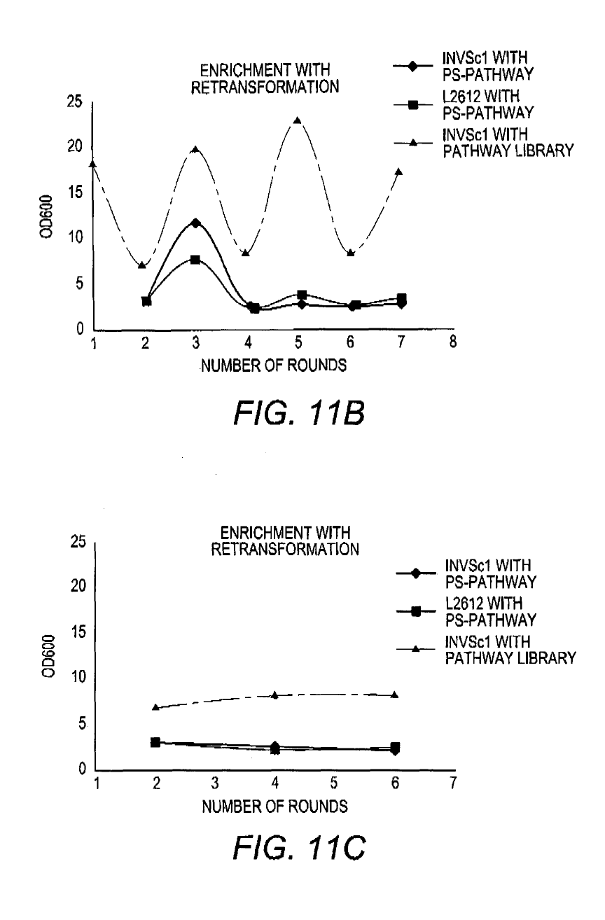

In order to remove the host cell adaptation that resulted from prolonged culture time due to serial transfer, two strategies were implemented for pathway library enrichment. In the first strategy, additional cultivation step in the SC-Ura media supplemented with 2% glucose was introduced after every two rounds of enrichment in the YP media supplement with 2% xylose to remove the host cell adaptation in xylose media. In the second strategy, yeast plasmids were isolated after every couple rounds of enrichment in the YP media and then retransferred into fresh host cells to eliminate the host adaptation. Using the first strategy, the pathway library was continuously enriched for nine rounds. Unfortunately, after retransformation, only marginal improvement was observed for the enriched mutant (FIG. 10). For the second strategy, the re-transformation step was initially introduced after every two rounds of enrichment. As shown in FIG. 11, yeast plasmids were isolated after two serial transfers of culture. The yeast plasmid was then transferred into E. coli. After propagating in E. coli, the library of plasmids were isolated and retransferred into fresh host cells. The final OD after two days of growth in shake flasks with xylose as the sole carbon source is shown in the figure. Obviously, though only two serial transfers happened before every retransformation, the host cells were adapted to the xylose media that resulted in faster cell growth. Unfortunately, this kind of improvement cannot be transferred with re-transformation of the mutant pathway into fresh host strains, and after every round of retransformation the growth rate of the library dropped back to the level before enrichment.

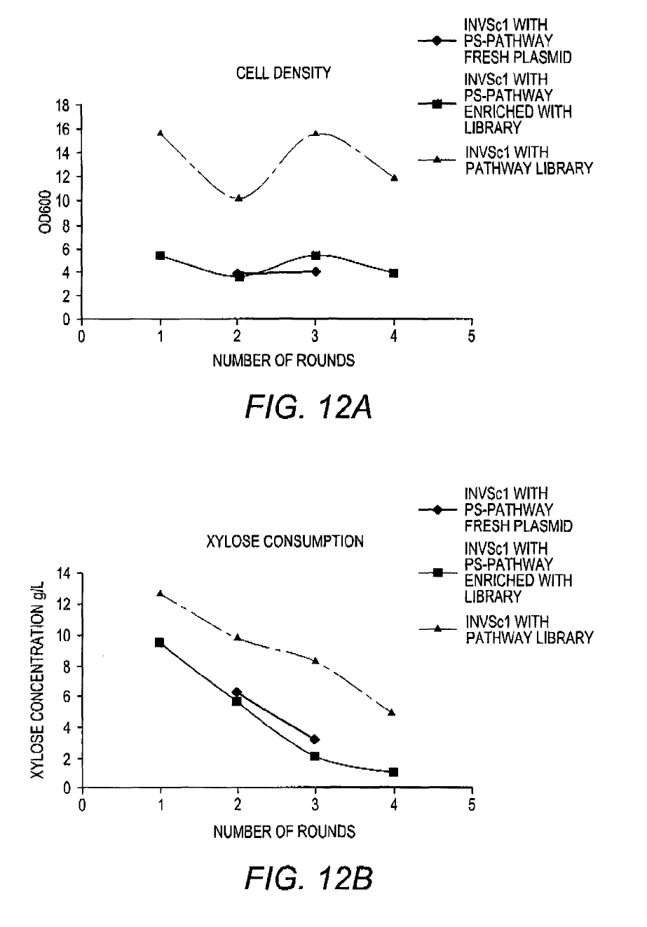

In an attempt to address the host adaptation problem within the enrichment process to the greatest extent, the second serial transfer was eliminated so that after every round of serial plasmid transfer of the pathway library would be first isolated from the yeast culture, propagated into E. coli, and then re-transferred into fresh yeast cells. FIG. 12 shows the final cell density and the xylose consumption in the YP with 2% xylose after every round of enrichment. After four rounds of enrichment, the growth rate of the mutant library remained at the same level while the xylose utilizing ability dramatically dropped. Consequently, after four rounds of re-transformation, the strains that were better at utilizing other nutrients in the rich media but not xylose were enriched. Since the main purpose of this study is to isolate mutant xylose utilization pathways that utilize xylose efficiently, this enrichment method was deemed to be ineffective.

### Example 8

### Screening of an Enzyme-Based Pathway Library

Since the enrichment method failed to identify more efficient xylose utilization pathways from the pathway library, a screening method was developed to facilitate the isolation of more efficient xylose utilization pathways. In order to reduce the amount of mutant pathways to be screened, an agar plate-based pre-screening method was used to identify the more efficient xylose utilizing pathways. To correlate the growth rate of the strain in xylose liquid medium with the colony size of the yeast strain containing that xylose utilization pathway on the agar plates with xylose as the sole carbon source, five yeast strains that harbored mutant xylose utilization pathways which exhibited different growth rates in liquid culture were spread upon SC-Ura plates supplemented with 2% xylose at the same colony density. The colony size distribution on these plates was then examined use a microscope. The microscope images were analyzed using the Image J software (NIH). Finally, colony size distributions were plotted with the growth rates in liquid culture under oxygen limited conditions.

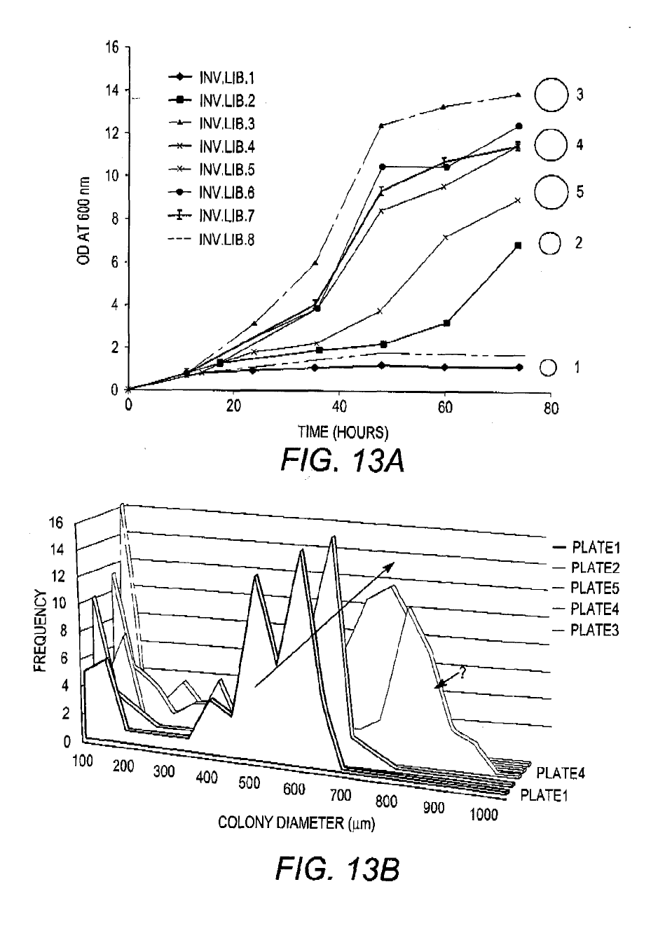

As shown in FIG. 13, yeast strains with higher growth rates tend to have larger colonies (except for the situation on plate #5 as indicated by the question mark in the right figure of FIG. 13). Therefore, we hypothesized that picking larger colonies on the agar plates, will likely enable us to find strains with a higher growth rate. This hypothesis was then incorporated into the new colony size based screening strategy to identify strains with high growth rate on xylose. Agar plates containing rich media supplemented with 2% xylose were also tested for the prescreening. Unfortunately, although cellular growth on agar plates containing rich media was faster compared to the synthetic drop-out media (SC-Ura), the differences in colonies size were not as obvious as those in the SC-Ura media. When the pathway library was spread on synthetic drop-out media plates, the size difference between the big colonies and small colonies could be readily identified by naked eyes. Yet, when the same library was spread on the rich media plates, the size differences among the strains harboring different mutant pathways were very small. More importantly, no colonies sized larger than the biggest colonies of the reference plate could be identified on the rich media plate with naked eyes. The selection of the host strain was also important for the colony size-based pre-screening strategy. The L2612 strain was used for the development for the pathway assembly strategy and for the primary characterization of the pathway libraries. However, when spread on agar plates, the colony size distribution of the L2612 strain harboring the same mutant pathway was too large. In this case, the colonies' sizes were not well correlated with the growth rates in liquid media. Fortunately, we were able to find an alternative host strain which has also been proven to be suitable for xylose fermentation (Hughes et al., Plasmid, 61, 22-38 (2009)). In subsequent studies, the INVSc1 strain (Invitrogen, Carlsbad, Calif.) was used as the host strain for pathway optimization. The colony size distribution of the INV.Sc1 strain hosting the same utilization pathway was quite uniform, making it convenient for the identification of strains that harbor more efficient mutant xylose utilizing pathways.

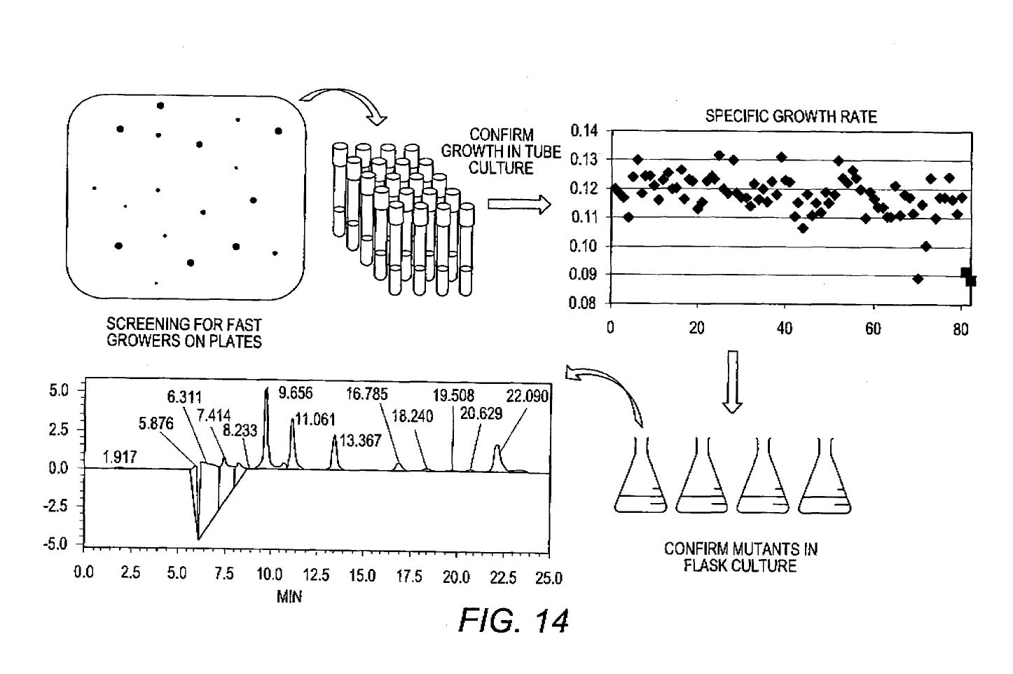

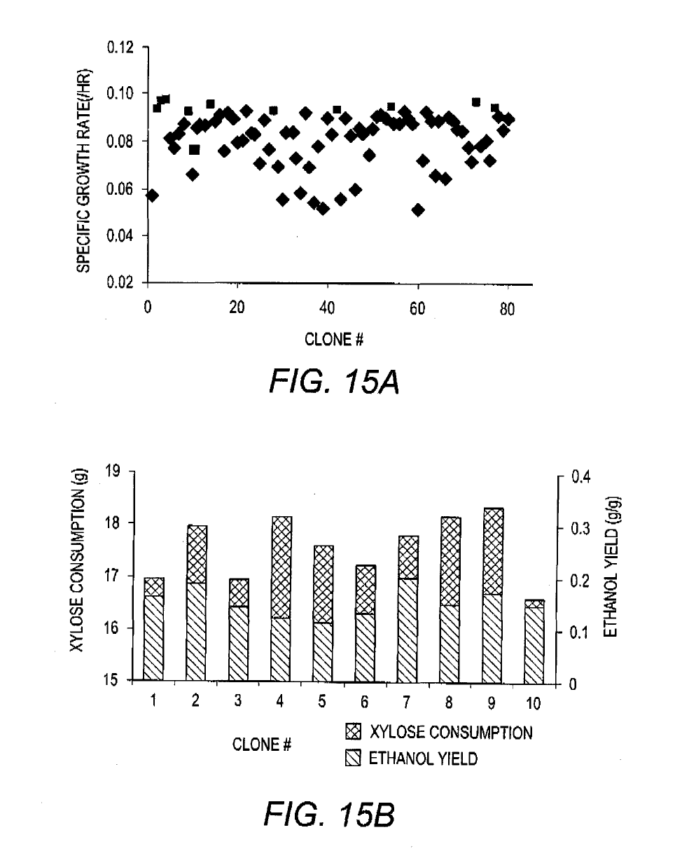

To identify more efficient mutant xylose utilization pathways, the pathway library was assembled using DNA fragments amplified from the helper plasmids. A small aliquot of the transformants were plated onto SC-Ura plates supplemented with 2% glucose in order to determine the library size. The rest of the transformants were used to inoculate a twenty-five milliliter liquid media of SC-Ura supplemented with 2% glucose. Frozen cell stocks were made from the liquid culture for later analysis. A small aliquot of the liquid culture was then washed with ddH₂O. Around 10⁵ cells were plated onto 24.5 cm by 24.5 cm square agar plates of SC-Ura supplemented with 2% xylose. At the same time, around 10⁴ cells harboring a reference pathway consisting of XR, XDH and XKS from P. stipitis were plated on regular fifteen centimeter round agar plates with the same media. The library plate and the reference plate were then incubated together and the colony sizes on both plates were checked daily. After around three days of incubation at 30° C., the differences among the colony sizes on the library plate and the reference plate gradually became obvious. Colonies on the library plate that were larger than the biggest colonies on the reference plate were then picked and inoculated first in one milliliter of SC-Ura liquid media supplemented with glucose for thirty-six hours. The liquid media was then used to inoculate a three milliliter liquid media of YP supplemented with 2% xylose to an initial OD of approximately 0.2. The tube cultures were then cultivated at 30° C. with 250 rpm agitation. The cell densities of the strains were measured after around 24 hours, 36 hours and 48 hours. The first two time points were used to determine the specific growth rate of mutant strains. The top ten strains with the highest growth rate were next subjected to another round of screening using fifty milliliter shake flask containing ten milliliter YP media supplemented with 2% xylose at 30° C. and 100 rpm agitation (oxygen limited condition). The flask cultures were then sampled at various time points and the strains found to display the highest xylose consumption and ethanol production rate were isolated. (FIG. 14)

Following the above procedure, libraries of xylose utilization pathways were also generated in the industrial strains ATCC4124 and Classic strain.

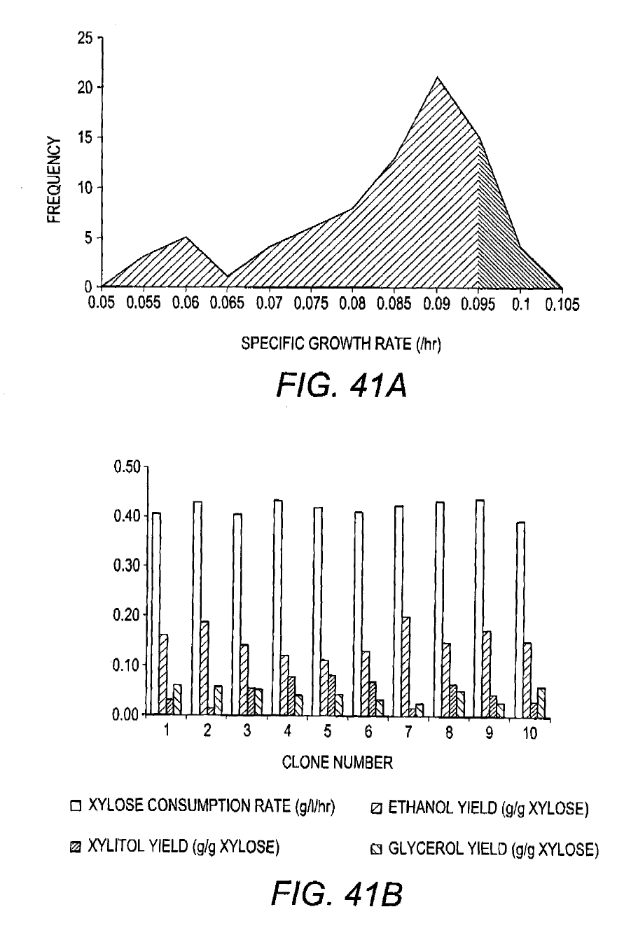

Each library was screened for efficient xylose-metabolic pathways based on the growth on xylose as a sole carbon source, ethanol yield, and minimal by-product formation. Clones formed distinctively large colonies on the selection plates were selected and subjected to a screening for fast growth on xylose liquid medium (FIG. 41A). Top 10 fast growers with highest specific growth rates were screened and tested for xylose fermentation (FIG. 41B).

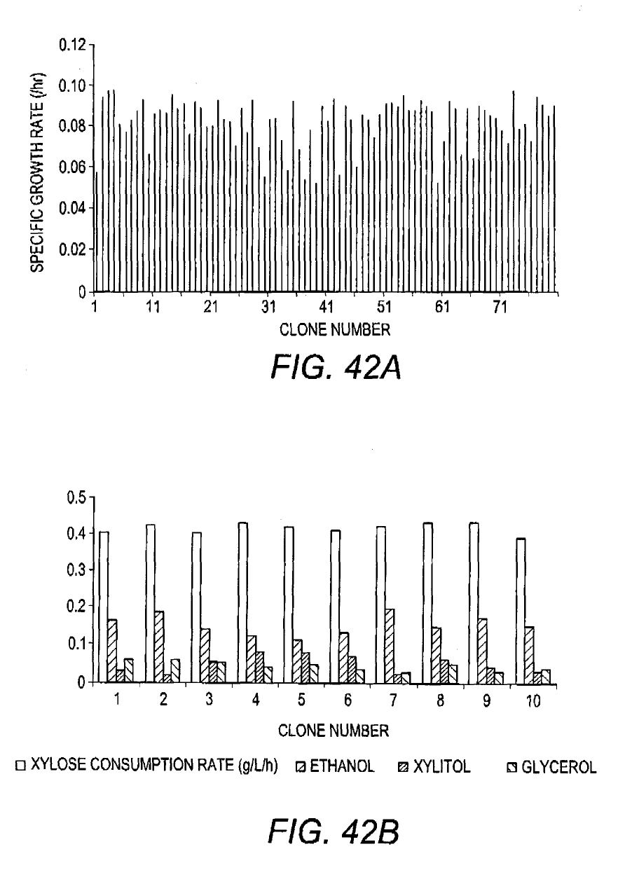

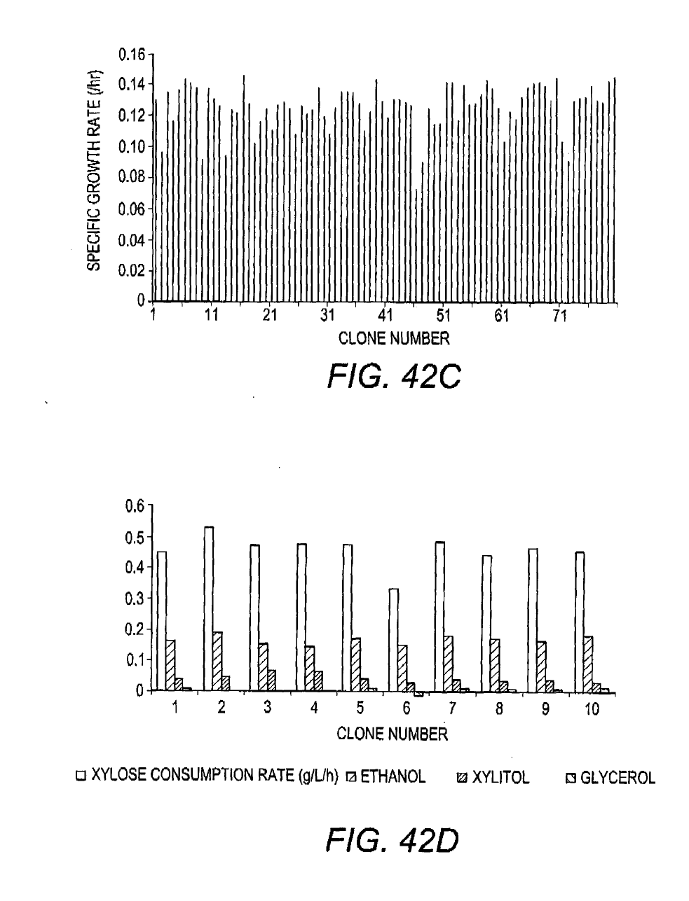

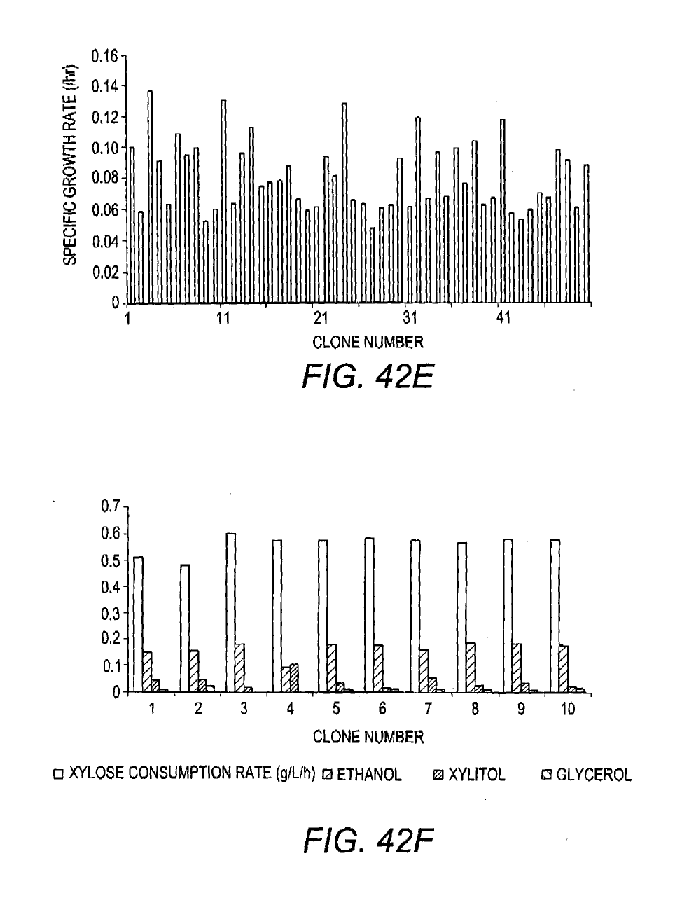

Various analyses indicated different metabolic features of various strains. The 10 fastest growers of the INVSc1 strain showed similar xylose consumption rate and growth in fermentation screening, but different profiles of by-product formation (FIG. 41B). For example, clone 2 and 5 had equivalent xylose consumption. But clone 5 showed higher xylitol and glycerol yields with lower ethanol yield than clone 2. The same observation was made in the screenings of all three strains (FIGS. 41B and 42). Clone 2 of INVSc1 contained a pathway consisting of Aspergillus nidulans XR, Candida albican XDH, and Saccharomyces cerevisiae XKS and was selected for InvSc1 (InvSc1-IL2 hereafter) for further characterization. Applying same criteria, clone 2 (ATCC-AL2) and clone 3 (Classic-CL3) were selected for ATCC 4124 and Classic strains, respectively. The screened 10 pathways for each strain contained unique combination of the enzymes and are summarized in Table 8-1.

TABLE 8-1
Sequence analysis of top 10 xylose utilization pathway
mutants from enzyme-based xylose utilization pathway screening
in the INVSc1, ATCC 4124, and Classic strains.

| | XR | XDH | XKS |
|---|---|---|---|
| InvSc1 | | | |
| s1 | A. nidulans | P. stipitis | P. anserina |
| s2 | A. nidulans | C. albicans | S. cerevisiae |
| s3 | P. stipitis | P. pastoris | P. anserina |
| s4 | N. crassa | Z. rouxii | A. niger |
| s5 | Z. rouxii | A. adeninivorans | K. lactis |
| s6 | A. nidulans | P. stipitis | P. anserina |
| s7 | N. crassa | A. adeninivorans | S. cerevisiae |
| s8 | N. crassa | N. crassa | A. niger |
| s9 | Z. rouxii | Z. rouxii | A. nidulans |
| s10 | P. stipitis | A. oryzae | N. haematococca |
| ATCC 4124 | | | |
| s1 | A. flavus | P. anserina | A. oryzae/flavus |
| s2 | P. guilliermondii | P. chrysogenum | A. oryzae |
| s3 | N. crassa | A. oryzae | P. pastoris |
| s4 | A. niger | A. niger | Z. rouxii |
| s5 | A. flavus | P. stipitis | P. guilliermondii |
| s6 | A. nidulans | A. nidulans | K. lactis |
| s7 | A. flavus | Z. rouxii | K. lactis |
| s8 | A. nidulans | C. dubliniensis | Z. rouxii |
| s9 | T. stipitatus | K. lactis | Z. rouxii |
| s10 | A. flavus | P. stipitis | Z. rouxii |
| Classic | | | |
| s1 | A. flavus | C. dubliniensis | N. haematococca |
| s2 | A. flavus | C. dubliniensis | S. cerevisiae |
| s3 | A. nidulans | A. niger | P. chrysogenum |
| s4 | A. flavus | A. niger | A. niger |
| s5 | N. crassa | A. niger | Z. rouxii |
| s6 | A. flavus | C. shehatae | N. haematococca |
| s7 | A. flavus | A. oryzae | A. niger |
| s8 | A. flavus | A. niger | C. tanophilus |
| s9 | A. flavus | P. guilliamondii | C. dubliniensis |
| s10 | T. stipitatus | C. shehatae | C. dubliniensis |

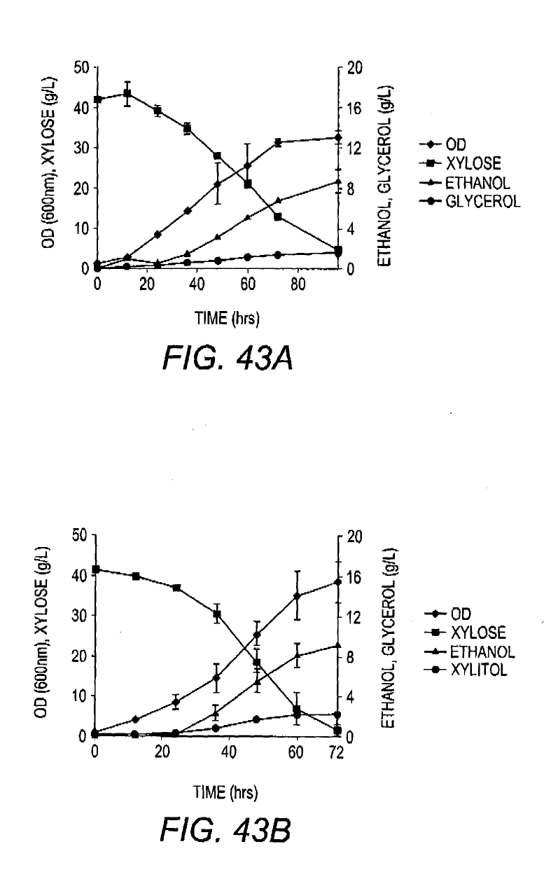

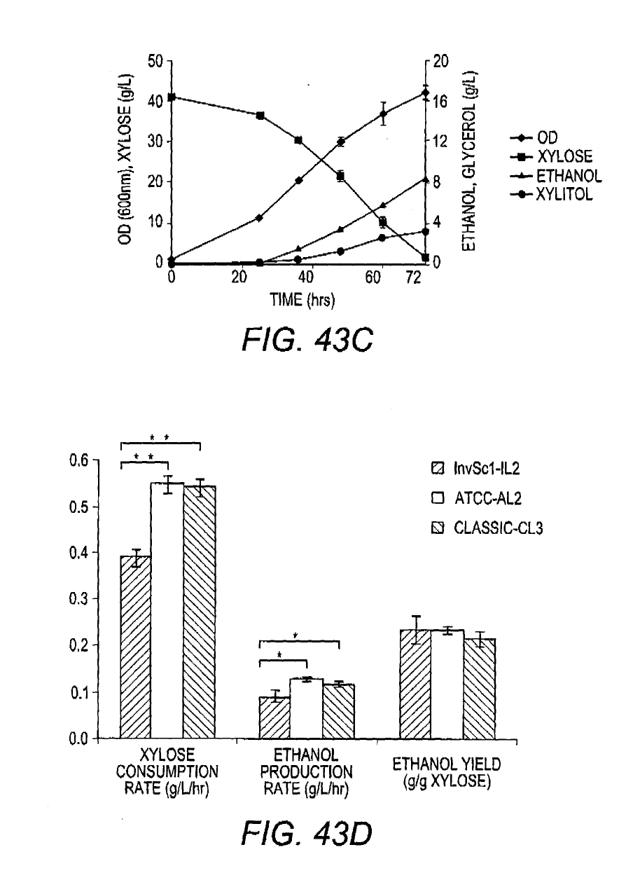

The two recombinants of the industrial strains were more efficient at xylose fermentation than the recombinant of InvSc1. InvSc1-IL2 required 96 hrs to consume 40 g/L of xylose while ATCC4127-AL2 and Classic-CL3 required 72 hrs with similar ethanol yields (FIG. 43A-D). ATCC4127-AS2 and Classic-CS3 showed significantly faster xylose consumption rates (0.55±0.02, 0.54±0.01 g/L/hr) than InvSc1-IS2 (0.39±0.00 g/L/hr, P<0.01) and ethanol production rates (0.13±0.00, 0.12±00 g/L/hr) than InvSc1-IS2 (0.09±0.01 g/L/h). All three recombinant showed comparable ethanol yields in the range of 0.20 and 0.23 g/g xylose (FIG. 43D).

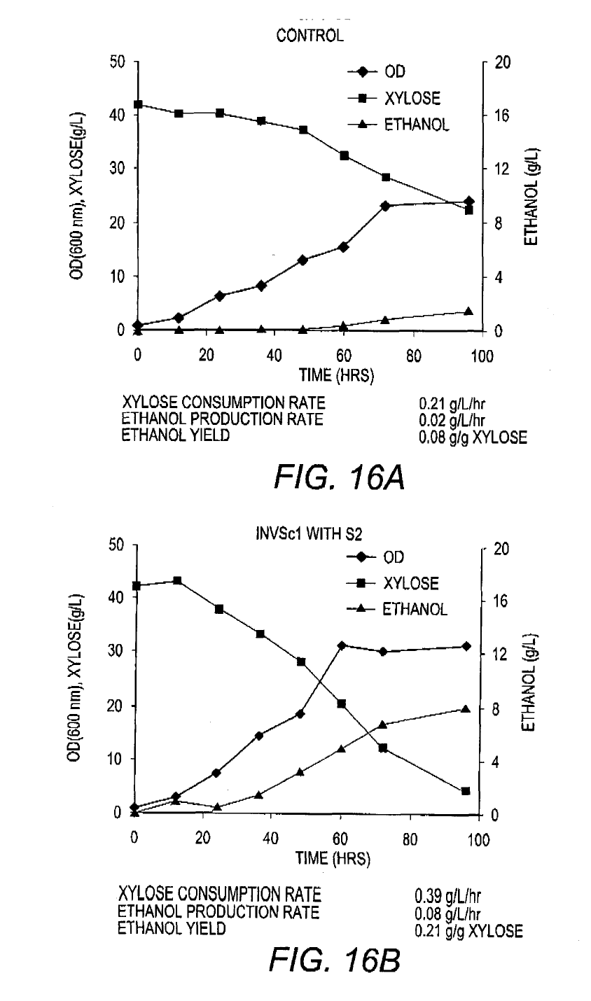

The mutant xylose utilization pathway InvSc1-IL2 was then compared with the reference pathway consisting of XR, XDH and XKS from P. stipitis in shake flask fermentation under oxygen limited conditions using rich media containing 4% xylose as sole carbon source. As shown in FIG. 16, the S2 pathway consumes xylose at a rate of 0.39 g/L/hour, while the reference pathway consumes xylose at a rate of 0.21 g/L/hour. The mutant pathway also exhibited a four-fold improvement at ethanol production rate and a 2.6-fold improvement in ethanol.

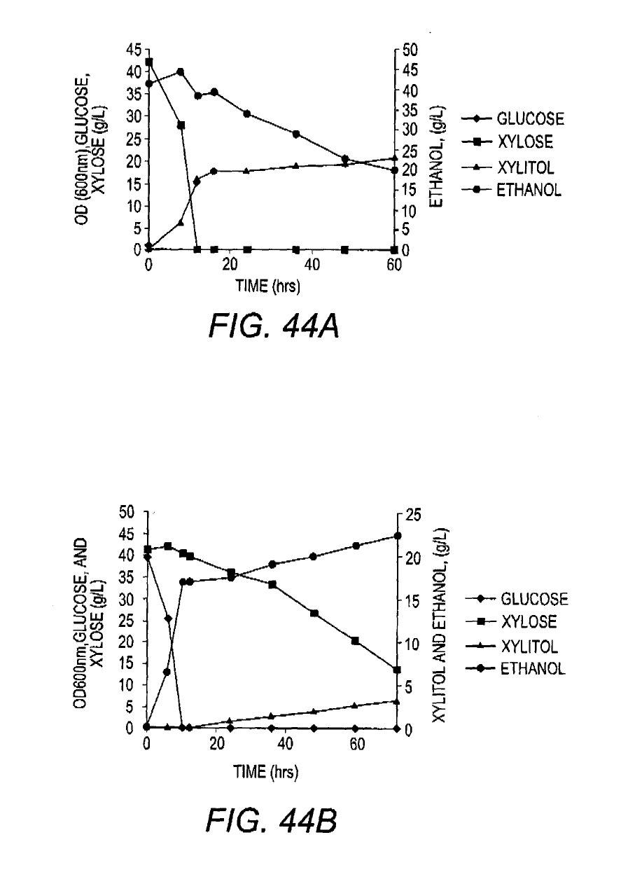

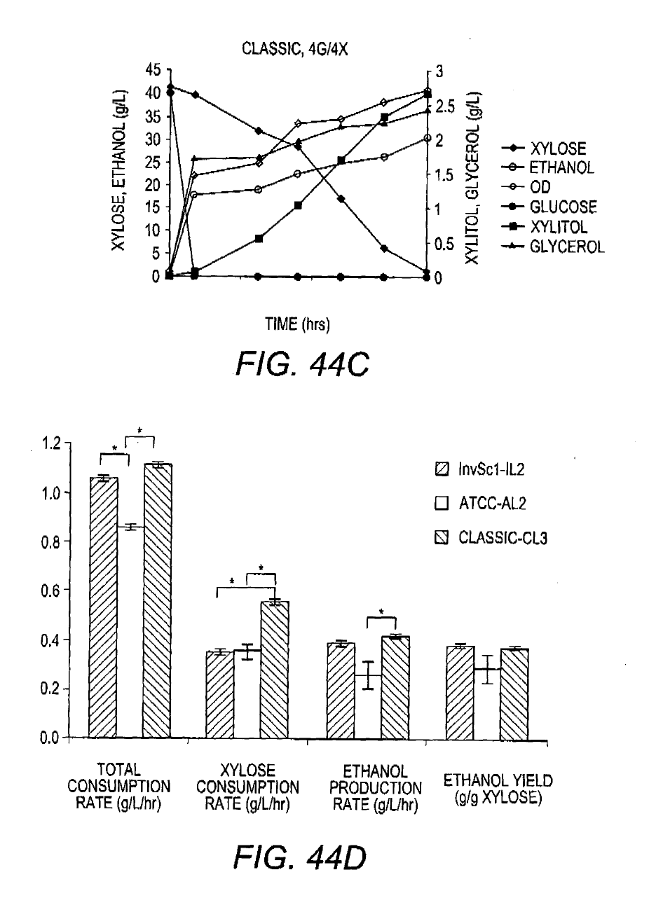

In cofermentation experiments (mixed sugar of 4% glucose and xylose). Classic-CL3 showed a substantially faster total sugar consumption than the other two recombinants (FIG. 44A-D). Classic-CL3 could consume 40 g/L xylose within 72 hrs in both single and cofermentation with 40 g/L glucose while InvSc1-CL2 and ATCC-CL2 required longer fermentation time (FIG. 44A-C). Total and xylose sugar consumption rates of Classic-CL3 were significantly faster than the other two recombinants (FIG. 44A, P<0.05). Xylose utilization efficiency of ATCC-CL2, which was equivalent to Classic-CL3 in xylose fermentation (0.55±0.02 g/L/hr), was significantly reduced by the presence of glucose and even lower than InvSc1-IL2 (0.35±0.03 g/L/hr, FIGS. 43D and 44A).

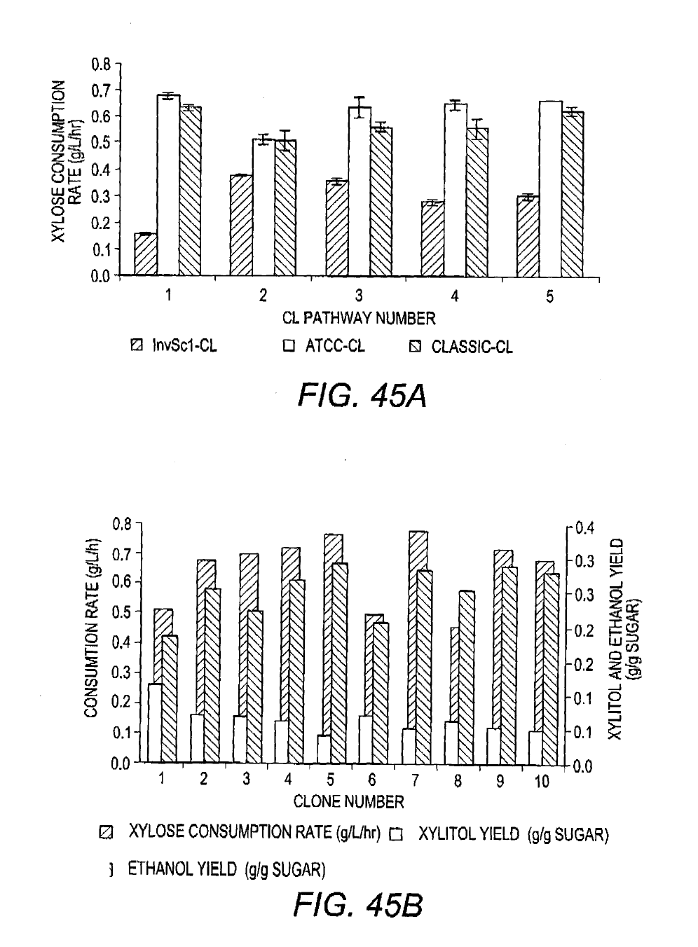

Strain background altered the optimal combinations of the enzymes in the xylose pathway. CL-1, 3, 5, 7, and 10 found in the screening of Classic strain library were transferred into InvSc1 and ATCC 4124 strains. All 5 pathways in ATCC 4124 strain were as efficient as in Classic strain. In InvSc1 strain, the xylose consumption rate and ethanol yield were significantly lower than in ATCC 4124 and Classic strains. The most noticeable difference was found in CL1 (FIG. 45A). CL1 showed the lowest xylose consumption rate and ethanol yield in InvSc1 (0.15 g/L/hr, 0.03 g/g xylose), and highest in ATCC 4124 (0.67 g/L/h, 0.24 g/g xylose) and Classic strains (0.62 g/L/h, 0.26 g/g xylose) These results suggest a strong dependency of the preferred enzymes and their combination on strain background.

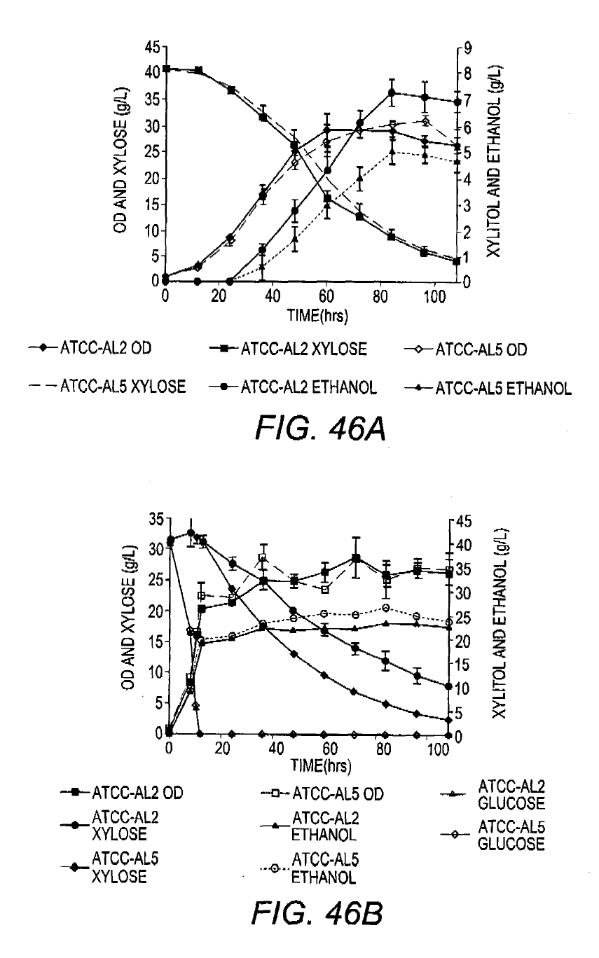

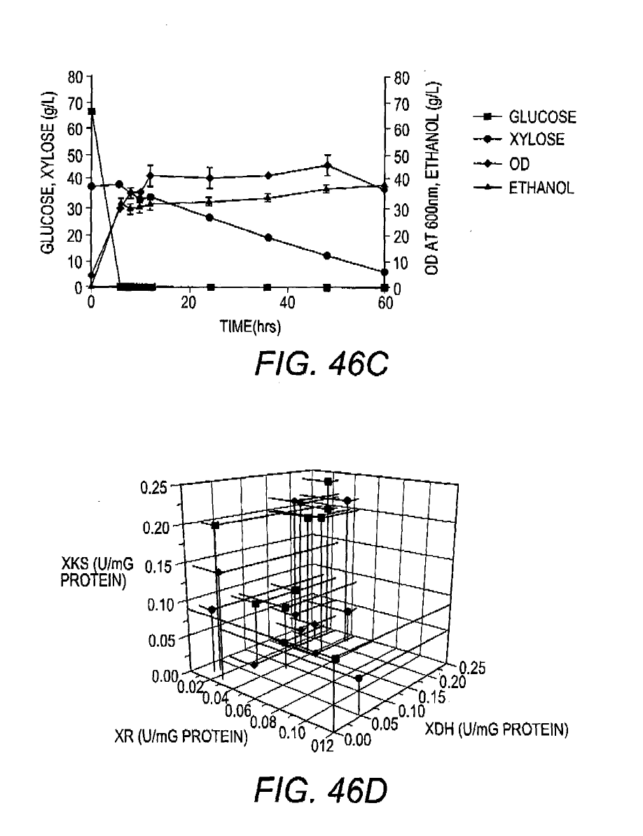

Starting from the same library, pathways optimal for different applications could be found by modifying the screening scheme. In the screening on the media containing sugar mixture (glucose and xylose) instead of xylose as a single carbon source, CL5 was more efficient in total sugar consumption and ethanol yield than CL2 which was superior in xylose fermentation (FIGS. 41B and 45B). In a comparison of the two pathways in xylose only and mixture of glucose and xylose, there was no difference in growth and xylose consumption in YPX media (FIG. 46A). CL2 and CL5 consumed glucose at the same rate producing same biomass in cofermentations. However, CL5 consumed xylose faster than CL2 after complete consumption of glucose (FIG. 46B) consistent with the difference found in the screenings.

Discussion Regarding Examples 1-8

As provided Examples 1-8, a pathway assembly strategy was developed for optimization of xylose utilization in S. cerevisiae. The three step xylose utilization pathway was randomly assembled on a single copy vector using enzyme homologues from various fungal species. The pathway library was assembled on plasmids for the inherent mobility of plasmids, as well as their ease of transformation and handling. A single copy vector was chosen as the backbone for pathway assembly instead of a multicopy vector in order to ensure that every mutant cell within the resultant pathway library would only contains a certain mutant pathway. If a multicopy vector had been used as a backbone in the pathway assembly—as 2 micro origin of replication would allow multiple plasmids to co-exist in a single mutant cell—the pathway responsible for the improved xylose utilization would have been much harder to identify. In this case, the mutant exhibiting faster xylose utilization would quite possibly have resulted from a collection of mutant pathways within the strain, a result which is very hard to analyze and transfer.

For the cloning of enzyme homologues and assembly of the pathway library, a recombination-based DNA assembler approach was used instead of the traditional restriction digestion and ligation-based method. For the cloning of enzyme homologues, application of the DNA assembler method eliminated the need to find restriction sites. Additionally, it should be noted that the strains used in these Examples as sources for cloning of enzyme homologues were not always identical to the strain specified in the database where the gene sequences were obtained due to the availability of strains in culture collections. When necessary, strains of the same species isolated from wood or agricultural waste were ordered as the target organisms for DNA cloning. Consequently, the gene sequences of the enzyme homologues in these particular strains may not be identical to the gene sequences in databases—in fact, DNA sequencing results of the cloned enzyme homologues usually differ from the database. (See the example of the cloned sequence of XKS from Pichia pastoris discussed in Example 9.) In this situation, a restriction digestion-ligation-based approach would fail even when restriction sites were chosen based on the DNA sequence of enzyme homologues found in the database, since the actual sequence of the amplified gene could very likely be different. For the assembly of the pathway library, the DNA assembler method was chosen due to its innate advantages in the rapid assembly of multi-step pathways. As shown in the assembly of a xylose utilization pathway consisting of a small subset of enzyme homologues, the efficiency of correct pathway assembly (˜100%) and the diversity of the resultant library generated from the DNA assembler-based method was satisfactory.

For efficient assembly of the xylose utilization pathway library, the scaffold for the xylose utilization pathway—namely the combination of promoters and terminators for each catalytic step—remained consistent throughout these Examples. A fixed scaffold provides many advantages for subsequent investigation. First of all, all three promoters used in these Examples have been tested in various nutrition and aeration conditions and the expression levels have been proven to be both similar and constitutive (unpublished data; see Example 9). As such, the difference in the expression level and enzyme activity should be mainly dependent upon the properties of the different enzyme homologues. Second, the fixed scaffold ensures that during the random assembly of the pathway, shuffling of different enzyme homologues only occurs within the homologues that correspond to the same catalytic step. In other words, all the resultant variant pathways in the library should have the complete three-gene pathway. Third, due to the fact that the length of yeast promoters and terminators have an average length of around 400 to 1000 bp, the promoter and terminator of the adjacent enzyme provides a fixed DNA sequence of around 1000 bp in length. In later steps, these fixed DNA sequences were included in both of the neighboring gene expression cassettes to generate longer homologous ends, which resulted in higher assembly efficiency for the library creation.

Different backbone vectors were used for the helper plasmid construct and the final assembly intentionally to reduce the amount of work involved in material preparation for pathway assembly. Since gene expression cassettes were amplified from pRS414 helper plasmids, which contain a different selection marker than the backbone vector pRS416 used in the final assembly, it is very unlikely that the trace amount of helper plasmids in the PCR mixture would result in false positive colonies in the assembly. Due to this, the DNA fragments with the correct size could be purified using simple PCR cleanup rather than a gel extraction. This design greatly reduced the amount of labor required for preparation of gene expression cassettes.

DNA fragments of the gene expression cassettes amplified from helper plasmids were then mixed together with linearized pRS416 shuttle vector at an equal DNA amount (in nanograms) for the combinatorial assembly of the pathway library. In this experiment, for two reasons, a lower molar amount of backbone DNA was used than protein-coding DNA. First, like in regular cloning work, more insert DNA was used than backbone DNA in order to ensure a high cloning efficiency. Second, less backbone was also used to avoid cyclization of the backbone by itself, inevitably decreasing the overall likelihood of false positive colonies and thus increasing the overall efficiency of assembly for all three catalytic steps.

After the screening, a heterologous xylose utilization pathway consisting of anXR, caXDH and scXKS was identified from a library. The activity and cofactor preference of the enzyme homologues in the selected pathway on single copy vector was determined later (Example 9). A relatively low activity of XR together with high activity of XDH and XKS may be a good combination of enzyme activities for xylose utilization in INVSc1 strain on single copy centromeric vector. A previous study has shown that a relatively low xylose reductase activity was desired for xylose utilization pathway to reduce the formation of xylitol (Eliasson et al. Enzyme Microbial Tech., 29, 288-297 (2001)). This result of the enzyme-based pathway optimization is consistent with the finding from previous metabolic engineering study for oxidoreductase xylose utilization pathway.

One problem of metabolic engineering for oxidoreductase xylose utilization pathway is the cofactor imbalance issue caused by the different cofactor preference of xylose reductase (XR) and xylitol dehydrogenase (XDH). To address this issue, a large amount of effort has been spent on heterologous expression of new XR and XDH homologous as well as engineering existing enzymes (Zeng et al., Biotech Letters, 31, 1025-1029 (2009); Zhang et al., App Biochem Microbiol, 46, 415-420 (2010); Krahulec et al., Microbial Cell Factories, 9 (2010); Zhang et al., J Microbiology, 47, 351-357 (2009); Kaneda et al., Bioscience Biotech Biochem, 75, 168-170 (2011); Biswas et al., App Microbiol Biotech, 88, 1311-1320 (2010); Krahulec et al., Biotech Journal, 4, 684-694 (2009)). Experiment results of this kind of approach have differed due to the different strain backgrounds and cofactor pairs used in the respective study (Zeng et al., Biotech Letters, 31, 1025-1029, (2009); Watanabe et al., Bioscience Biotech Biochem, 71, 1365-1369 (2007)). Aside from the cofactor imbalance issue, the relative activity of XR, XDH, and XKS is also a problem for efficient xylose assimilation (Eliasson et al. Enzyme Microbial Tech, 29, 288-297 (2001)). Although a lot of effort have been invested to optimize the activity level of the three enzymes, the results of a best balance of the activity of XR, XDH and XKS may also depend on different strain background and pathway construction strategies (Eliasson et al. Enzyme Microbial Tech, 29, 288-297 (2001); Matsushika and Sawayama, J. Bioscience and Bioengineering, 106, 306-309 (2008)). In the experiments of Examples 1-8, a large collection of enzyme homologues for all three genes of the heterologous oxidoreductase xylose utilization pathway in S. cerevisiae were surveyed. All enzyme homologues with different activity and cofactor preference were assayed in the same host strain on same expression vector under a same group of promoters. In contrast to the previous metabolic engineering strategies where a single enzyme was replaced or engineered at a time (Zeng et al., Biotech Letters, 31, 1025-1029 (2009); Zhang et al., App Biochem Microbiol., 46, 415-420 (2010); Krahulec et al., Microbial Cell Factories, 9 (2010); Zhang et al., J Microbiology, 47, 351-357 (2009); Kaneda et al., Bioscience Biotech Biochem, 75, 168-170 (2011); Biswas et al., App Microbiol Biotech, 88, 1311-1320 (2010); Krahulec et al., Biotech Journal, 4, 684-694 (2009)), in the Examples disclosed herein, a library of random assembly of all three catalytic enzymes was examined at one single trial. Since expression of enzyme homologous with the same catalytic activity from various species has been a general metabolic engineering approach for optimization of heterologous pathways, the enzyme-based pathway assembly strategy disclosed herein may be applied for engineering any heterologous pathway in a host cell for production of value-added compounds (Rathnasingh et al., Biotechnol Bioeng., 104, 729-739 (2009); Moon et al., Appl Environ Microbiol, 75, 589-595 (2009); Zhang et al., World J Microbiol Biotech, 22, 945-952 (2006)).

Unlike the traditional pathway optimization strategy, which relies on identifying the limiting step and then engineering a certain enzyme in that metabolic pathway (Zeng et al., Biotech. Letters, 31, 1025-1029 (2009); Zhang et al., App Biochem Microbiol, 46, 415-420 (2010); Krahulec et al., Microbial Cell Factories, 9 (2010); Zhang et al., J Microbiology, 47, 351-357 (2009); Kaneda et al., Bioscience Biotech Biochem., 75, 168-170 (2011); Biswas et al., App Microbiol Biotech, 88, 1311-1320 (2010); Krahulec et al., Biotech Journal, 4, 684-694 (2009)), our the combinatorial pathway assembly method disclosed herein provides a new strategy for pathway optimization. Instead of switching a certain enzyme within the pathway, a collection of enzyme homologues are shuffled and randomly assembled as building blocks for a library of pathways. Using this method, for example, all enzyme homologues that have been shown to improve the xylose utilization may be evaluated under the same scaffold in the same host strains.

Many complicated metabolic pathways can be optimized by applying the strategy presented in Examples 1-8, given a proper screening or selection method. Moreover, this strategy can also enable host strain-specific pathway optimization for tailor-making pathways for special strains with a particular metabolic background.

In the process of optimizing these pathways, a library of pathway assembly with diversified behavior was also generated. Given the well-defined scaffold using fixed promoters and terminators, the diversity of the pathway mutants mainly relies on the choice of different enzyme homologues. In other words, the pathway libraries generated using the strategy described in Examples 1-8 exhibit a controlled diversity. These kinds of libraries are very useful in the understanding of metabolic pathways. Regulation and interaction of metabolic pathways can be studied through approaches such as metabolic flux analysis and DNA microarray. The pathway library consisting of the different pathway enzymes under same group of promoters can also be used to study the effect of the activity and cofactor specificity of a certain enzyme on the overall pathway performance. Models of metabolic pathways can be generated using the data collected by studying mutants from the pathway library to understand and predict the response of the metabolic pathway to different enzyme homologous.

### Example 9

### Further Optimization of Pentose Utilization Pathways Using Promoters of Varying Strengths

The overall metabolic flux in xylose-utilizing S. cerevisiae strains was further optimized using a combinatorial pathway assembly approach employing promoters of varying strengths.

Activities of Enzyme Homologs in the Xylose Utilization Pathway

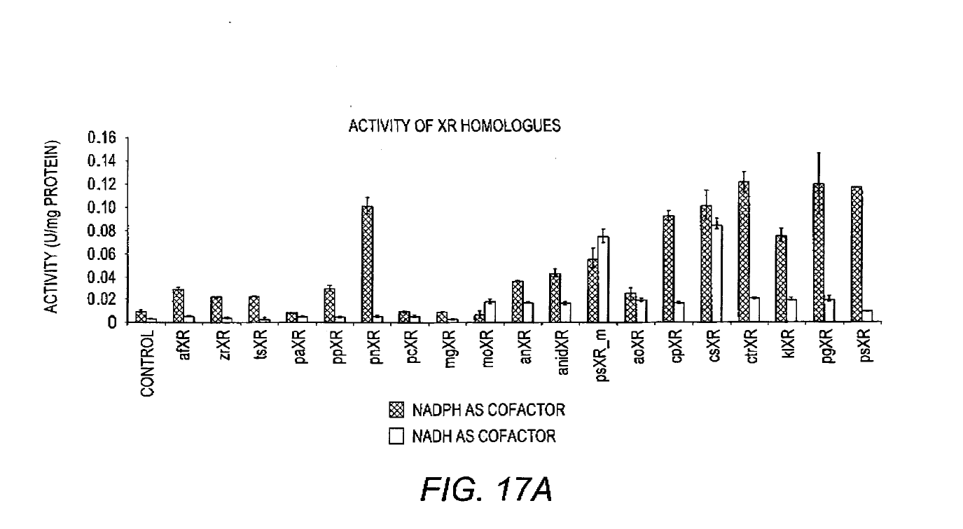

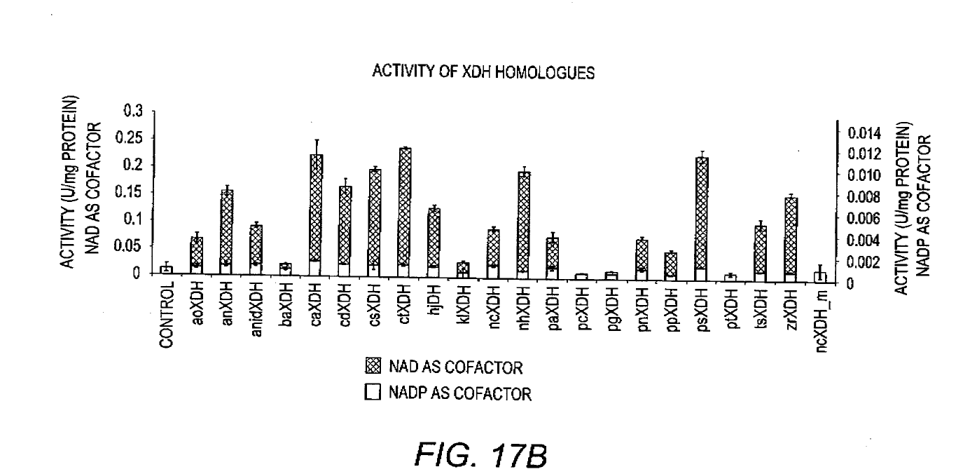

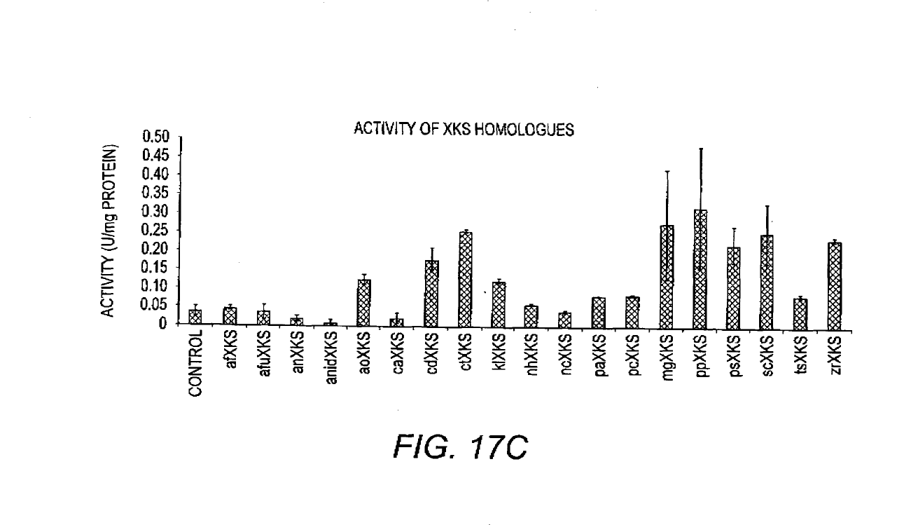

As shown in the previous Examples, twenty homologs of xylose reductase (XR), twenty-two homologs of xylitol dehydrogenase (XDH), and nineteen homologs of xylulokinase (XKS) were cloned for enzyme-based pathway optimization of the xylose utilization pathway. All enzyme homologs were cloned into a pRS414 single copy shuttle vector via DNA assembler. An enzyme activity check was then performed using the aforementioned constructs in order to determine the enzyme to be used for a promoter-based pathway assembly. For xylose reductases, the enzyme activity was determined using either 0.2 mM NADPH or NADH as a cofactor. Similarly, for xylitol dehydrogenases, enzyme activity was also determined using either 1 mM NAD⁺ or NADP⁺ as a cofactor. The activity of xylulokinases was measured using a Glycerol Kit (R-Biopharm, Darmstadt, Germany). Enzyme activities of all cloned enzyme homologous are shown in FIG. 17. Most of the xylose reductases disclosed herein have activity with NADPH as a cofactor. Only psXR from Scheffersomyces stipitis engineered for altered cofactor specificity (Watanabe et al., Bioscience Biotech Biochem, 71, 1365-1369 (2007)) and csXR from Candida shehatae have activity with NADH as a cofactor. Therefore, csXR, which exhibited a higher activity when compared to the psXR K270R mutant with both NADPH and NADH as a cofactor, was chosen to be the xylose reductase in later constructs. Similarly, ctXDH from Candida tropicalis, which displayed the highest activity using NAD⁺ as a cofactor, and ppXKS from Pichia pastoris were also chosen as the enzyme in all later constructs.

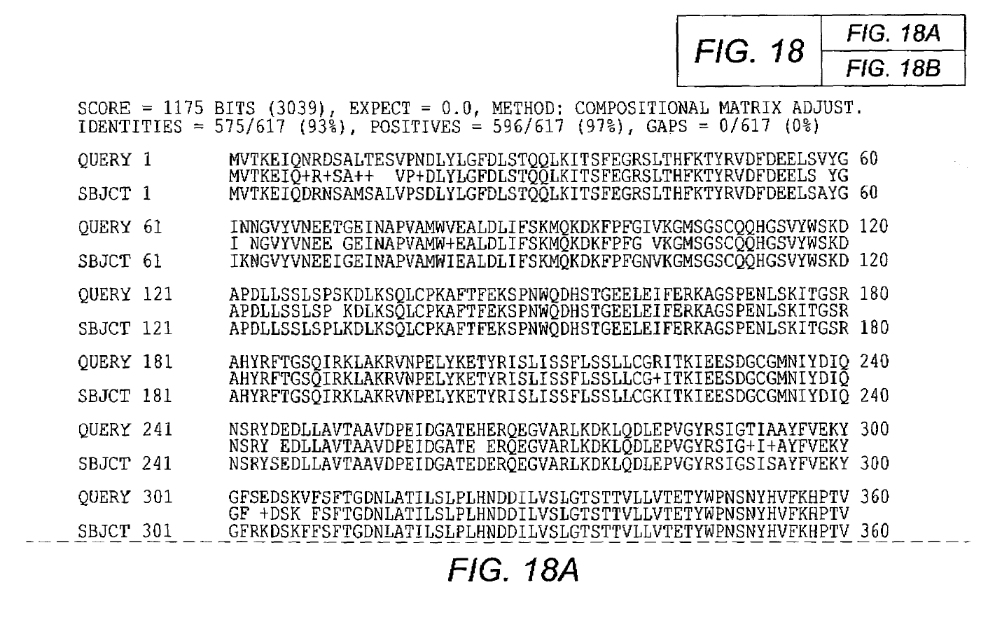

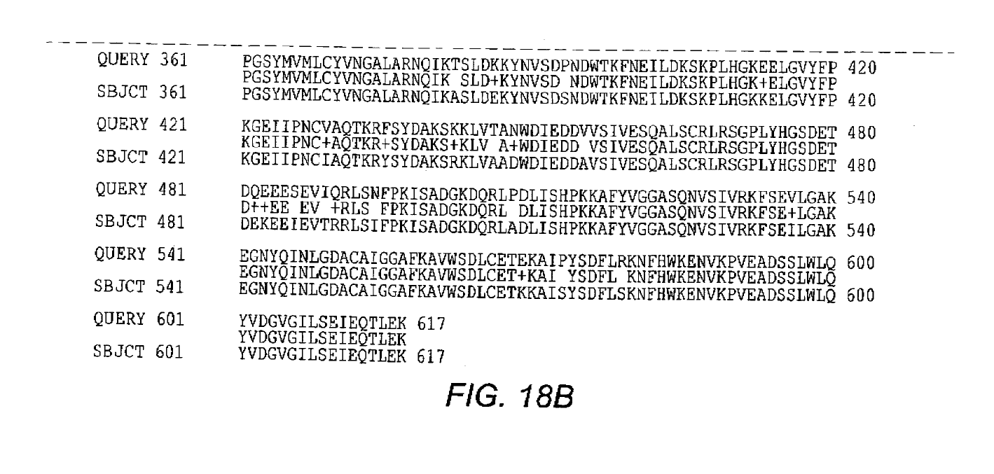

To facilitate high throughput cloning of enzyme homologs, a homologous recombination-based method was used for the construction of plasmids that contained the gene expression cassettes which were used for generation of DNA fragments for enzyme-based pathway optimization (described in Examples 1-8). The same plasmids were also used for examination of enzyme activities of these cloned homologs. As one of the advantages of the homologous recombination-based DNA assembler method, complete knowledge of the sequences of target gene was not necessary for the cloning work. To speed up the cloning of more than sixty enzyme homologs, the resultant constructs were simply checked by diagnostic PCR rather than DNA sequencing. However, since csXR, ctXDH, and ppXKS were used as enzymes for all the constructs of the promoter-based assembly, they were submitted for DNA sequencing prior to the construction of the pathway libraries. As a result, it was found that the cloned ctXDH displayed the same sequence as the cDNA sequence in the NCBI database. The cloned csXR has one missense mutation (G28D) when compared with the reference sequence available online. Surprisingly, the cloned ppXKS shows mutations scattered across its gene sequence when compared to the reference sequence. When the cloned gene sequence of ppXKS was translated into an amino acid sequence, the resulting protein is of the same length (i.e., non-truncated). The amino acid sequence of the cloned ppXKS was aligned with its reference sequence from NCBI to compare the sequence similarities (FIG. 18).

The cloned ppXKS only shares 93% sequence identity with its reference protein. To further verify that the origin of the cloned ppXKS is actually from cDNA isolated from Pichia pastoris and not due to contamination, the amino acid sequence of the cloned ppXKS was subjected to a BLAST search of the non-redundant protein sequence database at NCBI. The result from the BLAST search showed that the top hit with the highest score is indeed the xylulokinase from Pichia pastoris, indicating that the ppXKS we cloned is from Pichia pastoris cDNA and not contamination.

Creation of Promoter Mutants with Varying Strength

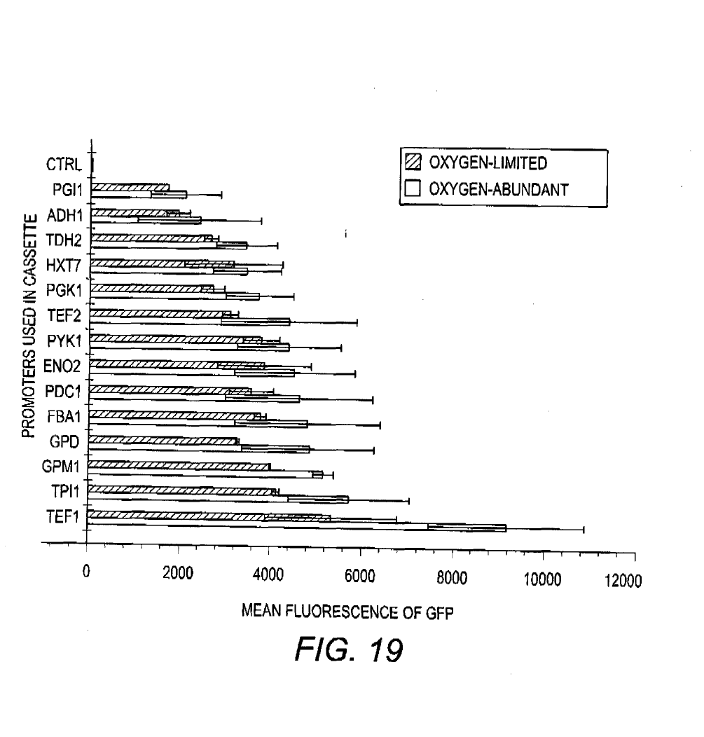

To create a library of promoters with varying strengths for the optimization of pathways, first, a group of yeast promoters were characterized under different growth conditions (FIG. 19). Promoter TEF1 (SEQ ID NO:112 of Example 2), ENO2 (SEQ ID NO:118), PDC1 (SEQ ID NO:119), TPI1 (SEQ ID NO:122), FBA1 (SEQ ID NO:120), and GPM1 (SEQ ID NO:121) were subjected to nucleotide analogue mutagenesis in the presence of 20 μM 8-oxo-2′-deoxyguanosine (8-oxodGTP) and 6-(2-deoxy-β-D-ribofuranosyl)-3,4-dihydro-8-pyrimido-[4,5-c][1,2]oxazin-7-one (dPTP), according to published methods (Alper et al., Proc Natl Acad Sci USA, 102:12678-12683, 2005, U.S. Publn. No. US 2007/0178505 of Fischer et al., and Nevoigt et al., App. Environ. Micro. 72, 5266-5273, 2006).

The nucleic acid sequence of the ENO2 promoter is set forth as SEQ ID NO: 118:
gtgtcgacgctgcgggtatagaaagggttctttactctatagtacctcctcgctcagcatctgcttcttcccaaagatgaacgcggcgttatgtc
actaacgacgtgcaccaacttgcggaaagtggaatcccgttccaaaactggcatccactaattgatacatctacacaccgcacgccttttttct
gaagcccactttcgtggactttgccatatgcaaaattcatgaagtgtgataccaagtcagcatacacctcactagggtagtttctttggttgtatt
gatcatttggttcatcgtggttcattaattttttttctccattgctttctggctttgatcttactatcatttggatttttgtcgaaggttgtagaat
tgtatgtgacaagtggcaccaagcatatataaaaaaaaaaagcattatcttcctaccagagttgattgttaaaaacgtatttatagcaaacgcaattg
taattaattcttattttgtatcttttcttcccttgtctcaatcttttatttttattttatttttcttttcttagtttctttcataacaccaagcaact
aatactataacatacaataata.
The nucleic acid sequence of the PDC1 promoter is set forth as SEQ ID NO: 119:
catgcgactgggtgagcatatgttccgctgatgtgatgtgcaagataaacaagcaaggcagaaactaacttcttcttcatgtaataaacacac
cccgcgtttatttacctatctctaaacttcaacaccttatatcataactaatatttcttgagataagcacactgcacccataccttccttaaaaacgt
agcttccagtttttggtggttccggcttccttcccgattccgcccgctaaacgcatatttttgttgcctggtggcatttgcaaaatgcataacctat
gcatttaaaagattatgtatgctcttctgacttttcgtgtgatgaggctcgtggaaaaaatgaataatttatgaatttgagaacaattttgtgttgtt
acggtattttactatggaataatcaatcaattgaggattttatgcaaatatcgtttgaatatttttccgaccctttgagtacttttcttcataattgc
ataatattgtccgctgcccctttttctgttagacggtgtcttgatctacttgctatcgttcaacaccaccttattttctaactattttttttttagct
catttgaatcagcttatggtgatggcacatttttgcataaacctagctgtcctcgttgaacataggaaaaaaaaatatataaacaaggctctttcact
ctccttgcaatcagatttgggtttgttccctttattttcatatttcttgtcatattcctttctcaattattattttctactcataacctcacgcaaaa
taacacagtcaaatcaatcaaa.
The nucleic acid sequence of the FBA1 promoter is set forth as SEQ ID NO: 120:
tccaactggcaccgctggcttgaacaacaataccagccttccaacttctgtaaataacggcggtacgccagtgccaccagtaccgttaccttt
cggtatacctcctttccccatgtttccaatgcccttcatgcctccaacggctactatcacaaatcctcatcaagctgacgcaagccctaagaaat
gaataacaatactgacagtactaaataattgcctacttggcttcacatacgttgcatacgtcgatatagataataatgataatgacagcaggatt
atcgtaatacgtaatagttgaaaatctcaaaaatgtgtgggtcattacgtaaataatgataggaatgggattcttctatttttcctttttccattcta
gcagccgtcgggaaaacgtggcatcctctctttcgggctcaattggagtcacgctgccgtgagcatcctctctttccatatctaacaactgagca
cgtaaccaatggaaaagcatgagcttagcgttgctccaaaaaagtattggatggttaataccatttgtctgttctcttctgactttgactcctcaaa
aaaaaaaaatctacaatcaacagatcgcttcaattacgccctcacaaaaacttttttccttcttcttcgcccacgttaaattttatccctcatgttgt
ctaacggatttctgcacttgatttattataaaaagacaaagacataatacttctctatcaatttcagttattgttcttccttgcgttattcttctgtt
cttctttttcttttgtcatatataaccataaccaagtaatacatattcaaa.
The nucleic acid sequence of the GPM1 promoter is set forth as SEQ ID NO: 121:
tagtcgtgcaatgtatgactttaagatttgtgagcaggaagaaaagggagaatcttctaacgataaacccttgaaaaactgggtagactacgc
tatgttgagttgctacgcaggctgcacaattacacgagaatgctcccgcctaggatttaaggctaagggacgtgcaatgcagacgacagatc
taaatgaccgtgtcggtgaagtgttcgccaaacttttcggttaacacatgcagtgatgcacgcgcgatggtgctaagttacatatatatatatat
atatatatatatatatatatagccatagtgatgtctaagtaacctttatggtatatttcttaatgtggaaagatactagcgcgcgcacccacacac
aagcttcgtcttttcttgaagaaaagaggaagctcgctaaatgggattccactttccgttccctgccagctgatggaaaaaggttagtggaacga
tgaagaataaaaagagagatccactgaggtgaaatttcagctgacagcgagtttcatgatcgtgatgaacaatggtaacgagttgtggctgtt
gccagggagggtggttctcaacttttaatgtatggccaaatcgctacttgggtttgttatataacaaagaagaaataatgaactgattctcttcct
ccttcttgtcctttcttaattctgttgtaattaccttcctttgtaattttttttgtaattattcttcttaataatccaaacaaacacacatattaca
ata.
The nucleic acid sequence of the TPI1 promoter is set forth as SEQ ID NO: 122:
tatatctaggaacccatcaggttggtggaagattacccgttctaagacttttcagcttcctctattgatgttacacctggacaccccttttctggca
tccagtttttaatcttcagtggcatgtgagattctccgaaattaattaaagcaatcacacaattctctcggataccacctcggttgaaactgacag
gtggtttgttacgcatgctaatgcaaaggagcctatatacctttggctcggctgctgtaacagggaatataaagggcagcataatttaggagttt
agtgaacttgcaacatttactattttcccttcttacgtaaatatttttctttttaattctaaatcaatctttttcaattttttgtttgtattctttt
cttgcttaaatctataactacaaaaaacacatacataaactaaaa.
The nucleic acid sequence of the GPM terminator is set forth as SEQ ID NO: 123:
gtctgaagaatgaatgatttgatgatttctttttccctccatttttcttactgaatatatcaatgatatagacttgtatagtttattatttcaaatt
aagtagctatatatagtcaagataacgtttgtttgacacgattacattattcgtcgacatcttttttcagcctgtcgtggtagcaatttgaggagta
ttattaattgaataggttcattttgcgctcgcataaacagttttcgtcagggacagtatgttggaatgagtggtaattaatggtgacatgacatgtt
atagcaataaccttgatgtttacatcgtagtttaatgtacaccccgcgaattcgttcaagtaggagtgcaccaattgcaaagggaaaagctgaatgg
gcagttcgaata.

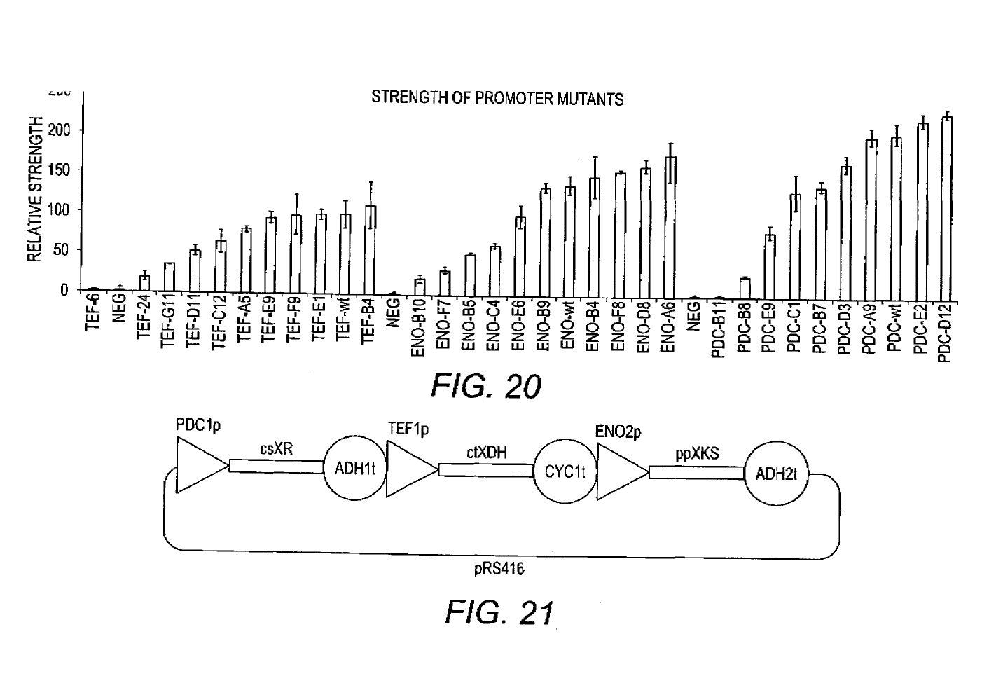

To facilitate the cloning and characterization of promoter mutants, a helper plasmid was constructed for the promoter engineering work. The PCR products of target promoters were cloned into this helper plasmid linearized with XhoI using the DNA assembler method (8). The strength of the promoter mutants was determined by measuring the fluorescent intensity of the GFP driven by promoter mutants using flow cytometry. Two strategies were used to isolate promoter mutants with varying strength. In the first strategy, colonies were randomly picked and inoculated into 96-well and fluorescent intensity was measured using a plate reader. The mutants were then divided into ten groups representing different promoter strength (i.e. 0˜10% of the wild type promoter, 10˜20% of the wild type promoter, and so on) according to the fluorescent intensity. Several mutants from each group were then cultivated in round bottom culture tubes and the fluorescent intensity was determined using flow cytometry. This strategy worked very successfully in finding mutants with moderate promoter strength. In order to find promoters with very low strength, such as those with strength lower than 20% of wild type promoters, or mutants with strength higher than that of the wild type, a mixed culture of promoter mutants was first sorted by Fluorescence-Activated Cell Sorting (FACS) to isolate mutants with very high or low fluorescent intensity. The cell culture obtained from cell sorting was then spread on SC-Leu plates supplemented with glucose. Colonies randomly picked from the plates were inoculated into liquid media and their fluorescent intensity was determined by flow cytometry. As expected, there is a higher possibility to obtain mutants with either very high or very low strength after the library was sorted. For the optimization of the xylose utilization pathway, three promoters mutant groups generated from wild type yeast promoters TEF1p, ENO2p, and PDC1p were created. The strength of the promoter mutants were then measured using the fluorescent intensity of GFP driven by promoter mutants. As shown in FIG. 20, around ten mutants with varying strength were isolated from each promoter.

Construction of Gene Expression Cassettes with Promoter Mutants

In order to investigate the efficiency of the pentose utilization pathways consisting of the same catalytic enzymes but with different expression profiles, a general scaffold for the three-gene xylose utilization pathway was designed.

This scaffold consists of csXR, ctXDH, and ppXKS. Specifically, csXR ORF was flanked with a PDC1 promoter and an ADH1 terminator, followed by ctXDH with a TEF1 promoter and a CYC1 terminator, and ppXKS with an ENO2 promoter and an ADH2 terminator. Similar to the scaffold in the enzyme-based pathway optimization design described in Examples 1-8, the scaffold for the pentose utilization pathways, namely the combination of enzymes and terminators for each catalytic step, remained consistent throughout this study (FIG. 21).

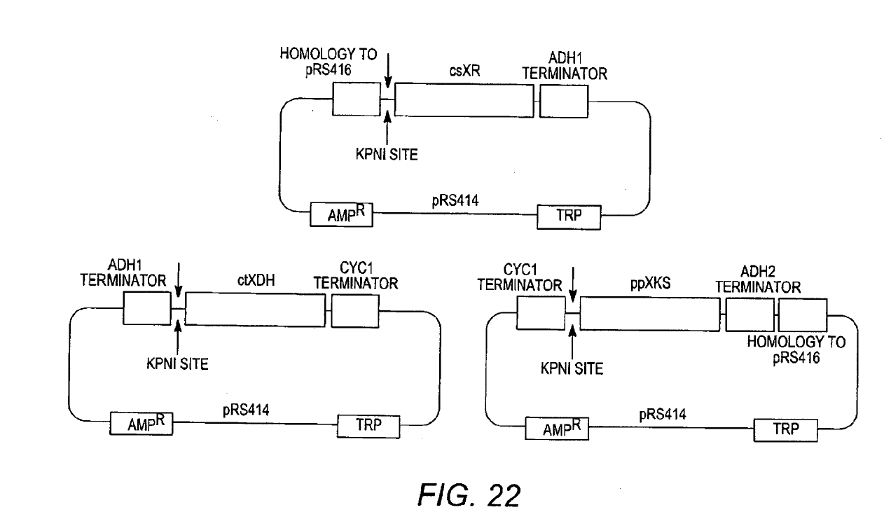

To facilitate the cloning of promoter mutants for pathway assembly, helper plasmids were constructed for each pathway gene in the xylose utilization pathway. In each helper plasmid, a DNA fragment (˜400 bp) homologous to the upstream adjacent sequence (usually the terminator of the previous pathway gene), a pathway enzyme, and a terminator were assembled into a pRS414 single copy plasmid using DNA assembler. A unique KpnI site was engineered between the DNA fragment homologous to the previous pathway gene and the target pathway gene to facilitate the linearization of the helper plasmids for the cloning of promoter mutants in the assembly of gene expression cassettes (FIG. 22). To clone promoter mutants into the helper plasmids for the construction of plasmids with full gene expression cassettes, the promoter mutants were amplified from pRS415-promoter mutant-GFP constructs and then transferred into the helper plasmids linearized with KpnI. The transformants were plated on SC-Trp solid media supplemented with 2% glucose. Single colonies were inoculated into SC-Trp liquid media supplemented with glucose. Yeast plasmids were isolated from the liquid culture and transferred into E. coli DH5α. Next, E. coli plasmids were isolated and diagnostic PCR was performed to confirm the cloning of promoter mutants using primers that anneal to regions both upstream and downstream of the promoter mutants.

To obtain the gene expression cassettes for random pathway assembly, PCR was used to amplify the whole gene expression cassette including the homologous region upstream to the promoter, the promoter itself, the target ORF, and the terminator. The sizes of the resultant fragments were confirmed using agarose gel electrophoresis, and then DNA fragments with the correct size were purified using a PCR purification kit. The concentrations of purified DNA fragments were determined using Nanodrop (Thermo Scientific, Wilmington, Del.). Similar to what was previously described in Examples 1-8, the vectors used in the creation of promoter mutants (pRS415), gene expression cassettes (pRS414), and the final pathway assembly (pRS416) were all different. Since different nutrition markers were used in these vectors, only a simple PCR cleanup was performed between different steps.

Assembly of Libraries Containing the Xylose Utilization Pathways Using DNA Assembler

To create a library of yeast strains containing the three-gene xylose utilization pathway, DNA fragments homologous to the adjacent sequences were mixed and transferred into S. cerevisiae with the linearized pRS416 shuttle plasmid for the INVSc1 strain. After DNA transformation, a small amount of transformants were spread on a SC-Ura plate supplemented with glucose to determine the library size. The rest of the transformants were first cultivated overnight in liquid SC-Ura medium supplemented with glucose and then washed and spread on a SC-Ura plate supplemented with 2% xylose for screening. When 100 ng of each fragment was used for the promoter-based pathway library assembly in the INVSc1 strain, a library of 10⁴ to 10⁵ transformants per transformation could be obtained.

Eight colonies were randomly picked from the promoter-based pathway library in the INVSc1 strain in order to determine the diversity among the resultant pathway mutants. These single colonies were inoculated first in SC-Ura medium supplemented with 2% glucose and then cultivated at 30° C. with 250 rpm agitation for 2 days. The cultures were then used to inoculate 125 mL un-baffled flasks containing 25 mL of YP medium supplemented with 2% xylose to an initial OD of 0.2. The flask cultures were grown at 30° C. and 100 rpm agitation (in oxygen limited conditions). Samples were drawn from the cultures at various time points for the measurement of cell density and the concentration of xylose and ethanol.

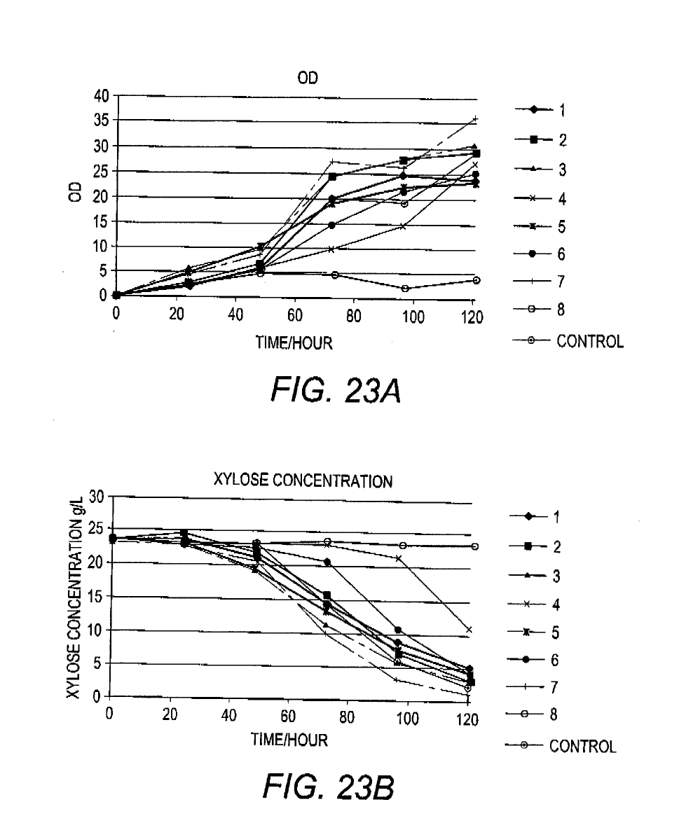

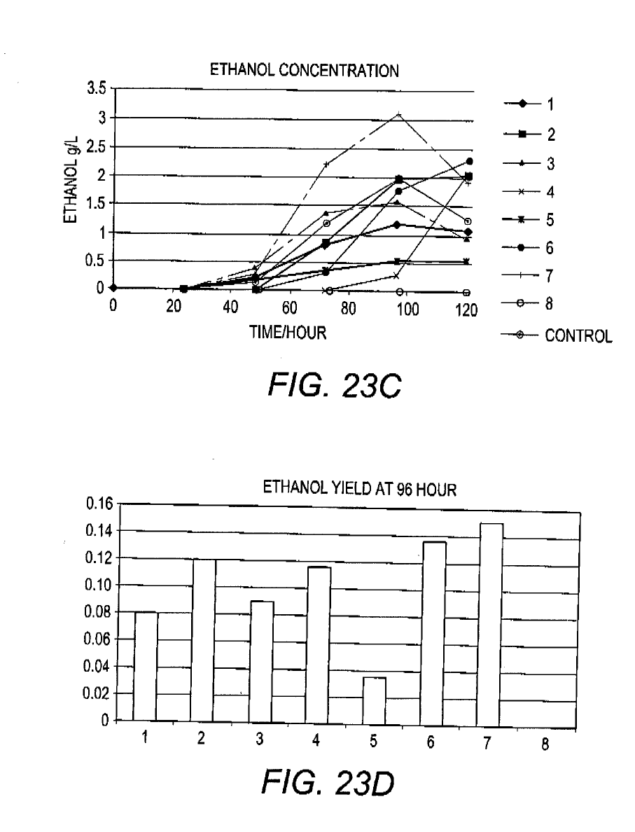

As shown in FIG. 23, when xylose was used as the sole carbon source, randomly picked mutants in the pathway library exhibited different fermentation performance in terms of overall growth rate, xylose consumption, and ethanol production, which indicates a high degree of diversity within the promoter-based pathway library.

The promoter-based pathway optimization was also performed using industrial S. cerevisiae strains as host strains. In this study, Still Spirits (Classic) Turbo Distiller's Yeast (which will be referred to simply as the “Classic” strain from here on) and S. cerevisiae ATCC 4124 were used as two model industrial yeast strains. Due to the lack of auxotrophic markers in industrial strains, a new single copy centromeric vector—namely pRS-KanMX, which carries the dominant selection marker of KanMX—was constructed to enable pathway engineering in industrial strains. The pRS-KanMX vector bears the same homologous region as the pRS416 vector used in previous assembly. Therefore, the same DNA fragments used for pathway assembly in the INVSc1 strain can be directly used for pathway assembly using pRS-KanMX as the backbone. After DNA transformation, the transformants need to be recovered in YPAD liquid medium overnight to increase their transformation efficiency. After recovery, a small amount of transformants were spread on a YPAD plate supplemented with glucose and 200 mg/L G418 in order to determine the library size. The remaining transformants were first cultivated overnight in liquid SC complete medium supplemented with glucose and 200 mg/L G418 and then washed and spread on a SC complete plate supplemented with 2% xylose for screening. The transformation efficiency of pathway assembly using industrial strains and the pRS-KanMX vector was lower than that of the assembly in the INVSc1 strain. Using 100 ng of each DNA fragment, a library size of 10³ to 10⁴ was achieved for industrial yeast strains.

Screening of Libraries of Pathways with Promoter Mutants

Similar to the screening of the enzyme-based pathway library in Examples 1-8, the promoter-based pathway library was also screened using a size-based colony prescreening followed by tube screening and flask screening. Using the INVSc1 strain as the host, the pathway was assembled using DNA fragments amplified from helper plasmids. A small aliquot of the transformants were plated onto SC-Ura plates supplemented with 2% glucose in order to determine the library size. The rest of the transformants were used to inoculate a 25 mL liquid media of SC-Ura supplemented with 2% glucose. Frozen cell stocks were made from the liquid culture for later analysis. A small aliquot of the liquid culture was then washed with ddH₂O and around 10⁵ cells were plated onto a 24.5 cm by 24.5 cm square agar plate of SC-Ura supplemented with 2% xylose. At the same time, around 10⁴ cells harboring a reference pathway consisting of csXR driven by a wild type PDC1 promoter, ctXDH driven by a wild type TEF1 promoter, and ppXKS driven by a wild type ENO2 promoter were plated on a regular 15 cm round agar plate with the same media. The library plate and the reference plate were then incubated together and examined daily. Colonies on the library plate that had grown to a size bigger than that of the largest colonies on the reference plate were then picked for later screening.

For library screening, eighty colonies that appeared larger than the biggest colonies on the reference plate were inoculated into a culture tube containing 1 mL of SC-Ura liquid media supplemented with 2% glucose and then grown at 30° C. with 250 rpm shaking for 36 hours. Next, 200 μL of culture was then spun down and resuspended in 200 μL of YP media supplemented with 2% xylose. Next, 120 μL of cell suspension was used to inoculate 3 mL of YP culture with 2% xylose in a culture tube. This step ensured that the tube cultures would have a starting OD of around 0.2. The tube cultures were then grown at 30° C. with 250 rpm agitation. The OD₆₀₀ of the tube culture was then taken after 24, 36, and 48 hours. The cell density at the first two time points were used to determine the specific growth rate while the 48 hour time point was taken to show the final biomass productivity of the strain.

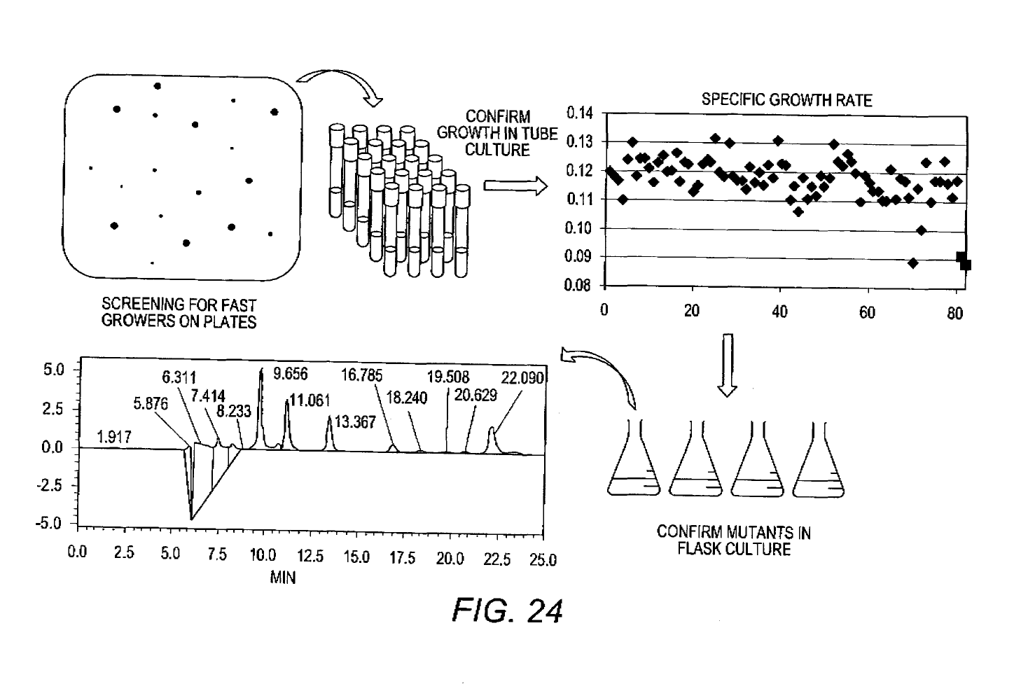

After the tube screening, the top ten strains that displayed a high growth rate were picked for later analysis. These fast growers were inoculated into 50 mL un-baffled flasks containing 10 mL of YP media supplemented with 2% xylose. The flask cultures were then grown at 30° C. with 100 rpm agitation to determine the xylose consumption and ethanol production of the mutant strains (FIG. 24).

For pathway assembly in the ATCC 4124 stain, eighty large colonies were inoculated into SC complete media supplemented with 2% glucose and 200 mg/L G418. The seed tubes were grown for 36 hours at 30° C. under 250 rpm of agitation. Next, 200 μL of seed culture was spun down and resuspended in YP medium supplemented with 2% glucose and 120 μL of the cell suspension was used to inoculate 3 mL YP media supplemented with 2% xylose in round bottom culture tubes. The same procedure was applied for the tube and flask screening of the industrial strain as that of the INVSc1 strain. After screening of the promoter-based pathway library, we noticed most of the big colonies picked from agar plate grew better when compared to the strains harboring the control pathways. As a consequence, a smaller number of colonies (only fifty colonies) were picked for the pathway screening of the Classic strains to reduce the amount of required labor.

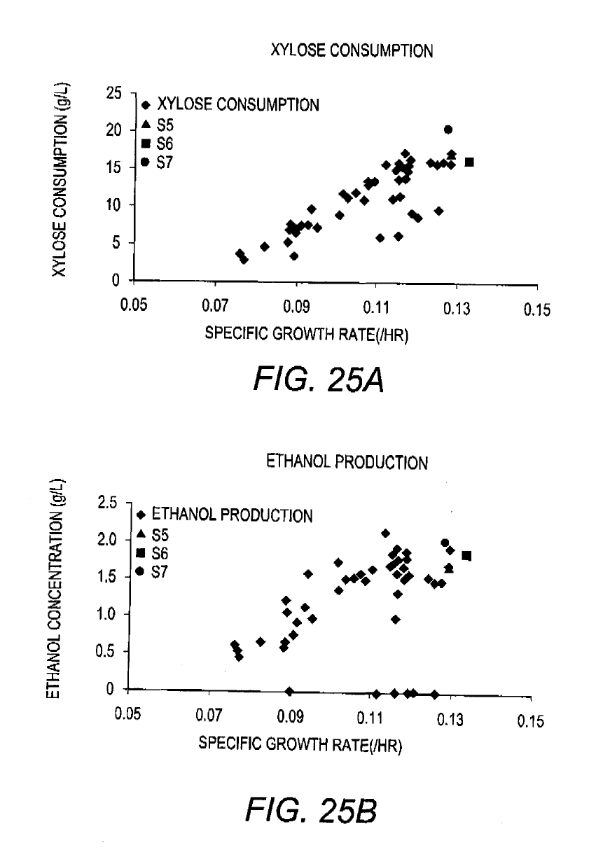

To further validate the screening strategy, 36-hour samples of the fifty tube cultures were analyzed using HPLC. It was found that the xylose consumption and ethanol production of mutant strains correlated well with cell growth rates. This indicated that the screening method is not only an effective strategy for finding faster growers, but also a valid method for finding fast xylose consumers and ethanol producers. After the shake flask-based screening of the top ten faster growers was completed, the top three ethanol producers were identified using shake flask cultures under the oxygen limited condition. The tube cultures of the top three ethanol producers were highlighted in dark black in FIG. 25. The results showed that the top three mutants from the shake flask based screening with oxygen limited conditions were also among the highest ethanol producers in the tube based screening using aerobic conditions. This result further validated the tube based prescreening step, as the growth in an aerobic tube culture is a good indicator of the xylose consumption and ethanol production ability of the mutant strains (FIG. 25).

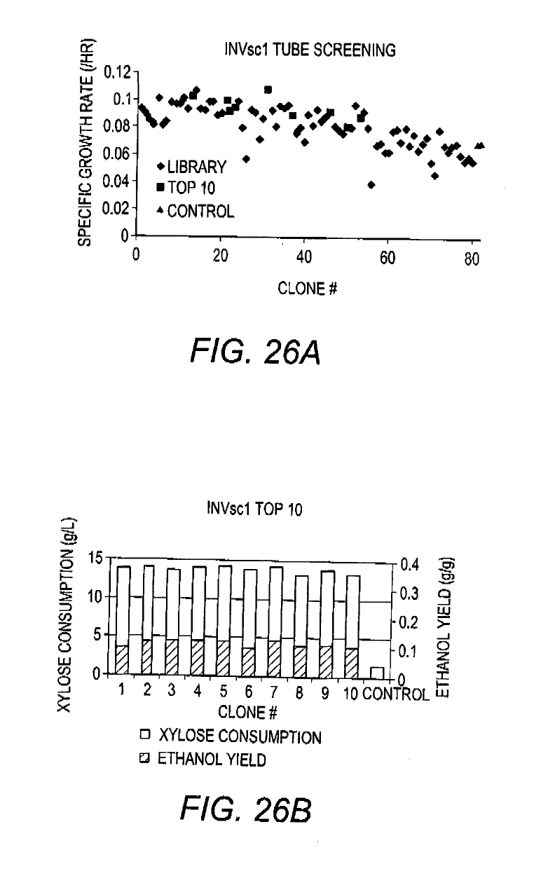

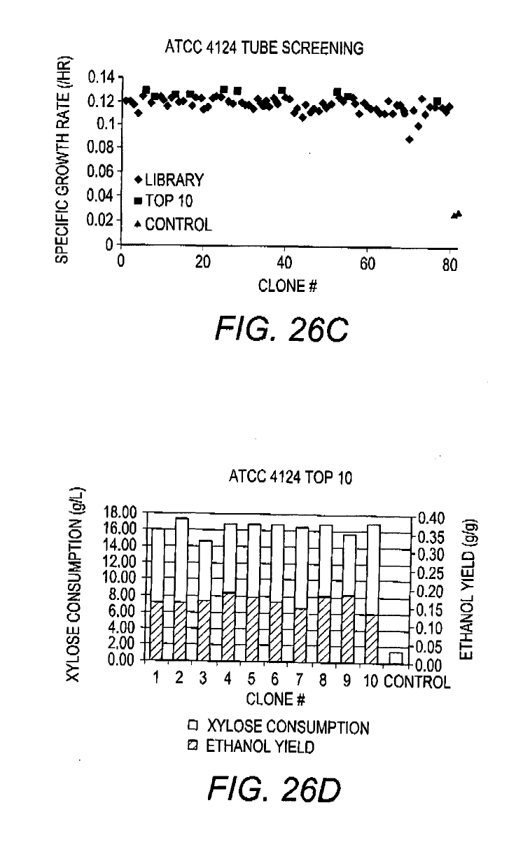

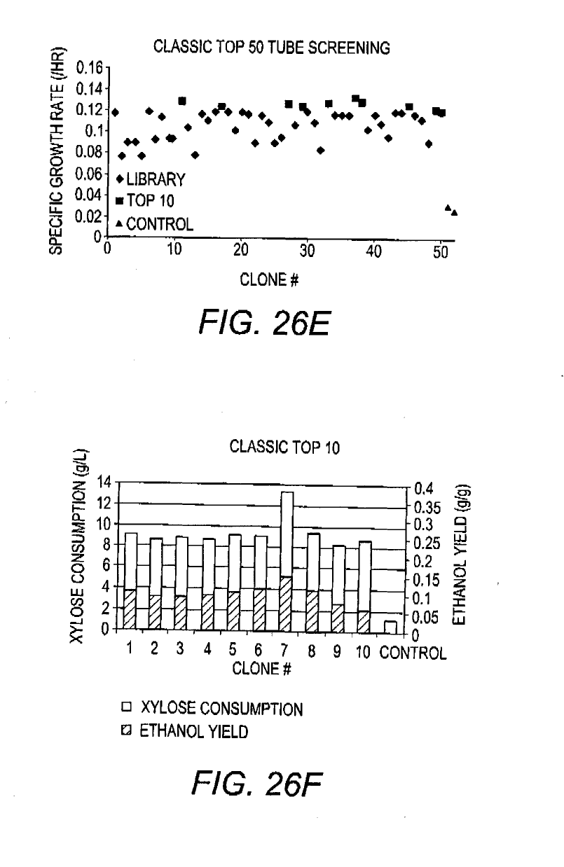

Using the screening strategy described above, eighty fast growers were screened from the promoter-based pathway assembly in the INVSc1 strain and in ATCC 4124, while fifty faster growers were screened again in the Classic turbo yeast. Specific growth rates of the tube cultures are shown in FIG. 26.

Characterization of Screened Mutant Strains

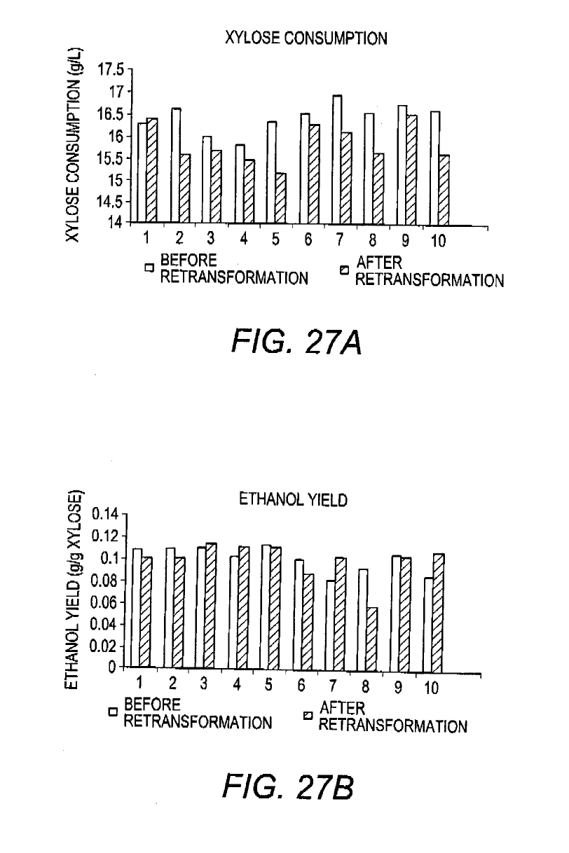

In the whole process of pathway screening, no prolonged incubation of longer than three days was used, which should limit the possibility of host strain adaptation. In order to further confirm that the improvement of xylose fermentation was indeed due to the pathway on the plasmids rather than host strain adaptation, plasmids of the top ten fastest growers from the INVSc1 library were isolated and retransferred back into fresh INVSc1 strains. The top ten fastest growers before and after retransformation were inoculated into 50 mL shake flasks containing 10 mL of YP media supplemented with 2% xylose to an initial OD of 0.2. The xylose consumption and ethanol production abilities of the strains before and after retransformation were compared. As shown in FIG. 27, the fermentation ability of the strains hosting the same pathways before and after retransformation were very similar, indicating that a minimum extent of host adaptation occurred during the screening process. In other words, the better xylose fermentation ability of the screened strains was from the plasmids bearing better xylose utilization pathways.

After the tube and flask based library screening, the top three fastest growers were selected for further analysis. From the promoter-based pathway library screened in the INVSc1 strain, the top three fastest growing strains were S3, S5 and S7. From the promoter-based pathway library screened in the ATCC 4124 strain, the top three fastest growing strains were S4, S8, and S9. In addition, the top three fastest growing strains screened in the Classic strain were S5, S6, and S7 (FIG. 26).

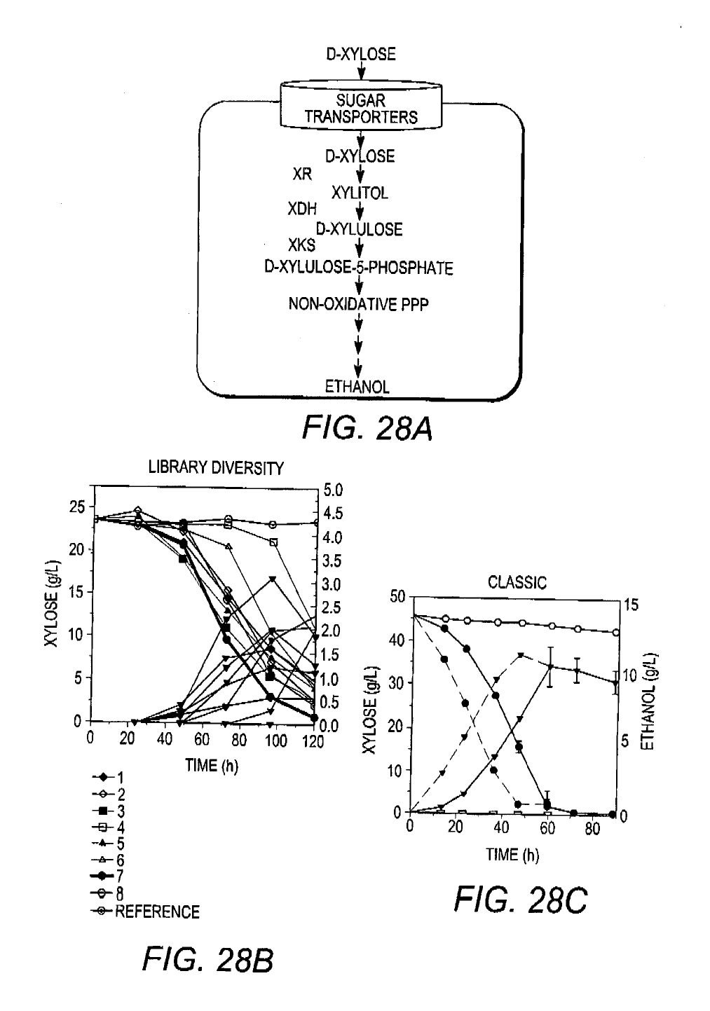

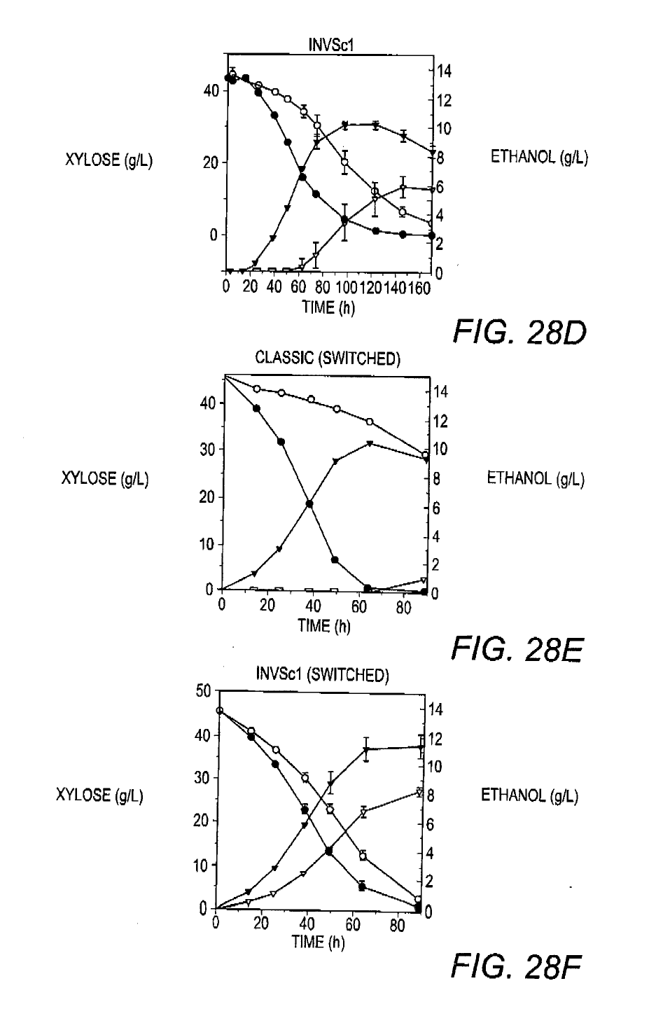

As shown in FIGS. 28C and 28D, for the xylose utilization pathway optimized in the laboratory strain INVSc1, the optimized mutant strain INV-X3 (INVSc1 S3) consumed xylose at 0.4 g/L/h and produced ethanol at 0.1 g/L/h, which was 1.7 times of the rate of the reference strain containing the same set of metabolic genes under the wild type promoters and improved the ethanol yield by more than 60% (0.25 g/g xylose for the optimized strains versus 0.16 g/g xylose for the reference strain) (Table 9-1). More impressively, after only one round of pathway optimization in the industrial strain named Classic Turbo Yeast, the CTY-X7 strain with an optimized pathway exhibited a xylose consumption rate of 0.92 g/L/h with an ethanol yield of 0.26 g/g xylose, which is close to the fastest xylose utilizing strain reported in literature (Ha et al. Proc Natl Acad Sci USA, 108, 504-509 (2011)) (Table 9-2). In contrast, the strain hosting the reference pathway with the same set of metabolic genes under the wild type promoters only consumed less than 9% of the total xylose and produced no ethanol in 88 hours. The top three optimized xylose utilization pathways from both the laboratory and industrial strains were isolated and the strengths of promoter mutants presented in the optimized constructs were determined using the green fluorescent protein as a reporter. The top three mutant promoters isolated from the laboratory strain (INVSc1) all exhibited around 50% of strength compared to the wild type TEF promoter for XR, while mutants isolated from the industrial strain (Classic Turbo Yeast) all exhibited around 130% relative strength for XR. The strength of promoter mutants for the XDH and XKS did not converge as well as the ones for XR, indicating that there might be multiple solutions to the optimized expression patterns for xylose utilization (Table 9-3).

TABLE 9-1
Xylose fermentation performance of optimized and reference strains. The
reference strains are csXR, ctXDH and ppXKS driven by wild type PDC1, TEF1 and ENO2
promoters in corresponding strains.

| | Laboratory | | | | | | |
|---|---|---|---|---|---|---|---|
| | strain | | | | | | |
| | (INVSc1) | | Industrial strain (Classic) | | | | |
| | INV-WT | INV-X3 | CTY-WT | CTY-WT | CTY-X7 | CTY-X7 | CTY-X7 |
| Seed culture | SCD | SCD | YPD | YPX | YPD | YPX | YPX |
| Initial OD | 1 | 1 | 10 | 2 | 10 | 2 | 10 |
| Xylose rate | 0.24 | 0.40 | 0.06 | 0.03 | 0.74 | 0.73 | 0.92 |
| (g xylose/l/hr) | | | | | | | |
| Ethanol | 0.04 | 0.10 | 0 | 0 | 0.17 | 0.17 | 0.24 |
| productivity | | | | | | | |
| (g ethanol/l/hr) | | | | | | | |
| Yield | 0.15 | 0.25 | 0 | 0 | 0.24 | 0.23 | 0.26 |
| (g ethanol/g xylose) | | | | | | | |

TABLE 9-2
Comparison of fermentation performance of optimized xylose utilizing
strains from this Example with top xylose fermenting strains in literature.

| Strain name | INV-X3 | CTY-X7 | DA24-16^{a} | MA-R4^{b} | MA-R^{b} |
|---|---|---|---|---|---|
| (Host strain) | (INVSc1) | (Classic) | (D452-2) | (IR-2) | (IR-2) |
| Xylose rate | 0.40 | 0.92 | 1.33 | 1.07 | 1.29 |
| (g xylose/l/hr) | | | | | |
| Ethanol | 0.10 | 0.24 | 0.65 | 0.36 | 0.50 |
| productivity | | | | | |
| (g ethanol/l/hr) | | | | | |
| Yield | 0.25 | 0.26 | 0.31~0.33 | 0.34 | 0.37 |
| (g ethanol/g | | | | | |
| xylose) | | | | | |

^{a}Ha, S. J., Galazka, J. M., Rin Kim, S., Choi, J. H., Yang, X., Seo, J. H., Louise Glass, N., Cate, J. H. and Jin, Y. S. Engineered Saccharomyces cerevisiae capable of simultaneous cellobiose and xylose fermentation. Proc Natl Acad Sci USA, 108, 504-509 (2011).
^{b}Matsushika, A., Inoue, H., Watanabe, S., Kodaki, T., Makino, K. and Sawayama, S. (2009) Efficient bioethanol production by a recombinant flocculent Saccharomyces cerevisiae strain with a genome-Integrated NADP(+)-dependent xylitol dehydrogenase gene. Applied and Environmental Microbiology, 75, 3818-3822.

The plasmids bearing the optimized pathways were isolated from the selected strains and submitted for DNA sequencing to identify the promoter mutants in these pathways. Surprisingly, many of the promoter mutants in the pathways were mutated when compared to the sequence of the promoter mutants originally introduced into the pathway library. In order to determine the expression profiles of the selected strains, the mutated promoters were cloned into the pRS415-GFP helper plasmid originally used to construct the promoter mutant library. The promoter strength of these mutated promoters was determined using flow cytometry (Table 9-3).

TABLE 9-3
DNA sequencing of the promoters in the fastest xylose utilizing strains.
Left: Sequence similarity with the reference promoter mutants and number of mutations in the
promoters. Right: Relative promoter strength of the promoter mutants. The strength of the wild
type TEF1 promoter was defined as 100.The best pathway mutant in each strain background is
marked in grey.

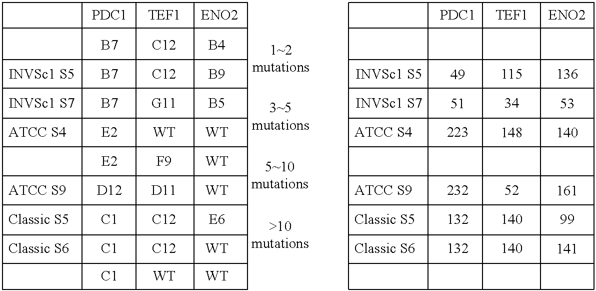

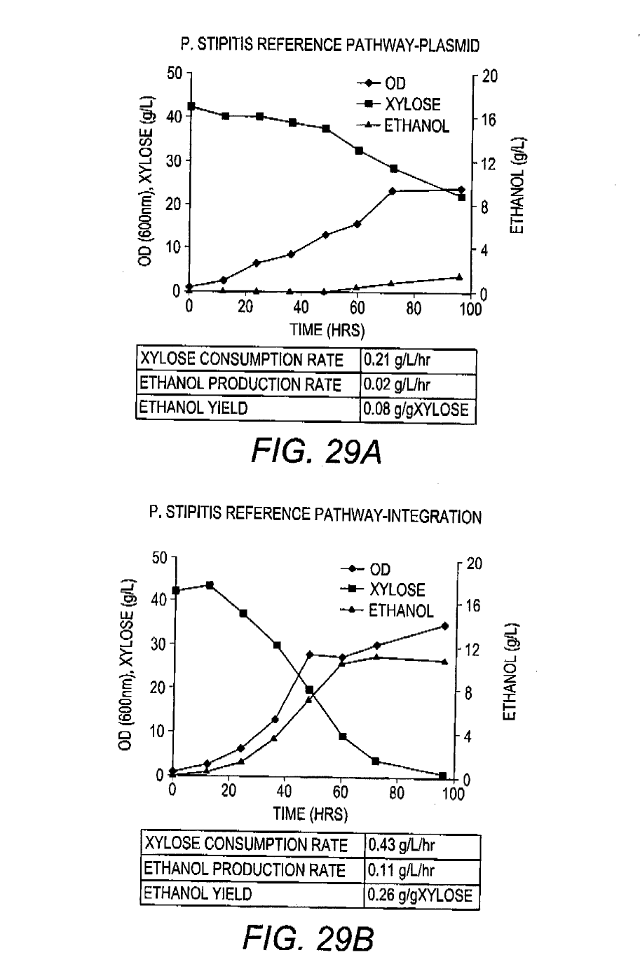

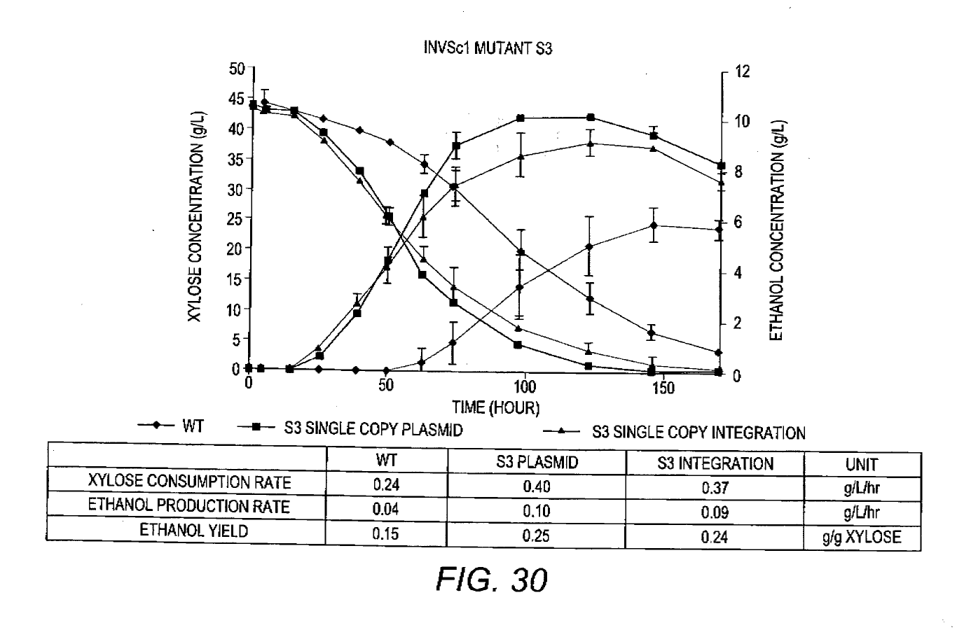

Our previous study showed that integration of the xylose utilization pathway into the chromosome would improve the xylose fermentation ability of the mutant strains (FIG. 29). To investigate the effect of its chromosomal integration, the xylose utilization pathway in the best mutant of the fastest growing INVSc1 strain (S3) was cloned into a pRS406 single copy integrative plasmid and integrated into the URA3 site of the INVSc1 strain. The fermentation behavior of the S2 pathway on a single copy centromeric plasmid and a single copy chromosomal integration was compared to the wild type pathway (WT) in the INVSc1 strain (FIG. 30).

Single colonies of INVSc1 strain harboring either a freshly retransferred S3 pathway on a plasmid (e.g. a single copy plasmid), a confirmed chromosomally integrated S3 pathway, or the control pathway were inoculated into 3 mL of SC-Ura liquid media supplemented with 2% glucose in round bottom culture tubes. The tube cultures were then grown at 30° C. and 250 rpm for 24 hours and used to inoculate 125 mL baffled shake flasks containing 25 mL of SC-Ura liquid media supplemented with 2% glucose as seed cultures. The seed cultures were grown for another 24 hours and then used to inoculate 250 mL un-baffled shake flasks containing 50 mL of YP media supplemented with 2% xylose to an initial OD of 1. The cultures were grown at 30° C. with 100 rpm agitation.

The control pathway consisting of a xylose utilizing pathway driven by the wild type PDC1, TEF1, and ENO2 promoters consumed 40 g/L xylose within 170 hours, while the S3 mutant strain consumed 40 g/L xylose within 100 hours. The xylose consumption rate, ethanol production rate, and ethanol yield were calculated using the 97.5 hour time point in FIG. 30. The S3 mutant strain consumed xylose and produced ethanol at 1.7 times of the rate of the strain containing the control pathway. The ethanol yield was also improved by more than 60% in the S3 mutant strain. Of note, the integration of the S3 pathway did not improve the fermentation performance.

The xylose fermentation ability of the best pathway mutants was also investigated in industrial strains. Single colonies of freshly retransformed industrial strains harboring mutant pathways were inoculated into 3 mL YPAD media supplemented with 2% and 200 mg/L G418. Tube cultures were grown at 30° C. under 250 rpm of agitation for 24 hours and then used to inoculate 125 mL baffled shake flasks containing YP media supplement with either 2% of glucose and 200 mg/L G418 or 2% xylose. The seed shake flask cultures were grown at 30° C. under 250 rpm agitation for another 24 hours and then used to inoculate 250 mL un-baffled shake flasks to an initial OD of 2 or 10.

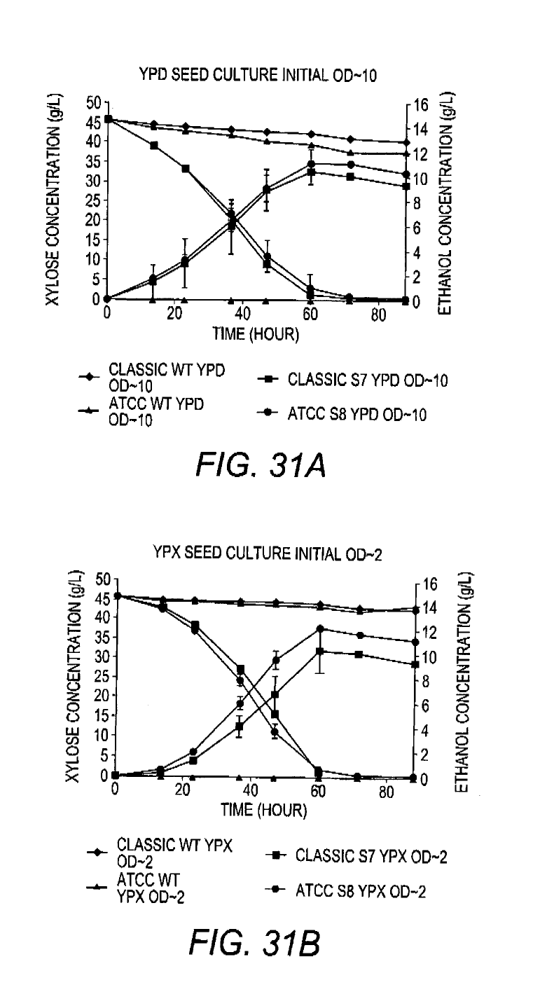

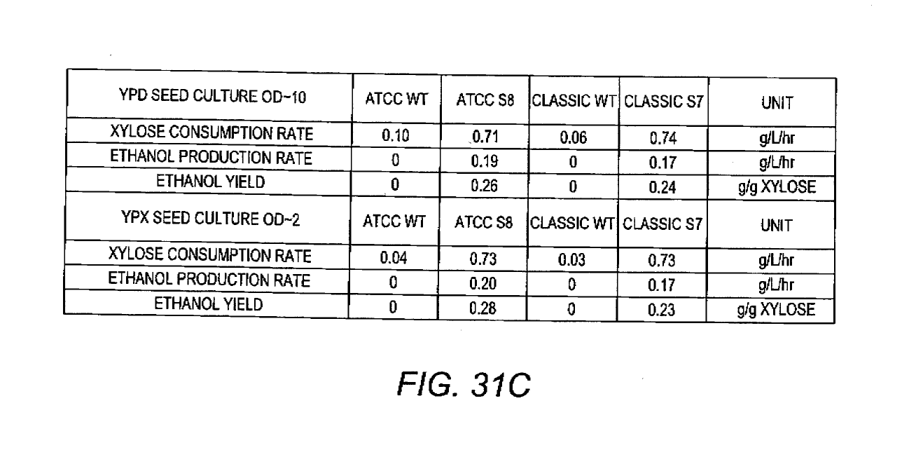

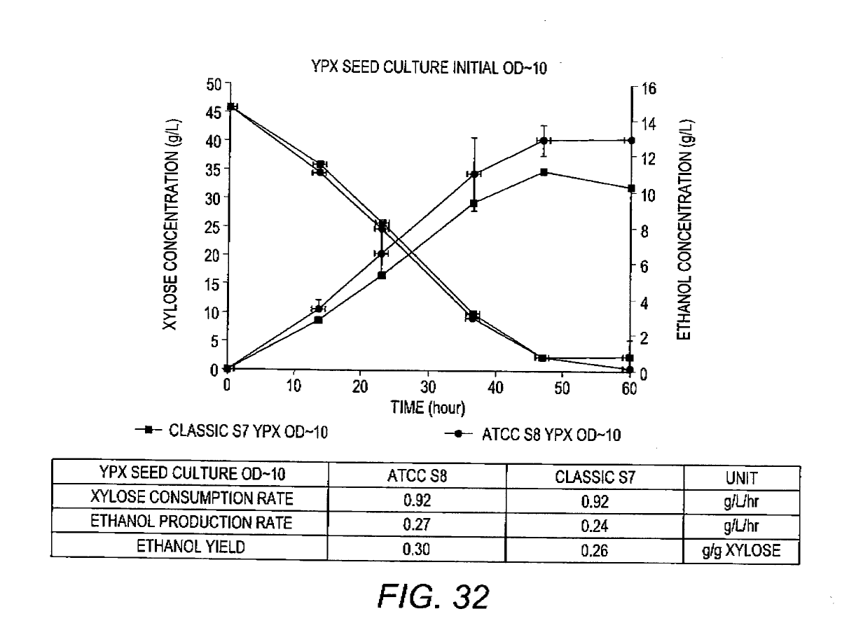

In industrial strains, the control pathway consisting of a pathway driven by the wild type PDC1, TEF1, and ENO2 promoters consumed less than 5 g/L xylose within 90 hours, while the industrial strains harboring the optimized mutant pathways consumed 40 g/L xylose within 60 hours. When xylose medium was used for seed culture and high initial OD was used, the industrial strains harboring the best mutant pathways can consume 40 g/L xylose within 48 hours. The xylose consumption rate, ethanol production rate, and ethanol yield were calculated using the 60 hour point for the fermentation of YPD seed culture with an initial OD of 10 and YPX seed culture with an initial OD of 2. Since the xylose was almost depleted at the 47.5 hour point of the fermentation with YPX seed culture and an initial OD of 10, the xylose consumption rate, ethanol production rate, and ethanol yield were calculated using the 47.5 hour time point. As shown in FIGS. 31 and 32, industrial strains with the optimized pathways consumed xylose and produced ethanol faster and with a higher yield when compared to the laboratory strain INVSc1.

Discussion Regarding Example 9

Balancing the metabolic flux of heterologous pathways is one of the key challenges in the metabolic engineering of microbial factories for the overproduction of desired compounds. Traditional approaches for optimization of metabolic pathways involve the identification of bottlenecks and branching points of metabolic pathways, followed by the overexpression and deletion of certain genes for either “debottlenecking” or “debugging” the pathways (Van Vleet and Jeffries, Current Opin in Biotech, 20, 300-36 (2009)). However, it is sometimes very hard to obtain optimal pathways using these approaches since strong overexpression and the deletion of genes only sample two extreme points in the space of gene expression. Alper and coworkers showed in their work that the fine-tuning of genetic expression for pathway optimization may be more effective, which can be achieved through the use of a series of promoters with varying strength (Alper et al, Proc Natl Acad Sci USA, 102, 12678-12683 (2005)). In this Example, three groups of promoter mutants with varying strength were created using nucleotide analogue mutagenesis. Around ten mutants were isolated for each promoter group and assembled together with a fixed set of metabolic enzyme homologues to form gene expression cassettes with varying expression levels. These gene expression cassettes were then used as building blocks for the assembly of libraries of xylose utilization pathways with different expression profiles in various S. cerevisiae strain backgrounds.

Due to the high degree of homology between the promoter mutants generated from the same template, homologous recombination inevitably occurred between different promoter mutants during the pathway assembly process. The recombination between promoter mutants resulted in the incorporation of mutated or chimeric promoter mutants into the assembled pathway library that was not present in the original promoter mutant libraries (Table 9-3). The existence of these chimeric mutants increased the number of possible combinations in the libraries.

The wide existence of mutated promoters in the top selected pathways indicated that the recombination rate between promoter mutants was very high. For example, in the top three mutants in the INVSc1 promoter-based library assembly, all three PDC1 promoter mutants were mutated. At the same time, some mutated promoters that were found in the top mutants also exhibited strengths exceeding the dynamic range of the promoter mutants originally isolated for the assembly. For example, in the top three mutants from the promoter-based pathway assembly in industrial strains, mutants of the TEF1 promoter with a relative strength of 150 were identified, yet the highest recorded strength of the TEF1 promoter mutants in the pre-selected library was only around 110.

It has been previously shown that different S. cerevisiae strains have distinct xylulose fermentation abilities due to their inherent capacities for pentose sugar metabolism (Matsushika et al, Bioresource Technology, 100, 2392-2398 (2009)). This is consistent with the promoter-based pathway optimization yielding different combinations of expression levels for xylose reductase, xylose dehydrogenase, and xylulokinase under different strain backgrounds. These differences in expression profiles may have resulted from the differences in the expression level of endogenous aldose reductases, the activity of endogenous xylulokinases, or the capacity of the downstream pentose phosphate pathways in the host strains. The different expression profiles may also have arisen from the distinct individual capabilities of cofactor regeneration and cell stress response in different types of host cells.

In some cases, mutant pathways generated as disclosed herein, the pathway optimization approach may be “strain-background-specific,” and the optimized pathway mutants can be regarded as pathways “tailor-made” for a specific strain. The promoter-based pathway assembly method may be used to optimize pathways in different strains background, as well as the same strain background under different fermentation or nutrition conditions.

It is commonly known that fermentation conditions used in the industrial production of biofuels are very different from the ones used in the shake flasks and fermenters of typical research laboratories (Cakar et al, FEMS Yeast Research, 5, 569-578 (2005)). The temperature, pH, inhibitor, and even starvation stress that exist in industrial fermentation processes can affect the metabolism of recombinant strains—possibly resulting in suboptimal performance of the strains in the industrial biofuel production. In these cases, the promoter-based pathway assembly method may be applied to balance the metabolic flux within the heterologous pathway in order to manufacture the best possible fit for the fermentation conditions in modern-day industrial applications.

Researchers have been working for decades to improve the fermentation ability of S. cerevisiae using xylose as a carbon source. After the introduction of functional xylose utilizing pathways from xylose assimilating yeast, such as P. stipitis into S. cerevisiae, numerous efforts have been made to modify both the heterologous and endogenous genes to optimize the xylose fermentation efficiency. In this Example, the xylose utilization pathway was optimized using a combinatorial pathway library containing pathway mutants with different expression profiles. Using this approach, the xylose utilization ability of the pathway containing wild type PDC1, TEF1, and ENO2 promoters was improved two-fold in the INVSc1 strain, while the xylose utilization rate was improved six-fold when industrial S. cerevisiae strains were used as the host. Of note, the dramatic improvement in this study was achieved by the optimization of the three gene heterologous xylose utilizing pathway in S. cerevisiae within eight months that did not rely on any knowledge of the vast previous metabolic engineering studies on xylose utilization. This method can be further applied to optimize the extended xylose utilization pathway, including the xylose transporter, endogenous pentose phosphate pathway genes, and other genes that might facilitate xylose utilization in S. cerevisiae.

In this Example, a promoter-based pathway assembly method was developed for the optimization of a three gene fungal xylose utilization pathway in S. cerevisiae. First, twenty XR homologues, twenty-two XDH homologues, and nineteen XKS homologues of xylulokinase were cloned and assayed for enzymatic activity. Enzyme homologues with high activity and matching cofactor specificity, namely csXR, ctXDH, and ppXKS, were selected to form the scaffold for combinatorial pathway assembly. At the same time, promoter mutants with varying strength were created using S. cerevisiae native promoters PDC1, TEF1, and ENO2 as templates. Around ten mutants with varying strength were pre-selected and assembled with the same set of enzyme homologues in order to generate building blocks for pathway assembly. The gene expression cassettes were then assembled into a pathway library of the same pathway enzymes with different expression profiles. This pathway library was screened in laboratory yeast strain INVSc1 and industrial yeast strains ATCC 4124 and Classic Turbo using colony size-based prescreening followed by tube and shake flask screening. After the screening, strains harboring the best pathway mutant in the INVSc1 strain background consumed xylose and produced ethanol at 1.7 times of the rate of the strain containing the control pathway with a more than 60% improvement of ethanol yield compared to the control strain harboring the same xylose utilization pathway driven by the wild type promoters, while the best pathway mutants identified in industrial strains achieved six-fold improvement of xylose fermentation ability compared to the control strain.

Unlike the traditional pathway optimization strategies, which rely on identifying the rate limiting step and then optimizing pathways by deletion and strong overexpression of certain genes (Alper et al., Proc Natl Acad Sci USA, 102:12678-12683, (2005)), the assembly method disclosed herein provides a new strategy for balancing metabolic flux in recombinant strains. Instead of overexpression and deletion of genes within the metabolic pathway, a series of promoter mutants with varying strength were shuffled. This act of promoter shuffling generated a library of pathways with different expression profiles. Using this method, thousands of combinations of gene expression levels for a multi-step metabolic pathway can be assembled and investigated.

Many complicated metabolic pathways may be optimized by the strategy presented in this Example, when a proper screening or selection method is available. Moreover, this newly developed strategy can also enable host strain-specific pathway optimization for tailor-making pathways for special strains with a particular metabolic background or under a specific growth condition.

In the process of optimizing these pathways, a library of xylose utilization pathways with diversified behaviors was also generated. Given the well-defined scaffold using fixed catalytic enzyme homologues, the diversity of the pathway mutants mainly relies on the different expression levels of genes involved in the pathway assembly. In other words, the pathway libraries generated using the strategy described in this Example exhibit a controlled diversity. These kinds of libraries are very useful in the understanding of metabolic pathways. Regulation and interaction of metabolic pathways can be studied through approaches such as metabolic flux analysis and DNA microarrays. The pathway library consisting of the same pathway enzymes with different expression profiles can also be used to study the effect of the perturbation of expression level of a certain enzyme on the overall pathway performance. Models of metabolic pathways can be generated using the data collected by studying mutants from the pathway library to understand and predict the response of the metabolic pathway to varying gene expression profiles.

### Example 10

### Further Optimization of Pentose Utilization Pathways Using Additional Genes and Promoters of Varying Strengths

Additional genes are utilized in some embodiments of the present disclosure. In particular, xylose-specific transporters, as well as endogenous transaldolase (TAL) and transketolase (TKL) are employed. TAL and TKL are endogenous in the sense that they are encoded by genes of S. cerevisiae. However, for efficient ethanol production from pentose sugars TAL and TKL are overexpressed from exogenously introduced expression cassettes. For the optimization of the xylose pathway having six components (XR, XDH, XKS, xylose transporter, TAL and TKL), six different promoters are used. A library of promoters of varying strengths is used to generate a library of a six component xylose pathway. In this library, the same combination of coding regions is employed (XR, XDH, XKS, xylose transporter, TAL and TKL), but their relative expression varies due to the utilization of different mutant promoters.

For optimization of the xylose/arabinose pathway having nine components (XR, XDH, XKS, xylose transporter, LAD, LXR, arabinose-specific transport, TAL and TKL), six different promoters are also used. The nine component xylose/arabinose pathway is expressed from two plasmids, so that the some of the promoters can be used repeatedly. In some embodiments, six expression cassettes employing six different promoters are included in a first plasmid, and three expression cassettes employing three different promoters are included on a second plasmid (e.g., at least three promoters are used twice. A promoter library with varying strengths is used to generate a library of multi-component pathways with different expression patterns.

The amino acid sequence of the An25 xylose-specific
transporter from N. crassa is set forth as SEQ ID NO: 90:
MAPPKFLGLSGRPLSLAVSTVATTGFLLFGYDQGVMSGIITAPAFNNFFTPTKDNSTMQG
LITAIYEIGCLIGAMFVLWTGDLLGRRRNIMVGAFIMALGVIIQVTCQAGSNPFAQLFVG
RVVMGIGNGMNTSTIPTYQAECSKTSNRGLLICIEGGVIAFGTLIAYWIDYGASYGPDDL
VWRFPIAFQLLFAIFICVPMFYLPESPRWLLSHGRTQEADKVIAALRGYEIDGPETIQERN
LIVDSLRASGGFGQKSTPFKALFTGGKTQHFRRLLLGSSSQFMQQVGGCNAVIYYFPILF
QDSIGESHNMSMLLGGINMIVYSIFATVSWFAIERVGRRRLFLIGTVGQMLSMVIVFACLI
PDDPMKARGAAVGLFTYIAFFGATWLPLPWLYPAEVNPIRTRGKANAVSTCSNWMFNF
LIVMVTPIMVDKIGWGTYLFFAVMNGCFLPIIYFFYPETANRSLEEIDIIFAKGFVENMSY
VTAAKELPHLTAEEIESYANKYGLVDRDSNGEGGNRHDEEKTRDRPDQSDSDSPAHVEI
DVVDEHGVESGFGDGINTKETR.
The amino acid sequence of the Xyp29 xylose-specific
transporter from P. stipitis is set forth as SEQ ID NO: 91:
MSSVEKSAETASYTSQVSASGSAKTNSYLGLRGHKLNFAVSCFAGVGFLLFGYDQGVM
GSLLTLPSFENTFPAMKASNNATLQGAVIALYEIGCMSSSLATIYLGDRLGRLKIIVIFIGCV
IVCIGAALQASAFTIAHLTVARIITGLGTGFITSTVPVYQSECSPAKKRGQIIIVIMEGSLIAL
GIAISYWIDFGFYFLRNDGLHSSASWRAPIALQCVFAVLLISTVFFFPESPRWLLNKGRTE
EAREVFSALYDLPADSEKISIQIEEIQAAIDLERQAGEGFVLKELFTQGPARNLQRVALSC
WSQIIVIQQITGINIITYYAGTIFESYIGMSPFMSRILAALNGTEYFLVSLIAFYTVERLGRRF
LLFWGAIAMALVMAGLTVTVKLAGEGNTHAGVGAAVLLFAFNSFFGVSWLGGSWLLP
PELLSLKLRAPGAALSTASNWAFNFMVVMITPVGFQSIGSYTYLIFAAINLLMAPVIYFL
YPETKGRSLEEMDIIFNQCPVWEPWKVVQIARDLPIMHSEVLDHEKNVIIKKSRIEHVENI
S.
The amino acid sequence of the Xyp32 arabinose-specific
transporter from P. stipitis is set forth as SEQ ID NO: 92:
MHGGGDGNDITEIIAARRLQIAGKSGVAGLVANSRSFFIAVFASLGGLVYGYNQGMFGQ
ISGMYSFSKAIGVEKIQDNPTLQGLLTSILELGAWVGVLMNGYIADRLGRKKSVVVGVF
FFFIGVIVQAVARGGNYDYILGGRFVVGIGVGILSMVVPLYNAEISPPEIRGSLVALQQLA
ITFGIMISYWITYGTNYIGGTGSGQSKASWLVPICIQLVPALLLGVGIFFMPESPRWLMNE
DREDECLSVLSNLRSLSKEDTLVQMEFLEMKAQKLFERELSAKYFPHLQDGSAKSNFLI
GFNQYKSMITHYPTFKRVAVACLIMTFQQWTGVNFILYYAPFIFSSLGLSGNTISLLASG
VVGIVMFLATIPAVLWVDRLGRKPVLISGAIIMGICHFVVAAILGQFGGNFVNHSGAGW
VAVVFVWIFAIGFGYSWGPCAWVLVAEVFPLGLRAKGVSIGASSNWLNNFAVAMSTPD
FVAKAKFGAYIFLGLMCIFGAAYVQFFCPETKGRTLEEIDELFGDTSGTSKMEKEIHEQK
LKEVGLLQLLGEENASES ENSKADVYHVEK.
The amino acid sequence of the transaldolase (TAL) of
S. cerevisiae is set forth as SEQ ID NO: 93:
MSEPAQKKQKVANNS LEQLKASGTVVVADTGDFGSIAKFQPQDSTTNPSLILAAAKQPT
YAKLIDVAVEYGKKHGKTTEEQVENAVDRLLVEFGKEILKIVPGRVSTEVDARLSFDTQ
ATIEKARHIIKLFEQEGVSKERVLIKIASTWEGIQAAKELEEKDGIHCNLTLLFSFVQAVA
CAEAQVTLISPFVGRILDWYKSSTGKDYKGEADPGVISVKKIYNYYKKYGYKTIVMGAS
FRSTDEIKNLAGVDYLTISPALLDKLMNSTEPFPRVLDPVSAKKEAGDKISYIDDESKFRF
DLNEDAMATEKLSEGIRKFSADIVTLFDLIEKKVTA.
The amino acid sequence of the transketolase (TKL) of
S. cerevisiae is set forth as SEQ ID NO: 94:
MAQFSDIDKLAVSTLRLLSVDQVESAQSGHPGAPLGLAPVAHVIFKQLRCNPNNEHWIN
RDRFVLSNGHSCALLYSMLHLLGYDYSIEDLRQFRQVNSRTPGHPEFHSAGVEITSGPLG
QGISNAVGMAIAQANFAATYNEDGFPISDSYTFAIVGDGCLQEGVSSETSSLAGHLQLGN
LITFYDSNSISIDGKTSYSFDEDVLKRYEAYGWEVMEVDKGDDDMESISSALEKAKLSK
DKPTIIKVTTTIGFGSLQQGTAGVHGSALKADDVKQLKKRWGFDPNKSFVVPQEVYDY
YKKTVVEPGQKLNEEWDRMFEEYKTKFPEKGKELQRRLNGELPEGWEKHLPKFTPDDD
ALATRKTSQQVLTNMVQVLPELIGGSADLTPSNLTRWEGAVDFQPPITQLGNYAGRYIR
YGVREHGMGAIMNGISAFGANYKPYGGTFLNFVSYAAGAVRLAALSGNPVIVVVATHD
SIGLGEDGPTHQPIETLAHLRAIPNMHVWRPADGNETSAAYYSAIKSGRTPSVVALSRQN
LPQLEHSSFEKALKGGYVIHDVENPDIILVSTGSEVSISIDAAKKLYDTKKIKARVVSLPD
FYTFDRQSEEYRFSVLPDGVPIIVISFEVLATSSWGKYAHQSFGLDEFGRSGKGPEIYKLFD
FTADGVASRAEKTINYYKGKQLLSPMGRAF.

As described in Example 6, this eight-component pathway library is enriched using serial transfers under selective conditions. The pathway with optimal metabolic flux becomes dominant after enrichment. The eight-component arabinose/xylose utilization pathway is optimized in this way in both laboratory and industrial yeast strains.

### Example 11

### Construction of Expression Systems for Pentose Utilization Pathway Engineering in Industrial S. cerevisiae Strains

In order to introduce, characterize, and optimize pathways in industrial strains, dominant drug-resistant selection markers were investigated in several industrial S. cerevisiae strains (Table 11-1). Using pRS416 as a backbone, dominant drug-resistant markers KanMX (Walker et al., FEMS Yeast Res, 4:339-347, 2003), AUR1-c (HashidoOkado et al., Mol Gen Genetics, 251:236-244, 1996), CAT (Hadfield et al., Gene, 45:149-158, 1986), and YAP1 (Akada et al., Yeast, 19:17-28, 2002) were used to construct a single copy expression vector. The drug resistance of these markers was tested in Still Spirits Turbo Yeast Classic (Classic) and Alcotec Turbo Super Yeast (Super) from Homebrewing company, as well as S. cerevisiae Type II from Sigma Aldrich. KanMX, YAP1 and AUR1-c markers all worked in these strains. CAT did not work in a first attempt using 1.5 g/L chloramphenicol, it has since because the chloramphenicol concentration used for selection is dependent upon the strain background and therefore must be optimized.

The nucleic acid sequence of AUR1-c is set forth as SEQ ID NO: 124:
atggcaaaccctttttcgagatggtttctatcagagagacctccaaactgccatgtagccgatttagaaacaagtttagatccccatcaaacgtt
gttgaaggtgcaaaaatacaaacccgctttaagcgactgggtgcattacatcttcttgggatccatcatgctgtttgtgttcattactaatcccgc
accttggatcttcaagatccttttttattgtttcttgggcactttattcatcattccagctacgtcacagtttttcttcaatgccttgcccatcct
aacatgggtggcgctgtatttcacttcatcgtactttccagatgaccgcaggcctcctattactgtcaaagtgttaccagcggtggaaacaatttt
atacggcgacaatttaagtgatattcttgcaacatcgacgaattcctttttggacattttagcatggttaccgtacggactatttcattatggggc
cccatttgtcgttgctgccatcttattcgtatttggtccaccaactgttttgcaaggttatgcttttgcatttggttatatgaacctgtttggtgt
tatcatgcaaaatgtctttccagccgctcccccatggtataaaattctctatggattgcaatcagccaactatgatatgcatggctcgcctggtgg
attagctagaattgataagctactcggtattaatatgtatactacatgtttttcaaattcctccgtcattttcggtgcttttccttcactgcattc
cgggtgtgctactatggaagccctgtttttctgttattgttttccaaaattgaagcccttgtttattgcttatgtttgctggttatggtggtcaac
tatgtatctgacacaccattattttgtagaccttatggcaggttctgtgctgtcatacgttattttccagtacacaaagtacacacatttaccaat
tgtagatacatctcttttttgcagatggtcatacacttcaattgagaaatacgatatatcaaagagtgatccattggctgcagattcaaacgatat
cgaaagtgtccctttgtccaacttggaacttgactttgatcttaatatgactgatgaacccagtgtaagcccttcgttatttgatggatctacttc
tgtttctcgttcgtccgccacgtctataacgtcactaggtgtaaagagggcttaa.
The nucleic acid sequence of KanMX is set forth as SEQ ID NO: 125:
atgggtaaggaaaagactcacgtttcgaggccgcgattaaattccaacatggatgctgatttatatgggtataaatgggctcgcgataatgtc
gggcaatcaggtgcgacaatctatcgattgtatgggaagcccgatgcgccagagttgtttctgaaacatggcaaaggtagcgttgccaatga
tgttacagatgagatggtcagactaaactggctgacggaatttatgcctcttccgaccatcaagcattttatccgtactcctgatgatgcatggtt
actcaccactgcgatccccggcaaaacagcattccaggtattagaagaatatcctgattcaggtgaaaatattgttgatgcgctggcagtgttc
ctgcgccggttgcattcgattcctgtttgtaattgtccttttaacagcgatcgcgtatttcgtctcgctcaggcgcaatcacgaatgaataacggt
ttggttgatgcgagtgattttgatgacgagcgtaatggctggcctgttgaacaagtctggaaagaaatgcataagcttttgccattctcaccgga
ttcagtcgtcactcatggtgatttctcacttgataaccttatttttgacgaggggaaattaataggttgtattgatgttggacgagtcggaatcgc
agaccgataccaggatcttgccatcctatggaactgcctcggtgagttttctccttcattacagaaacggctttttcaaaaatatggtattgataa
tcctgatatgaataaattgcagtttcatttgatgctcgatgagtttttctaa.
The nucleic acid sequence of CAT is set forth as SEQ ID NO: 126:
atggagaaaaaaatcactggatataccaccgttgatatatcccaatggcatcgtaaagaacattttgaggcatttcagtcagttgctcaatgtac
ctataaccagaccgttcagctggatattacggcctttttaaagaccgtaaagaaaaataagcacaagttttatccggcctttattcacattcttgc
ccgcctgatgaatgctcatccggaattccgtatggcaatgaaagacggtgagctggtgatatgggatagtgttcacccttgttacaccgttttc
catgagcaaactgaaacgttttcatcgctctggagtgaataccacgacgatttccggcagtttctacacatatattcgcaagatgtggcgtgtta
cggtgaaaacctggcctatttccctaaagggtttattgagaatatgtttttcgtctcagccaatccctgggtgagtttcaccagttttgatttaaa
cgtggccaatatggacaacttcttcgcccccgttttcaccatgggcaaatattatacgcaaggcgacaaggtgctgatgccgctggcgattcag
gttcatcatgccgtctgtgatggcttccatgtcggcagaatgcttaatgaattacaacagtactgcgatgagtggcagggcggggcgtaa.
The nucleic acid sequence of YAP1 is set forth as SEQ ID NO: 127:
atgagtgtgtctaccgccaagaggtcgctggatgtcgtttctccgggttcattagcggagtttgagggttcaaaatctcgtcacgatgaaatag
aaaatgaacatagacgtactggtacacgtgatggcgaggatagcgagcaaccgaagaagaagggtagcaaaactagcaaaaagcaaga
tttggatcctgaaactaagcagaagaggactgcccaaaatcgggccgctcaaagagcttttagggaacgtaaggagaggaagatgaagg
aattggagaagaaggtacaaagtttagagagtattcagcagcaaaatgaagtggaagctacttttttgagggaccagttaatcactctggtga
atgagttaaaaaaatatagaccagagacaagaaatgactcaaaagtgctggaatatttagcaaggcgagatcctaatttgcatttttcaaaaaa
taacgttaaccacagcaatagcgagccaattgacacacccaatgatgacatacaagaaaatgttaaacaaaagatgaatttcacgtttcaatat
ccgcttgataacgacaacgacaacgacaacagtaaaaatgtggggaaacaattaccttcaccaaatgatccaagtcattcggctcctatgcc
tataaatcagacacaaaagaaattaagtgacgctacagattcctccagcgctactttggattccctttcaaatagtaacgatgttcttaataacac
accaaactcctccacttcgatggattggttagataatgtaatatatactaacaggtttgtgtcaggtgatgatggcagcaatagtaaaactaaga
atttagacagtaatatgttttctaatgactttaattttgaaaaccaatttgatgaacaagtttcggagttttgttcgaaaatgaaccaggtatgtg
gaacaaggcaatgtcccattcccaagaaacccatctcggctcttgataaagaagttttcgcgtcatcttctatactaagttcaaattctcctgctt
taacaaatacttgggaatcacattctaatattacagataatactcctgctaatgtcattgctactgatgctactaaatatgaaaattccttctccg
gttttggccgacttggtttcgatatgagtgccaatcattacgtcgtgaatgataatagcactggtagcactgatagcactggtagcactggcaata
agaacaaaaagaacaataataatagcgatgatgtactcccattcatatccgagtcaccgtttgatatgaaccaagttactaatttttttagtccgg
gatctaccggcatcggcaataatgctgcctctaacaccaatcccagcctactgcaaagcagcaaagaggatataccttttatcaacgcaaatctg
gctttcccagacgacaattcaactaatattcaattacaacctttctctgaatctcaatctcaaaataagtttgactacgacatgttttttagagat
tcatcgaaggaaggtaacaatttatttggagagtttttagaggatgacgatgatgacaaaaaagccgctaatatgtcagacgatgagtcaagttta
atcaagaaccagttaattaacgaagaaccagagcttccgaaacaatatctacaatcggtaccaggaaatgaaagcgaaatctcacaaaaaaat
ggcagtagtttacagaatgctgacaaaatcaataatggcaatgataacgataatgataatgaagtcgttccatctaaggaaggctctttactaa
ggtgttcggaaatttgggatagaataacaacacatccgaaatactcagatattgatgtcgatggtttatgttccgagctaatggcaaaggcaaa
atgttcagaaagaggggttgtcatcaatgcagaagacgttcaattagctttgaataagcatatgaactaa.

TABLE 11-1
Comparison of Dominant Drug-Resistance Markers

| Marker | Drug | Gene Origin |
|---|---|---|
| KanMX | G418 | Tn903 |
| | (200 mg/L) | |
| AUR1-c | Aureobasidin A | AUR1-c mutation |
| | (0.5 mg/L) | |
| CAT | Chloramphenicol | Tn9 |
| | (1-5 g/L) | |
| YAP1 | Cerulenin/Cyclohexamide | YAP1 native |
| | (5 mg/L) | |

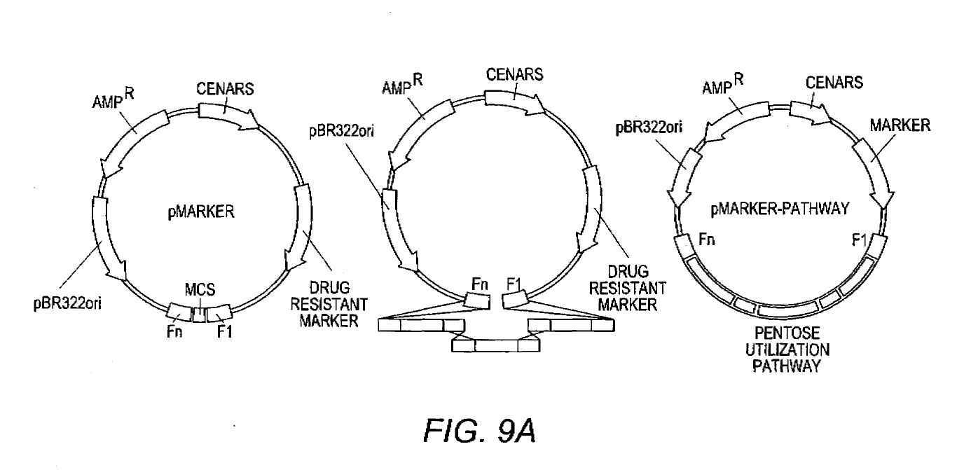

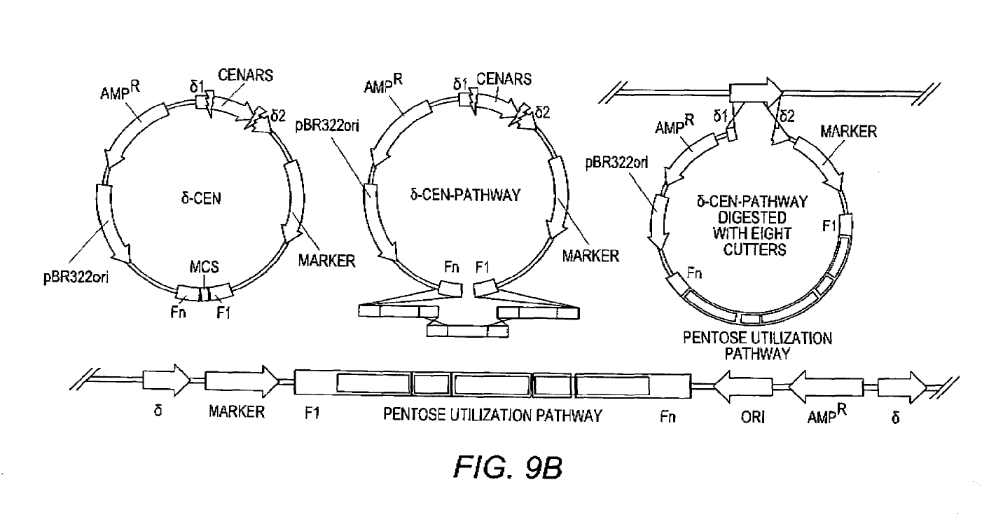

Using dominant drug-resistant markers with confirmed functionality, different expression systems were designed for the assembly and validation of pentose utilization pathways in an industrial yeast strain. First, single copy plasmid based expression vectors were designed using the pRS416 shuttle vector as a template. The uracil auxotrophic marker was replaced with different dominant selection markers. The resultant expression vectors retained the pBR322 origin of replication for propagation in E. coli, the CEN.ARS for maintenance of a single copy plasmid in yeast, the multiple cloning sites (MCS) for linearization of the vector, and the homology region flanking MCS for introduction of the pathway via DNA assembler (FIG. 9).

Next, an integrative vector was designed for multicopy integration of pathways into δ-sites of S. cerevisiae using the reusable KanMX marker flanked by loxP sites. In this vector, yeast CEN.ARS was flanked by spliced δ-sequences and rare restriction cutting sites were engineered in between the δ-sequence and CEN.ARS. A full pentose utilization pathway can be introduced into this vector through a one step DNA assembly method. Digestion with the restriction enzymes corresponding to the rare cutting sites produces a linearized integrative plasmid flanked by δ-sequences for multicopy integration due to the loss of the yeast CEN.ARS (FIG. 10). The rare restriction enzyme used to excise the CEN.ARS from this construct is PmeI purchased from New England Biolabs, which recognizes the 8 bp recognition sequence gtttaaac, which is shown in bold in SEQ ID NO:128.

The nucleic acid sequence of the deltal-CEN.ARS-delta2 fragment is set forth as
SEQ ID NO: 128:
Tggaagctgaaacgtctaacggatcttgatttgtgtggacttccttagaagtaaccgaagcacaggcgctaccatgagaattgggtgaatgtt
gagataattgttgggattccattgttgataaaggctataatattaggtatacagaatatactagaagttctcgtttaaacggtccttttcatcacg
tgctataaaaataattataatttaaattttttaatataaatatataaattaaaaatagaaagtaaaaaaagaaattaaagaaaaaatagtttttgt
tttccgaagatgtaaaagactctagggggatcgccaacaaatactaccttttatcttgctcttcctgctctcaggtattaatgccgaattgtttca
tcttgtctgtgtagaagaccacacacgaaaatcctgtgattttacattttacttatcgttaatcgaatgtatatctatttaatctgcttttcttgt
ctaataaatatatatgtaaagtacgctttttgttgaaattttttaaacctttgtttatttttttttcttcattccgtaactcttctaccttcttta
tttactttctaaaatccaaatacaaaacataaaaataaataaacacagagtaaattcccaaattattccatcattaaaagatacgaggcgcgtgta
agttacaggcaagcgatccgtccgtttaaacctcgaggatataggaatcctcaaaatggaatctgcaattctacacaattctataaatattattat
catcattttatatgtttatattcattgatcctattacattatcaatccttgcgtttcagcttccactaatttagatgactatttctcatcatttgc
gtcatcttctaacaccgtatatgataatatactagtaatgtaaatactagttagtagatgatagttgatttctattccaaca.

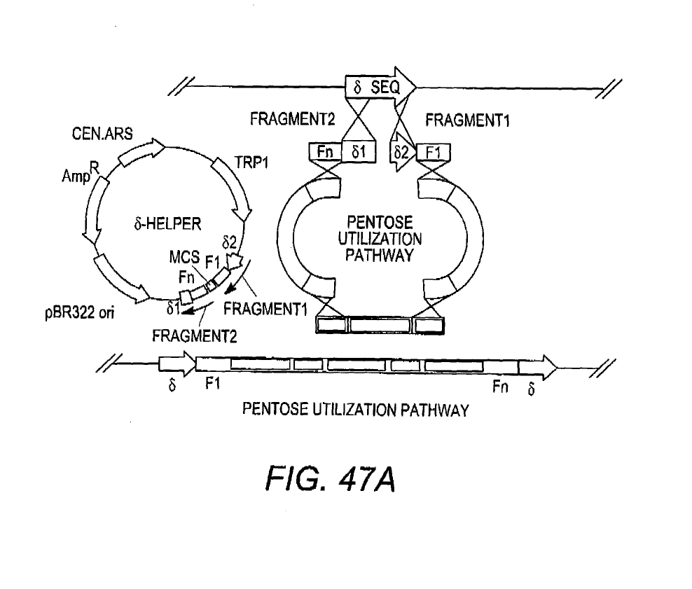

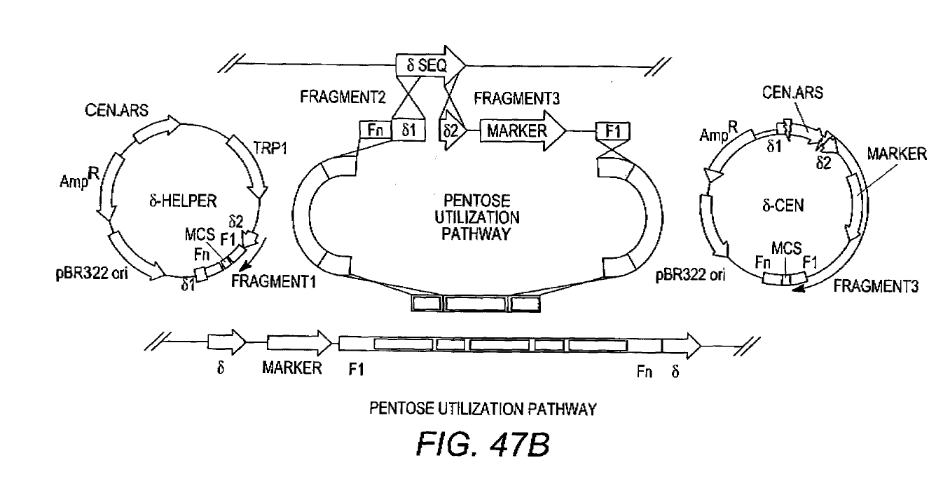

Finally, helper plasmids were designed to permit the cloning-free, multicopy genomic δ-integration of pentose utilization pathways industrial strains transformed with DNA fragments. Of note, chromosomal integration of the pentose utilization pathway does not necessarily require a separate positive selection marker, since growth on pentose sugars can serve as a positive selection pressure (Ho et al., Appl Environmental Microbiol, 64:1852-1859, 1998). DNA fragments containing δ-sequence and homology regions used for recombination cloning were co-transferred into industrial yeast strains with pentose utilization pathway components in order to affect multicopy integration of the pathway into the yeast genome (FIG. 47A). To assess the performance of industrial strains with a single integrated copy of a pentose pathway, the pAUR101 integrative vector from Clontech was used. The pAUR1010 integrative vector is suitable for introducing a single copy of a pentose pathway into the AUR site of industrial yeast strains.

The strategy of engineering the pentose utilization pathway in industrial yeast strains is a two step process. First, the pentose utilization pathway is optimized in laboratory strains through promoters-based and/or enzyme homologues-based DNA assembly. Next, the optimized pathways are introduced into industrial strains on a single copy plasmid, single copy integration or multicopy integration. Fermentation performance of the resulting recombinant industrial strains is subsequently investigated. Second, if the single copy or multicopy integration system proves to be a highly efficient method for pathway assembly, then libraries of pathways are directly assembled in industrial strains. The resultant pathway library is selected using growth conditions mimicking industrial fermentation conditions with lignocellulosic hydrolysate as the substrate. In this case, the pathway is optimized with the industrial ethanol production strains under industrial conditions, resulting in strains that should theoretically be better able to perform lignocellulosic ethanol fermentation under current industrial conditions

### Example 12

### Combinatorial Design and Optimization of Highly Efficient Cellobiose Utilization Pathways in Saccharomyces cerevisiae

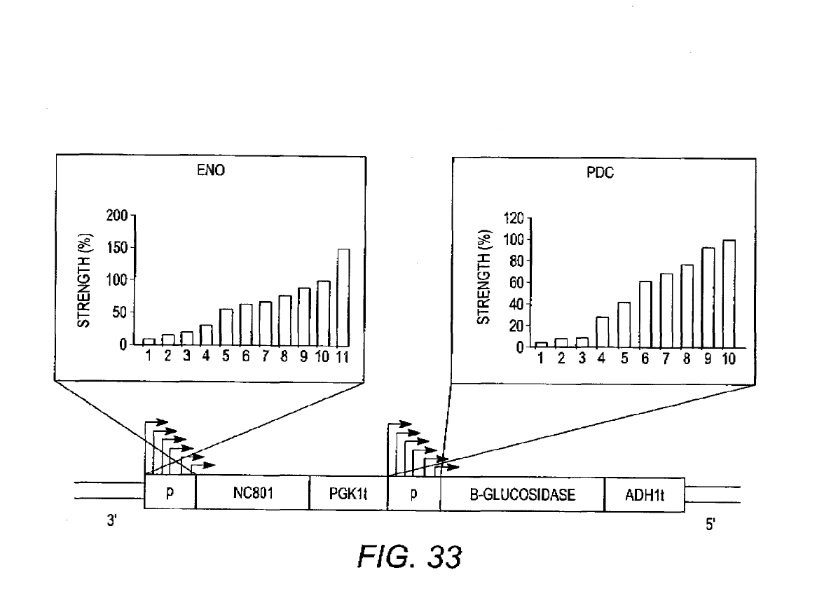

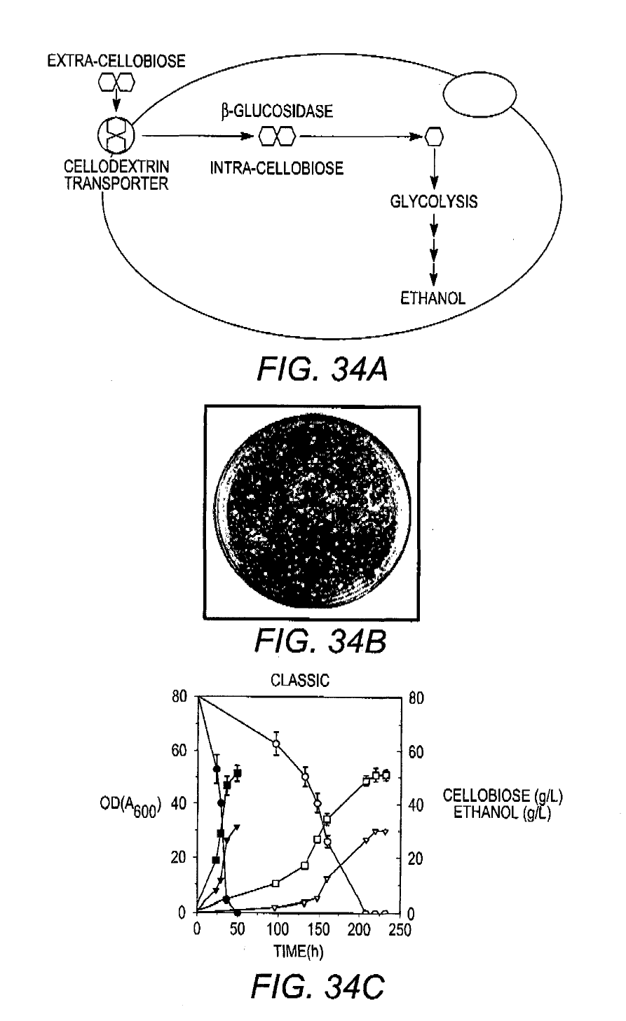

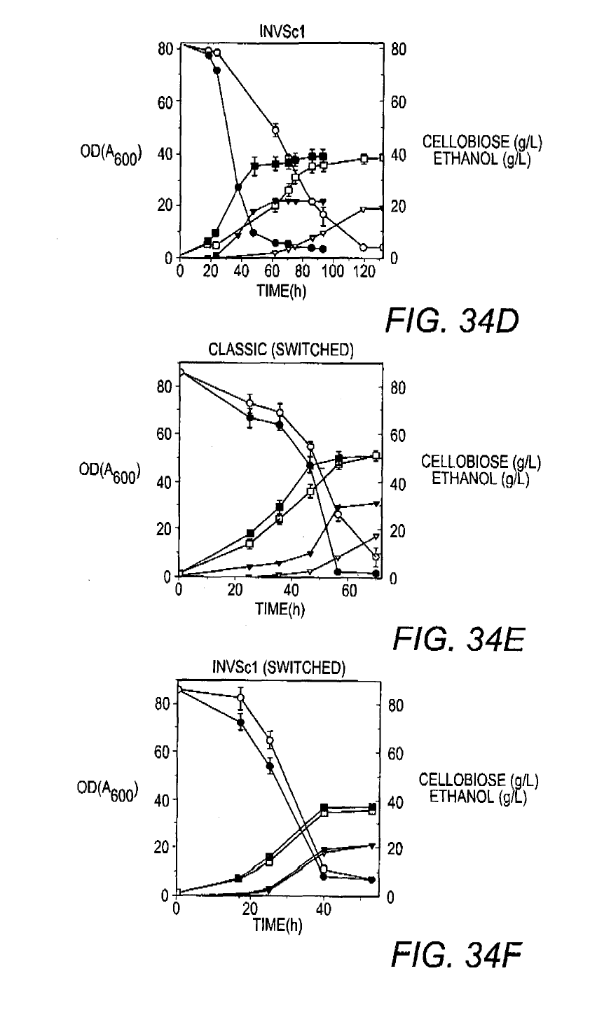

A novel combination of a cellodextrin transporter and a β-glucosidase was found to be capable of utilizing cellobiose in yeast (Li et al., Mol BioSyst 6, 2129-2132 (2010)). Cellobiose can be transported into the cell by the cellodextrin transporter and subsequently catalyzed by β-glucosidase to glucose which can be used by cells. (FIG. 34A) The purpose of this project was to optimize this two-protein pathway by balancing the metabolic flux in the cellobiose utilizing pathway. The ENO and PDC promoters were selected to control the expression of the cellodextrin transporter and β-glucosidase genes, respectively (FIG. 33).

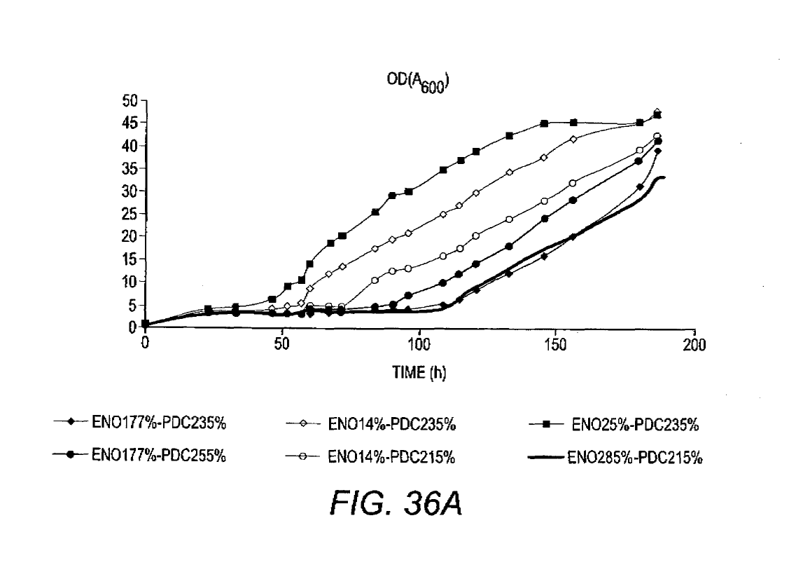

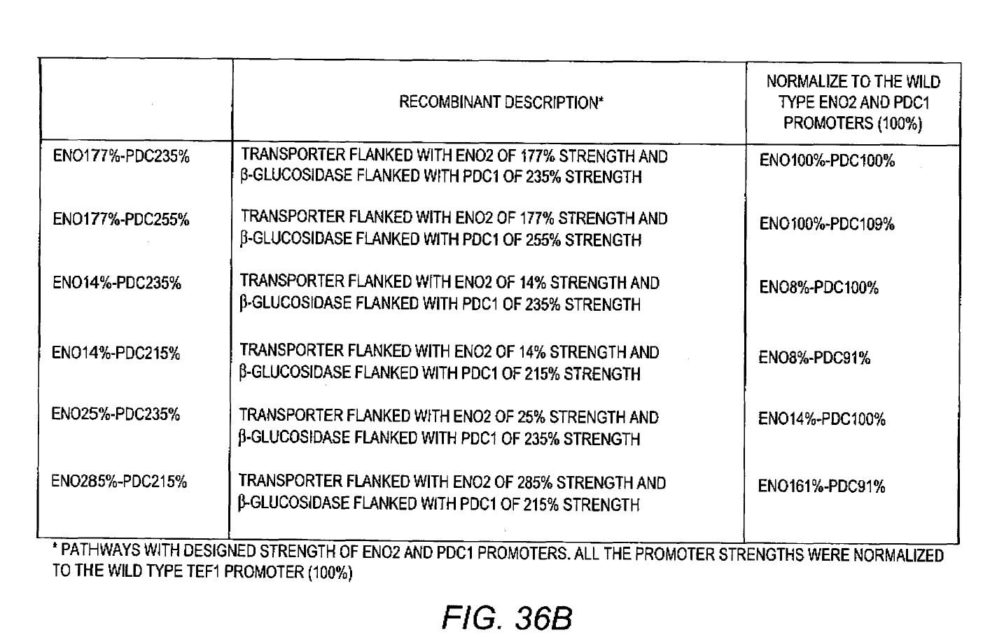

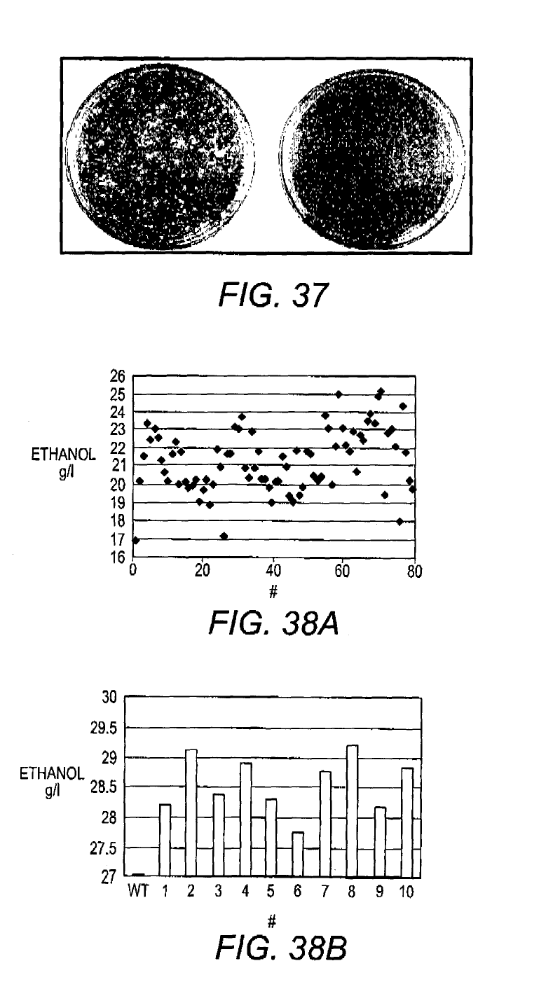

To optimize the cellobiose utilization pathway, the cellobiose transporter gene (cdt-1) and the β-glucosidase gene from Neurospora crassa (gh1-1) were assembled into a single copy expression vector under mutants of PDC1 and ENO2 promoters, respectively. To confirm the library diversity, a number of mutant cellobiose pathways consisting of different combinations of promoter mutants were first constructed and introduced into the Classic Turbo Yeast industrial strain. As expected, the resulted mutants exhibited very different cellobiose fermentation ability due to the different expression levels of the sugar transporter and the β-glucosidase (FIG. 36). A library of cellobiose utilizing pathways derived from combinations of ten ENO2 promoter mutants and eleven PDC 1 promoter mutants were assembled in the laboratory and industrial S. cerevisiae strains, respectively. The strains harboring the pathway library were then screened using a colony-size-based screening method and fast cellobiose utilizing mutant pathways were identified for both laboratory and industrial strains (FIGS. 34C and D; FIG. 37). For the Classic Turbo Yeast industrial strain, the best optimized strain CTY-059 exhibited a 5.4-fold higher cellobiose consumption rate compared to the reference strain harboring the same cellobiose pathway under the control of the wild type promoters (0.39 g/L/h to 2.12 g/L/h) and a 5.3-fold higher ethanol productivity of 0.74 g/L/h. Similarly, for the INVSc1 laboratory strain, the best optimized strain INV-C3 exhibited a 2.1-fold higher cellobiose consumption rate (0.70 g/L/h to 1.50 g/L/h) and a 2.3-fold higher ethanol productivity (0.37 g/L/h) compared to the reference pathway (Table 12-1). After analyzing the promoter mutants present in all optimized strains, it was observed that, all of the five cellobiose utilizing mutant pathways in the INVSc1 strain are identical, consisting of an ENO mutant with an approximately 144% relative strength for the cellobiose transporter and an PDC1 mutant with 235% relative strength for β-glucosidase. Eight of ten cellobiose utilizing mutant pathways in the Classic Turbo Yeast strain contained the same ENO promoter mutant (Table 12-2).

TABLE 12-1
Summary of cellobiose fermentation performance. Two different shake-
flasks, 125 mL and 250 mL, were used in fermentations.

| | | | | | (INVSc1)- | | (INVSc1)- | | |
|---|---|---|---|---|---|---|---|---|---|
| | (Classic)-125 | | (Classic)-250 | | 125 | | 250 | | D452-2 |
| | WT | CYT-C59 | WT | CYT-C59 | WT | INV-C3 | WT | INV-C3 | Ha et al.^{a} |
| Cellobiose | 0.36 | 1.60 | 0.39 | 2.18 | 0.60 | 1.54 | 0.7 | 1.5 | 1.67 |
| consumption rate | | | | | | | | | |
| (g cellobiose/L/hr) | | | | | | | | | |
| Ethanol productivity | 0.14 | 0.65 | 0.14 | 0.74 | 0.16 | 0.51 | 0.16 | 0.37 | 0.7 |
| (g ethanol/L/hr) | | | | | | | | | |
| Yield | 0.42 | 0.44 | 0.37 | 0.39 | 0.32 | 0.37 | 0.23 | 0.27 | 0.42 |
| (g ethanol/g | | | | | | | | | |
| cellobiose) | | | | | | | | | |

^{a}Ha, S. J., Galazka, J. M., Rin Kim, S., Choi, J. H., Yang, X., Seo, J. H., Louise Glass, N., Cate, J. H. and Jin, Y. S. (2010) Engineered Saccharomyces cerevisiae capable of simultaneous cellobiose and xylose fermentation. Proc Natl Acad Sci USA, 108, 504-509 (2011).

TABLE 12-2
DNA sequencing results of the best optimized cellobiose utilizing
strains. Plasmids from top ten Classic stains and top five INVSc1
strains were isolated and sequenced to identify the mutant ENO2
and PDC1 promoters in the cellobiose utilization pathways.

| | | Classic (10)¹ | INVSc1(5)² |
|---|---|---|---|
| ENO2 | ENO-133% | 2 | 5 |
| | ENO-144% | 8 | |
| | PDC-76% | 4 | |
| PDC1 | PDC-137% | 1 | |
| | PDC-235% | 5 | 5 |

Note:
¹Totally 10 colonies were selected in the third round of Classic library screening.
²Totally 5 colonies were selected in the third round of INVSc1 library screening.

| | | Normalize to the wild |
|---|---|---|
| | | type ENO2 and PDC1 |
| | Recombinant description* | promoters (100%) |
| ENO133% | Transporter flanked with | ENO75% |
| | ENO2 of 133% strength | |
| ENO144 | Transporter flanked with | ENO81% |
| | ENO2 of 144% strength | |
| PDC76% | β-glucosidase flanked | PDC32% |
| | with PDC1 of 76% strength | |
| PDC137% | β-glucosidase flanked | PDC58% |
| | with PDC1 of 137% strength | |
| PDC235% | β-glucosidase flanked | PDC100% |
| | with PDC1 of 235% strength | |

*Pathway with designed strength of the ENO2 and PDC1 promoters. All the promoter strengths were normalized to wild type TEF1 promoter (100%)

Strains, Media, and Cell Cultivation

Saccharomyces cerevisiae strain INVSc1 (MATα his3D1 leu2 trp1-289 ura3-52 MATAlpha his3D1 leu2 trp1-289 ura3-52) was purchased from Invitrogen. Still Spirits (Classic) Turbo Distiller's Yeast was purchased from Homebrew Heaven (Everett, Wash.). Escherichia coli DH5α (Cell Media Facility, University of Illinois at Urbana-Champaign, Urbana, Ill.) was used for recombinant DNA manipulation. Yeast strains were cultivated in either synthetic dropout media (0.17% Difco yeast nitrogen base without amino acids and ammonium sulfate, 0.5% ammonium sulfate, 0.083% amino acid drop out mix) or YPA media (1% yeast extract, 2% peptone, 0.01% adenine hemisulfate) supplemented with sugar as carbon source. E. coli strains were cultured in Luria broth (LB) (Fisher Scientific, Pittsburgh, Pa.). S. cerevisiae strains were cultured at 30° C. and 250 rpm for aerobic growth, and 30° C. and 100 rpm for oxygen limited conditions. E. coli strains were cultured at 37° C. and 250 rpm unless specified otherwise. All restriction enzymes were purchased from New England Biolabs (Ipswich, Mass.). All chemicals were purchased from Sigma Aldrich or Fisher Scientific.

Plasmid and Strain Construction

Most of the cloning work was done using the yeast homologous recombination mediated DNA assembler method¹. DNA fragments flanked with regions homologous to adjacent DNA fragments were generated with polymerase chain reaction (PCR) and all the DNA fragments were purified and co-transformed into S. cerevisiae along with the backbone. To confirm the correct clones from transformants, yeast plasmids were isolated using a Zymoprep II yeast plasmid isolation kit (ZYMO Research, Irvine, Calif.) and transferred into E. coli. Plasmids from E. coli were then isolated and confirmed using diagnostic PCR.

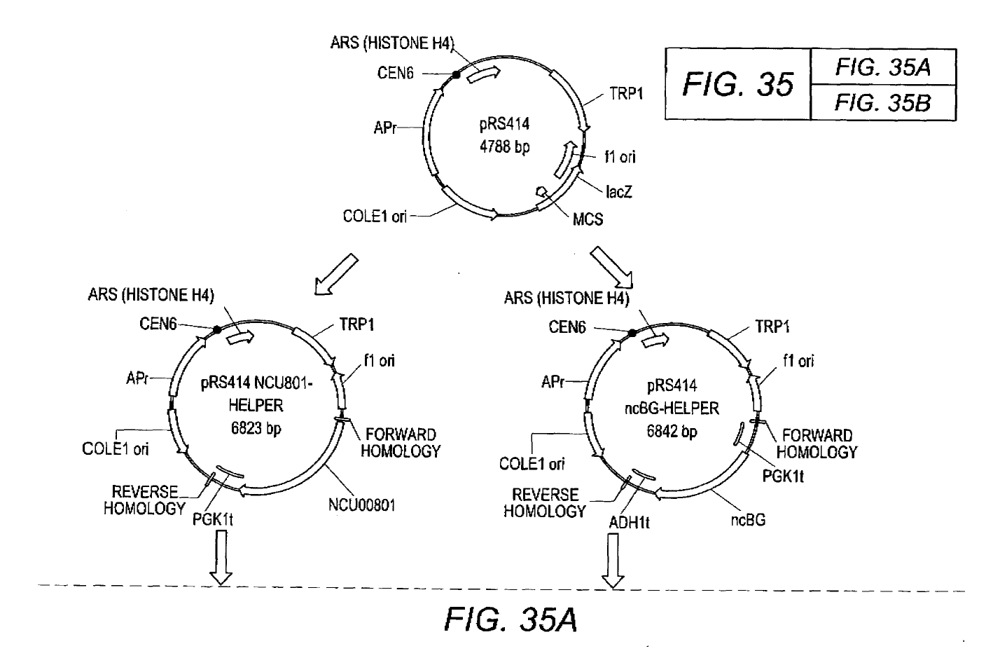

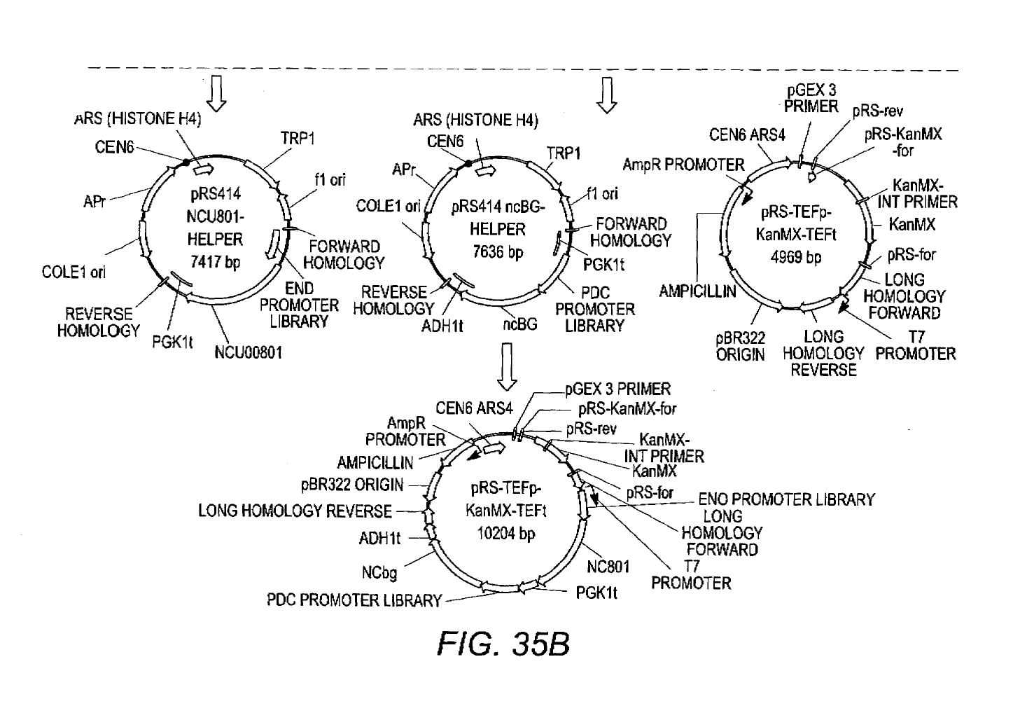

For optimization of cellobiose pathways, the pRS414 plasmid (New England Biolabs, Ipwich, Mass.) was used to create two helper plasmids containing a cellobiose transporter gene and a β-glucosidase gene, respectively. As shown in FIG. 35, the pRS414 plasmid was digested by BamHI and XhoI. Subsequently, the cellobiose transporter gene cdt-1 (GenBank Accession number XM_{—}958708) from Neurospora crassa with the PGK1 terminator at C-terminus, as well as the β-glucosidase gene gh1-1 (GenBank Accession number XM_{—}951090) from N. crassa with the ADH1 terminator at C-terminus, were assembled separately into the digested pRS414 vector using the DNA assembler method and transformed into L2612 strain. Yeast transformants were then cultured in SC medium, and plasmids were extracted using Zymoprep™ Yeast Plasmid MiniprepII (ZYMO Research, Irvine, Calif.) and transformed into E. coli DH5α. E. coli transformants were then cultivated in LB medium to isolate plasmids using the QIAprep Spin Miniprep Kit (QIAGEN, Germantown, Md.). Confirmed plasmids were named as pRS414-NC801-Helper and pRS414-NCbg-Helper, respectively.

11 ENO2 mutants and 10 PDC1 mutants with varying strengths were selected from the whole promoter library for this study (FIG. 20). 11 ENO2 promoter mutants were assembled separately into EcoRI-linearized pRS414-NC801-Helper plasmids in front of the cellobiose transporter gene cdt-1, while 10 PDC 1 mutants were assembled separately into EcoRI-linearized pRS414-NCbg-Helper plasmid in front of the β-glucosidase gene gh1-1. PCR was used to amplify each gene expression cassette consisting of the mutant promoter, the target gene, and the terminator. The resulting 21 DNA fragments were subsequently assembled into the SalI-NotI double digested pRS-kanMX single copy plasmid using the DNA assembler method. The resulting plasmids were transformed into a host of interest.

Fermentation and HPLC Analysis

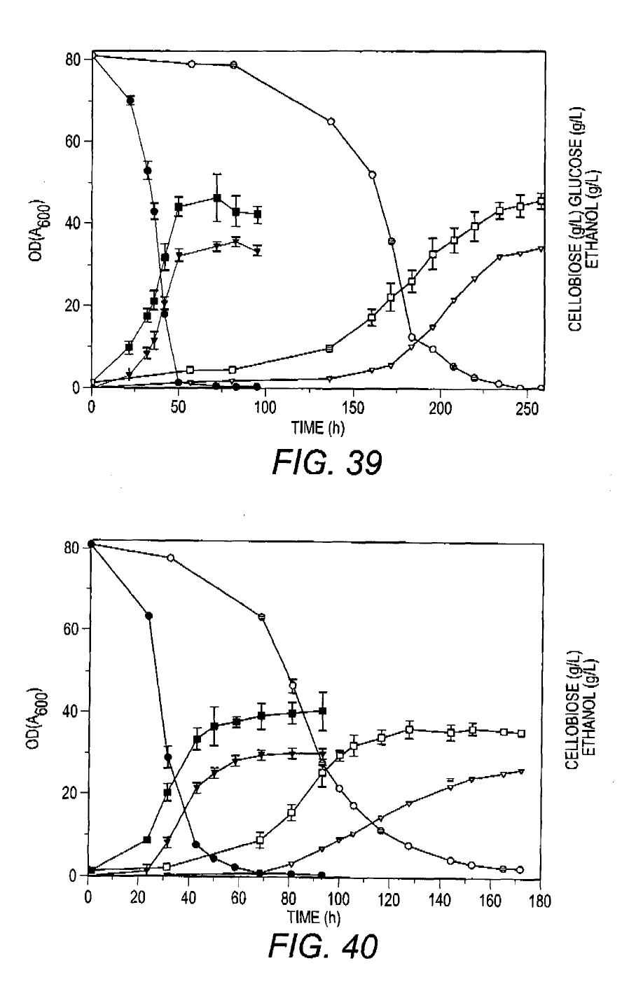

For cellobiose utilization pathways optimization, yeast fermentations were performed in YP medium containing 20 g/L glucose (YPAD) or 80 g/L cellobiose (YPAC). A single colony was inoculated into 3 mL of the YPAD medium and grown up at 30° C. and 250 rpm overnight for seed cells. In the case of no pre-culture fermentation to avoid any adaptation, seed cells were directly transferred into 25 mL of the YPAD medium in a 250 mL baffled shake-flask at 30° C. and 250 rpm to collect enough cells for further fermentation. In the case of pre-culture, seed cells were inoculated into 25 mL of the YPAC medium in a 250 mL shake flask at 30° C. and 250 rpm to obtain enough cells for further main culture. Cells at the middle of exponential phase from YPAD or YPAC medium were harvested and inoculated into 50 mL of the YPAC medium after two times washing using sterilized water. The main cultivation was carried out in a 125 mL or 250 mL unbaffled shake-flask, which is an oxygen limited condition, at 30° C. and 100 rpm with the starting OD of 1 (FIGS. 39 and 40).

Cell densities of the samples were measured using a Cary 300 UV-Visible spectrophotometer (Agilent Technologies, Santa Clara, Calif.) after a proper dilution at a wave length of 600 nm. The samples were then centrifuged and the supernatants were diluted 5 to 10 times before HPLC analysis. An HPLC system equipped with a reflex index detector (Shimadzu Scientific Instruments, Columbia, Md.) was used to analyze the concentrations of cellobiose, glucose, and ethanol in the broth. To separate all the metabolites mentioned above, an HPX-87H column (BioRad, Hercules, Calif.) was used following the manufacturer's manual using 5 mM sulfuric acid as the mobile phase at a flow rate of 0.6 mL/min at 65° C. The HPLC chromogram was analyzed using the LC solution Software (Shimadzu Scientific Instruments, Columbia, Md.).

Library Screening

To screen for fast cellobiose utilizing mutants, all transporter gene cassettes containing 11 ENO2 promoter mutants and β-glucosidase cassettes containing 10 PDC1 promoter mutants were mixed and assembled into SalI-NotI digested pRS-kanMX plasmid and transformed into the host strain (industrial strain Classic or laboratory INVSc1) and spread on YPAD (With 200 mg/L G418) agar plate. All transformants were then diluted to appropriate cell densities and spread on YPAC agar plate. After 30 hours, 80 big colonies were picked up (some colonies were significantly larger than others and than colonies on a reference plate, as shown in FIG. 37) and inoculated into 2 mL YPAD medium in 15 mL tube and shake at 30° C. and 250 rpm. At exponential phase, cells were collected and transferred into 5 mL YPAC medium in 15 mL tube with start OD of 1 and grown under the same condition. Samples were taken two times from late of exponential phase (OD≈50-70), OD and ethanol concentration were measured. Top 10 stains with the highest ethanol concentrations from tube screening were pre-cultured in YPAD and then transferred into 10 mL of YPAC medium in 50 mL shake-flask with start OD of 1 and grown at 30° C. and 100 rpm. Samples were taken two times from late of exponential phase, OD and ethanol concentration were measured (FIG. 38).

DNA Sequencing

After the second round of screening, the top 10 Classic stains and the top five INVSc1 strains were selected for DNA sequencing (Table 12-2). Eight of the ten ENO promoters in the Classic strains had the same sequence, but that sequence did not match any of these 11 pre-selected ENO mutant promoters. The remaining two also had the same sequence. Five of the five ENO promoters in the INVSc1 strains were the same.

Fermentation Studies

The two best strains, including the Classic strain #59 and the INVSc1 strain #3, were cultivated and compared with the wild type strains of Classic and INVSc1 that contained the native ENO and PDC promoters. The fermentation conditions are: 50 mL YPAC medium in 125 mL shake-flask at 30° C. and 100 rpm. 98.5% of 81 g/L cellobiose was consumed by the Classic strain #59, whereas the corresponding wild type Classic strain took 230 hours (FIG. 39).

Compared to the wild type Classic strain, a 4.6-fold of cellobiose consumption rate was observed for the Classic strain #59 (0.352 g/L/h to 1.62 g/L/h). The highest ethanol concentration in the fermentation of the Classic strain #59 was 35.55 g/L, corresponding to an ethanol yield of 0.439 g/g. It is very similar to that in the wild type Classic strain (34.21 g/L), corresponding to an ethanol yield of 0.422 g/g. However, the ethanol productivity of the Classic strain #59 (0.646 g/L/h) was 4.7 fold higher than that of the wild type Classic strain (0.138 g/L/h).

For the INVSc1 strain #3, 95% of 81 g/L cellobiose was consumed in 55 hours whereas the corresponding wild type INVSc1 strain took 115 hours. The highest ethanol concentration of the INVSc1 strain #3 was 31.94 g/L, corresponding to an ethanol productivity of 0.45 g/L/h and an ethanol yield of 0.39 g/L. (FIG. 40).

The above results clearly show that the ethanol productivity was significantly improved both for the Classic and INVSc1 strains through the promoter-based pathway engineering approach.

### Example 13

### Optimized Pathways May be Strain-Specific

During the sequence analysis of optimized xylose or cellobiose utilizing pathways, it was observed that the optimized expression patterns of the pathways consisting of the same set of metabolic genes may differ significantly in different strain backgrounds. To further investigate whether pathways with different expression patterns are optimal for a particular strain background, the best optimized mutant pathways found in the laboratory and industrial strains were exchanged and their distinct fermentation abilities indicated that the optimized pathways were strain-specific (FIGS. 28E and 28F; FIGS. 34E and 34F). Pathway optimization may vary with a particular host cell strain, resulting from different expression levels of endogenous genes involved in the pathway, availability of cofactors, and/or stress responses. It has been frequently observed that the choice of host strains of the same species could affect the behavior of the same heterologous pathway significantly, which poses an obstacle for transferring of well-established metabolic pathways between different host strains (Matsushika et al., Bioresource Technology, 100, 2392-2398 (2009)). Consequently, the ability to tailor-make metabolic pathways rapidly in different strain background is highly desirable in pathway engineering.

The results of the Examples disclosed herein demonstrate that the methods disclosed herein for optimizing metabolic pathways are an efficient approach to tailor-make pathways for biofuel production from lignocellulosic biomass independent of knowledge of the vast previous metabolic engineering studies on xylose utilization. In one round, a recombinant xylose-utilizing industrial strain with 69% of the xylose consumption rate of the fastest xylose utilization strain ever reported was constructed (ranked 4^{th} overall). Similarly, in one round, a recombinant cellobiose utilizing industrial strain with the highest cellobiose consumption rate and ethanol productivity ever reported in literature was constructed. The methods disclosed herein are very efficient for construction of a library of pathways with different expression patterns. Coupled with a proper screening/selection method, the methods disclosed herein can be used for simultaneous optimization of expression levels in various metabolic pathways. The methods disclosed herein can not only be used to balance the metabolic flux through a multiple-step pathway for production of a value-added compound but also generate libraries of metabolic pathways and gene circuits with varying expression patterns for metabolic engineering and synthetic biology.

1-51. (canceled)

52. A host cell comprising a nucleic acid comprising coding regions of a xylose reductase, a xylitol dehydrogenase, and a xylulokinase, wherein each of said coding regions is in operable combination with a heterologous promoter and a heterologous terminator, and wherein each of said coding regions is from a different species.

53. The host cell of claim 52, wherein said xylose reductase coding region is of A. nidulans, said xylitol dehydrogenase coding region is of C. albicans, and said xylulokinase coding region is of S. cerevisiae.

54. The host cell of claim 53, wherein said A. nidulans xylose reductase coding region encodes a polypeptide comprising an amino acid sequence at least 90% identical to SEQ ID NO: 19, said C. albicans xylitol dehydrogenase coding region encodes a polypeptide comprising an amino acid sequence at least 90% identical to SEQ ID NO: 24, and said S. cerevisiae xylulokinase coding region encodes a polypeptide comprising an amino acid sequence at least 90% identical to SEQ ID NO: 49.

55. The host cell of claim 52, wherein said xylose reductase coding region is of P. guilliermondii, said xylitol dehydrogenase coding region is of P. chrysogenum, and said xylulokinase coding region is of A. oryzae.

56. The host cell of claim 55, wherein said P. guilliermondii xylose reductase coding region encodes a polypeptide comprising an amino acid sequence at least 90% identical to SEQ ID NO: 7, said P. chrysogenum xylitol dehydrogenase coding region encodes a polypeptide comprising an amino acid sequence at least 90% identical to SEQ ID NO: 30, and said A. oryzae xylulokinase coding region encodes a polypeptide comprising an amino acid sequence at least 90% identical to SEQ ID NO: 60.

57. The host cell of claim 52, wherein said xylose reductase coding region is of A. nidulans, said xylitol dehydrogenase coding region is of A. niger, and said xylulokinase coding region is of P. chrysogenum.

58. The host cell of claim 57, wherein said A. nidulans xylose reductase coding region encodes a polypeptide comprising an amino acid sequence at least 90% identical to SEQ ID NO: 19, said A. niger xylitol dehydrogenase coding region encodes a polypeptide comprising an amino acid sequence at least 90% identical to SEQ ID NO: 36, and said P. chrysogenum xylulokinase coding region encodes a polypeptide comprising an amino acid sequence at least 90% identical to SEQ ID NO: 47.

59. The host cell of claim 52, wherein said xylose reductase coding region is of C. shehatae, said xylitol dehydrogenase coding region is of C. tropicalis, and said xylulokinase coding region is of P. pastoris.

60. The host cell of claim 59, wherein said C. shehatae xylose reductase coding region encodes a polypeptide comprising an amino acid sequence at least 90% identical to SEQ ID NO: 3, said C. tropicalis xylitol dehydrogenase coding region encodes a polypeptide comprising an amino acid sequence at least 90% identical to SEQ ID NO: 38, and said P. pastoris xylulokinase coding region encodes a polypeptide comprising an amino acid sequence at least 90% identical to SEQ ID NO: 50.

61. The host cell of claim 52, wherein the nucleic acid further comprises coding regions of a xylose-specific transporter, a transaldolase and a transketolase, wherein each of said coding regions is in operable combination with a unique heterologous promoter and a unique heterologous terminator, and wherein said coding regions are from at least two different species.

62. The host cell of claim 52, wherein the nucleic acid further comprises coding regions of an L-arabitol 4-dehydrogenase, and a L-xylulose reductase, wherein each of said coding regions is in operable combination with a unique heterologous promoter and a unique heterologous terminator, and wherein said coding regions are from at least two different species.

63. The host cell of claim 52, wherein the nucleic acid further comprises coding regions of an L-arabitol 4-dehydrogenase, and a L-xylulose reductase, a xylose-specific transporter, an arabinose-specific transporter, a transaldolase and a transketolase wherein each of said coding regions is in operable combination with a unique heterologous promoter and a unique heterologous terminator, and wherein said coding regions are from at least two different species.

64. The host cell of claim 52, wherein said host cell grows anaerobically on xylose and/or arabinose as a main carbon source at a greater rate than a parental yeast strain from which it was derived and which lacks said vector.

65. The host cell of claim 52, wherein said host cell is a microorganism selected from the group consisting of Saccharomyces cerevisiae, Saccharomyces monacensis, Saccharomyces bayanus, Saccharomyces pastorianus, Saccharomyces carlsbergensis, Saccharomyces pombe, Kluyveromyces marxiamus, Kluyveromyces laths, Kluyveromyces fragilis, Pichia stipitis, Sporotrichum thermophile, Candida shehatae, Candida tropicalis, Neurospora crassa, Trichoderma reesei and Zymomonas mobilis.

66. A method for production of ethanol comprising culturing the host cell of claim 52 in a composition comprising xylose and/or arabinose, under conditions suitable for the production of ethanol.

67. The method of claim 66, wherein the composition comprising xylose and/or arabinose comprises plant biomass hydrolysate.

68. The method of claim 66, further comprising recovering the ethanol from the culture medium.
